(19) United States
(12) Patent Application Publication
(10) Pub. No.: US 2004/0009577 A1
(43) Pub. Date: Jan. 15, 2004

(54) Bacterial strain typing

(76) Inventors: Indira Kudva, Revere, MA (US); Stephen B. Calderwood, Wellesley, MA (US); Frederick M. Ausubel, Newton, MA (US)

Correspondence Address: CLARK & ELBING LLP, 101 FEDERAL STREET, BOSTON, MA, 02110, US

(21) Appl. No.: 10/418,837

(22) Filed: Apr. 18, 2003

Related U.S. Application Data
(63) Continuation-in-part of application No. PCT/US01/44963, filed on Nov. 1, 2001.
(60) Provisional application No. 60/244,973, filed on Nov. 1, 2000.

(51) Int. Cl. C12N 1/20

(52) U.S. Cl. 435/252.3

(57) ABSTRACT

The invention features a method for typing the strain of a bacterial isolate, the method including the steps of: (a) providing genomic DNA from a bacterial isolate; (b) performing a polymerase chain reaction on the genomic DNA using a first and second primer to amplify genomic DNA comprising a restriction nuclease restriction site, thereby producing an amplicon having the restriction site; and (c) characterizing the amplicon of step (b), thereby typing the strain of the bacterial isolate. The invention also features a kit for distinguishing between bacterial strains comprising a set of primer pairs which, when used in a PCR reaction of genomic DNA from a sample of a bacterial isolate amplify DNA across a site for a restriction endonuclease, the amplified DNA being polymorphic between strains of the bacteria.

### CROSS REFERENCE TO RELATED APPLICATIONS

This application is a continuation-in-part of International Application No. PCT/US01/44963, filed Nov. 1, 2001, published in English under PCT article 21(2), currently pending, which claims benefit of U.S. provisional application No. 60/244,973, filed Nov. 1, 2000, each of which are hereby incorporated by reference.

### BACKGROUND OF THE INVENTION

The invention relates to bacterial strain typing.

In higher plants and animals, the identification of strains or varieties within a species is a relatively straight forward proposition, since the phenotypic characteristics of the organisms can be examined. However, it is often difficult to appropriately compare different isolates or strains of bacterial species, since their morphological characteristics may often be similar or the difference may only be evident in response to specific environmental conditions. Nevertheless, knowledge about the origin, relatedness, and evolution of bacterial species is an important area of inquiry, both for epidemiological purposes as well as for the understanding of the evolution and population dynamics of bacterial cultures. This concern becomes of particular importance when the genetics of human pathogens is considered.

For example, many outbreaks of infection, particularly those that are food-borne, now affect patients nearly simultaneously in several different states or even different countries. Rapid detection of these widespread outbreaks may limit spread of disease by allowing identification and withdrawal of the common source of infection from the marketplace. Development of a rapid, reproducible, and easily comparable strain typing system for closely related bacterial strains such as enterohemorrhagic E. coli O157:H7 has been a particular challenge. This serotype of E. coli emerged as a highly virulent pathogen in the early 1980s and has subsequently caused several major outbreaks in the United States, Europe, and Japan, as well as a large number of sporadic infections (Kaper and O'Brien, Escherichia coli O157:H7 and other Shiga Toxin-Producing E. coli Strains Washington, D.C., ASM Press (1998); Griffin et al., Ann. Intern. Med. 109, 705-712 (1988)). Clinical disease in humans manifests most commonly as bloody diarrhea (hemorrhagic colitis), which can progress to the hemolytic-uremic syndrome or thrombotic thrombocytopenic purpura (Griffin and Tauxe, Epidemiol. Rev. 13,60-98 (1991)).

Comparison of two or more isolates of a given bacterial species to determine if they are the same or different is a key step in many epidemiologic, phylogenetic and population studies. Delineation of isolates of specific human pathogens into distinct related strains, for example, allows epidemiologists to define outbreaks and to trace the spread of a particular strain in a population (Arbeit, Manual of Clinical Microbiology, ASM Press, pp. 190-208 (1995); Musser, Emerg. Infect. Dis. 2, 1-17 (1996)). Strains of a particular bacterial species may diverge from each other by acquisition or loss of mobile genetic elements, by point mutation, or by other genetic events such as insertions, deletions, or inversions (Arbeit, Manual of Clinical Microbiology, ASM Press, pp. 190-208 (1995)). Some bacterial species, such as Helicobacter pylori, are comprised of highly divergent strains that have undergone substantial genetic drift, and even conserved genes in such strains may differ by numerous point mutations (Salau et al., FEMS Microbiol. Lett. 161, 231-239 (1998)). On the other hand, other bacteria such as the O157:H7 serotype of Escherichia coli, are highly clonal, with individual strains containing fewer genetic differences (Wang et al., Nucleic Acids Res. 21, 5930-5933 (1993); Whittam et al., J. Infect. Dis. 157, 1124-1133 (1988)) (Wang et al., Nucleic Acids Res. 21: 5930-5933 (1993); Whittam, Emerg. Infect. Dis. 4: 615-617. (1998)). A number of approaches, both phenotypic and genotypic, have been used to examine the relatedness of different isolates of a given bacterial species or serotype, both for epidemiologic purposes as well as to gather insights into the mechanisms of microbial evolution (Musser, Emerg. Infect. Dis. 2, 1-17 (1996); Hill et al., Clin. Microbiol. Newslett. 17, 137-142 (1995)). However, most of these systems for strain typing are limited because of lack of typeability, reproducibility, discriminatory power, ease of interpretation, or ease of performance (Arbeit, Manual of Clinical Microbiology, ASM Press, pp. 190-208 (1995)).

Examples of phenotypic methods for strain typing include biotyping (carbohydrate fermentation and antimicrobial susceptibility pattern), serotyping, whole cell fatty acid profiling, phage typing, bacteriocin typing, and multilocus enzyme electrophoresis (MLEt) (Arbeit, Manual of Clinical Microbiology, ASM Press, pp. 190-208 (1995); Steele et al., Appl. Environ. Microbiol. 63, 757-760 (1997)). Of these, MLEE, based on variations in electrophoretic mobilities of enzymes encoded by housekeeping genes, is the most discriminating and has been used to study the population genetics of different bacterial species with reproducible results (Selander et al., Appl. Environ. Microbiol 51, 873-884 (1986); Wang et al., Nucleic Acids Res. 21, 5930-5933 (1993); Pupo et al., Infect. Immun. 65: 2685-2692 (1997)). MLEE, however, is a labor intensive and expensive procedure, and may fail to distinguish alleles encoding different enzymes with the same mobility. In addition, MLEE is time-consuming, limiting its applicability in disease outbreaks, where rapidity may help limit spread of the disease (Arbeit, Manual of Clinical Microbiology, ASM Press, 190-208 (1995)). The other phenotypic methods often suffer from poor discriminative power and/or failure to type all strains (Arbeit, Manual of Clinical Microbiology, ASM Press, pp. 190-208 (1995)).

Genotypic methods for strain typing have been used increasingly in recent years. Some of the earlier methods used included restriction enzyme analysis of plasmid and chromosomal DNA (Arbeit, Manual of Clinical Microbiology, ASM Press, pp. 190-208 (1995)), but spontaneous loss of plasmids and overlapping DNA bands led to confounding patterns, causing these procedures to be replaced with more refined molecular techniques based on Southern blot hybridization and the polymerase chain reaction (PCR) (Arbeit, Manual of Clinical Microbiology, ASM Press, pp. 190-208 (1995); Hill et al., Clin. Microbiol. Newslett. 17, 137-142 (1995); Olive and Bean J. Clin. Microbiol. 37, 1661-1669 (1999)). Southern blot hybridization can be used to detect restriction fragment length polymorphisms (RFLP) for specific genes, and includes procedures such as ribotyping, insertion sequence (IS) typing, and virulence gene profiling (Arbeit, Manual of Clinical Microbiology, ASM Press, pp. 190-208 (1995); Olive and Bean J. Clin. Microbiol. 37, 1661-1669 (1999); Mead and Griffin, Lancet 352, 1207-1212 (1998); Thompson et al., J. Clin. Microbiol. 36, 1180-1184(1998)). Similarly, PCR-based techniques, such as restriction enzyme analysis of PCR products, PCR-based-locus-specific RFLP, repetitive extragenic palindromic element PCR (Rep-PCR), random amplified polymorphic DNA assay (RAPD), and amplified fragment length polymorphism (AFLP) have all been used for strain typing (Savelkoul et al., J. Clin. Microbiol. 37, 3083-3091 (1999); Wang et al., Nucleic Acids Res. 21, 5930-5933 (1993); Johnson and O'Bryan Clin. Diagn. Lab. Immunol. 7: 265-273 (2000); Olive and Bean, J. Clin. Microbiol. 37, 1661-1669 (1999); Arbeit, Manual of Clinical Microbiology, ASM Press, pp. 190-208 (1995); Mead and Griffin, Lancet 352, 1207-1212 (1998)). Nucleotide sequence analysis and multilocus sequence typing (MLST) are newer approaches, coupled to the rise in genomic sequencing (Olive and Bean, J. Clin. Microbiol. 37, 1661-1669 (1999); Feil et al., Mol. Biol. Evol. 16, 1496-1502 (1999); Maiden et al., Proc. Natl. Acad. Sci. U.S.A 95, 3140-3145 (1998)).

Currently, the molecular technique considered to be the most reliable and applicable system for strain typing of several bacterial species is pulsed-field gel electrophoresis (PFGE) (Tenover et al., J. Clin. Microbiol. 33, 2233-2239 (1995); Olive and Bean, J. Clin. Microbiol. 37, 1661-1669 (1999)). In this procedure, genomic DNA is digested with a rare cutting restriction endonuclease and PFGE is used to separate the resulting high molecular size fragments. The distinctive profiles generated enable differentiation of strains in a reproducible manner. Not all strains, however, are typeable by PFGE. The inability to type certain strains has been ascribed to methylation of restriction sites, degradation of DNA in agarose plugs, or other technical problems (Johnson et al., Appl. Environ. Microbiol. 61, 2806-2808 (1995); Murase et al., Curr. Microbiol. 38: 48-50 (1999); Harsono et al., Appl. Environ. Microbiol. 59, 3141-3144 (1993)). While all these molecular techniques may provide precise data, they are either expensive or time consuming to perform, lack sufficient discriminatory power, or require specialized equipment. Application of MLST and nucleotide sequence analysis techniques to strain typing depends on accurate identification of polymorphic sites in the genome for comparison (Olive and Bean, J. Clin. Microbiol. 37, 1661-1669 (1999); Feil et al., Mol. Biol. Evol. 16, 1496-1502 (1999)). The most important drawback of PFGE is that the comparison of results for isolates analyzed at different locations or times (and hence on different gels) requires sophisticated pattern recognition computer software (Olive and Bean, J. Clin. Microbiol. 37, 1661-1669 (1999)). As mentioned above, however, PFGE has certain limitations as a strain typing system, including time needed for analysis and the difficulty in comparing patterns of resolved bands between isolates analyzed on different gels. PFGE has also not given any specific insights into the mechanisms by which strains of E. coli O157:H7 differ from each other or evolve over time.

Although several tools are available for strain typing of bacterial isolates most of these are limited by either lack of typeability, reproducibility, discriminatory power, ease of interpretation, ease of performance, or cost effectiveness, which are the criteria for evaluating typing systems. Accordingly, a need exists in the art for the development of new approaches to bacterial strain typing.

### SUMMARY OF THE INVENTION

In general, the invention features a method for typing the strain of a bacterial isolate. The method includes the steps of: (a) providing genomic DNA from a bacterial isolate; (b) performing a polymerase chain reaction on the genomic DNA using a first and second primer to amplify genomic DNA including a restriction nuclease restriction site, thereby producing an amplicon having the restriction site; and (c) characterizing the amplicon of step (b), thereby typing the strain of the bacterial isolate. In preferred embodiments, the method of the invention further includes performing a polymerase chain reaction on genomic DNA of a reference strain of a bacterial isolate using the first and second primers of step (b) to amplify genomic DNA of the reference strain of the bacterial isolate, and wherein step (c) is carried out by characterizing the amplicon of the reference strain of the bacterial isolate with the amplicon of step (b). In preferred embodiments, the reference strain of the bacterial isolate is E. coli O157:H7 strain 86-24. In other preferred embodiments, the method of the invention further includes digesting the amplicon of step (b) with a restriction nuclease that digests the amplicon at the restriction site and where step (c) is carried out by charactering the digestion products.

In yet other preferred embodiments, the method of the invention further includes performing a polymerase chain reaction on genomic DNA of a reference strain of a bacterial isolate using the first and second primers of step (b) to amplify genomic DNA of the reference strain of the bacterial isolate and digesting the amplicon of the reference strain with the restriction nuclease, and where step (c) is carried out by characterizing the digestion products of the cleaved amplicon. One preferred reference bacterial strain used in the method is E. coli O157:H7 strain 86-24.

In yet other preferred embodiments, the typing method involves selecting a restriction site that occurs infrequently in the genome of the bacterial isolate. The method also involves the use of a restriction nuclease such as XbaI or AvrII that cleaves rarely within the genome of the bacterial isolate. In still other preferred embodiments, the method involves generating an amplicon of step (b) that includes a PCR fragment having at least 200-400 bp. In other preferred embodiments, the method involves the use of a pathogenic bacterial strain (for example, E. coli O157:H7).

In still other preferred embodiments, the typing methods involve determining whether an amplicon is present in the bacterial isolate that is not present in the reference strain; an amplicon is absent in the bacterial isolate that is present in the reference strain; or there is an alteration in the size of the amplicon between the bacterial isolate and the reference strain. In other embodiments of the typing method, the digestion identifies a single nucleotide polymorphism (e.g., identifies an additional site of restriction nuclease cleavage in the amplicon). In still other embodiments, the amplicon is digested with at least two restriction nucleases (e.g., XbaI and AvrII).

In another aspect, the invention features a method for identifying a pair of primers for typing a bacterial strain, the method involves the steps of: (a) providing genomic DNA of a bacterial strain; (b) fragmenting the genomic DNA of the bacterial strain into at least two fragments, where the fragments include a restriction enzyme site flanked by 5′ and 3′ regions of DNA; (c) identifying a first primer that hybridizes to the 5′ region flanking the restriction site and a second primer that hybridizes to the 3′ region flanking the restriction site, where the first and second primers amplify genomic DNA of the bacterial strain having the restriction site; (d) performing a polymerase chain reaction (PCR) on the genomic DNA of the bacterial strain using the first and second primers of step (c) to amplify genomic DNA of the bacterial strain, thereby producing an amplicon; (e) providing a second genomic DNA, the second genomic DNA being from a reference bacterial strain; (f) performing a polymerase chain reaction (PCR) on the reference genomic DNA using the first and second primers of step (c) to amplify genomic DNA of the reference bacterial strain, thereby producing an amplicon; (i) comparing the amplicons of step (d) and step (f), where a difference between the amplicons of steps (d) and (f) identifies the pair of primers as a pair of primers for typing the bacterial strain.

In preferred embodiments, the method further includes digesting the amplicons of step (d) and step (f) with a restriction nuclease that cleaves the amplicons at the restriction site, and further comparing the digested amplicons of step (d) and (f), wherein a difference between the products of the digested amplicons of steps (d) and (f) further identifies the pair of primers for typing the bacterial strain. Exemplary restriction sites useful in the method are those that occur infrequently in the genome of the bacterial strain. Similarly, a restriction nuclease useful in the method includes enzymes that cleave rarely within the genome of the bacterial strain, for example, XhaI or AvrII.

In other preferred embodiments, the difference between the bacterial strain and the reference strain is the presence of an amplicon in the bacterial strain that is not present in the reference strain; is the absence of an amplicon present in the reference strain; or is a difference in the size of the amplicons. In another embodiment, the digestion identifies a single nucleotide polymorphism (e.g., an additional site of restriction endonuclease cleavage in the amplicon). In another embodiment, the restriction nuclease is XbaI or AvrII. In another embodiment, the amplicon is digested with at least two restriction nucleases (e.g., XbaI and AvrII).

In preferred embodiments, the bacterial typing method involves a polymerase chain reaction that amplifies an amplicon of step (c) that includes at least 200-400 bp. The method is especially useful for analyzing pathogen bacterial strains such as E. coli O157:H7. In other preferred embodiments, the reference bacterial strain of step (e) is E. coli O157:H7 strain 86-24.

In other aspects, the invention features a kit for distinguishing between bacterial strains. The kit of the invention includes a set of primer pairs which, when used in a PCR reaction of genomic DNA from a sample of the bacteria amplify DNA across a restriction site for a restriction nuclease, the amplified DNA being polymorphic between strains of the bacteria. In preferred embodiments, the primers are prepared according to the methods disclosed herein.

In yet another aspect, the invention includes a bacterial strain typing profile, the typing profile produced according to any one of the methods described herein. In preferred embodiments, the typing profile is depicted on an agarose gel or a dot blot or microarray.

In yet another aspect, the invention features a microarray comprising at least two amplicons of a pathogenic bacterial strain. In one embodiment, the microarray contains a collection of amplicons (e.g., five, ten, twenty, thirty, forty, fifty, sixty, seventy, eighty, ninety, one hundred, two hundred, three hundred, four hundred, five hundred, or one thousand amplicons, or fragments thereof). In another embodiment, the amplicons, or fragments thereof, are produced as described in any of the above aspects. In one preferred embodiment, the pathogenic bacterial strains are strains of E. coli O157:H7.

In a related aspect, the invention features a method for typing a strain of a bacterial isolate, the method involves the steps of: (a) providing genomic DNA fragments from a bacterial isolate; (b) detectably labeling the fragments; (c) contacting the microarray described in the previous aspect with the detectably labeled fragments; and (d) determining the binding pattern of the fragments to the microarray; thereby typing the strain of the bacterial isolate. In one embodiment, the bacterial strain is a strain of E. coli O157:H7. In another embodiment, the isolate is from a patient, a food source, soil, or a water source.

In a related aspect, the invention features a method of making a microarray, the method involves the steps of: (a) providing genomic DNA from at least one bacterial strain; (b) performing a polymerase chain reaction (PCR) on the genomic DNA of the bacterial strain using a first and second primer to amplify genomic DNA of the bacterial strain, thereby producing an amplicon; and (c) affixing the amplicon to a solid support. In one embodiment, the amplicon is a polymorphic nucleic acid molecule, or a fragment thereof. In another embodiment, the bacterial strain is E. coli O157:H7.

In another aspect, the invention features a method for typing a strain of a bacterial isolate, the method involving the steps of: (a) providing genomic DNA from a bacterial isolate; (b) performing a polymerase chain reaction on the genomic DNA using a first and second primer to amplify genomic DNA containing a restriction nuclease restriction site; and (c) assaying for the presence or absence of the amplicon of step (b), thereby typing the strain of the bacterial isolate.

The methods disclosed herein provide a straightforward means for strain typing bacteria and provide numerous advantages over current typing systems. For example, the methods of the invention provide a route for analyzing any number of bacterial isolates recovered from virtually any source, including clinical samples and food. The strain typing methods described herein are relatively simple and inexpensive to perform. Moreover, the methods can be performed in any laboratory with a thermocycler and other common laboratory materials. In addition, the methods can be performed the very day an isolate is recovered from a sample. Interpretation of typing results is also relatively straightforward as strains are typed on a characteristic profile determined by the presence or absence of amplicons. Strain typing results obtained using the disclosed methods are typically available in a few hours and are highly reproducible.

By “microarray” is meant an organized collection of at least two nucleic acid molecules affixed to a solid support. Microarrays include, for example, 2, 5, 10, 25, 50, 75, 100, 250, or 500 nucleic acid molecules.

By “collection” is meant a group having more than one member. A group may be composed of 2, 5, 10, 25, 50, 75, 100, 250, or 500 amplicons.

By “amplicon” is meant a polymorphic nucleic acid molecule, or fragment thereof, produced via a nucleic acid amplification step, such as a polymerase chain reaction.

By “polymorphic nucleic acid molecule” is meant a nucleic acid molecule, or fragment thereof, that is present in one bacterial strain, but that is not present in a reference strain, for example, a reference strain, such as E. coli O157: H7 strain 933, or 86-24.

By “fragment” is meant a portion of a nucleic acid molecule (e.g., an amplicon). In some embodiments, the portion is 10, 15, 18, 20, 21, 22, 23, 24, 25, 26, 27, 28, 29, 30, 40, 50, 60, 70, 80, 90, 100, 250, 500, 750, or 1000 nucleotides.

By “typing profile” is meant a reliable representation of polymorphic traits that identifies a bacterial strain. For example, a microarray or dot blot having a characteristic bacterial hybridization pattern or an agarose gel having a distinctive banding pattern that identifies a bacterial strain.

Other features and advantages of the invention will be apparent from the following description of the preferred embodiments thereof, and from the claims.

### BRIEF DESCRIPTION OF THE DRAWINGS

FIG. 1A shows a comparison of pO157 DNA from E. coli O157:H7 strain 933, representative isolates G5303 and G5323 and strain 86-24. Identical regions are shown in black and the inserts that differed between the strains, in white. The insertions in isolates G5303 and G5323 are identical, but differed from that in strain 86-24. The insertion in strain 86-24 contained an XbaI site. Fragment IK8 (in gray), amplified by primer pair IK8A/B, mapped to a region of unknown function within pO157 DNA from strain 86-24. This region occurs as a 635 bp insertion, relative to this region in strain 933. The sequence at the point of insertion is indicated and is identical in all strains shown.

FIG. 1B shows the original primers (shown in bold) and additional primers used for further analysis of the polymorphisms between strains. Primers are in direct alignment with the regions in pO157 DNA from strain 86-24 used to design them.

FIG. 1C shows the agarose gel electrophoresis pattern of amplicons derived using the primer pairs described in FIG. 1B. The pattern generated depicts the polymorphism between strains 86-24 and 933 diagrammed in FIG. 1A. “M” refers to molecular size marker (100 bp DNA ladder; NEB) and “+” or “−” respectively designates the presence or absence of an amplicon.

FIG. 2A shows a diagrammatic representation of XbaI-restriction site-polymorphisms identified in E. coli O157 strains that are attributable to a substitution-insertion in a lysogenic bacteriophage. Lysogenic phage DNA from E. coli O157:H7 strain 86-24 and strain 933 were compared. Identical regions are shown in black and regions that differed between the two strains in white. Strain 933 contains a 2,091 bp substitution-insertion containing an XbaI restriction site, between the N and cI genes, in place of a 1,439 bp fragment without an XbaI site in strain 86-24. Fragment IKB3 (in gray), amplified by the primer pair IKB3A/B, mapped to the substituted region within phage 933W from strain 933. Sequence flanking the substitution-insertion is identical between the two strains. Original primers (shown in bold) and additional primers used for further analysis of this polymorphism between strains are depicted. Primers are in direct alignment with the regions in phage 933W used to design them.

FIG. 2B shows a diagrammatic representation of XbaI-restriction site-polymorphisms identified in E. coli O157 strains that are attributable to a chromosomal deletion-substitution. Chromosomal DNA segments from E. coli O157:H7 isolates G5295 and G5296 and strain 933 were compared. Identical regions are shown in black and regions that differed between the strains in white. Fragment IK118 (in gray), amplified by primer pair IK118A/B, mapped to a chromosomal region at an O-island-backbone junction in strain 933, and contained an XbaI restriction site in the O-island sequence. Isolates G5295 and G5296 have a deletion-substitution in this region, substituting a different segment of DNA at the same location in place of the sequence containing an XbaI restriction site in strain 933. Original primers (shown in bold) and additional primers used for further analysis of this polymorphism between strains are depicted. Primers are in direct alignment with the regions in the DNA from strain 933 used to design them.

FIG. 3 is a schematic representation showing a protocol for the design of Polymorphic Amplified Typing Sequences (PATS) primer pairs. Genomic DNA fragments derived from E. coli O157: H7 strains 86-24 and 933, containing an XbaI restriction site, were selectively cloned into pBluescribe. DNA was initially fragmented using Sau3AI (strain 86-24) or NlaIII (strain 933) restriction enzymes and self-ligated. The circularized DNA was then digested with the restriction enzyme XbaI to linearize only fragments containing an internal XbaI site. Cloning of these fragments resulted in plasmids of varying sizes that were prefixed pIK. Insert sequences were determined and used to design PATS primer pairs, shown as divergent block arrows, which flank XbaI restriction sites in the bacterial genome. “MCS” refers to the multiple cloning site.

FIG. 4 shows a representative agarose gel electrophoresis pattern of amplicons generated from E. coli O157:H7 isolates using PATS and virulence gene primer pairs. Presence or absence of amplicons was isolate specific. Lanes 1-12 show the PCR results of six isolates, obtained using PATS primer pair IK127A/B; the odd number lanes are before XbaI digestion and the even lanes, after digestion. Amplicons, when present, always digested with restriction enzyme XbaI into two fragments. Lanes 14-17 show the PCR results of a single isolate (G5299), obtained using virulence gene primer pairs, stx₁F/R, stx₂F/R, eaeF/R, and hlyAF/R. These amplicons lacked an XbaI restriction site and were not digested with this enzyme (not shown). “M” refers to molecular size marker (100 bp DNA ladder; NEB).

FIGS. 5A and 5B show a phylogenetic analysis of E. coli O157:H7 isolates using PATS and PFGE data. Dendrograms were constructed using the unweighted pair-group method with arithmetic mean (UPGMA). PFGE gels were analyzed using Molecular Analyst Fingerprinting Plus software (Bio-Rad) and the data was exported as a band matching table so that the two sets of data could be analyzed by the same method. FIG. 5A shows a PATS dendrogram. PATS profiles resolved the isolates into four major clusters. A genetic distance of <0.1 between each PATS cluster suggests a clonal lineage for these isolates. The genetic distance is indicated in increments of 0.01 below the dendrogram. FIG. 5B shows a PFGE dendrogram. PFGE profiles resolved the isolates into smaller clusters and showed greater genetic distance between the isolates.

FIG. 6 shows the PFGE patterns of the 44 E. coli O157:H7 isolates from 22 outbreaks. Isolate numbers are indicated above the gel. Note that isolates G5312, G5311, G5306, G5305, G5290, and G5289 could not be typed by PFGE (and are grouped together at the bottom of FIG. 5B). The lambda DNA ladder standard for PFGE applications (Bio-Rad) was used. Molecular size in kilobase (Kb) is shown to the right.

FIG. 7 shows multiplex PCR and DNA dot-blot assays to detect PATS polymorphisms between strains. Target-amplicons were derived from E. coli O157:H7 control strains 86-24 and 933, using each of the eight indicated PATS primer pairs individually. Probe-amplicons were obtained from each of a total of ten isolates, using seven of the eight XbaI PATS primer pairs in a multiplex PCR reaction and a separate PCR reaction with primer pair IKB5A/B. These probe-amplicons were hybridized to nylon membrane strips containing 2.5 μl of each purified target-amplicon. The hybridization patterns seen on the dot blot autoradiographs matched the corresponding PATS profiles determined above.

FIG. 8 shows the DNA sequence (SEQ ID NO: 1) of the O-islands residing within the genomic sequence of E. coli O1575:H7 that are not found in the sequence of the non-pathogenic E. coli strain K12.

FIGS. 9A-9C show phylogenetic analyses of E. coli O157:H7 isolates using PATS data. Dendrograms were constructed using the unweighted pair-group method with arithmetic mean (UPGMA). FIG. 9A shows a dendrogram that was constructed using PATS data from the XbaI primers. FIG. 9B shows a dendrogram that was constructed using PATS data from the AvrII primers. FIG. 9C shows a dendrogram that was constructed by combining PATS data from the XbaI, AvrII, and virulence gene primers. This approach divided the isolates into smaller clusters showing an increase in the discriminatory ability of PATS.

### DETAILED DESCRIPTION OF THE INVENTION

The present invention is directed toward a method to efficiently and accurately type strains of bacteria, particularly pathogenic bacteria. The methodology is based on the discovery that strains of Escherichia coli O157:H7 differ from each other primarily by insertions or deletions of nucleic acid sequences and the identification of genomic DNA sequences around each site for a restriction endonuclease which cuts rarely (perhaps 10 to 100 times) within the genome of an organism. PCR amplification of DNA containing the restriction cleavage site is used to determine the presence, absence, or mutation of the restriction site. Such changes are indicative of genetic variation, and a molecular subtyping method can be based upon the detection of such genetic variation.

At least two approaches are contemplated for deriving the information for such a strain typing method. Both methods are intended to define genomic sequence information centering on the cleavage site for the restriction endonuclease.

In the first approach, small DNA fragments (optimally 200-300 base pairs), each containing the restriction cleavage site, are cloned, using a method involving two different restriction endonucleases. The fragments are created by digesting the whole genomic DNA of the organism with a restriction endonuclease that cuts the genome many times. The small fragments are then allowed to re-circularize by self-ligation. Then the small fragments are digested using a rare restriction endonuclease, which cuts and linearizes only the fragments containing the cleavage site for that endonuclease. The linearized fragments are then sequenced to determine the sequence of the DNA flanking the cleavage site.

The second approach is available for those organisms for which the whole genomic sequence is available. In that event, a computer search algorithm can be used to identify all sequences containing the cleavage site as well as the flanking sequences.

By whichever approach is used, once the cleavage site and the flanking sequence is known, PCR primers are designed to amplify two to four hundred base pair inserts which would cross over the location of the restriction endonuclease cleavage site. Such PCR primers can be used on genomic DNA of samples of the organism to amplify the DNA of the organism extending across the cleavage site. Then, if desired, a simple analysis of the products of digestion of the PCR products with the rare restriction endonuclease permits strain typing of the organism. Alternatively, the presence or absence of a PCR product (i.e., an amplicon) is monitored.

In the first example of the method described herein, forty XbaI restriction endonuclease sites were identified in strains of Escherichia coli O157:H7, and forty pairs of primers were designed to amplify genomic sequences stretching across those sites. A panel of strains of the bacterial species was then collected. Genomic DNA from the panel of 44 samples of E. coli O157:H7 was isolated, and the primers were used to amplify PCR products containing each of the forty sites for each of the strains in the panel. A comparison was then done to determine which primer pairs were diagnostic of variations between the strains. As it turned out, eight pairs of primers were polymorphic between the strains and could be used to distinguish strains in the collection from each other. This exercise demonstrated that it is possible to design a relatively convenient and accurate method of strain typing of bacterial pathogens based on this technique.

In the next example of the method described herein, primers flanking A vrII sites were designed using the genome sequence of E. coli O157:H7. The primer pairs were used to amplify DNA flanking thirty-three sites in the O157 genome.

Seven pairs of primers were polymorphic between strains of E. coli O157:H7, and could be used to distinguish the strains. When these seven polymorphic AvrI primer pairs were used in combination with the eight polymorphic XbaI primer pairs and the primer pairs amplifying four virulence genes (stx₁, stx₂, eae, hlyA), the PATS typing system distinguished between many more of the bacterial strains than either the XbaI primers or AvrII primers used individually.

The techniques described above were used specifically to identify a method for typing strains of E. coli O157:H7, a human pathogenic bacteria. As is described in the Examples found below, the rare base cutters XbaI and AvrII were utilized to design a strain typing method for E. coli O157:H7.

In the initial design of the strain typing method for E. coli O157:H7, a collection of 44 strains were collected to be used to test the primer pairs designed to amplify across the XbaI sites. The forty primer pairs were used to create PCR reactions with DNA from each of the members of the panel. The presence or absence of the PCR products (i.e., amplicons) was then monitored. It was determined that eight pairs of the primers produced polymorphic results between the strains of O157:H7 in the collection. As is discussed below, those primers permitted identification and typing of the various strains of E. coli O157:H7, both for epidemiological purposes and for the study of the genetic evolution of the pathogen. The sequences of the eight pairs of primers demonstrated here to be useful for differentiating between strains of E. coli O157:H7 are shown in Table 1A. These eight primer pairs are located on larger segments of genomic DNA which are present or absent in different stains of E. coli O157:H7. It is contemplated that any primer pairs with these larger genomic regions will work equally well to distinguish amongst the strains.

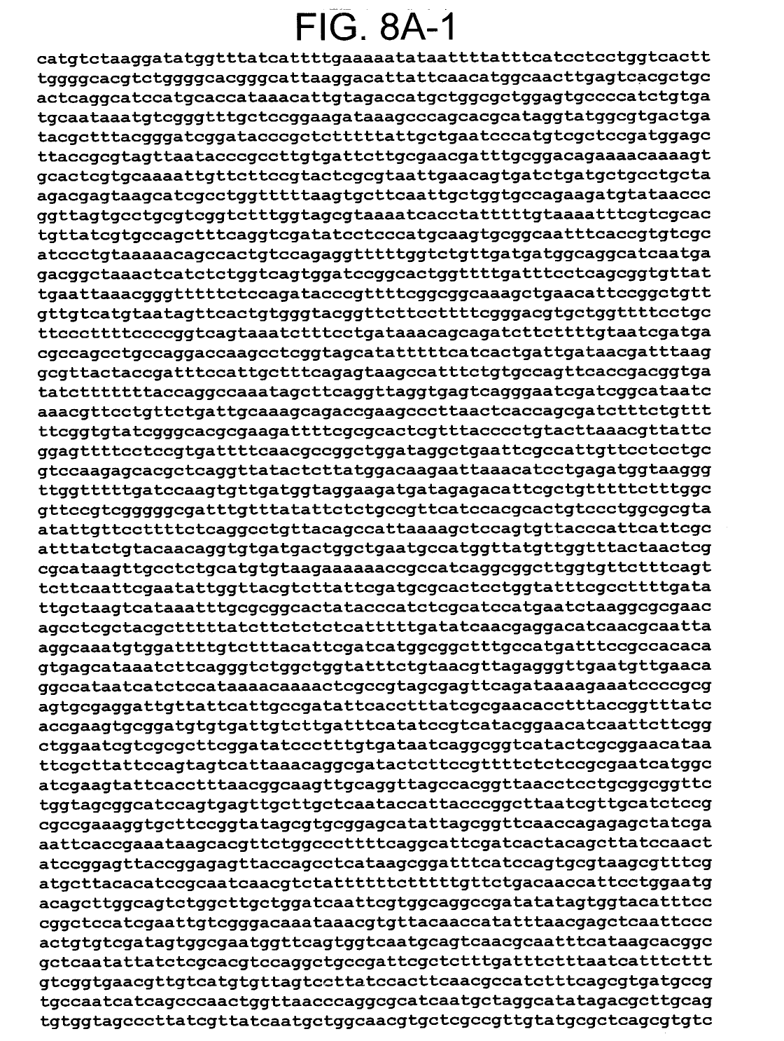

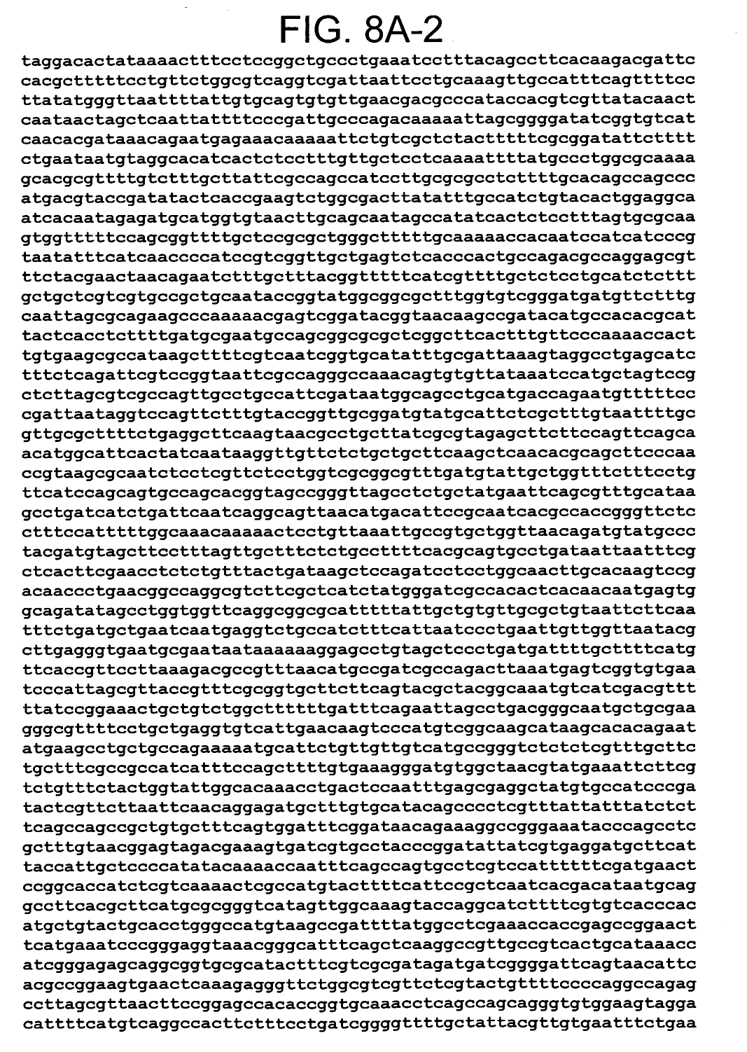

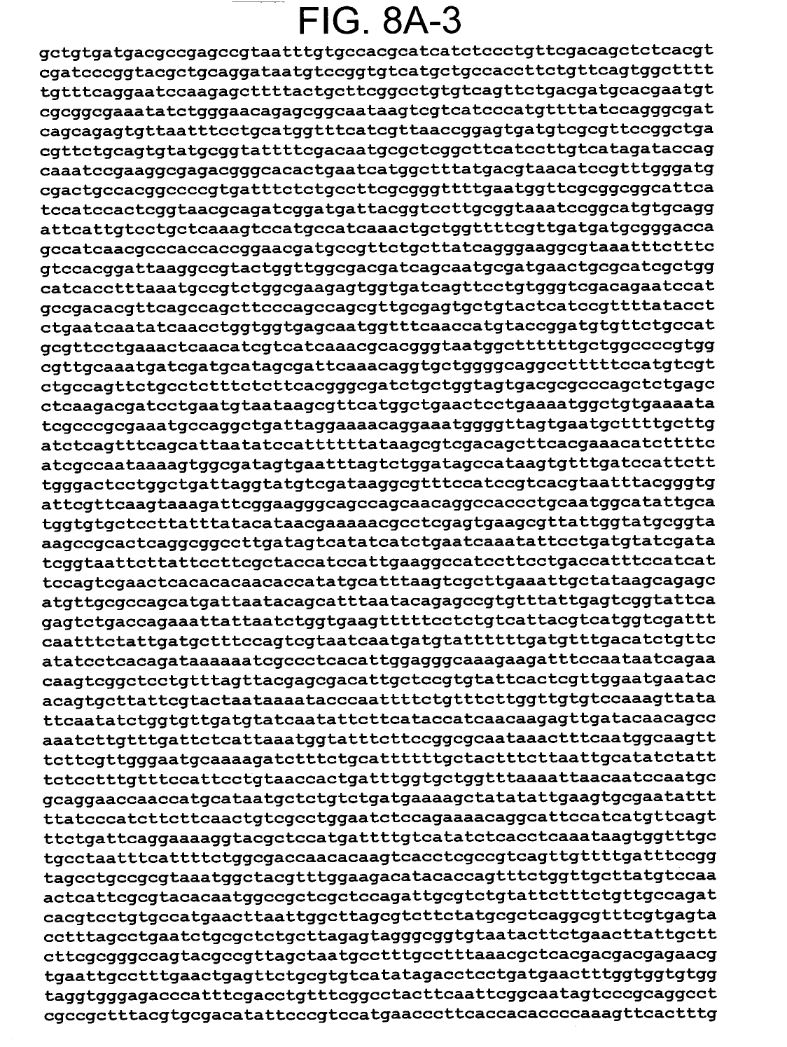

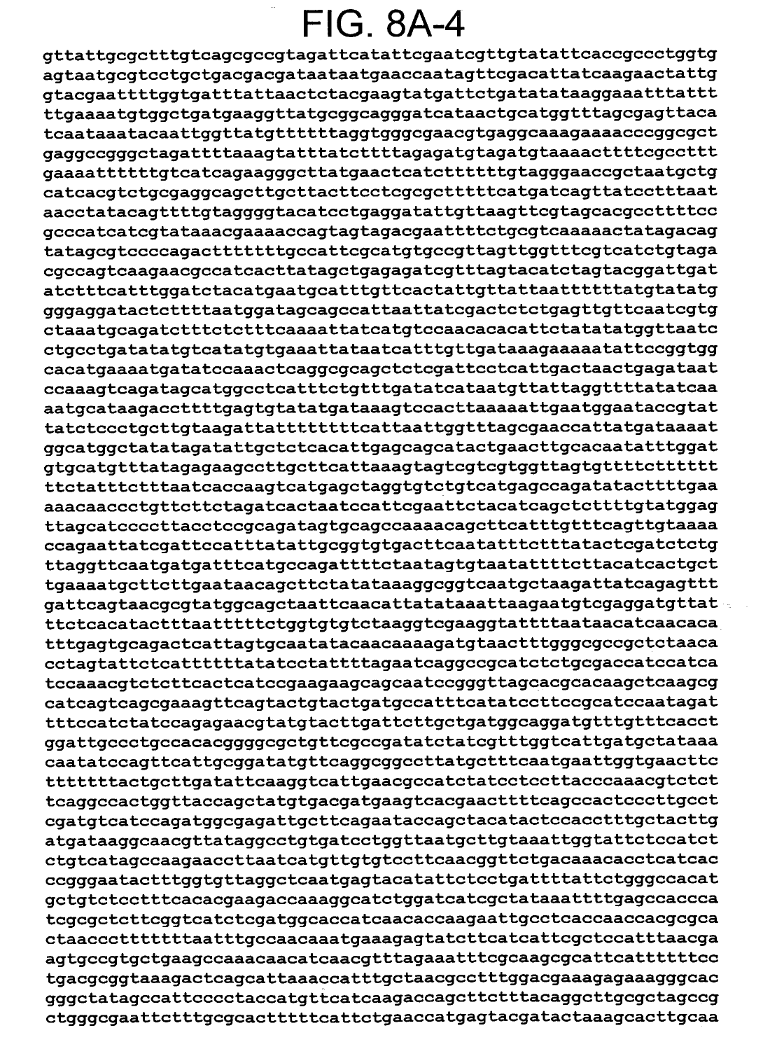

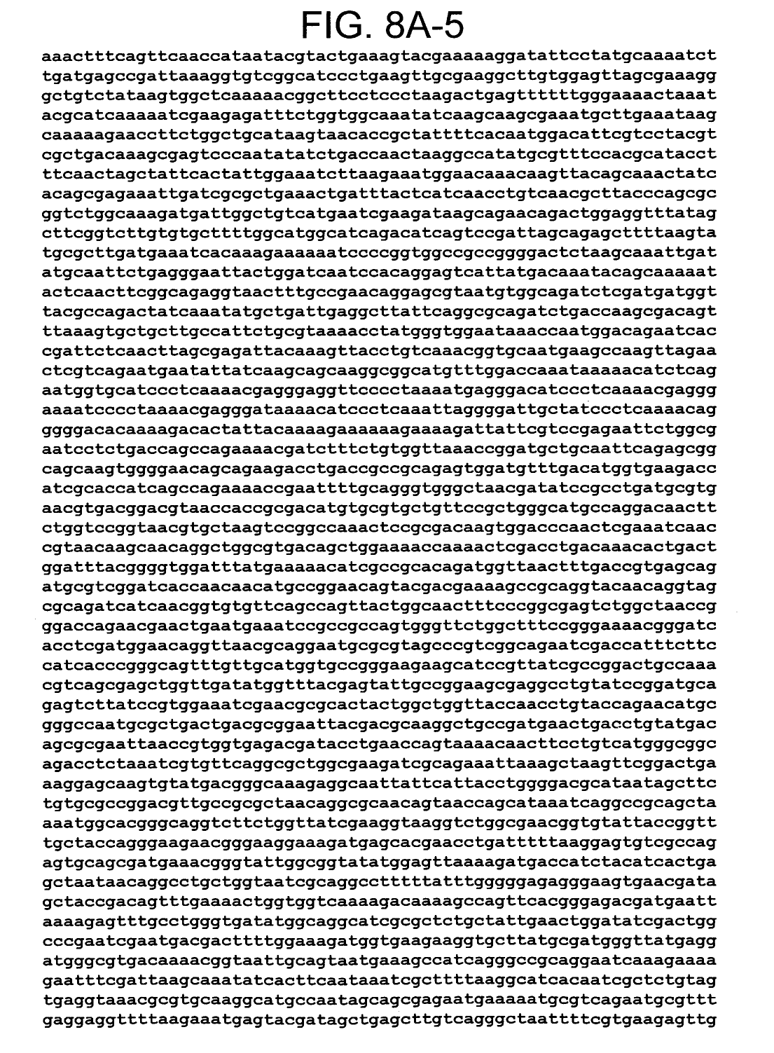

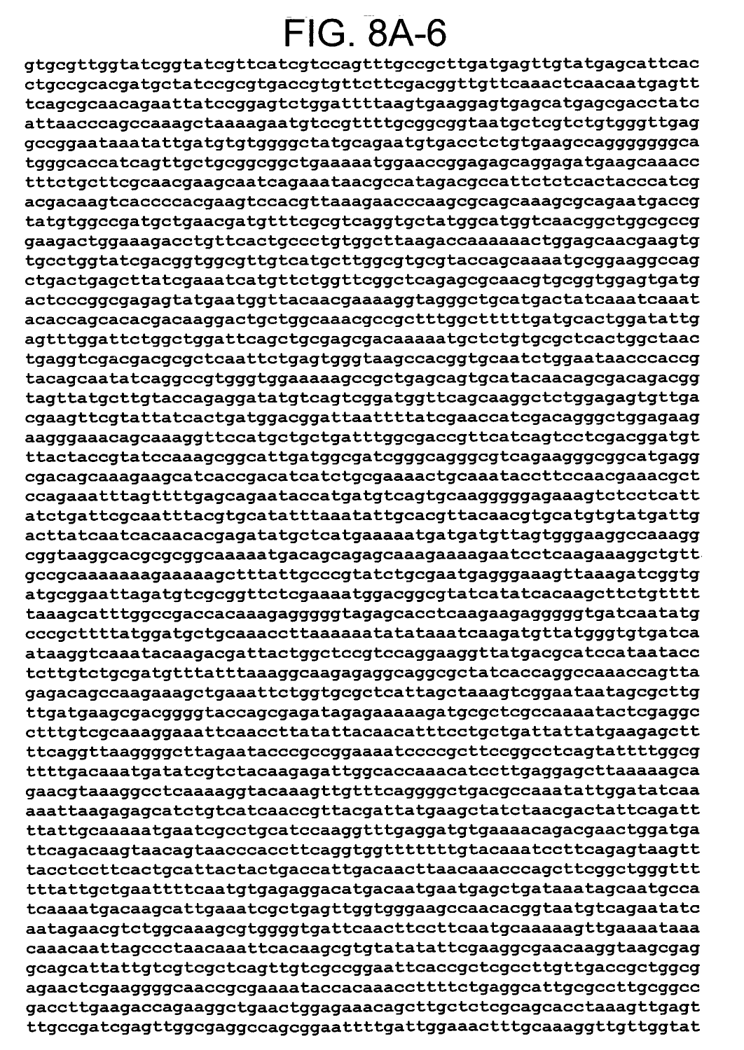

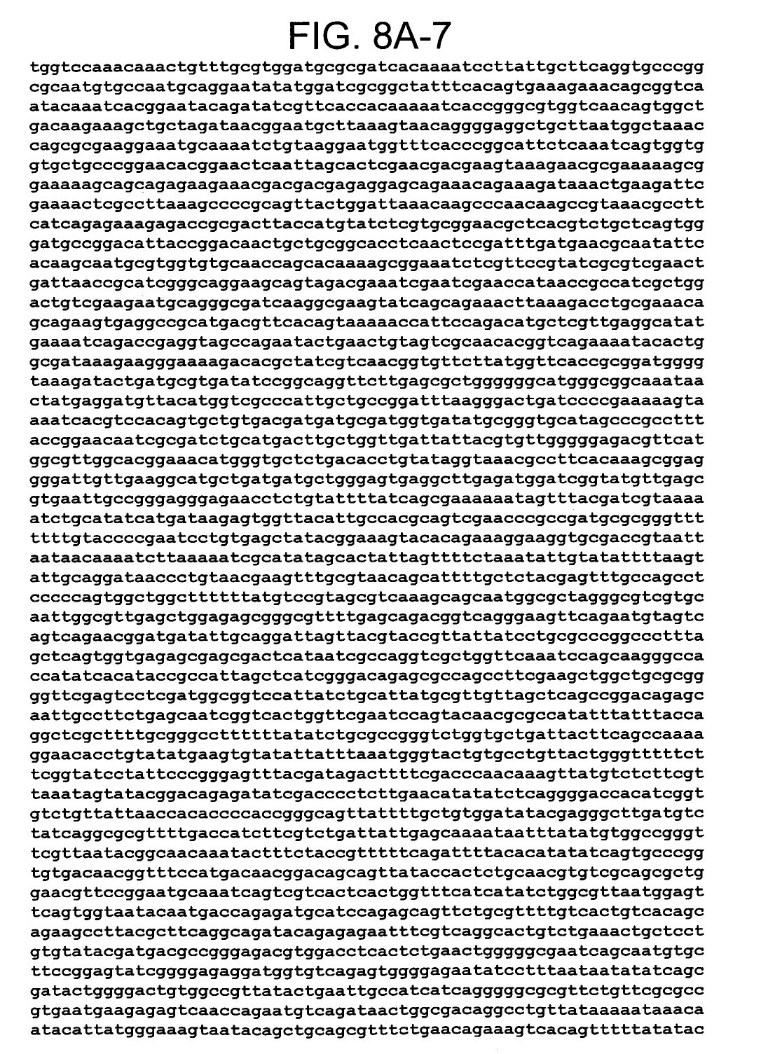

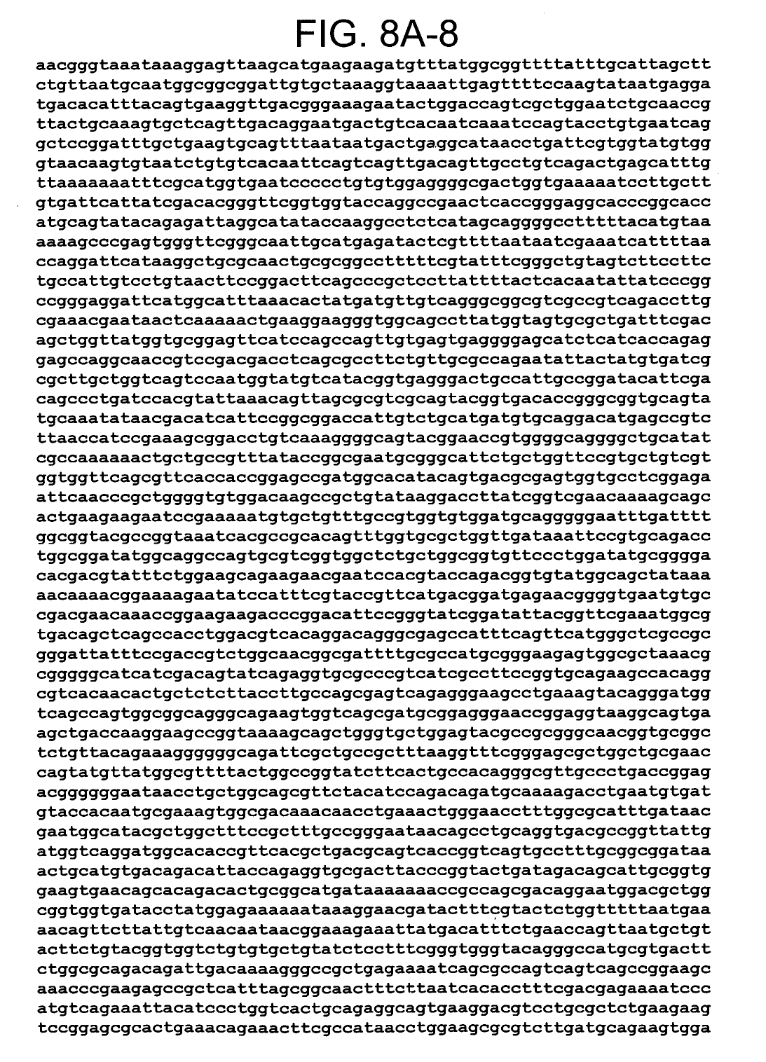

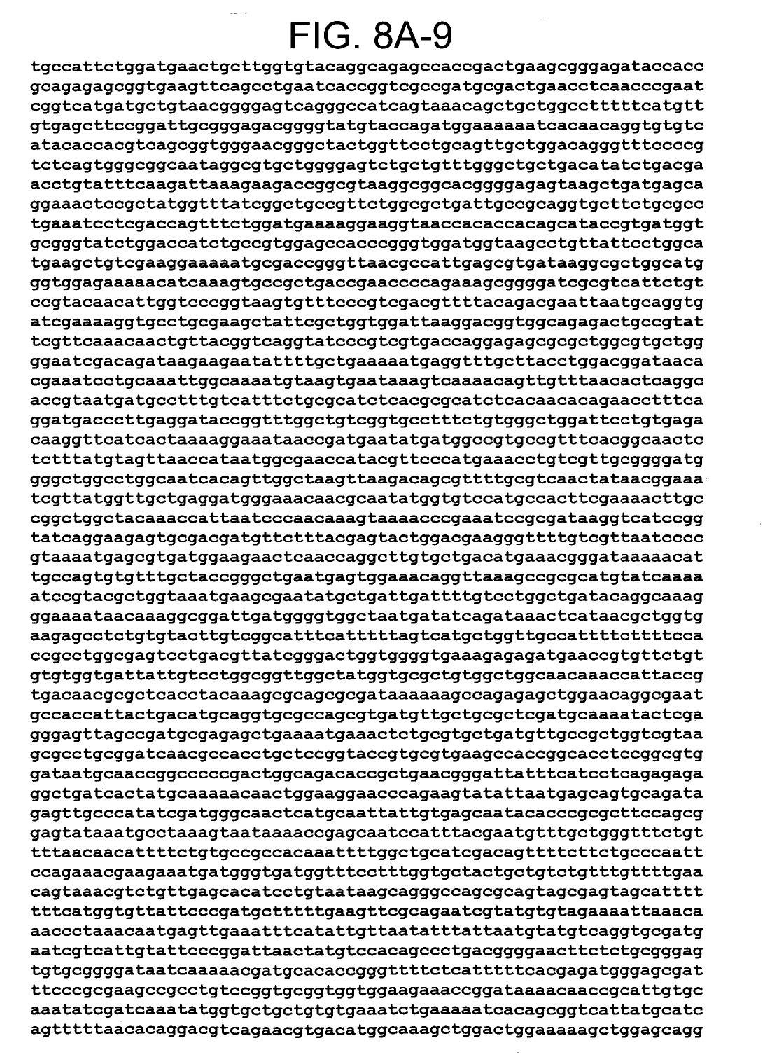

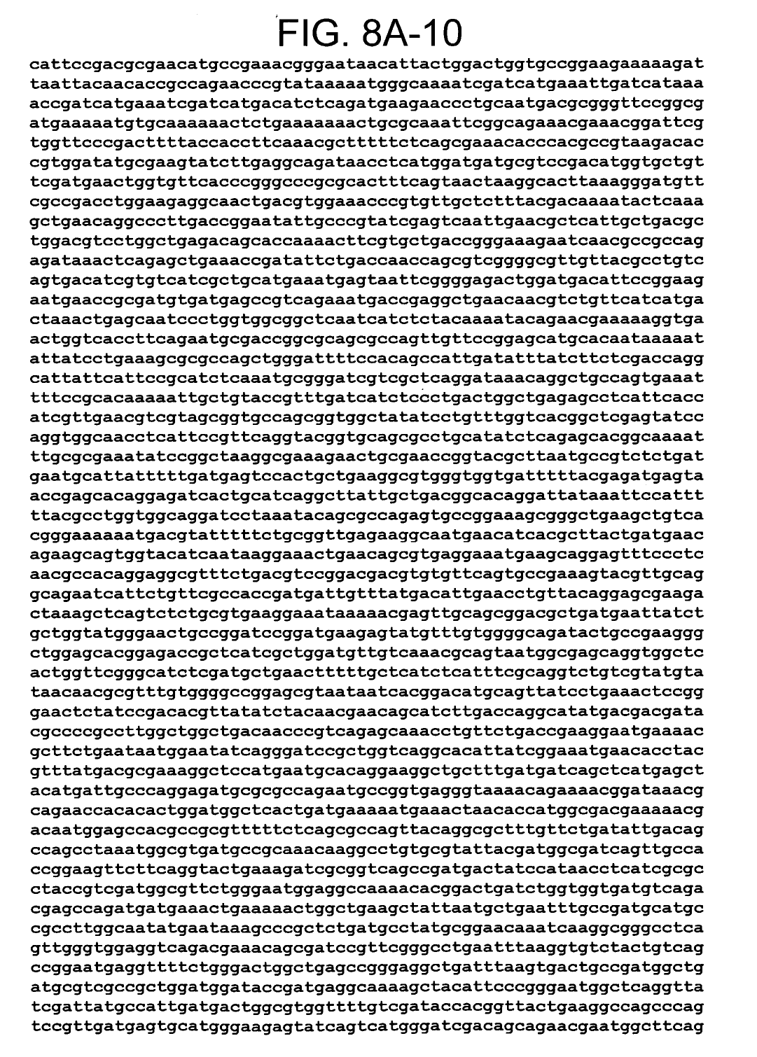

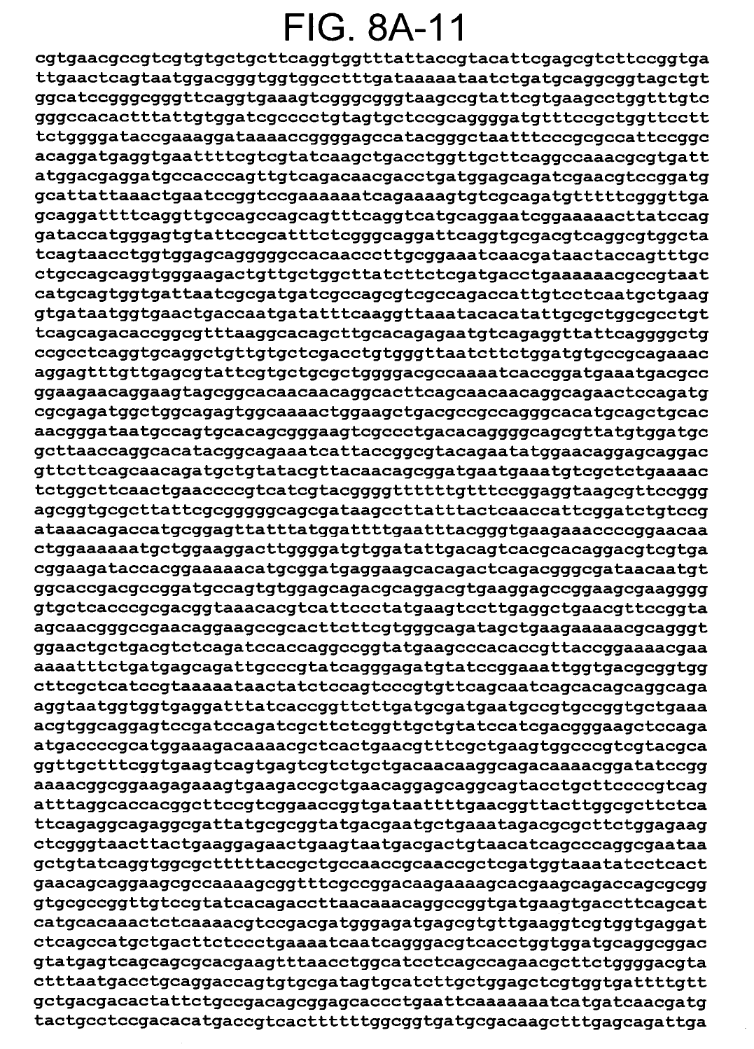

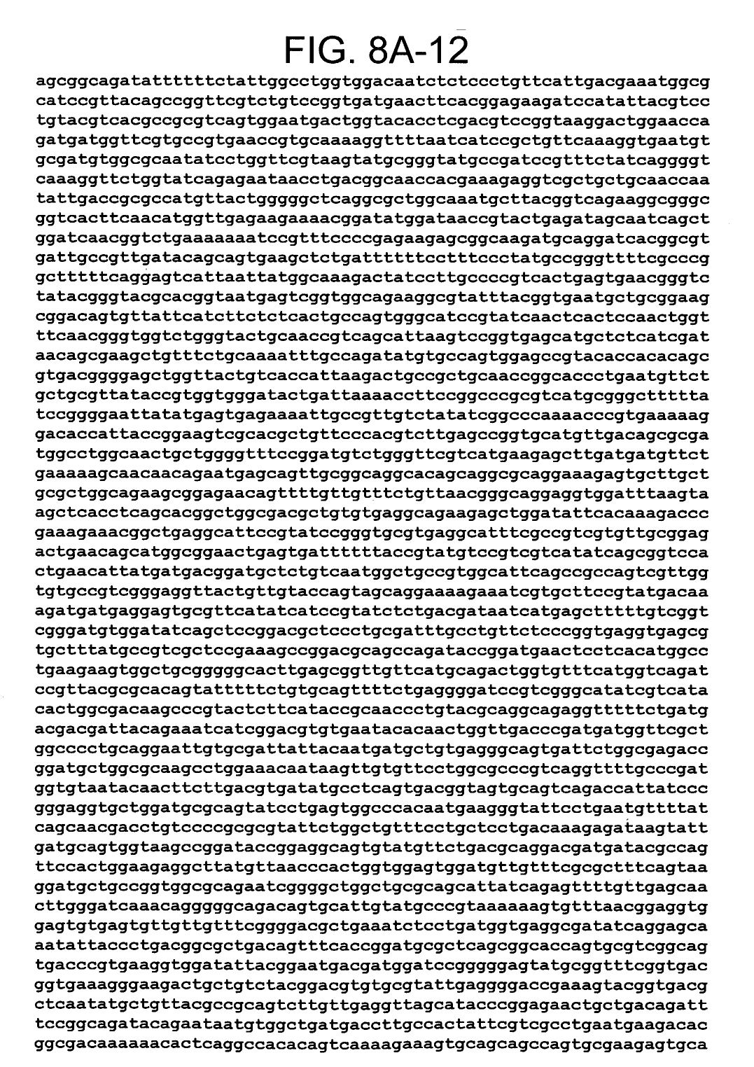

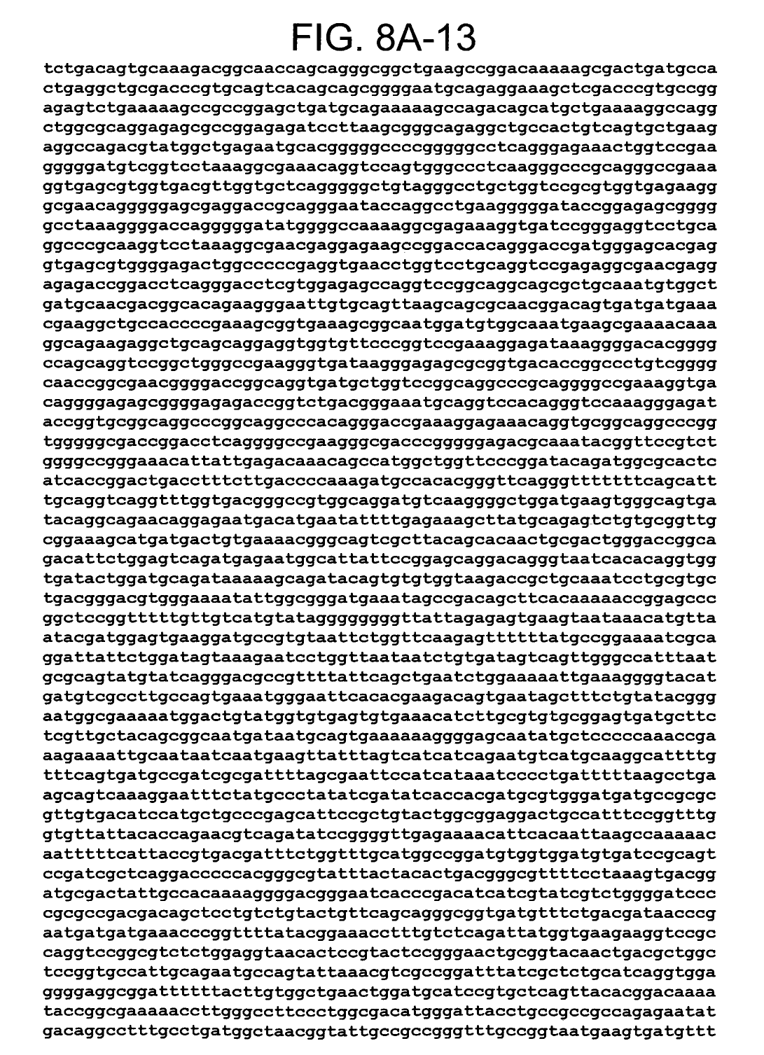

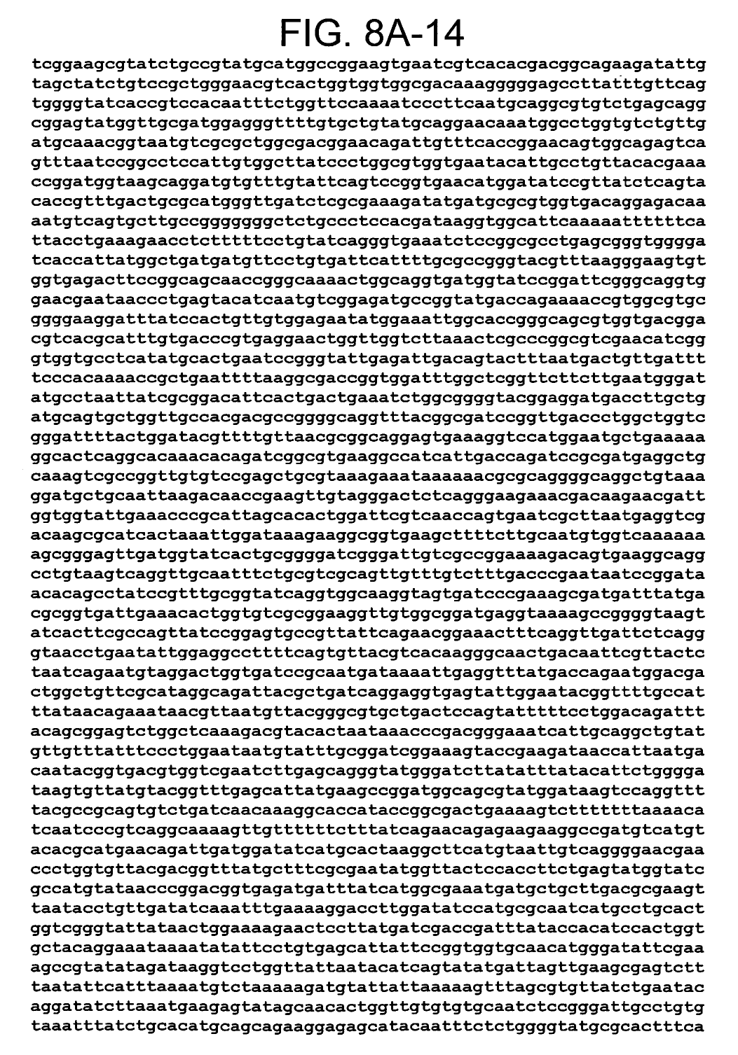

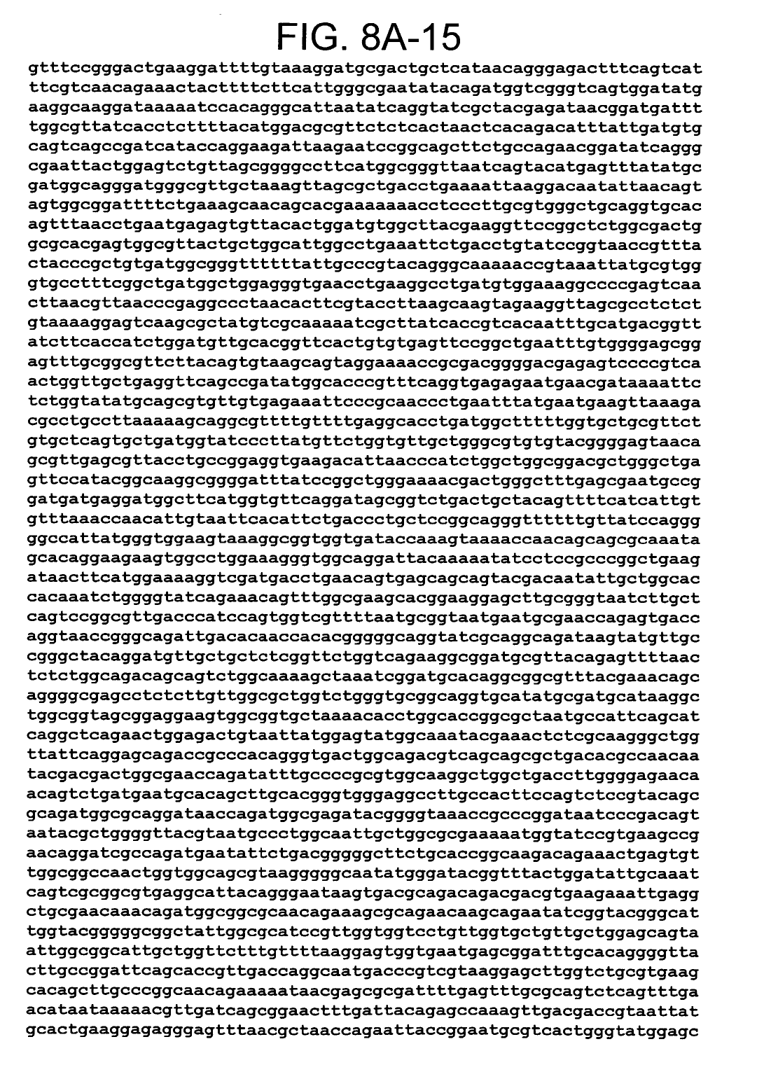

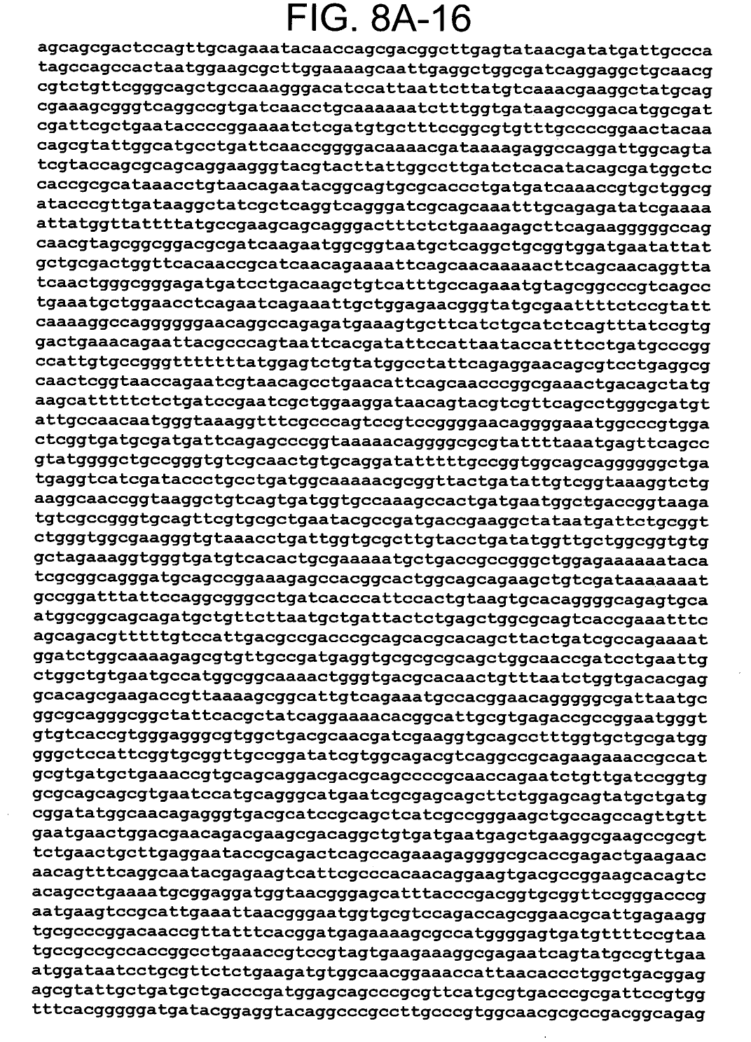

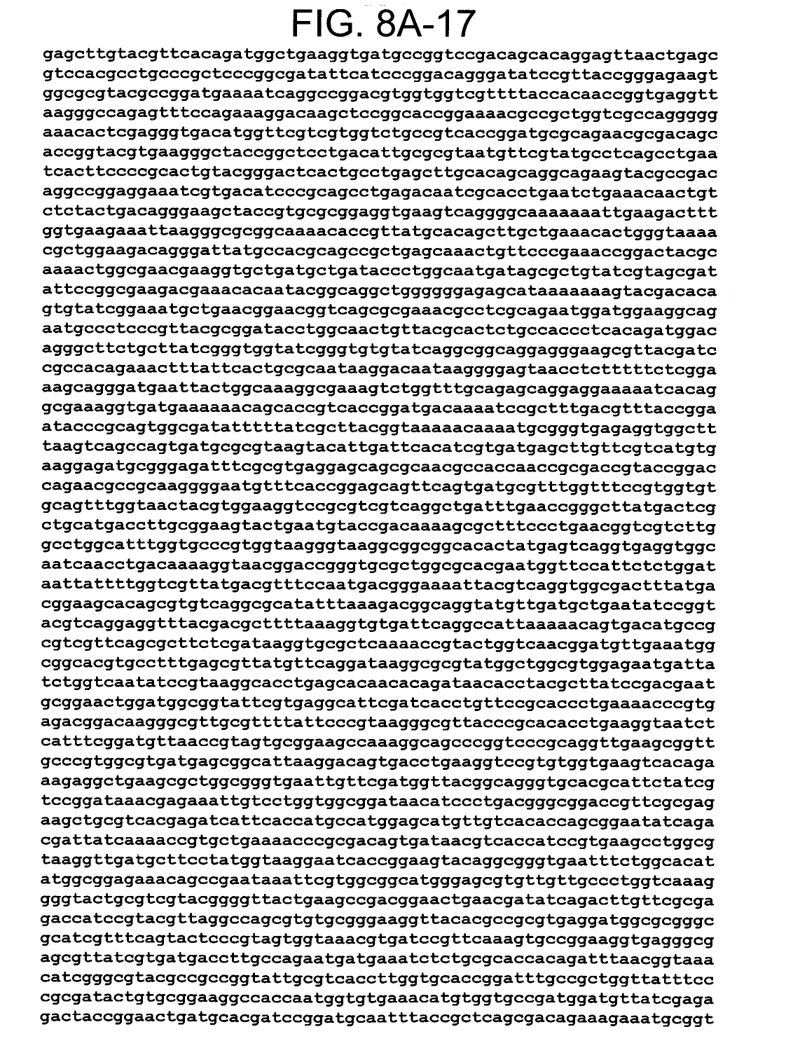

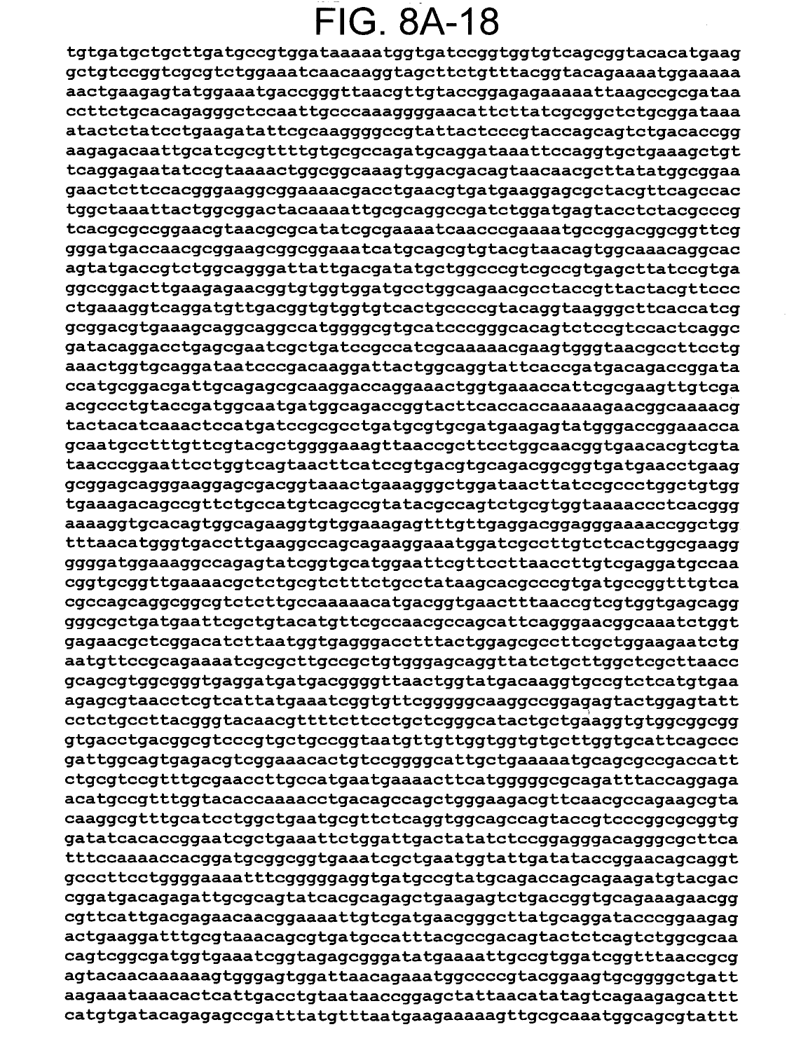

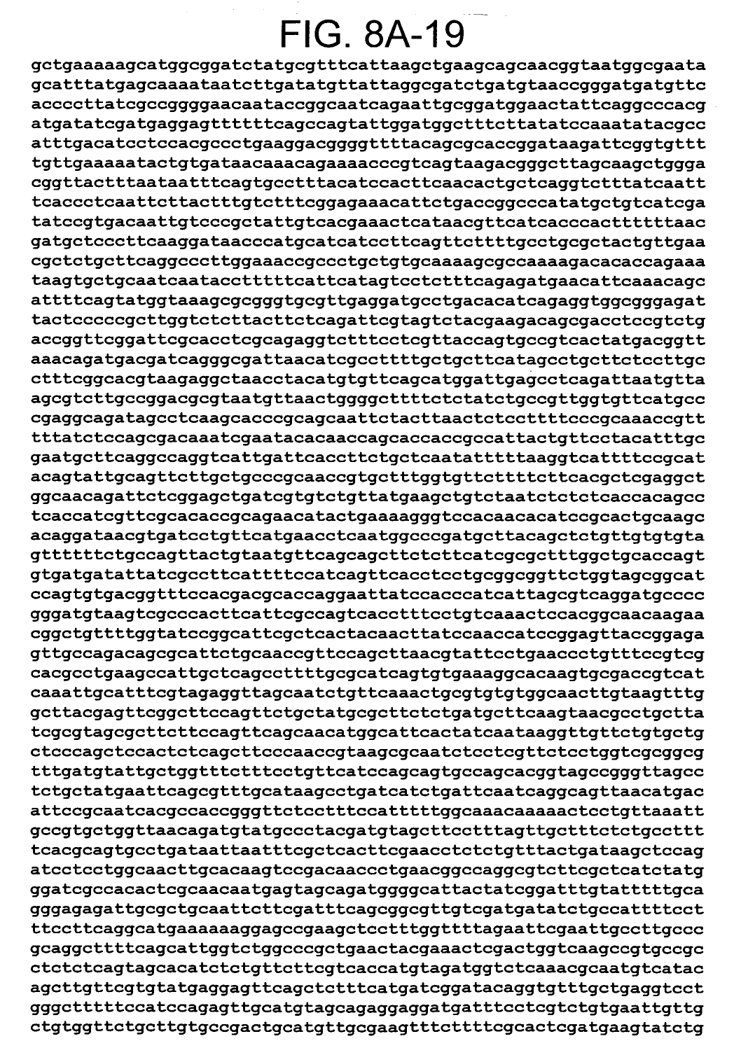

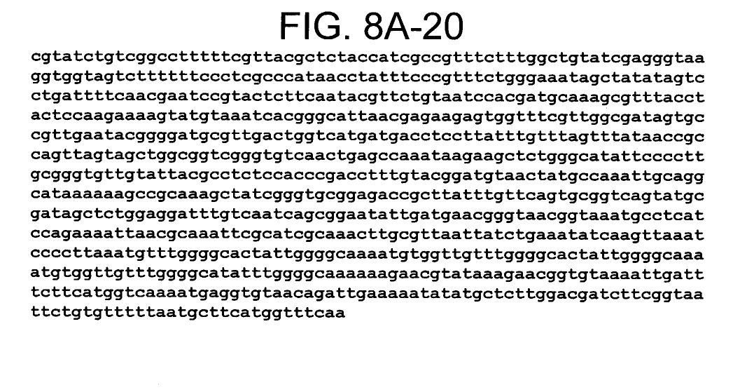

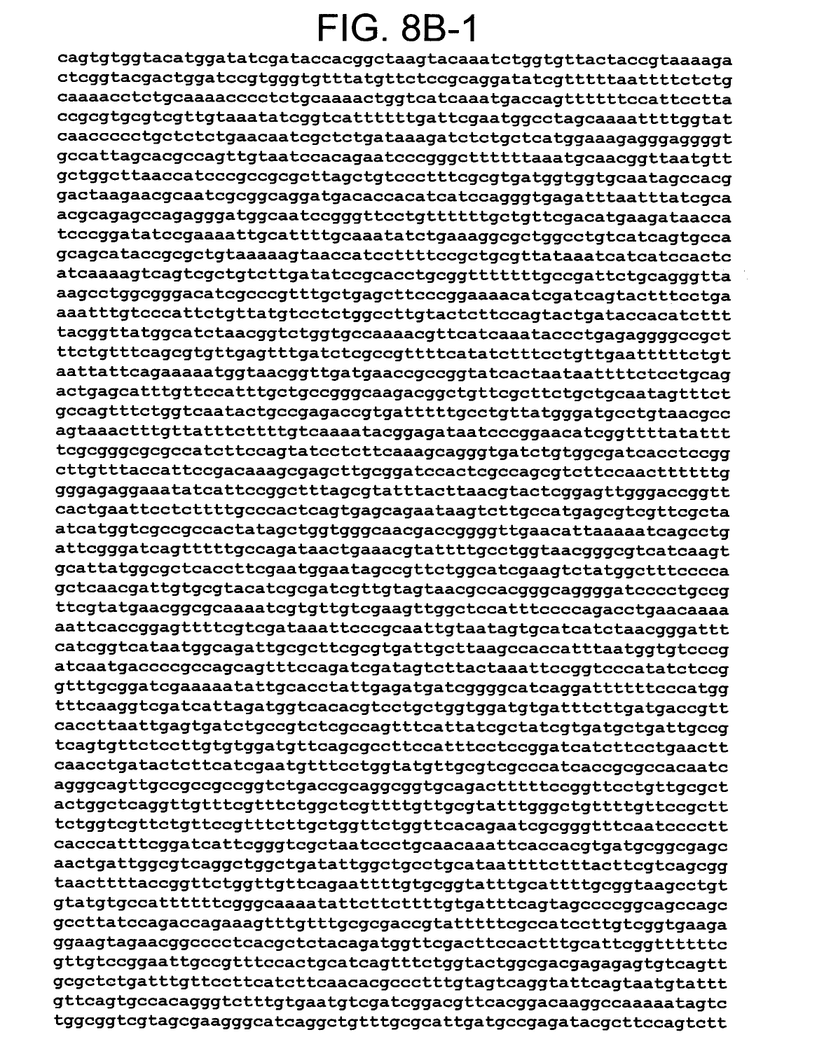

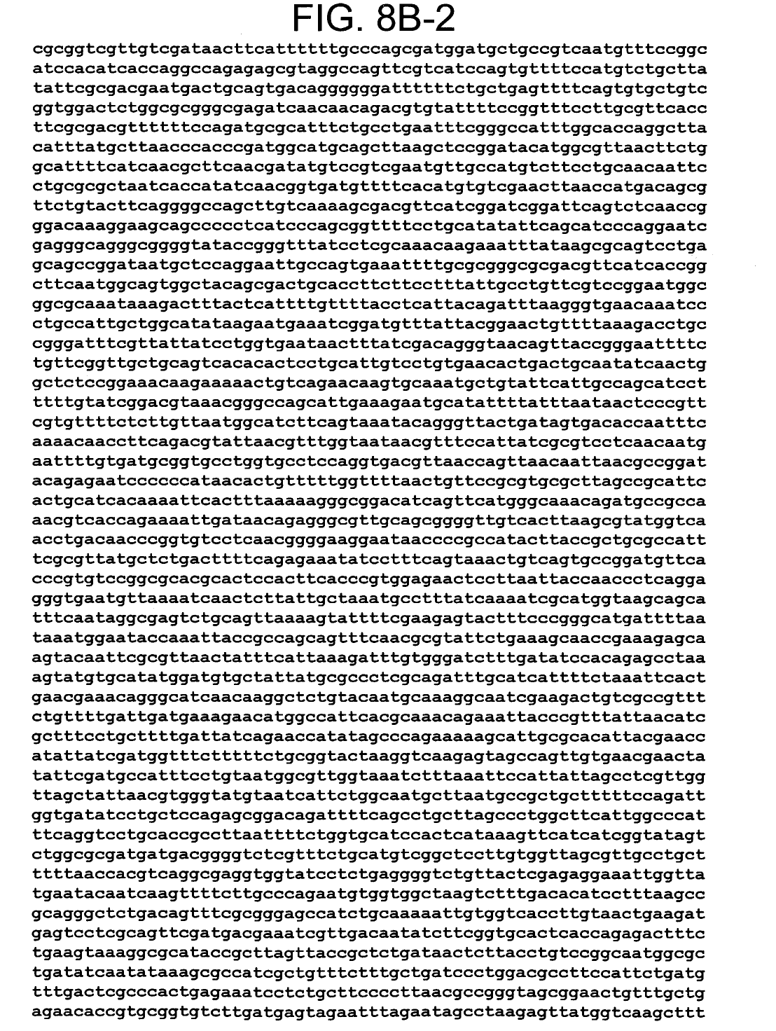

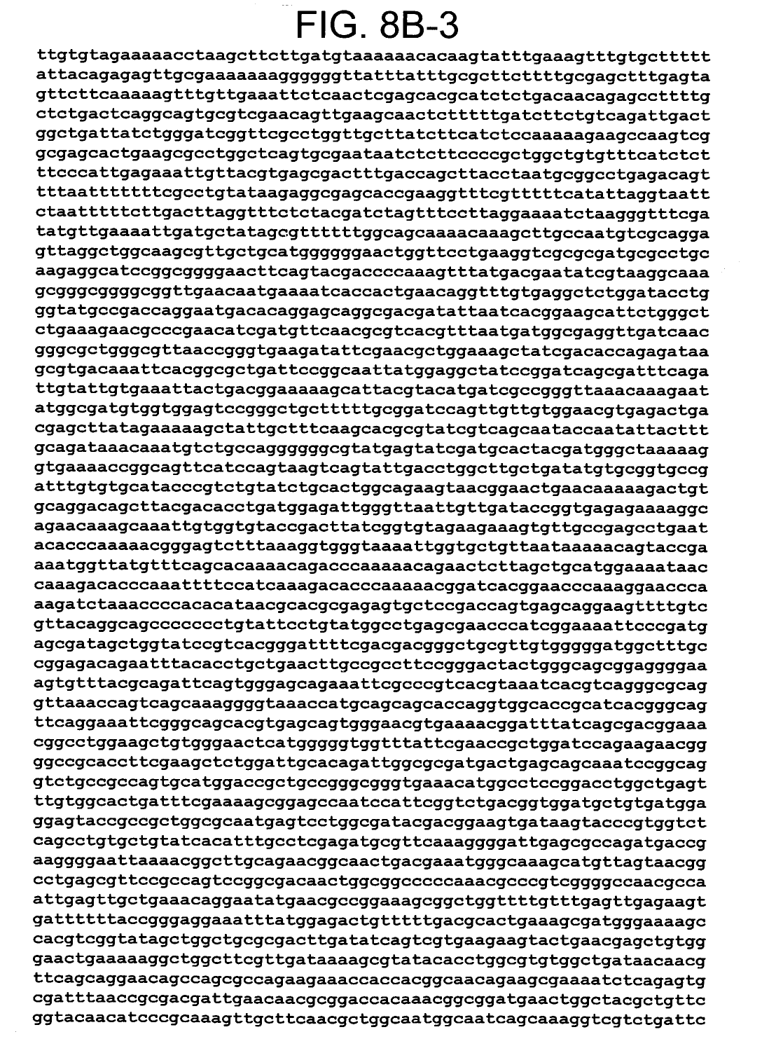

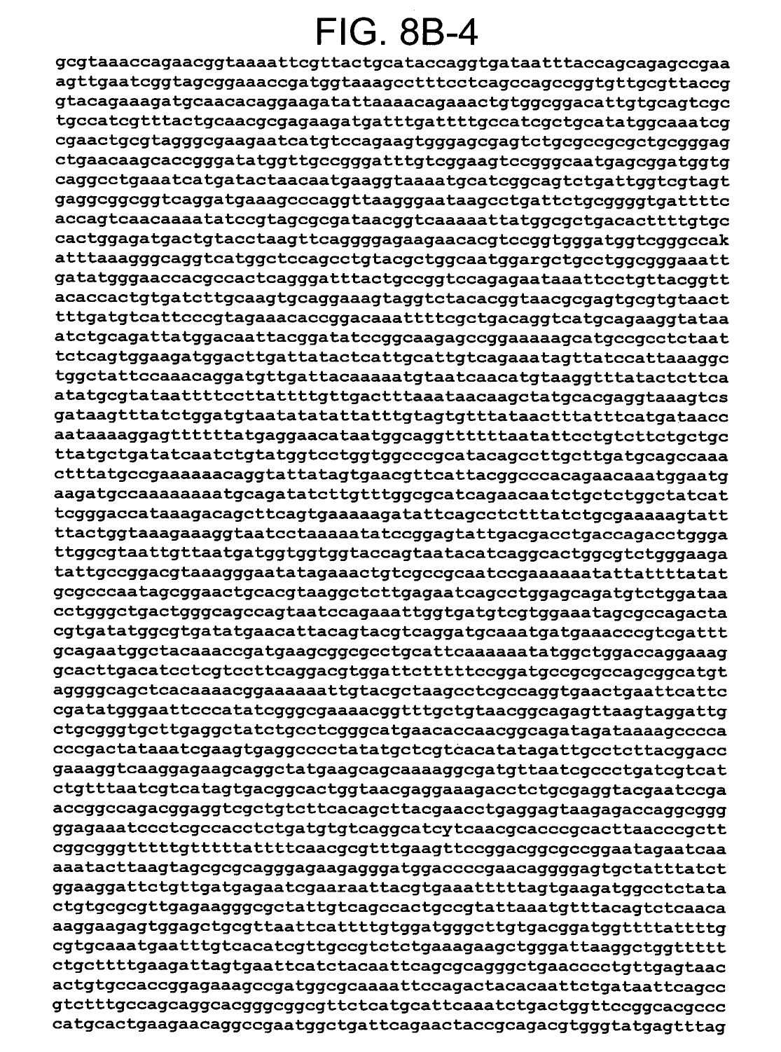

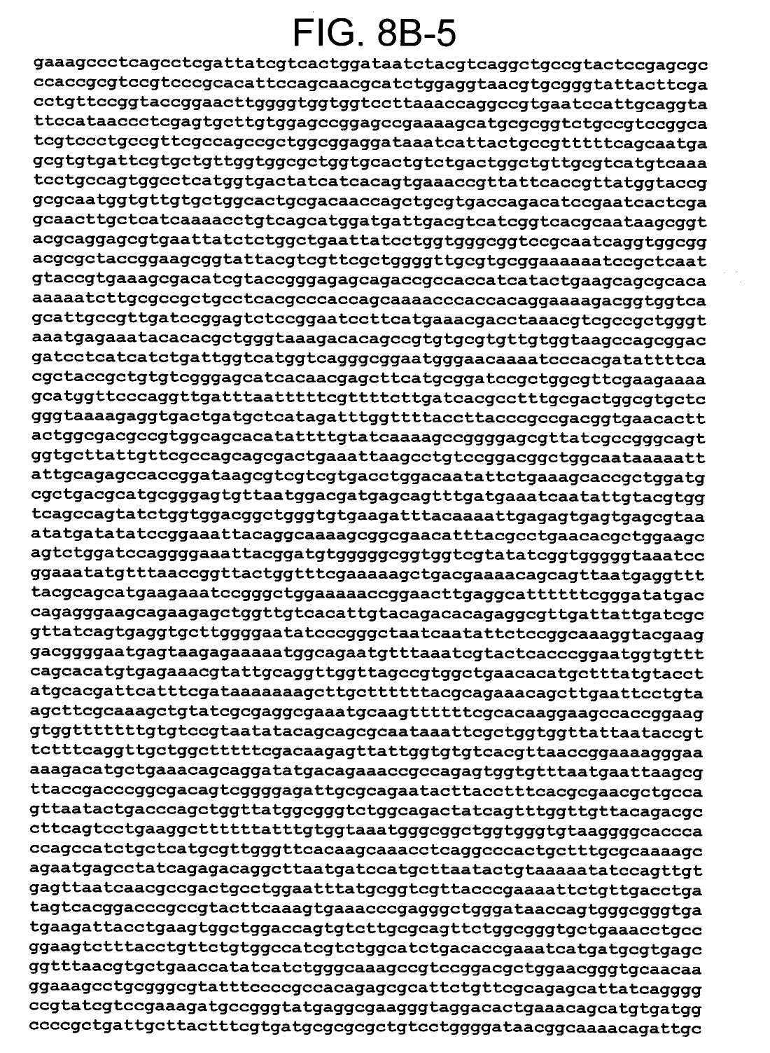

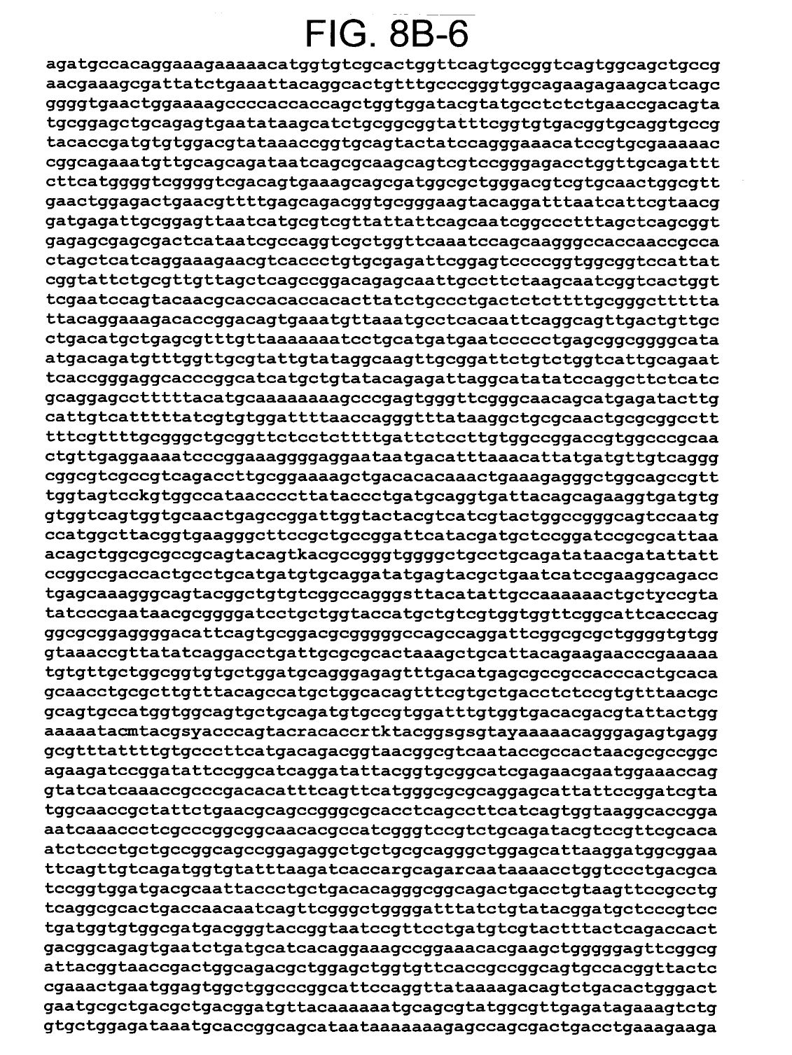

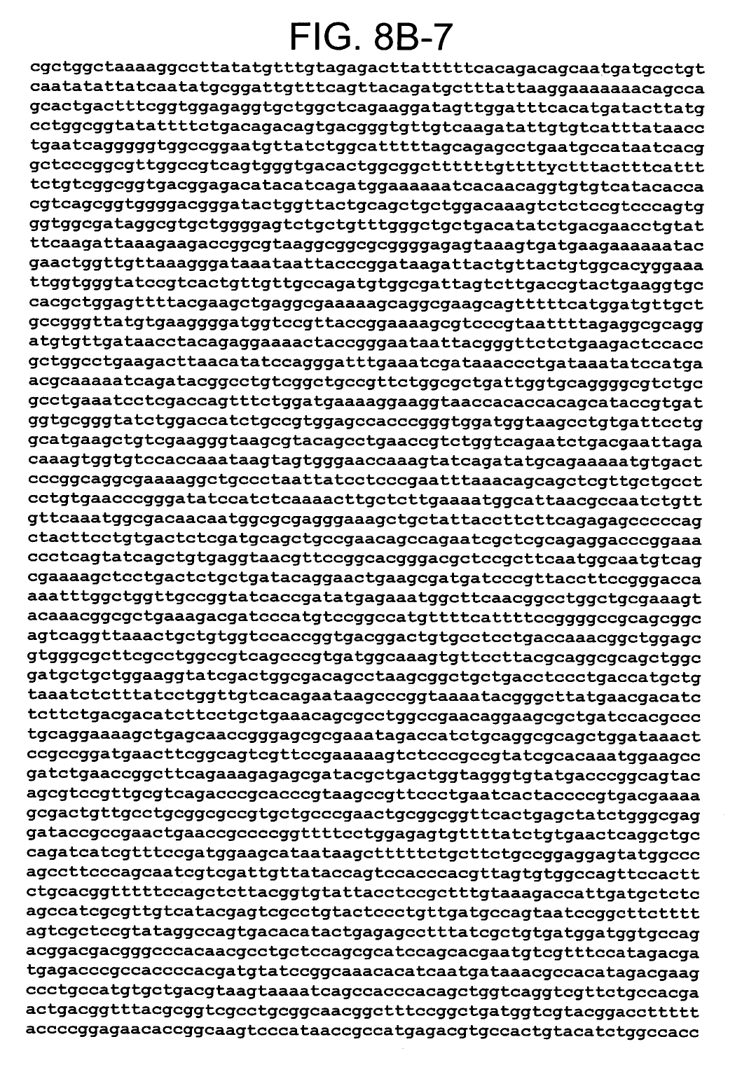

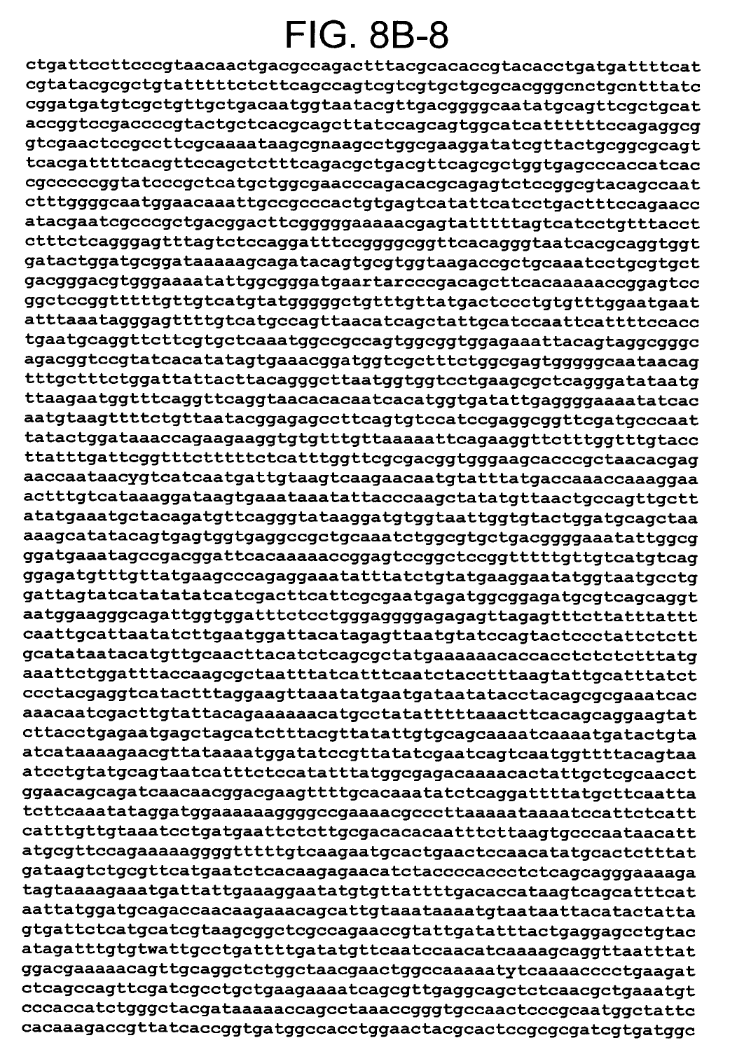

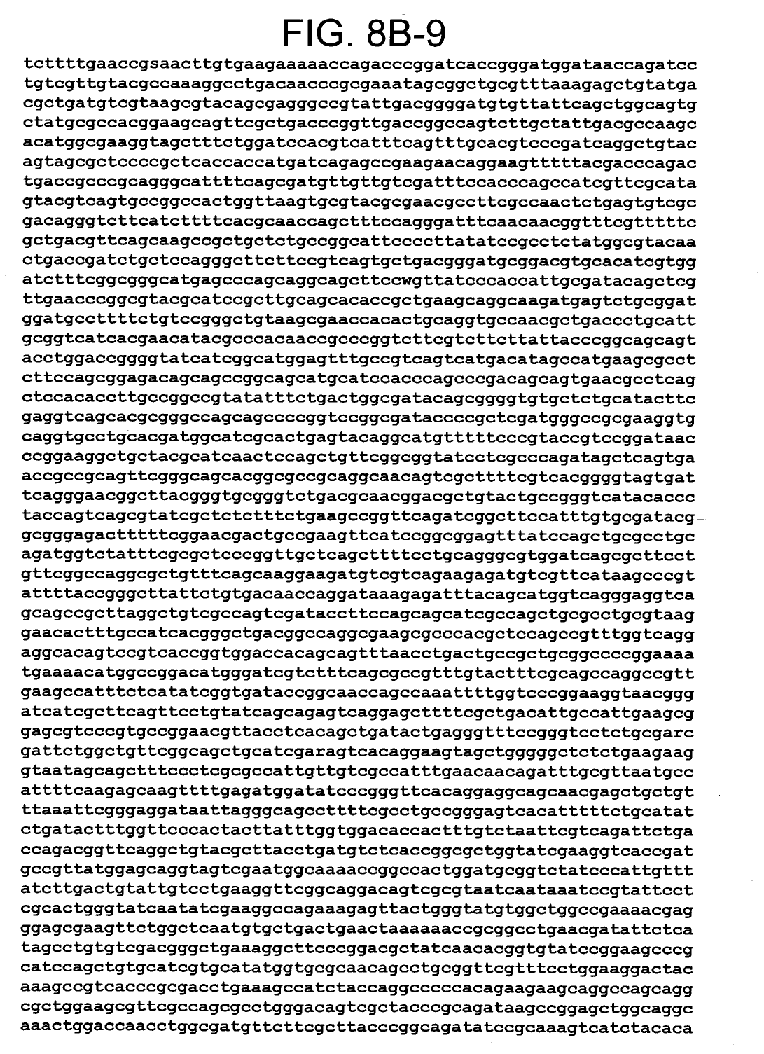

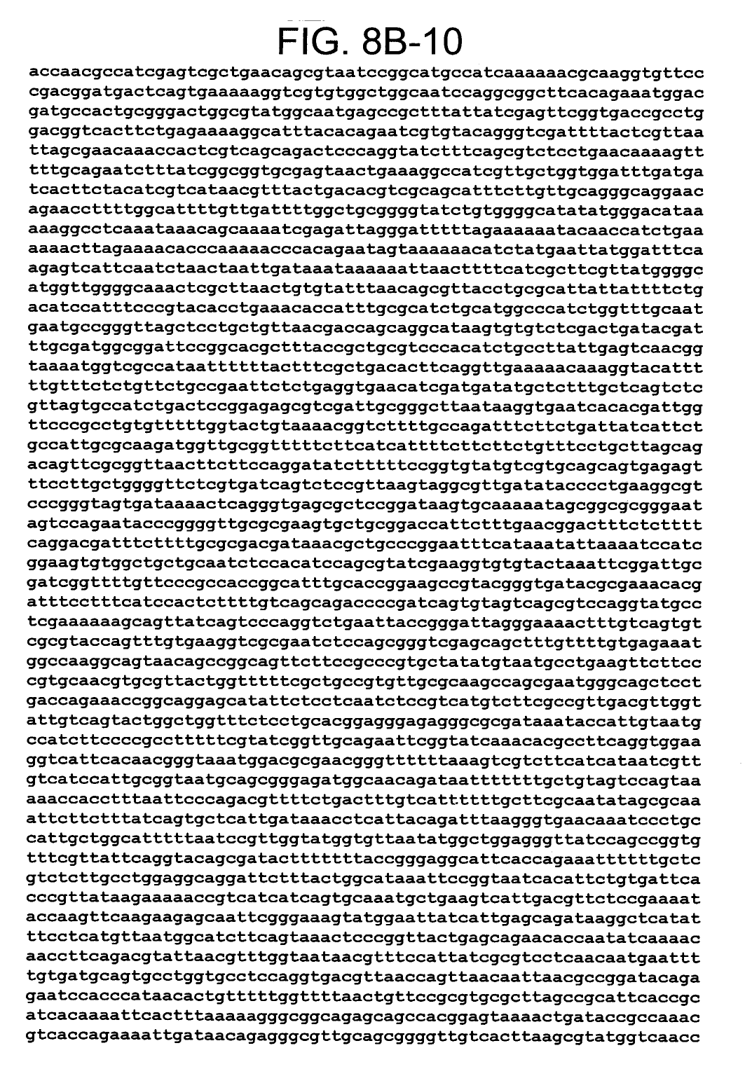

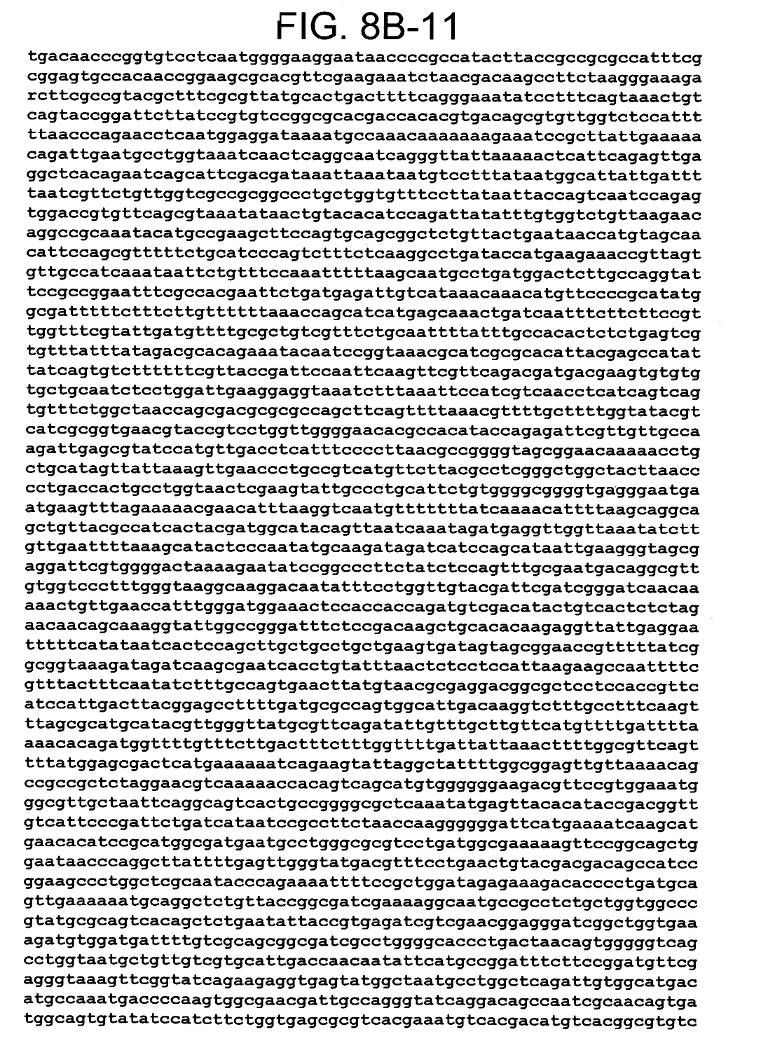

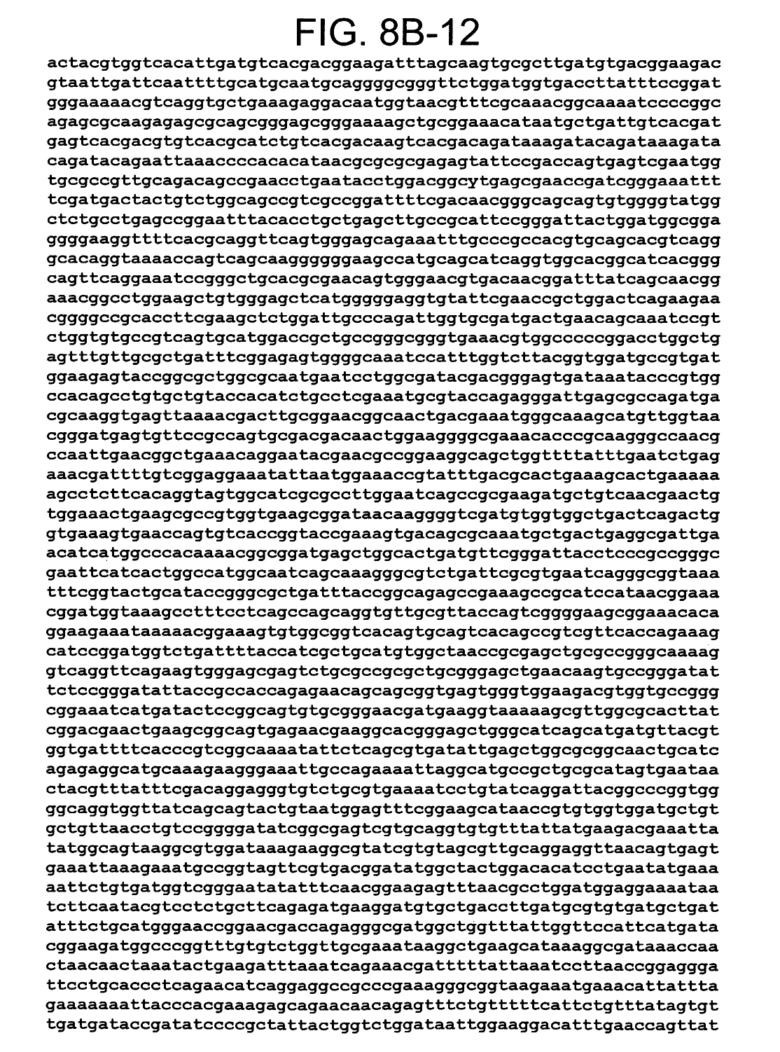

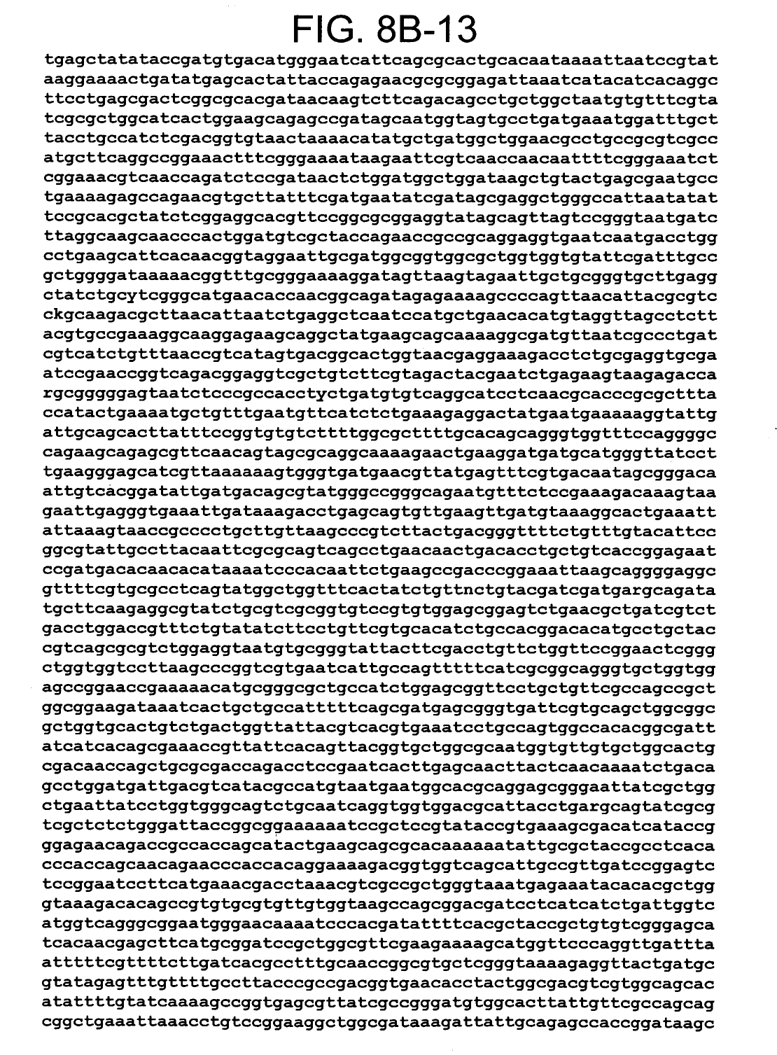

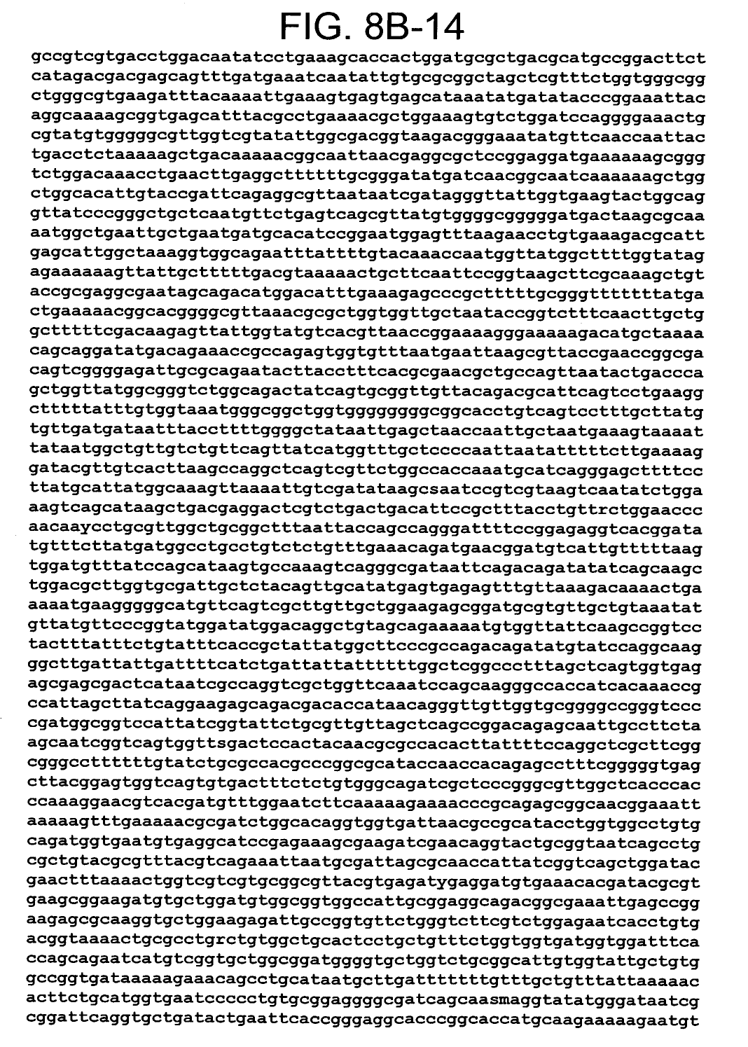

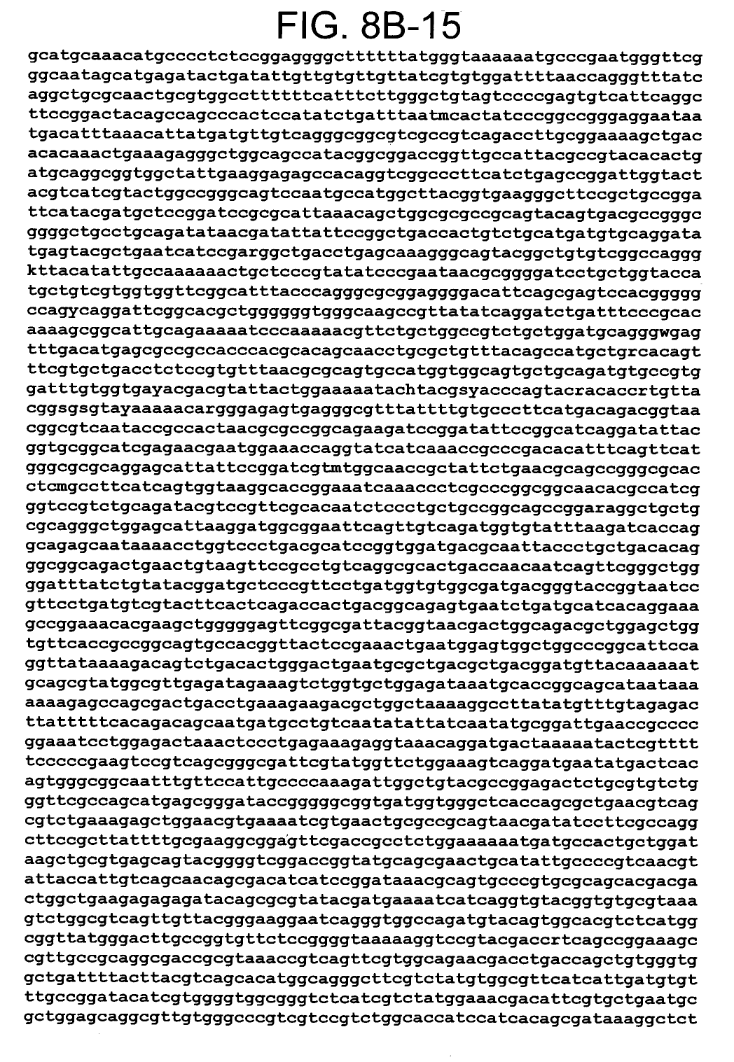

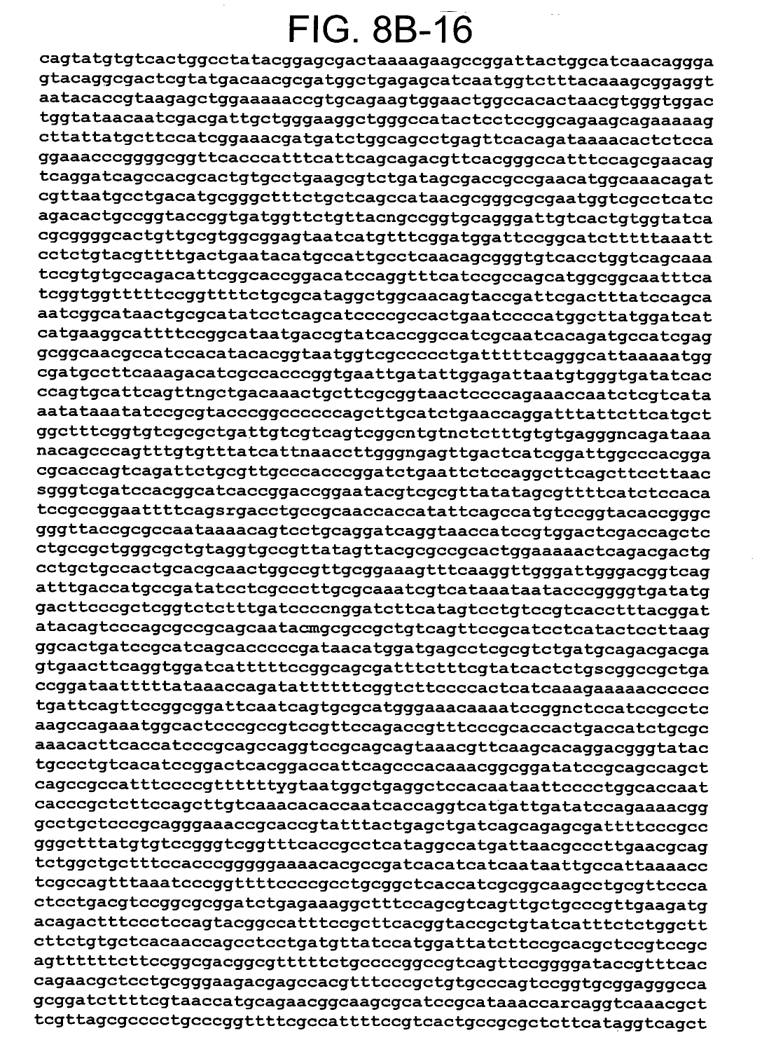

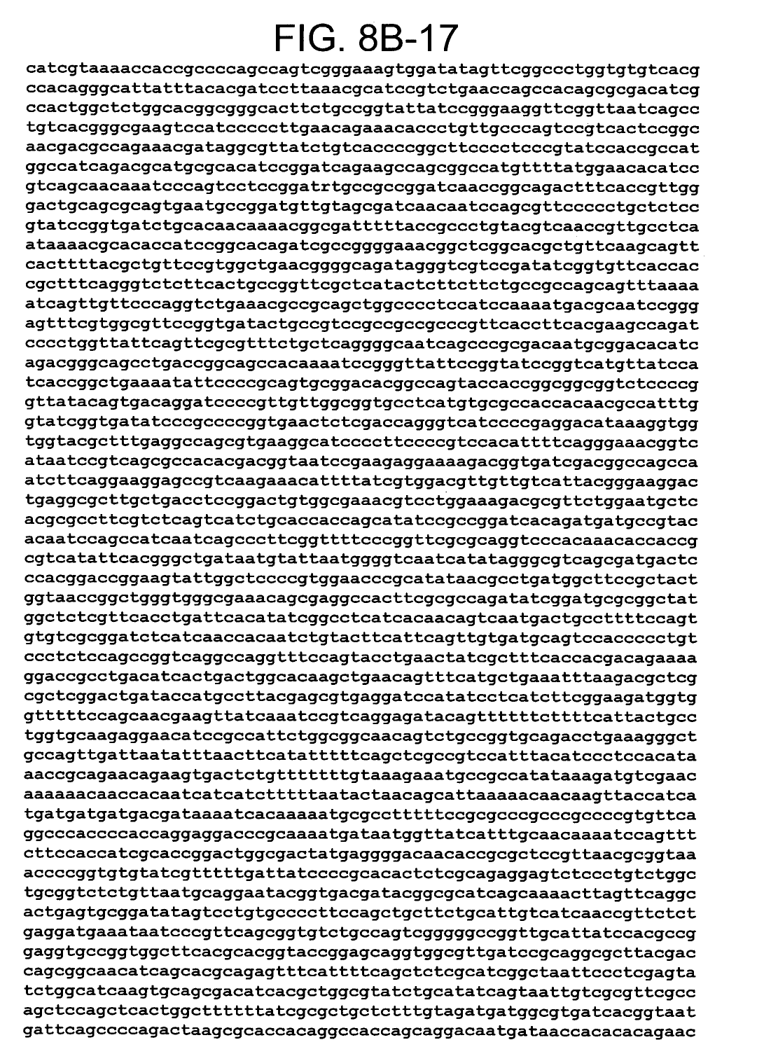

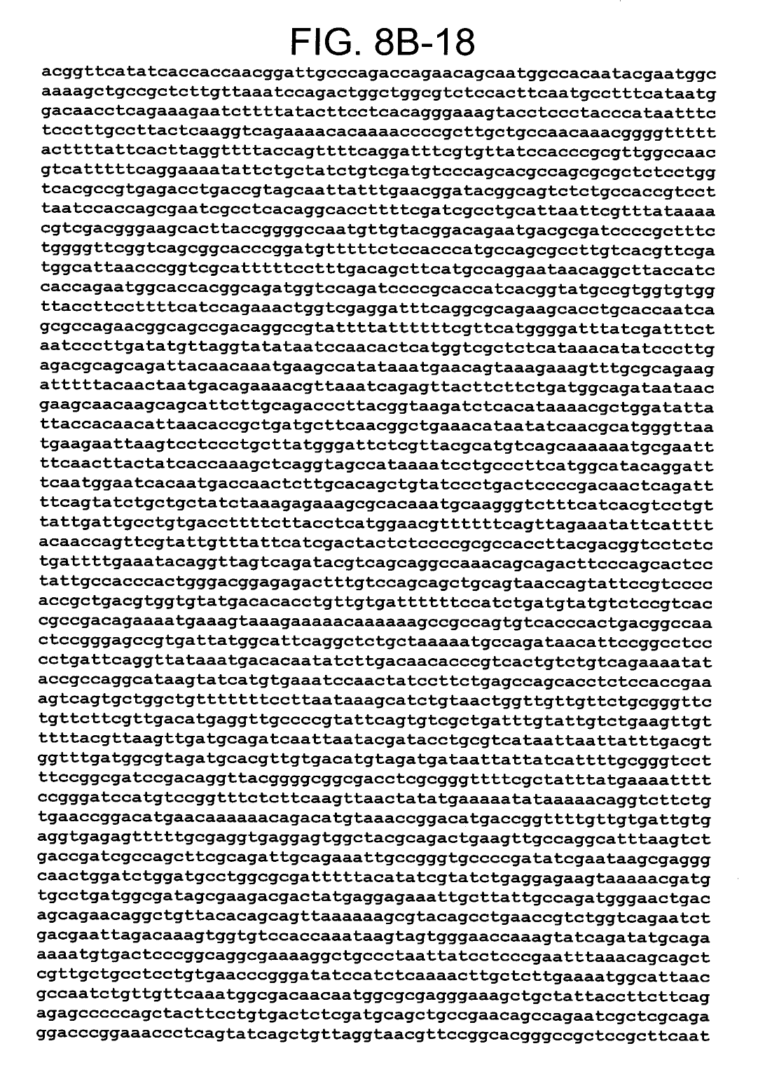

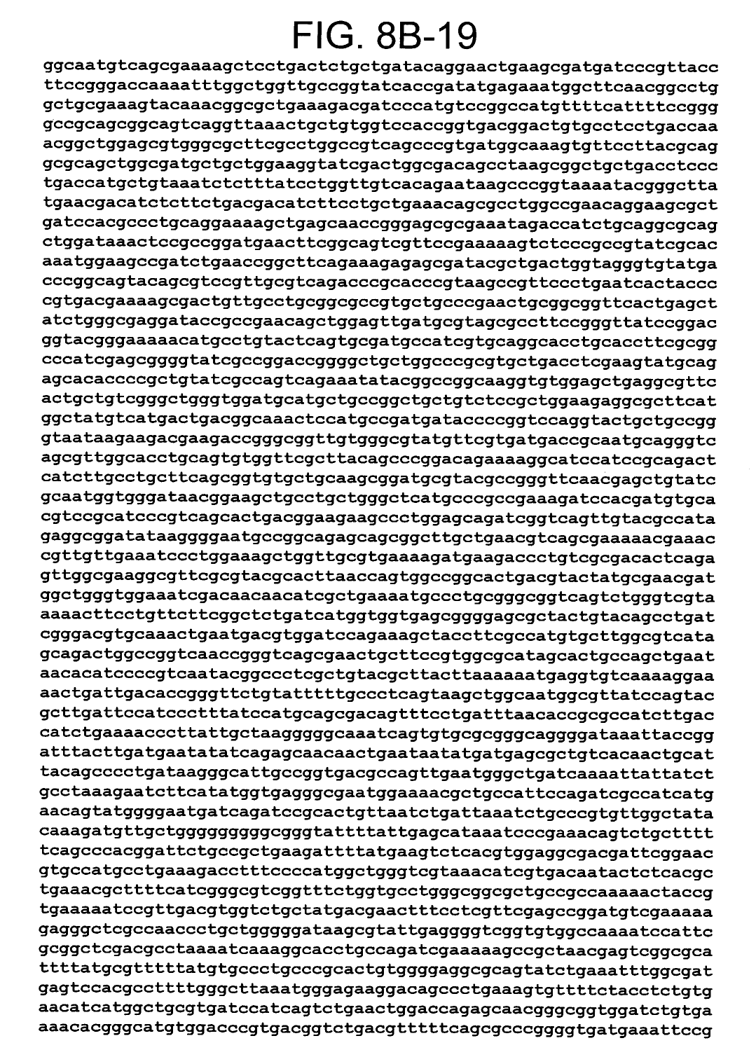

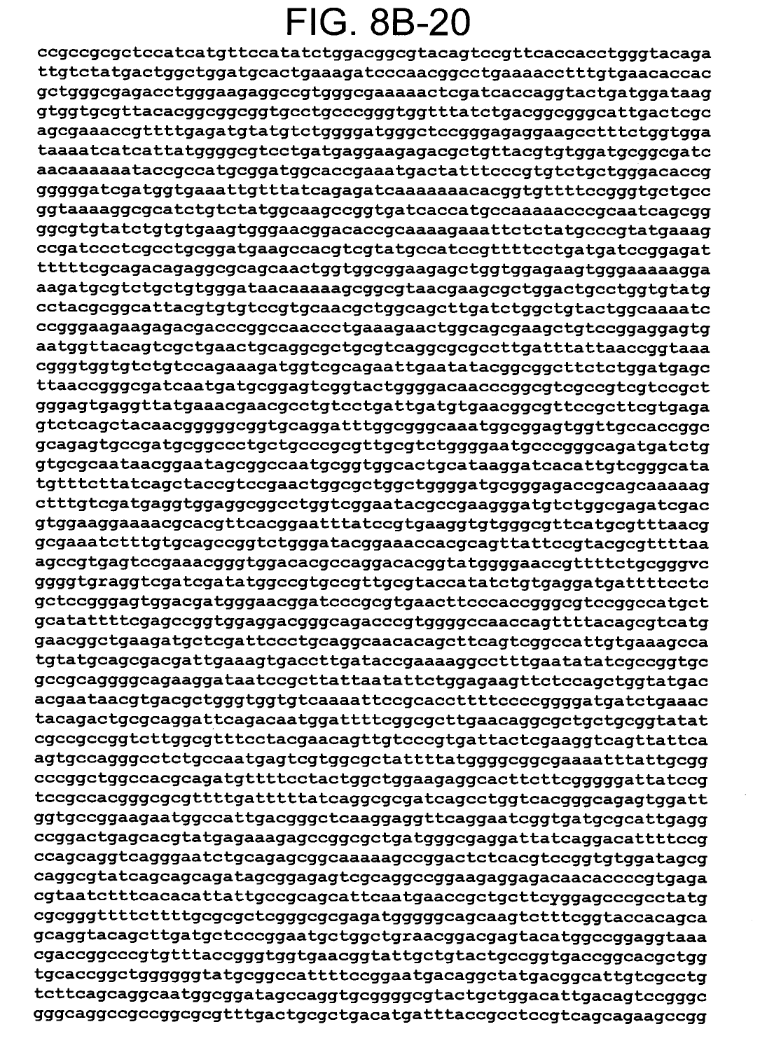

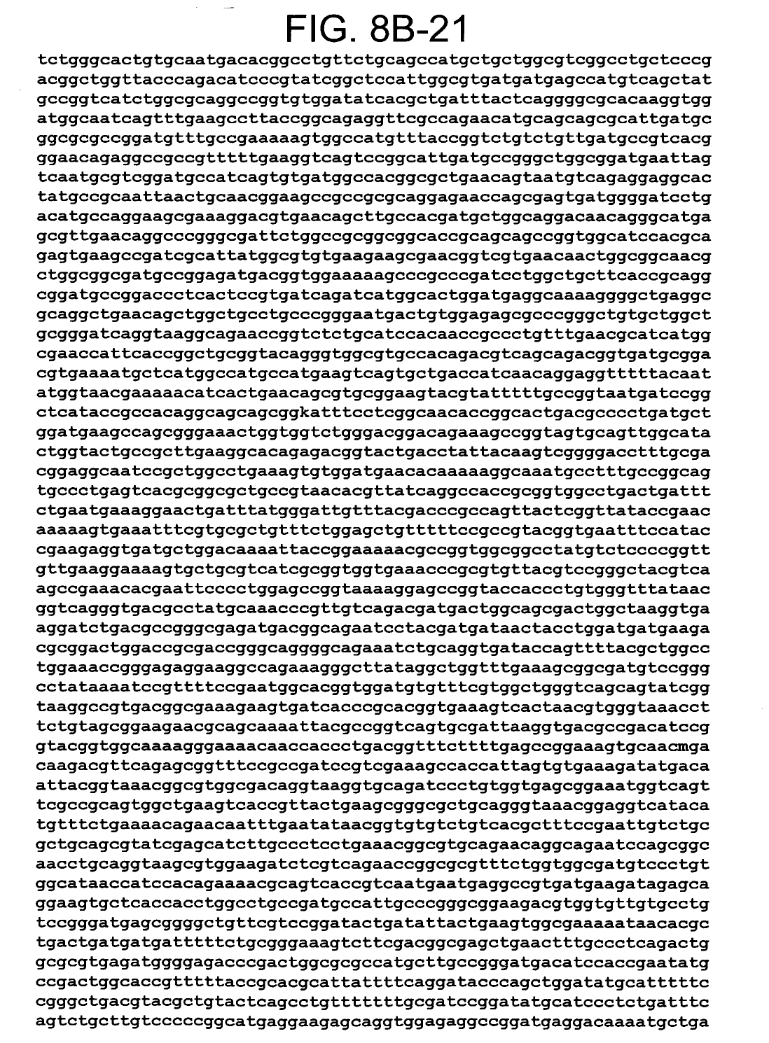

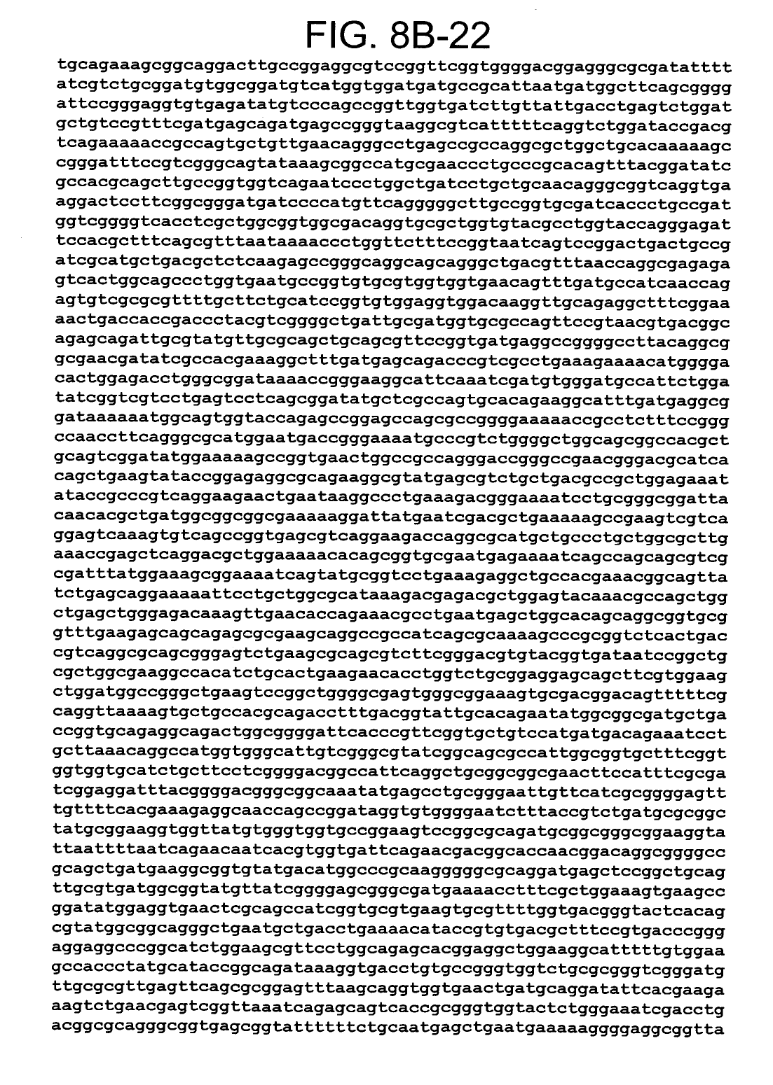

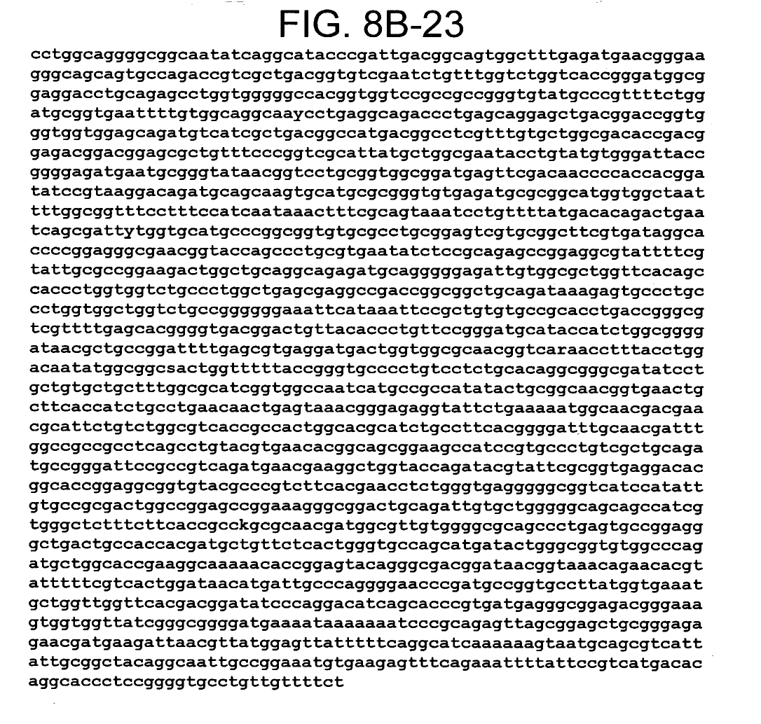

In addition, the sequence of the larger genomic regions, referred to as O-islands, since these are islands of DNA sequence that lie within the genomic sequence of E. coli O157:H7 that are not found in the sequence of the non-pathogenic E. coli strain K12 are described in FIG. 8.

We also designed primer pairs to amplify DNA flanking thirty-three sites in the O157 genome for another rare cutting restriction enzyme, AvrII. Of these sites, we identified seven that were polymorphic between E. coli O157:H7 isolates. In the case of the AvrII sites, polymorphisms were due to insertions, deletions, or single nucleotide polymorphisms (SNPs). The SNPs occurred either within the AvrII site itself, resulting in loss of the site, or in sequences near the site, resulting in the creation of an additional AvrII site. Of the 7 polymorphic AvrII sites, 5 were in O-islands and 2 were in the backbone (sequences shared with E. coli K12). Using the primer pairs specific for DNA flanking these 7 polymorphic AvrII sites with the primer pairs specific for the 8 polymorphic XbaI sites and the four virulence genes (stx₁, stx₂, eae, hlyA), made the bacterial typing system described herein highly discriminatory for distinguishing strains of O157.

While this method is exemplified in the Examples described herein with the strain typing of E. coli O157:H7, it is contemplated that this method will work equally well for typing other species or sub-species of bacteria. Exemplary art-recognized bacteria include, without limitation, foodbome pathogens, non-O157 E. coli, Salmonella species, Listeria (such as Listeria monocytogenes), Shigella species, Yersinia enterocolitica), Vibrio species, hospital acquired pathogens (such as Enterococcus), and agents of bioterrorism (such as Bacillus anthracis). Other exemplary bacteria include the gram-positive such as Clostridium spp., Staphylococcus spp., Streptococcus spp. and the gram-negative bacteria such as Acinetobacter spp, Bacteroides spp., Bordetella pertussis, Borrelia burgdorferi, Campylobacter spp., Chlamydia trachomatis, Coxiella burnetti, Enterobacter spp., Haemophilus influenzae, Klebsiella spp., Legionella pneumophila, Mycobacterium spp., Neisseria spp., Proteus mirabilis, Pseudomonas spp., Xanthomonas spp., and Yersinia spp (such as Yersinia pestis). While the rare base cutter XbaI has been shown to work well here, it is also contemplated that this method will work equally well with other restriction endonucleases that cut genomic DNA infrequently. Other such useful art-recognized restriction nucleases include, without limitation, AvrII, SfiI, PacI, NotI, Sse 83871, SrfI, SgrAI, BglII, SpeI, AseI, RsrII, SmaI, SalI, ApaI, CspI, SacII, BlnI, I-Ceul, SwaI, and DpnI. Such restriction enzymes may be used alone or in any combination, for example, according to the methods described herein.

The following examples are intended to illustrate, not limit, the scope of the invention.

### EXAMPLE 1

### Strains of Escherichia coli O157:H7 Differ from Each Other Primarily by Insertions or Deletions, not by Single Nucleotide Polymorphisms

The recent emergence of Escherichia coli O157:H7 as a human pathogen may correlate with a hypermutable state and plasticity of the O157 genome. The genetic events related to variations between strains of E. coli O157:H7 from human outbreaks, which differed from each other by pulsed-field gel electrophoresis patterns following XbaI digestion, were investigated. As is discussed below, this analysis demonstrated that differences between strains of O157:H7 were due to small polymorphic insertions or deletions containing XbaI sites, rather than to single nucleotide polymorphisms in the XbaI sites themselves.

The ability of E. coli O157:H7 to acquire foreign DNA sequences contributes to the plasticity of its genome (Boerlin, Cell. Mol. Life Sci. 56, 735-741 (1999)). To determine whether the plasticity of the O157 genome is due to hypermutability, a non-biased technique that determines nucleotide sequences flanking each XbaI restriction enzyme site in the O157:H7 genome and compares these sequences between different strains was performed. The enzyme XbaI was chosen as this is most commonly used to generate pulsed-field gel electrophoresis (PFGE) typing profiles currently used for differentiating isolates of E. coli O157:H7 (Harsono et al., Appl. Environ. Microbiol. 59, 3141-3144 (1993)). The results of this analysis are described below.

Results

XbaI Restriction site Polymorphism in E. coli O157 Strains.

A total of 40 XbaI sites were identified between the genomes of E. coli O157:H7 reference strains 86-24 and 933. Primer pairs were designed that flank each of these 40 XhaI sites and that amplify approximately 200-400 bp sized fragments containing these sites. Control experiments were set up to test these primer pairs with colony lysates of strains 86-24 and 933, in a hotstart-touchdown PCR reaction. The presence or absence of an amplicon, as well as the presence or absence of an XbaI site within each amplicon, was assessed by PCR, XbaI digestion, and agarose gel electrophoresis. The majority of the primer pairs (36 of 40) amplified XbaI-containing DNA fragments of equal size from both strains. However, there were four exceptions: two primer pairs derived from strain 933 failed to yield an amplicon with strain 86-24. Likewise, two primer pairs derived from strain 86-24 did not yield amplicons when strain 933 DNA was used as the template.

In addition, these 40 primer pairs were used to analyze 44 E. coli O157:H7 isolates, two isolates each from 22 different outbreaks collected by the Centers for Disease Control and Prevention (CDC). Thirty-two of the 40 primer pairs produced identical results in all 44 isolates, with any particular pair generating an amplified product of identical size and containing an internal XbaI site. None of the 40 primer pairs generated an amplified product that lacked an XbaI site, indicating that none of the 44 O157:H7 isolates contained a single nucleotide polymorphism or SNP in any of the 40 XbaI sites. On the other hand, eight primer pairs depicted in Table 1A (below) produced polymorphic results across the isolate set, amplifying identically sized products with an XbaI site in some isolates but failing to amplify any product in others.

TABLE 1

| | | | | | |
|---|---|---|---|---|---|
| | CAPS | Cloning/ | | | Tm |
| No. | Primer | Source | Length | Sequence (5′→3′) | (° C.) |

| | | | | | | |
|---|---|---|---|---|---|---|
| 1 | IK8A | Sau3Al/ | 24 | GATCTTCTTTTTTAGAGCGCCTTG | (SEQ ID NO:2) | 68 |
| | IK8B | strain 8624 | 24 | TGCCTGAGTTCACAGATAAAACAC | (SEQ ID NO:3) | 68 |
| 2 | IK25A | Sau3Al/ | 24 | GCGTAATGACTTAATGATTTTCGT | (SEQ ID NO:4) | 64 |
| | IK25B | strain 8624 | 24 | CATCACATTCCTGACGCAGTGCTT | (SEQ ID NO:5) | 72 |
| 3 | IK114A | NlaIII/ | 24 | GAGAATATTATCAGCGACTTGATA | (SEQ ID NO:6) | 64 |
| | IK114B | strain 933 | 24 | CTAGATCAACTGAGACAGATTATA | (SEQ ID NO:7) | 64 |
| 4 | IK118A | NlaIII/ | 20 | CATGATTGGCTGGCGTCCCT | (SEQ ID NO:8) | 64 |
| | IK118B | strain 933 | 20 | ACCAATGAAATGAGTTCAGA | (SEQ ID NO:9) | 54 |
| 5 | IK123A | NlaIII/ | 24 | TGAAAGTAAACGAAAATTGGCTTC | (SEQ ID NO:10) | 64 |
| | IK123B | strain 933 | 24 | AAAGAATATCCGGCCCTTCTATCT | (SEQ ID NO:11) | 68 |
| 6 | IK127A | NlaIII/ | 24 | ATGTTGAGTATATTGGGCAAGACA | (SEQ ID NO:12) | 66 |
| | IK127B | strain 933 | 24 | GAAATATCGATAACAGACGCTCTC | (SEQ ID NO:13) | 68 |
| 7 | IKB3A | Strain 933/ | 24 | GAGAAGCCTTGCTTCATTAAAGTA | (SEQ ID NO:14) | 66 |
| | IKB3B | Blattner | 24 | ATGAAGCTGTTTTGGCTGCACTAT | (SEQ ID NO:15) | 68 |
| 8 | IKB5A | Strain 933/ | 24 | ATCTGAAAGATCTGCATTTGATAT | (SEQ ID NO:16) | 62 |
| | IKB5B | Blattner | 24 | GATTGTAAGCTAATATCAGCTCAT | (SEQ ID NO:17) | 64 |

In these latter cases, the presence or absence of an amplicon by PCR correlated with the presence or absence of a hybridizing fragment by Southern blot analysis of genomic DNAs isolated from the corresponding isolates, using control PCR amplicons as probes (data not shown). A single exception was observed with one amplicon (IK8) as a probe. This fragment hybridized to genomic DNA isolated from all 44 isolates, irrespective of whether an amplified product was obtained from any particular isolate using the IK8A/B PCR primer pair. Further evaluation revealed that one of the IK8 primers (IK8B) corresponded to the 5′ end of the IS629tnp gene, which is widely distributed over the O157 genome (see below).

The DNA sequences amplified by the 40 primer pairs were analyzed using the Genbank database (BLAST search program, NCBI) and the E. coli O157:H7 strain 933 genome sequence database (University of Wisconsin). Of the 40 O157:H7 XbaI-containing genome sequences amplified by the primer pairs, 18 were homologous to E. coli strain K-12 genome sequences (referred to as backbone sequences (Perna et al., Nature 409, 463-466 (2001)) and 22 were in regions of the O157:H7 chromosome not shared with K-12, referred to as O-islands (SEQ ID NO.: 1) (Perna et al., Nature 409, 463-466 (2001)). The majority of these O-islands (19 of 22) occurred as distinct inserts interrupting homology to the K-12 genome at the site of insertion. Three of the O-islands replaced other sequences at the same site on the K-12 genome. All of the eight polymorphic regions that were present in some but not in other E. coli O157:H7 isolates were localized to O-islands, compared to 14 of the 32 amplified sequences that were conserved across all isolates tested (p<0.01), suggesting that the major genetic differences between O157:H7 strains occur in O-island sequences.

Three of the eight polymorphic regions were analyzed in more detail to gain insight into the mechanisms underlying strain differences. Additional primers were designed either from 933 or 86-24 genomic sequences to amplify regions upstream, downstream, or across the polymorphic region being evaluated. The various amplicons were purified, assessed for the presence or absence of an internal XbaI site, and sequenced. This analysis confirmed that all three regions examined, defined by primer pairs IK8A/B, IKB3A/B, and IKI 18A/B, were polymorphic in different O157:H7 isolates because of small insertions or deletions that contained XbaI sites, rather than because of single nucleotide polymorphisms or SNPs in the XbaI sites themselves.

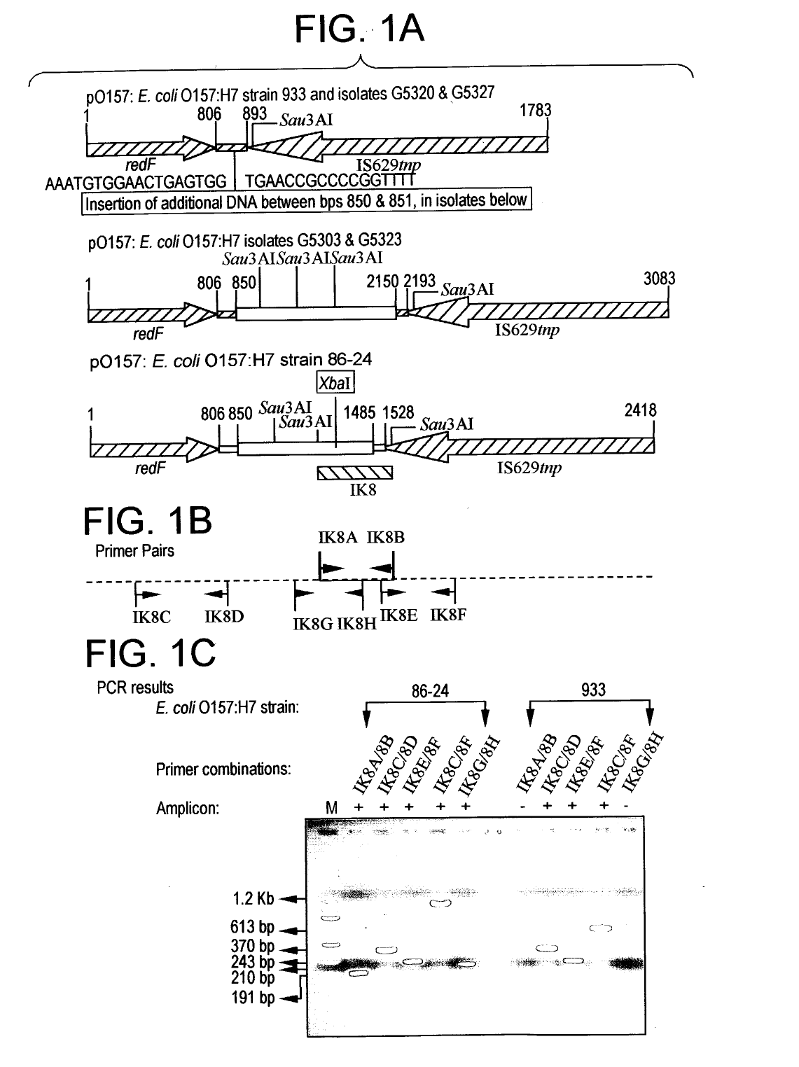

For example, polymorphism between isolates for the XbaI-containing fragment amplified by IK8A/B was a consequence of a small insertion in the virulence plasmid. Using the primer pair IK8A/B, an amplicon was obtained from E. coli O157:H7 strain 86-24 but not from strain 933. As shown in FIG. 1A, this amplicon, referred to as IK8, specifically extended from a region of unknown function into a transposase gene (IS629tnp) located on the virulence plasmid, pO157, in strain 86-24 (Genbank Accession no. AB011549) (Makino et al., DNA Res. 5, 1-9 (1998). The region of unknown function occurred as a 635 bp insertion in the DNA between the resolvase (redf) and IS629tnp genes in strain 86-24, compared to the sequence of the same region in plasmid pO157 from E. coli O157:H7 strain 933 (FIG. 1A; Genbank Accession no. AF074613) (Burland et al., Nucleic Acids Res. 26, 4196-4204 (1998)); the insertion in strain 86-24 contained an XbaI site.

Primer pairs IK8C/D, IK8E/F, and IK8G/H were designed to amplify sections of redF and IS629tnp, and the insertion in strain 86-24 for further analysis (FIG. 1B). Identical amplicons were obtained from strains 86-24 and 933 using the first two sets of primers, indicating conservation of the respective genes on both plasmids (FIG. 1C); these amplicons were not cleaved with XbaI. On the other hand, an amplicon was obtained with IK8G/H only from strain 86-24 (FIG. 1C) and it contained an XbaI site (data not shown). The primer combination of IK8C/F was used to amplify the entire length of this region in both strains. The size difference in the resulting amplicons (1.2 kb from strain 86-24 and 613 bp from strain 933) confirmed the earlier observation that pO157 from strain 86-24 contained a 635 bp insertion between bp 850 and 851 of pO157 in strain 933 (FIG. 1A). BLAST search analysis revealed no homologies for the inserted sequence in strain 86-24.

These same primer pairs were used to analyze four additional isolates of E. coli O157:H7, G5320, G5327, G5303, and G5323, randomly chosen from the CDC isolates that did not yield an amplicon with primer pair IK8A/B. Amplicons derived from isolates G5320 and G5327, using primer pair IK8C/F, were of the same size as that from strain 933 (613 bp) indicating the absence of an insertion (FIG. 1A). Using these primers, amplicons generated from isolates G5303 and G5323 revealed a 1.3 kb insert, but this insert did not contain an internal XbaI site (FIG. 1A). Failure to obtain amplicons from isolates G5303 and G5323 with primer pairs IK8A/B and IK8G/H showed that isolates G5303 and G5323 contained a different insertion than that in 86-24. The sequences flanking the point of insertion were, however, identical for all isolates tested, including 86-24, G5303, and G5323 (FIG. 1A). BLAST search analysis revealed that the insert in isolates G5303 and G5323 had 99% homology to three open reading frames (ORFs), L0013, L0014, and L0015, in the LEE pathogenicity island of E. coli O157:H7 strain 933 (Perna, N. T. et al. Infect. Immun. 66, 3810-3817 (1998)). These three ORFs comprise ISEc8 in strain 933, an insertion element similar to ISRm14 present in Rhizobium and Agrobacterium plasmids (Schneiker et al., Curr. Microbiol. 39, 274-281 (1999)); however, the homologous insert in isolates G5303 and G5323 contained only part of the L0015 ORF and not the complete IS element. The G+C content was determined for the sequences shared between all isolates (shown as filled-in black arrows and bars in FIG. 1A; 51%), the inserted sequence in strain 86-24 (33%), and the inserted sequence in strains G5303 and G5323 (55%). The G+C content of E. coli K-12 is 50.8% (Boerlin, Cell. Mol. Life Sci. 56, 735-741 (1999); Blattner et al., Science 277, 1453-1474 (1997)). The lower G+C content of the insert in strain 86-24 is suggestive of a possible heterologous origin (Boerlin, Cell. Mol Life Sci. 56, 735-741 (1999); Blattner et al., Science 277, 1453-1474 (1997)). The higher G+C content of the insert in G5303 and G5323 reflects the possible origin of this sequence from the Rhizobium and Agrobacterium genomes of high G+C (57 to 63%) composition (Nisslein et al., Appl. Environ. Microbiol. 64, 1283-1289 (1998)). These observations suggested that polymorphisms between different strains of E. coli O157:H7 reflect the acquisition or loss of small, discrete segments of DNA in the genome, at least some of which may be of heterologous origin.

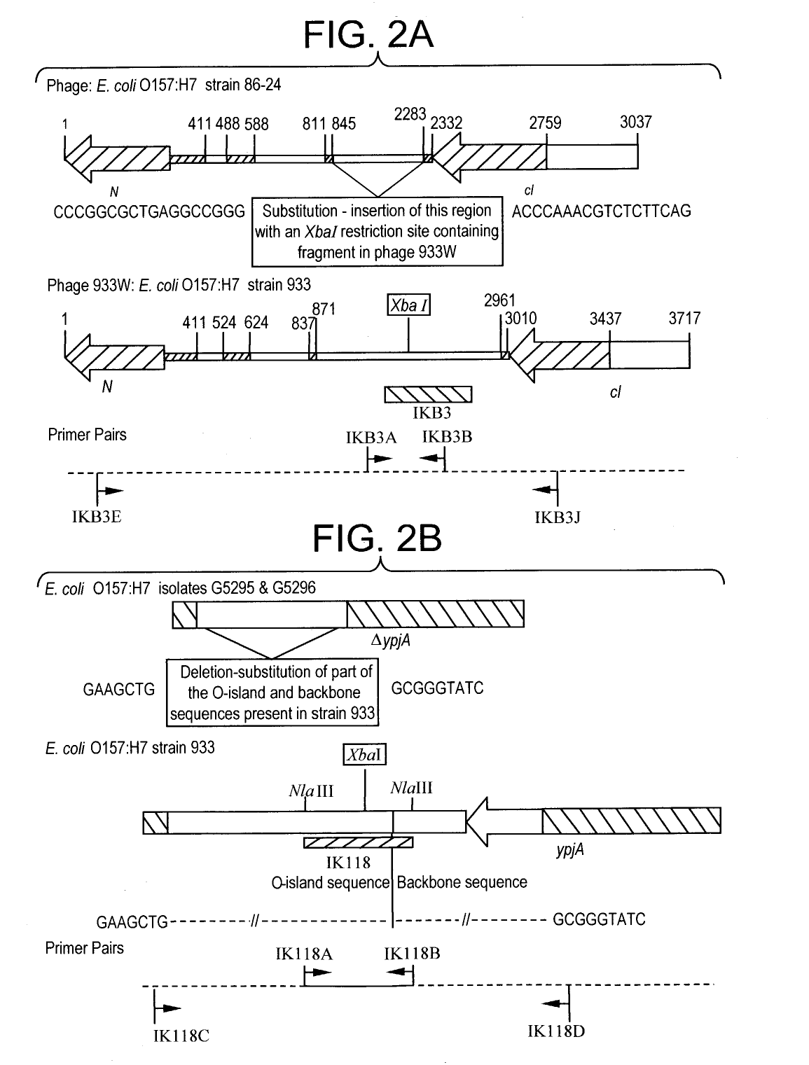

Similar analysis of the XbaI-containing fragment amplified by IKB3A/B linked the polymorphism in this region to a substitution-insertion in a lysogenic bacteriophage. Using the primer pair IKB3A/B, an amplicon was obtained from E. coli O157:H7 strain 933 but not from strain 86-24. This amplicon, referred to as IKB3, was mapped to the lysogenic bacteriophage 933W in strain 933 (Genbank Accession no. AF125520) (Plunkett et al., J. Bacteriol. 181, 1767-1778 (1999)). As shown in FIG. 2A, the IKB3 sequence overlapped a 2,091 bp insertion, containing an internal XbaI site, which was present between the anti-terminator protein (N) and repressor protein (cI) genes in phage 933W. This insertion replaced a 1,439 bp sequence, located at exactly the same site on a similar bacteriophage in E. coli O157:H7 strain 86-24, but which lacked an XbaI site (FIG. 2A); hence, this region was referred to as a substitution-insertion. Four additional isolates, G5290, G5325, G5296, and G5301, chosen randomly from the CDC isolates that did not yield an amplicon with primer pair IKB3A/B, were analyzed using a primer pair IKB3E/J that would amplify the entire length of this substitution-insertion (FIG. 2A). No amplicons were obtained from isolates G5325, G5296, and G5301 (Table 1), indicating that this region in these isolates is even more divergent than 86-24 from 933. This was confirmed by additional PCR reactions using primer pairs designed to amplify various segments of the region between IKB3E and IKB3J, which also failed to yield amplicons from the three isolates (data not shown). In contrast, the primer pair IKB3E/J yielded an amplicon from isolate G5290 that was identical in size to that from strain 86-24 (Table 1) and lacked an XbaI site. Thus, this region has at least three variants in the E. coli O157:H7 population studied.

TABLE 1
Further analysis of the region surrounding the sequence amplified
by the primer pair IKB3A/B.

| | Amplicons derived from E. coli O157 isolates: | | | | | |
|---|---|---|---|---|---|---|
| Primer pairs | 86-24 | 933 | G5290 | G5325 | G5296 | G5301 |
| IKB3A/B3B | — | 193 bp^{a} | — | — | — | — |
| IKB3E/B3J | 2.6 kb^{b} | 3.2 kb^{a} | 2.6 kb^{b} | — | — | — |

^{a}Amplicons contained an XbaI restriction site.
^{b}Amplicon with a different sequence compared to strain 933 and lacking an XbaI restriction site.

Analysis of a third XbaI-containing fragment amplified by IKI 18A/B, which also differed between isolates, demonstrated a polymorphism linked to a deletion-substitution in the chromosome. Using the primer pair IKI 18A/B, an identical amplicon containing an XbaI site was obtained from most E. coli O157:H7 strains/isolates tested. This amplicon, referred to as IK118, was mapped to a chromosomal DNA segment in E. coli O157:H7 strain 933 that extended across a junction between O-island and backbone sequences (FIG. 2B). The backbone sequence contained the putative transport gene, ypjA (Genbank Accession no. AE000350) (Perna et al., Infect. Immun. 66, 3810-3817 (1998); Rudd, Microbiol. Mol. Biol. Rev. 62, 985-1019 (1998)). While this entire region, along with its XbaI site, was conserved in most of the E. coli O157:H7 isolates/strains tested, no amplicons were obtained from isolates G5295 and G5296 using IK 18A/B.

E. coli O157:H7 strain 933 and isolates G5295 and G5296 were analyzed using the primer pair IK118C/D that amplifies across part of the O-island and backbone sequence into the 3′ end of ypjA (FIG. 2B). A 1.5 kb amplicon containing an XbaI site was obtained from strain 933. In contrast, isolates G5295 and G5296 had replaced this 1.5 kb region with a different 1 kb of sequence, which lacked an XbaI site, did not contain any ORFs, and contained a deletion of the 3′ end of ypjA (FIG. 2B). Hence, this region is referred to as a deletion-substitution. The deletion-substitution in G5295 and G5296 may have been caused by the excision of a prophage in these isolates. Cryptic prophage genes have been identified in the O-island region adjacent to this O-island-backbone junction in E. coli O157:H7 strain 933 (Table 2) (Pema et al., Nature 409, 463-466 (2001)).

TABLE 2

| Amplicon | Length of | | | |
|---|---|---|---|---|
| derived from | associated O- | | | |
| E. coli | island in E. coli | Position of Xbal | | |
| O157:H7 | O157:H7 strain | site from one end | Description of O- | Relation of O-island to |
| isolates | 933 | of O-island | island | E. coli K-12 genome |
| B3 | 61,664 bp | 11,088 bp | Stx2-encoding | Insertion |
| | | | prophage BP-933W | |
| 118 | 21,681 bp | 21,637 bp | Cryptic prophage CP- | Replaces unrelated |
| | | | 933Y | sequences in K-12 |
| B5 | 49,798 bp | 36,431 bp | Cryptic prophage CP- | Partial homology to |
| | | | 933R | cryptic prophage Rac of |
| | | | | K-12 |
| 114 | 44,434 bp | 8,367 bp | Large island adjacent | Replaces unrelated |
| | | | to leuX; includes a | sequences in K-12 |
| | | | putative site-specific | |
| | | | integrase/recombinase, | |
| | | | several IS elements, | |
| | | | putative helicases and | |
| | | | numerous unknowns | |
| 123 | 80,502 bp | 35,859 bp | Cryptic prophage CP- | Replaces unrelated |
| | | | 933O | sequences in K-12 |
| 127 | 21,120 bp | 19,318 bp | Cryptic prophage CP- | Insertion |
| | | | 933T | |

In addition to IK8, IKB3, and IK118, the remaining five regions polymorphic between isolates were also found in O-islands absent in the K-12 genome. Six of the 8 polymorphic regions (IKB3, IK118, IKB5, IK114, IK123, and IK127) were present in strain 933 and the availability of the genome sequence of this strain allowed us to determine the properties of the O-islands containing these six regions (Table 2). The remaining two polymorphic regions were present in strain 86-24, but not in the sequenced strain 933, the larger genomic context therefore remained undefined.

The observations concerning the differences between strains of E. coli O157:H7 are consistent with the conclusion that the high frequency of mutation among E. coli and Salmonella pathogens is due to their existence in a state of transient or permanent hypermutability, which can affect both the acquisition of heterologous sequences as well as point mutations (LeClerc et al., Science, 274, 1208-1211 (1996)). Specifically, the presence or absence of polymorphic XbaI sites in the O157 genome was found to be a consequence of the insertion or deletion of discrete segments of DNA in the genome, rather than SNPs in individual XbaI sites. The inserted sequences containing the polymorphic XbaI sites were quite small and usually neither encoded a functional open reading frame nor disrupted a pre-existing open reading frame. An exception was the deletion-substitution observed in isolates G5295 and G5296, which resulted in the loss of 327 bp in the 3′ end of ypjA. However, this deletion did not apparently affect either the viability or pathogenicity of these isolates as they were recovered from human infection. The inserted sequences analyzed were not intact insertion sequences, transposons, or bacteriophages. However, several of the inserted sequences were found within O-islands that contained nearby cryptic prophage genes (Table 2), suggesting that phage-mediated events may underlie their acquisition or loss. The inserted sequences were consistently found in intergenic regions. Sequences that characterize mutational hot spots or other composition variations (van Belkum et al., Microbiol. Mol. Biol. Rev. 62, 275-293 (1998)) were not observed in the sequences flanking the insertion points, although each set of insertions occurred at exactly the same nucleotide position between strains. The analysis of O-islands in the strain 933 genome that contain these polymorphic sequences further indicates that the major events driving evolution of the E. coli O157:H7 genome are not point mutational events, but rather insertions/deletions of discrete DNA sequences.

Detailed Materials and Methods

Described below are detailed materials and methods relating to the above-described experiments. In the case of the XbaI sites, these experiments show that strains of Escherichia coli O157:H7 differ from each other primarily by insertions or deletions, not by single nucleotide polymorphisms.

Bacteria.

E. coli O157:H7 strain 86-24, streptomycin resistant and originally isolated from a human in a Washington State outbreak, was kindly provided by Dr. A. D. O'Brien. E. coli O157:H7 strain 933, a human isolate from a Michigan State outbreak, was obtained from the American Type Culture Collection (ATCC, Manassas, Va.) which has it banked as ATCC 43895. Strain 933 is the O157 isolate that has been sequenced at the University of Wisconsin-Madison, Madison, Wis. In addition, 44 isolates of E. coli O157:H7, two each from 22 different outbreaks collected by the CDC, Atlanta, Ga., were also included in this study. The isolates from different outbreaks had different PFGE patterns suggesting genetic heterogeneity amongst them. The CDC numbers assigned to these isolates were as follows: G5320, G5327; G5323, G5326; G5321, G5322; G5324, G5325; G5283, G5284; G5285, G5286; G5287, G5288; G5289, G5290; G5291, G5292; G5293, G5294; G5295, G5296; G5297, G5298; G5317, G5318; G5299, G5300; G5301, G5302; G5303, G5304; G5305, G5306; G5307, G5308 (Garden); G5309, G5310 (Meat); G5311, G5312; and G5313, G5314; G5315, G5316. Forty-two of the 44 isolates were isolated from human clinical cases.

Design of Primer Pairs Amplifying E. coli O157:H7XbaI Sites.

Genomic DNA from E. coli O157:H7 strains 86-24 and 933 was initially fragmented using Sau3AI (strain 86-24) or NlaIII (strain 933), followed by self-ligation. The circularized DNA was digested with the restriction enzyme XbaI to linearize only fragments containing an internal XbaI site. These fragments were selectively cloned into pBluescribe (Stratagene USA, LaJolla, Calif.) and sequenced. Insert sequences were used to design twenty-two primer pairs flanking different XbaI restriction sites; these were prefixed IK. An additional eighteen primer pairs, with the prefix IKB, were designed using the E. coli O157:H7 strain 933 genomic sequence being assembled at the University of Wisconsin-Madison, Madison. Wis. Additional information on the design of primers is provided in Example 2 (below).

PCR Conditions for Primer Pairs Amplifying XbaI Sites.

Colony lysates were prepared by boiling colonies suspended in sterile distilled water, followed by centrifugation at 4° C. Each E. coli O157:H7 strain template was tested with each individual primer pair in separate reactions. PCR was carried out on the GeneAmp PCR system 2400 thermal cycler (PE Biosystems, Foster City, Calif.), using 10 μl of colony lysate, 200 pmoles of each primer, 800 μM dNTPs, 1× diluted Ex Taq enzyme buffer, and 2.5 units of TaKaRa Ex Taq™ DNA polymerase. The hot start PCR technique was employed in which the polymerase was added only after preheating the rest of the PCR mix (Dieffenbach, C. W. & Dveksler, G. S., eds., PCR Primer—A Laboratory Manual, Cold Spring Harbor Laboratory Press, NY, 1995). This technique was used in combination with a Touchdown PCR profile (Don et al., Nucleic Acids Res. 19, 40008 (1991)). To create this profile, the regular PCR program was modified as follows: an amplification segment of 20 cycles was set where the annealing temperature started at 73° C., to touchdown at 53° C. at the end of those cycles. Then, another amplification segment of 10 cycles was set, using the last annealing temperature of 53° C. Each reaction was done in triplicate.

Evaluation of XbaI Amplicons.

Amplicons obtained by PCR were purified using the QIAQUICK PCR purification kit and digested with XbaI to confirm the presence of an XbaI site within the amplicon. Undigested and digested DNA fragments were resolved on a 4% agarose gel prepared with a combination of 3% Nusieve GTG agarose (FMC BioProducts, Rockland, Me.) and 1% agarose (Shelton Scientific Inc., Shelton, Conn.) and stained with ethidium bromide. Sequencing of purified amplicons was done at the DNA Sequencing Core Facility, Department of Molecular Biology, Massachusetts General Hospital. This facility uses ABI Prism DiTerminator cycle sequencing with AmpliTaq DNA polymerase FS and an ABI 377 DNA sequencer (Perkin-Elmer Applied Biosystems Division, Foster City, Calif.) for this purpose.

Southern Blots.

DNA was fractionated by agarose gel electrophoresis, transferred to Hybond-N+ membranes (Amersham Pharmacia Biotech, Inc., Piscataway, N.J.), U.V. crosslinked to the membrane using a Stratalinker (Stratagene), and hybridized with the appropriate probe, labeled using the ECL direct nucleic acid labeling and detection system (Amersham Pharmacia). Hybridization at 42° C. and post-hybridization washing of blots was done as per the ECL kit manual. Autoradiographs were prepared by exposure of processed blots to Kodak Scientific Imaging X-OMAT AR film (Eastman Kodak Company, Rochester, N.Y.).

Data Analysis.

Statistical analysis was performed using the EpiInfo6 (CDC) software. The significance of differences in proportions was calculated with χ² test, or Fisher's exact test if the size of any cell was ≦5. DNA %G+C was determined using the Wisconsin Package Version 10.0, Genetics Computer Group (GCG), Madison, Wis.

### Example 2

### Polymorphic Amplified Typing Sequences Provide a Novel Approach to Escherichia coli O157:H7 Strain Typing

As is discussed above, E. coli O157:H7 strains have been shown to differ from each other by a series of small insertions or deletions of DNA, some of which contain recognition sites for restriction enzymes. These insertions and deletions determine the complement of XbaI restriction sites in the genome of a given strain and hence detection of these XbaI-containing sequences should provide information comparable to PFGE following XbaI digestion. Below, the potential of directly detecting these polymorphic sequences by designing a new, simple strain typing system for E. coli O157:H7, which has been termed polymorphic amplified typing sequences or PATS, is demonstrated.

As is described above in Example 1, using two reference O157 strains, a total of forty genomic sequences that contained XhaI sites were used to generate 40 primer pairs that flanked each individual XbaI site. These primer pairs were then used to amplify 200-400 bp fragments of the surrounding genomic DNAs. In particular, these primer pairs were tested with 44 O157 isolates, two each from 22 different outbreaks investigated by the Centers for Disease Control. Of the 40 primer pairs, 32 amplified identical XbaI-containing fragments from all 44 isolates, whereas eight produced polymorphic results between isolates, amplifying identical XbaI-containing fragments from some but producing no amplicons from others. As is described in more detail below, the 44 isolates were differentiated into 14 groups based on which of the eight polymorphic amplicons were detected; phylogenetic analysis divided the isolates into four major clusters. PATS correctly identified 21 of 22 outbreak pairs as identical or highly related, compared to 14 of 22 identified as such by PFGE; PATS also was able to type isolates from three outbreaks that were untypeable by PFGE. However, PATS was less sensitive than PFGE in discriminating between outbreaks. These data demonstrated that PATS provided a simple procedure for strain typing not only O157, but also other bacteria.

Results

PATS Primer Pairs.

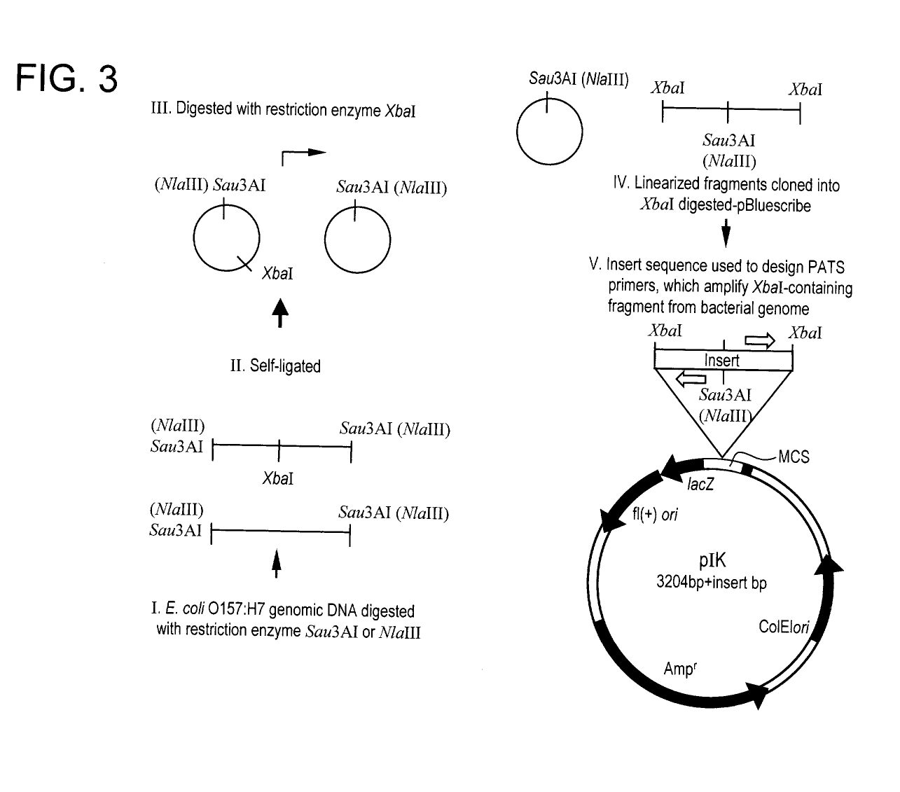

PATS primer pairs to 40 XbaI sites (and flanking DNA sequences) between the genomes of E. coli O157:H7 strains 86-24 and 933 were prepared as follows. (A) Using Sau3AI-digested, genomic fragments of E. coli O157:H7 strain 86-24 (FIG. 3): Recombinant plasmids pIK1-100 containing E. coli O157:H7 strain 86-24 inserts, derived by digestion of genomic DNA by Sau3AI and recovery of inserts containing individual XhaI sites were constructed (FIG. 3). Duplicates among these were eliminated by Southern blot analysis prior to sequencing (data not shown) and insert sequences were used to design primer pairs that flanked the genomic XbaI restriction sites. Of these 100 plasmids, twelve were found to possess distinct, non-overlapping insert sequences. Primer pairs IK1A/B, IK2A/B, IK8A/B, IK10A/B, IK12A/B, IK18A/B, IK23A/B, IK25A/B, IK38A/B, IK39A/B, IK51A/B, and IK56A/B were derived from these insert sequences. Numbers used to label primer pairs match the pIK plasmid used to design them.

(B) Using NlaIII-digested genomic fragments of E. coli O157:H7 strain 933 (FIG. 3): Similar to the construction of plasmids pIKI-100, plasmids pIK101-150 contained inserts from E. coli O157:H7 strain 933, derived by digestion of genomic DNA by NlaIII and recovery of inserts containing individual XbaI sites (FIG. 3). These 50 plasmids were analyzed as above and ten of these were found to contain unique insert sequences. Primer pairs IK111A/B, IK114A/B, IK116A/B, IK117A/B, IK118A/B, IK123A/B, IK127A/B, IK131A/B, IK142A/B, and IK148A/B were derived from these insert sequences.

(C) Using the genome sequence of E. coli O157:H7 strain 933: Of the DNA fragments containing XbaI sites identified by sequencing of the E. coli O157:H7 strain 933, 18 did not match sequences already identified in pIK1-150. Sequences of these 18 fragments were used to design 18 additional PATS primer pairs designated with IKB numbers (IKB1A/B, IKB3A/B, IKB4A/B, IKB5A/B, IKB6A/B, IKB7A/B, IKB8A/B, IKB9A/B, IKB10A/B, IKB13A/B, IKB14A/B, IKB15A/B, IKB16A/B, IKB17A/B, IKB18A/B, IKB19A/B, IKB20A/B, and IKB21A/B), thereby increasing the overall total of PATS primer pairs to forty.

PATS Primer Pairs Amplify Sequences in the E. coli O157:H7 Genome Containing XbaI Restriction Sites.

Control PCR experiments were set up to test the PATS primer pairs, using colony lysates and genomic DNA of E. coli O157:H7 strains 86-24 and 933 as templates. The PATS primer pairs amplified DNA fragments (one amplicon per primer pair) containing a single XbaI restriction site, from templates corresponding to the E. coli O157:H7 strain used to design them. Identical results were obtained with both the lysate and purified DNA templates (data not shown).

The majority of the PATS primer pairs amplified XbaI-containing DNA fragments of identical size from both control strains. However, there were four exceptions. PATS primer pairs IK114A/B and IKB3AIB, derived from strain 933, failed to yield an amplicon with strain 86-24. Likewise, PATS primer pairs IK8A/B and IK25A/B, derived from strain 86-24, failed to amplify when strain 933 DNA was used as the template. Thus, the PATS primer pairs were able to establish a discriminating profile between the two strains, based on the presence or absence of amplicons.

PATS Primers Provide a Strain Typing System for E. coli O157:H7.

The ability of the 40 PATS primer pairs to discriminate E. coli O157:H7 isolates in a reproducible manner was assessed. To enhance the profile for each isolate being typed, primer pairs derived from four virulence genes (stx₁, stx₂, eae, and hlyA), often (but not always) found in E. coli O157:H7, were also included in the PATS typing system. Based on results obtained with the control strains, colony lysates were used as templates for PCR and the presence/absence of amplicons, as well as the presence/absence of an XbaI site within each amplicon, was assessed by agarose gel electrophoresis. Results were recorded using the digits 0, 1, or 2, indicating the absence of an amplicon, the presence of an amplicon without an XhaI site, and the presence of an amplicon with an XbaI site, respectively.

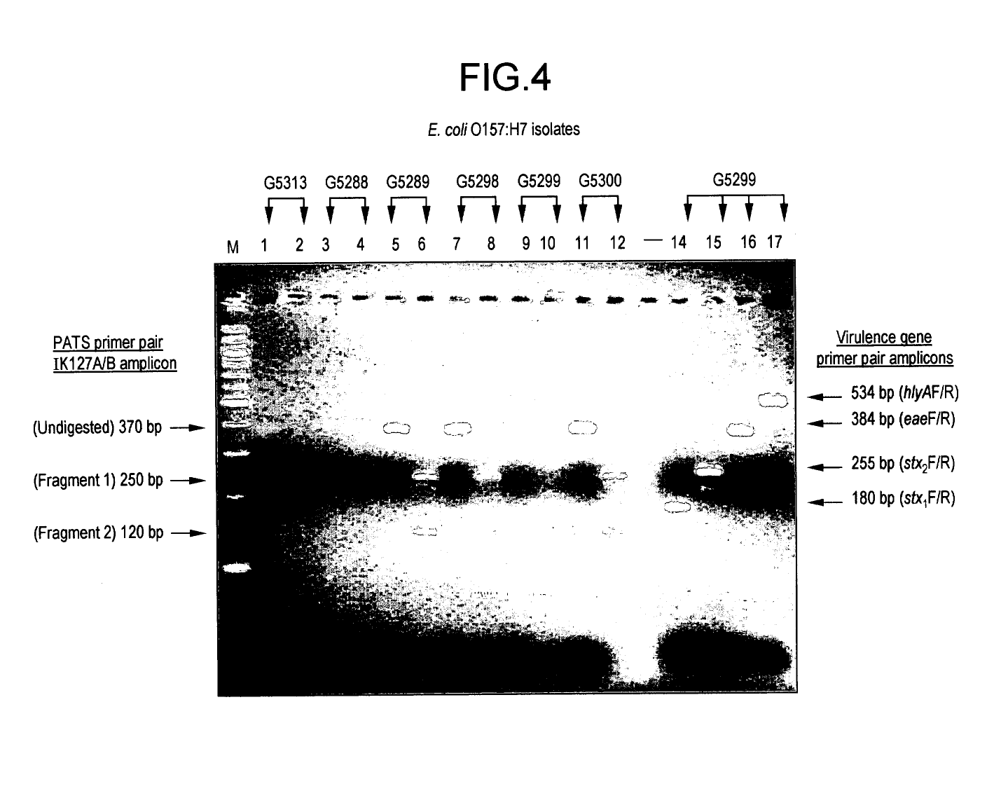

Forty-four isolates of E. coli O157:H7, two each from 22 different outbreaks (Table 3), were analyzed using this typing system. The presence or absence of an XbaI site within each amplicon was assessed by agarose gel electrophoresis. A representative agarose gel electrophoresis pattern of undigested and XbaI-digested amplicons obtained from some of the isolates is shown in FIG. 4. All amplicons derived using the PATS primer pairs had a score of 0 or 2; i.e. all isolates that had an amplicon with a given primer pair always had an internal XbaI site in the amplicon, as seen originally in the control strain used to design the PATS primers. Amplicons obtained with the virulence gene primer pairs had a score 0 or 1. Based on the score assigned to each amplicon obtained from every isolate-primer pair combination tested, the 44 E. coli O157:H7 isolates were differentiated into 14 PATS types, arbitrarily designated A through N (Table 5). The most common PATS types were E and G. The reproducibility of this typing system was demonstrated by the consistency of profiles obtained in three separate analyses of the 44 outbreak isolates.

TABLE 3
Summary of E. coli O157:H7 isolates used in this study

| | | Outbreak | Outbreak | |
|---|---|---|---|---|
| Isolates | Description/Source | number | location | Year |
| 86-24 | Human; Sm^{r} strain; Dr. A. D. | NA^{a} | NA | NA |
| | O'Brien, personal communication | | | |
| 933 | Human; American Type Culture | NA | NA | NA |
| | Collection | | | |

From the Center for Disease Control:

| | | | | |
|---|---|---|---|---|
| G5320, G5327 | Human | 1 | Michigan | 1982 |
| G5323, G5326 | Human | 2 | Oregon | 1982 |
| G5321, G5322 | Human | 3 | Nebraska | 1984 |
| G5324, G5325 | Human | 4 | North Carolina | 1984 |
| G5283, G5284 | Human | 5 | North Carolina | 1986 |
| G5285, G5286 | Human | 6 | Washington | 1986 |
| G5287, G5288 | Human | 7 | Washington | 1986 |
| G5289, G5290 | Human | 8 | Washington | 1986 |
| G5291, G5292 | Human | 9 | Utah | 1987 |
| G5293, G5294 | Human | 10 | Wisconsin | 1988 |
| G5295, G5296 | Human | 11 | Minnesota | 1988 |
| G5297, G5298 | Human | 12 | Minnesota | 1988 |
| G5317, G5318 | Human | 13 | Missouri | 1990 |
| G5299, G5300 | Human | 14 | Idaho | 1990 |
| G5301, G5302 | Human | 15 | Montana | 1991 |
| G5303, G5304 | Human | 16 | Massachusetts | 1991 |
| G5305, G5306 | Human | 17 | Nevada | 1992 |
| G5307, G5308 | Human, Garden | 18 | Maine | 1992 |
| G5309, G5310 | Human, Meat | 19 | Washington | 1993 |
| G5311, G5312 | Human | 20 | Oregon | 1993 |
| G5313, G5314 | Human | 21 | Oregon | 1993 |
| G5315, G5316 | Human | 22 | Oregon | 1993 |

^{a}NA, not applicable.

The typing patterns observed for these isolates and control strains of E. coli O157:H7 were further verified via Southern blot analysis (data not shown). The presence or absence of an amplicon by PCR corresponded with the presence or absence of a hybridizing fragment in genomic DNA, using the control PCR amplicon as a probe (data not shown). A single exception was observed when the IK8A/B amplicon was used as a probe. This fragment hybridized to DNA from all strains by Southern blot, irrespective of the PCR result. As is described in Example 1, the IK8A/B amplicon partially overlaps the IS629tnp gene, which is widely distributed over the O157 genome.

XbaI sites that differ between different O157:H7 strains are located on inserted or deleted O157-specific sequences.

The only differences in the PATS profiles between strains occurred with eight of the 40 PATS primer pairs, which amplified regions of the E. Coli O157:H7 genome that were polymorphic between strains (Tables 4 and 5); that is, these eight primer pairs failed to yield an amplification product in some of the strains tested. These eight PATS primer pairs included IK8A/B, IK25A/B, IK114A/B, IK118A/B, IK123A/B, IK127A/B, IKB3A/B, and IKB5A/B. Regions amplified by the remaining 32 PATS primer pairs were conserved across all strains tested (Tables 4 and 5); that is, for each of these 32 primer pairs, all strains tested had an identically sized PCR product with a conserved XbaI site. As is described in Example 1, the eight PATS primer pairs that yielded polymorphic results between strains, amplified regions of DNA that were inserted or deleted between strains and were all localized in so-called O-island sequences, which are specific to the 0157 genome and not found in E. coli K-12 (Table 4).

TABLE 4
E. coli O157 genomic regions amplified by the 40 PATS primer
pairs designed flanking XbaI restriction enzyme sites.

| Regions conserved across all | | Regions polymorphic | |
|---|---|---|---|
| strains | | between strains | |
| | Location in E. coli | | Location in E. coli |
| Primer Pair | O157 genome | Primer Pair | O157 genome |
| IK1A/B | Backbone^{a} | IK8A/B | O-island |
| IK2A/B | Backbone | IK25A/B | O-island |
| IK10A/B | Backbone | IK114A/B | O-island |
| IK12A/B | Backbone | IK118A/B | O-island |
| IK18A/B | O-island^{b} | IK123A/B | O-island |
| IK23A/B | Backbone | IK127A/B | O-island |
| IK38A/B | Backbone | IKB3A/B | O-island |
| IK39A/B | O-island | IKB5A/B | O-island |
| IK51A/B | Backbone | | |
| IK56A/B | Backbone | | |
| IK111A/B | O-island | | |
| IK116A/B | Backbone | | |
| IK117A/B | Backbone | | |
| IK131A/B | Backbone | | |
| IK142A/B | O-island | | |
| IK148A/B | Backbone | | |
| IKB1A/B | O-island | | |
| IKB4A/B | Backbone | | |
| IKB6A/B | O-island | | |
| IKB7A/B | O-island | | |
| IKB8A/B | O-island | | |
| IKB9A/B | O-island | | |
| IKB10A/B | O-island | | |
| IKB13A/B | O-island | | |
| IKB14A-1/B-1 | Backbone | | |
| IKB15A/B | O-island | | |
| IKB16A/B | Backbone | | |
| IKB17A/B | O-island | | |
| IKB18A/B | Backbone | | |
| IKB19A/B | Backbone | | |
| IKB20A/B | O-island | | |
| IKB21A/B | Backbone | | |

^{a}Backbone, DNA sequences homologous to E. coli K-12 genome.
^{b}O-island DNA sequences unique to E. coli O157 genome.

TABLE 5
PATS profiles of E. coli O157:H7 isolates.

| | PCR amplification and XbaI restriction digestion patterns of amplicons obtained using | | | | | | | | | | | | | | | | |
|---|---|---|---|---|---|---|---|---|---|---|---|---|---|---|---|---|---|
| PATS | 40 PATS - 4 virulence gene primer pairs^{b} | | | | | | | | | | | | | | | | |
| type^{a} | IK1 | IK2 | IK8 | IK10 | IK12 | IK18 | IK23 | IK25 | IK38 | IK39 | IK51 | IK56 | IK111 | IK114 | IK116 | IK117 | IK118 |
| Control | 2 | 2 | 2 | 2 | 2 | 2 | 2 | 2 | 2 | 2 | 2 | 2 | 2 | 0 | 2 | 2 | 2 |
| Control | 2 | 2 | 0 | 2 | 2 | 2 | 2 | 0 | 2 | 2 | 2 | 2 | 2 | 2 | 2 | 2 | 2 |
| A | 2 | 2 | 2 | 2 | 2 | 2 | 2 | 2 | 2 | 2 | 2 | 2 | 2 | 0 | 2 | 2 | 2 |
| B | 2 | 2 | 0 | 2 | 2 | 2 | 2 | 0 | 2 | 2 | 2 | 2 | 2 | 2 | 2 | 2 | 2 |
| C | 2 | 2 | 2 | 2 | 2 | 2 | 2 | 0 | 2 | 2 | 2 | 2 | 2 | 2 | 2 | 2 | 2 |
| D | 2 | 2 | 2 | 2 | 2 | 2 | 2 | 0 | 2 | 2 | 2 | 2 | 2 | 2 | 2 | 2 | 2 |
| E | 2 | 2 | 2 | 2 | 2 | 2 | 2 | 0 | 2 | 2 | 2 | 2 | 2 | 2 | 2 | 2 | 2 |
| F | 2 | 2 | 2 | 2 | 2 | 2 | 2 | 0 | 2 | 2 | 2 | 2 | 2 | 2 | 2 | 2 | 2 |
| G | 2 | 2 | 2 | 2 | 2 | 2 | 2 | 0 | 2 | 2 | 2 | 2 | 2 | 2 | 2 | 2 | 2 |
| H | 2 | 2 | 2 | 2 | 2 | 2 | 2 | 0 | 2 | 2 | 2 | 2 | 2 | 2 | 2 | 2 | 0 |
| I | 2 | 2 | 0 | 2 | 2 | 2 | 2 | 0 | 2 | 2 | 2 | 2 | 2 | 2 | 2 | 2 | 2 |
| J | 2 | 2 | 2 | 2 | 2 | 2 | 2 | 0 | 2 | 2 | 2 | 2 | 2 | 2 | 2 | 2 | 2 |
| K | 2 | 2 | 2 | 2 | 2 | 2 | 2 | 2 | 2 | 2 | 2 | 2 | 2 | 0 | 2 | 2 | 2 |
| L | 2 | 2 | 2 | 2 | 2 | 2 | 2 | 0 | 2 | 2 | 2 | 2 | 2 | 2 | 2 | 2 | 2 |
| M | 2 | 2 | 2 | 2 | 2 | 2 | 2 | 0 | 2 | 2 | 2 | 2 | 2 | 2 | 2 | 2 | 2 |
| N | 2 | 2 | 0 | 2 | 2 | 2 | 2 | 0 | 2 | 2 | 2 | 2 | 2 | 2 | 2 | 2 | 2 |

| | PCR amplification and XbaI restriction digestion patterns of amplicons obtained using | | | | | | | | | | | | | | | |
|---|---|---|---|---|---|---|---|---|---|---|---|---|---|---|---|---|
| PATS | 40 PATS - 4 virulence gene primer pairs^{b} | | | | | | | | | | | | | | | |
| type^{a} | IK123 | IK127 | IK131 | IK142 | IK148 | IKB1 | IKB3 | IKB4 | IKB | IKB6 | IKB7 | IKB8 | IKB9 | IKB10 | IKB13 | IKB14 |
| Control | 2 | 2 | 2 | 2 | 2 | 2 | 0 | 2 | 2 | 2 | 2 | 2 | 2 | 2 | 2 | 2 |
| Control | 2 | 2 | 2 | 2 | 2 | 2 | 2 | 2 | 2 | 2 | 2 | 2 | 2 | 2 | 2 | 2 |
| A | 2 | 2 | 2 | 2 | 2 | 2 | 0 | 2 | 2 | 2 | 2 | 2 | 2 | 2 | 2 | 2 |
| B | 2 | 2 | 2 | 2 | 2 | 2 | 2 | 2 | 2 | 2 | 2 | 2 | 2 | 2 | 2 | 2 |
| C | 2 | 2 | 2 | 2 | 2 | 2 | 0 | 2 | 0 | 2 | 2 | 2 | 2 | 2 | 2 | 2 |
| D | 2 | 2 | 2 | 2 | 2 | 2 | 0 | 2 | 0 | 2 | 2 | 2 | 2 | 2 | 2 | 2 |
| E | 2 | 2 | 2 | 2 | 2 | 2 | 2 | 2 | 2 | 2 | 2 | 2 | 2 | 2 | 2 | 2 |
| F | 2 | 0 | 2 | 2 | 2 | 2 | 2 | 2 | 2 | 2 | 2 | 2 | 2 | 2 | 2 | 2 |
| G | 2 | 2 | 2 | 2 | 2 | 2 | 0 | 2 | 2 | 2 | 2 | 2 | 2 | 2 | 2 | 2 |
| H | 2 | 2 | 2 | 2 | 2 | 2 | 0 | 2 | 0 | 2 | 2 | 2 | 2 | 2 | 2 | 2 |
| I | 0 | 2 | 2 | 2 | 2 | 2 | 0 | 2 | 2 | 2 | 2 | 2 | 2 | 2 | 2 | 2 |
| J | 0 | 2 | 2 | 2 | 2 | 2 | 0 | 2 | 2 | 2 | 2 | 2 | 2 | 2 | 2 | 2 |
| K | 0 | 2 | 2 | 2 | 2 | 2 | 0 | 2 | 2 | 2 | 2 | 2 | 2 | 2 | 2 | 2 |
| L | 2 | 0 | 2 | 2 | 2 | 2 | 0 | 2 | 2 | 2 | 2 | 2 | 2 | 2 | 2 | 2 |
| M | 2 | 2 | 2 | 2 | 2 | 2 | 2 | 2 | 0 | 2 | 2 | 2 | 2 | 2 | 2 | 2 |
| N | 0 | 2 | 2 | 2 | 2 | 2 | 2 | 2 | 2 | 2 | 2 | 2 | 2 | 2 | 2 | 2 |

| | PCR amplification and XbaI restriction digestion patterns of amplicons obtained using | | | | | | | | | | | |
|---|---|---|---|---|---|---|---|---|---|---|---|---|
| PATS | 40 PATS - 4 virulence gene primer pairs^{b} | | | | | | | | | | | |
| type^{a} | IKB15 | IKB16 | IKB17 | IKB18 | IKB19 | IKB20 | IKB21 | stx₁ | stx₂ | eae | hlyA | Isolates^{c} |
| Control | 2 | 2 | 2 | 2 | 2 | 2 | 2 | 0 | 1 | 1 | 1 | E. coli O157:H7 strain |
| | | | | | | | | | | | | 86-24 |
| Control | 2 | 2 | 2 | 2 | 2 | 2 | 2 | 1 | 1 | 1 | 1 | E. coli O157:H7 strain |
| | | | | | | | | | | | | 933 |
| A | 2 | 2 | 2 | 2 | 2 | 2 | 2 | 0 | 1 | 1 | 1 | G5289, G5290, |
| | | | | | | | | | | | | G5311, G5312 |
| B | 2 | 2 | 2 | 2 | 2 | 2 | 2 | 1 | 1 | 1 | 1 | G5320, G5327 |
| C | 2 | 2 | 2 | 2 | 2 | 2 | 2 | 1 | 1 | 1 | 1 | G5317, G5324, G5325 |
| D | 2 | 2 | 2 | 2 | 2 | 2 | 2 | 0 | 1 | 1 | 1 | G5283, G5284, |
| | | | | | | | | | | | | G5307, G5308 |
| E | 2 | 2 | 2 | 2 | 2 | 2 | 2 | 1 | 1 | 1 | 1 | G5285, G5286, |
| | | | | | | | | | | | | G5287, G5293, |
| | | | | | | | | | | | | G5294, G5300, |
| | | | | | | | | | | | | G5315, G5321, |
| | | | | | | | | | | | | G5322, G5326 |
| F | 2 | 2 | 2 | 2 | 2 | 2 | 2 | 1 | 1 | 1 | 1 | G5288, G5299 |
| G | 2 | 2 | 2 | 2 | 2 | 2 | 2 | 1 | 1 | 1 | 1 | G5291, G5292, |
| | | | | | | | | | | | | G5297, G5298, |
| | | | | | | | | | | | | G5301, G5302, |
| | | | | | | | | | | | | G5309, G5310, G5316 |
| H | 2 | 2 | 2 | 2 | 2 | 2 | 2 | 0 | 1 | 1 | 1 | G5295, G5296 |
| I | 2 | 2 | 2 | 2 | 2 | 2 | 2 | 1 | 1 | 1 | 1 | G5303 |
| J | 2 | 2 | 2 | 2 | 2 | 2 | 2 | 1 | 1 | 1 | 1 | G5304 |
| K | 2 | 2 | 2 | 2 | 2 | 2 | 2 | 1 | 1 | 1 | 1 | G5305, G5306 |
| L | 2 | 2 | 2 | 2 | 2 | 2 | 2 | 1 | 1 | 1 | 1 | G5313, G5314 |
| M | 2 | 2 | 2 | 2 | 2 | 2 | 2 | 1 | 1 | 1 | 1 | G5318 |
| N | 2 | 2 | 2 | 2 | 2 | 2 | 2 | 1 | 1 | 1 | 0 | G5323 |

^{a}PATS types are designated arbitrarily with different letters.
^{b}Prefixes of each PATS primer pair A/B and virulence gene primer pair F/R are indicated.
0, no amplicon;
1, amplicon without Xba I site;
2, amplicon with XbaI site PATS primer pairs producing polymorphic results between strains are shown in bold
^{c}Isolates of E. Coli O157:H& from various outbreaks (see Table 3) that fell within a given PATS type.

Phylogenetic Analysis of PATS Profiles Suggests a Clonal Lineage for E. coli O157:H7 Isolates.

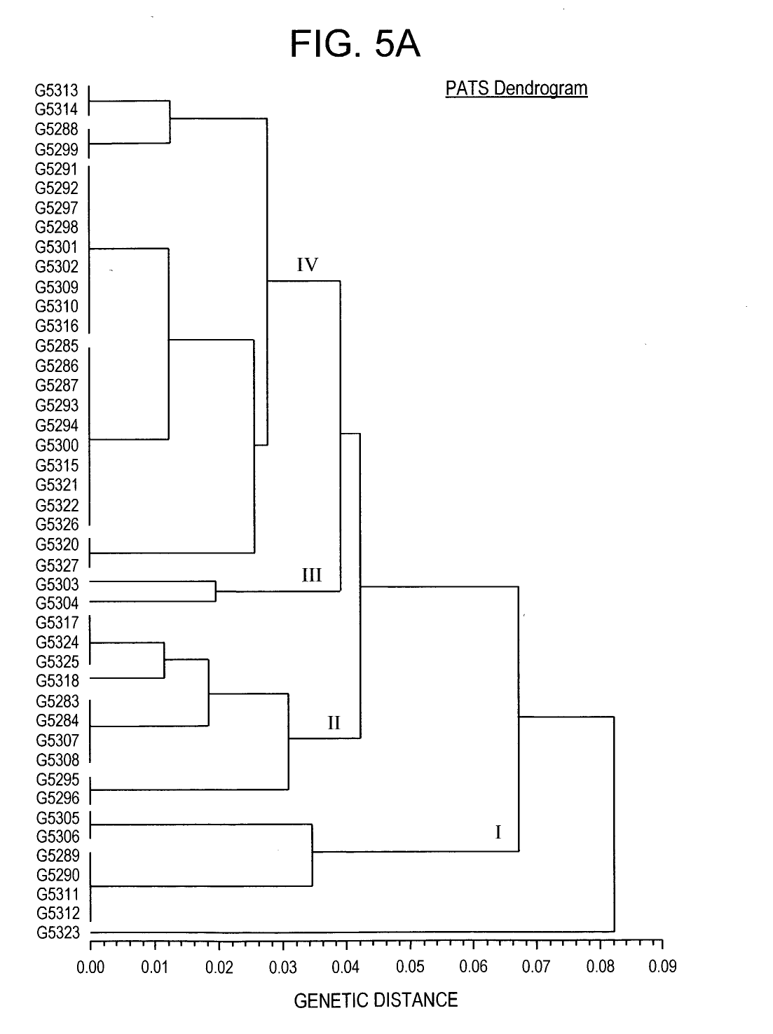

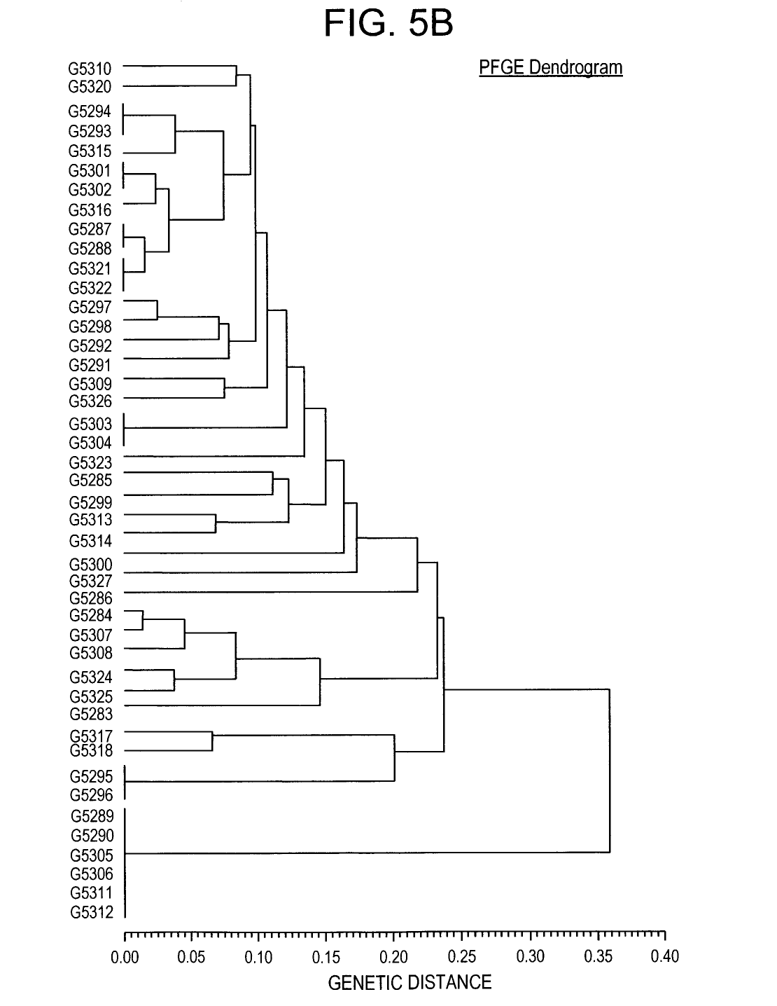

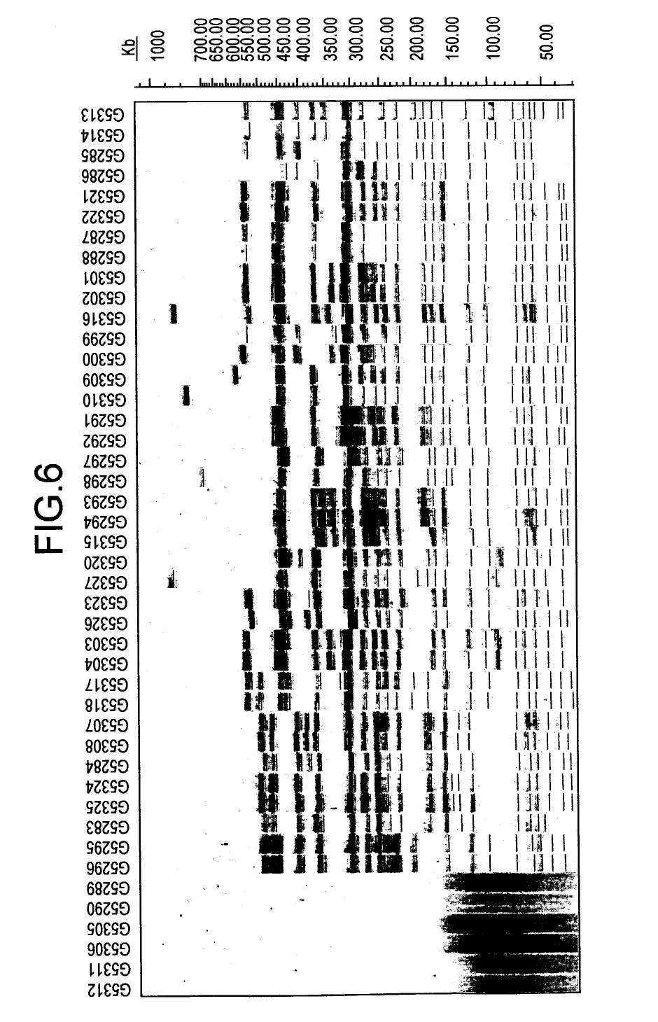

Based on the PATS profiles, the 44 E. coli O157:H7 isolates were grouped into four major phylogenetic clusters (FIG. 5A). A genetic distance of <0.1 between each cluster was suggestive of clonal relatedness. A closer analysis of the paired isolates from each outbreak was carried out. The PATS profile type was identical for the two isolates from 16 of the 22 outbreaks; as an example, isolates G5321 and G5322 belonging to outbreak number 3, shared the PATS profile type E (Tables 3 and 5; FIG. 5A). Isolates from five additional outbreaks (outbreaks 7, 13, 14, 16, and 22) had highly related PATS types, with only one polymorphism between the paired-isolates; for instance, isolates G5303 and G5304, belonging to outbreak 16, had the PATS profile types I and J respectively, differing only by the IK8 fragment polymorphism (Tables 3 and 5; FIG. 5A). The remaining two isolates, G5323 and G5326 from outbreak 2, were different due to multiple polymorphisms (Tables 3 and 5; FIG. 5A); these isolates also had substantially different PFGE patterns (FIG. 6) and so may not, in fact, be related isolates. Overall, the PATS typing system was able to correctly relate pairs of isolates from an outbreak for at least 21 of the 22 outbreaks (95%) tested (100% if isolates G5323 and G5326 are excluded). Some isolates from different outbreaks shared a common PATS type, leading to the larger clusters seen in the dendrogram (FIG. 5A), further supporting the clonal descent of these isolates.

PFGE, the most commonly used current standard for typing E. coli O157:H7, was also used to categorize the 44 isolates from the CDC (FIG. 6). The PATS dendrogram was compared with the PFGE dendrogram for the isolates in order to evaluate the potential of these two techniques in relating/discriminating outbreak-associated E. coli O157:H7. Phylogenetic analysis based on PFGE profiles resolved the 44 CDC isolates into smaller clusters with greater genetic distance between them than PATS. PFGE identified pairs from six outbreaks (outbreaks 3, 7, 10, 11, 15, and 16) as identical. For example, isolates G5321 and G5322 from outbreak 3 shared the same PFGE pattern (Table 3, FIGS. 5B and 6). Sixteen isolates from eight outbreaks (outbreaks 4, 5, 9, 12, 13, 14, 18, and 21) were classified as probably related (differences of 1-3 bands), as defined by Tenover et al (J. Clin. Microbiol. 33, 2233-2239 (1995)). For instance, the PFGE patterns of isolates G5317 and G5318, from outbreak 13, differed by one band (Table 3, FIGS. 5B and 6). Ten isolates from five outbreaks (outbreaks 1, 2, 6, 19, and 22) were possibly related (differences of 4-6 bands). For example, isolates G5320 and G5327 from outbreak 1 differed by four bands in the PFGE pattern (Table 3, FIGS. 5B and 6). Six isolates from three outbreaks (outbreaks 8, 17, and 20) were untypeable by PFGE (a common problem in PFGE typing) (Table 3, FIGS. 5B and 6). These six isolates were all typeable by PATS and fell into a distinctive cluster (cluster 1 on FIG. 5A).

PFGE was more discriminatory than PATS, with no overlaps in patterns between different outbreaks. However, PFGE matched fewer E. coli O157:H7 within outbreaks (pairs from 14 of 22 outbreaks were classified as identical or probably related) and was unable to type six isolates, thereby increasing the complexity of interpretation. In contrast, PATS typed all 44 isolates and matched 21 of 22 outbreak pairs as identical or related.

DNA Dot Blots can Effectively Detect PATS Amplicons.

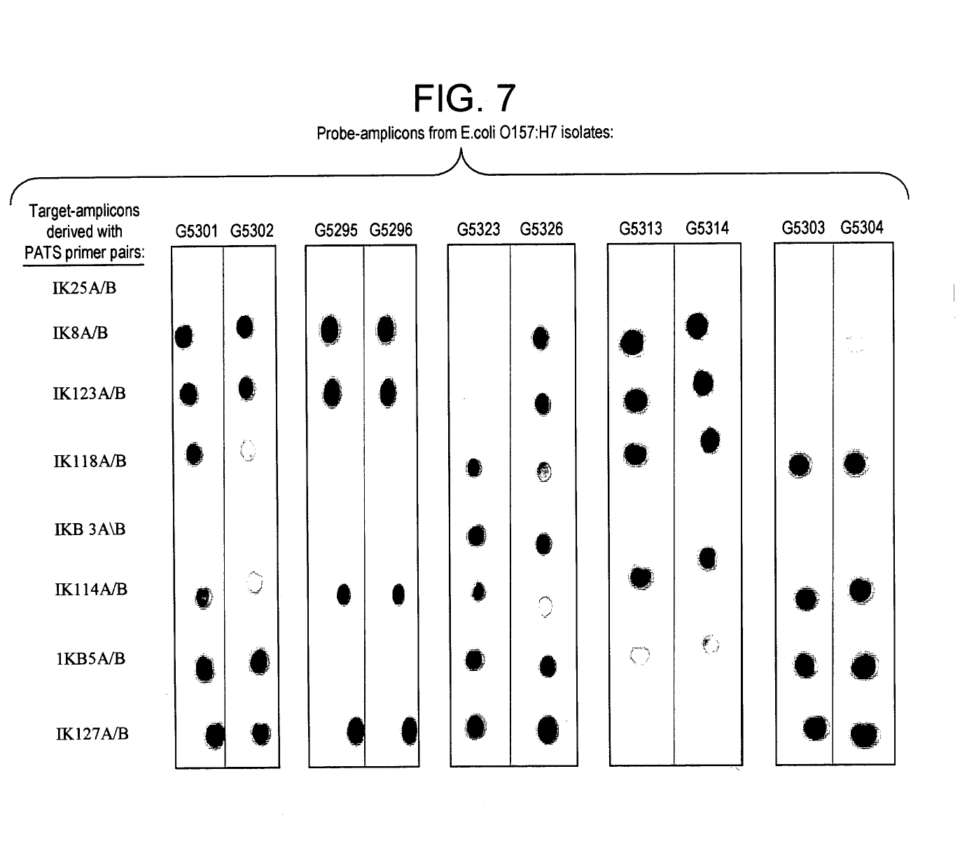

A dot blot assay to detect PATS amplicons was developed, to assess the feasibility of automating the PATS typing system. Eight PATS primer pairs that amplified polymorphic regions in the O157 genome were selected for the assay, as these were critical to the discriminatory power of PATS (Tables 4 and 5). Using these primer pairs, target-amplicons were derived from E. coli O157:H7 strain 86-24 or 933 and were spotted on nylon filters. Multiplex PCR was utilized to synthesize the probe amplicons to further expedite the assay. Of the eight primer pairs, seven were successfully used in a multiplex reaction. Primer pair IKB5A/B failed to produce sufficient quantities of its amplicon when used in combination with the other seven primer pairs, irrespective of the template. Altering the primer concentrations, template concentrations, annealing temperatures, extension times, number of cycles and various additives did not alter the performance of IKB5A/B. Hence, the probe-amplicon from this primer pair was synthesized in a separate single primer pair PCR and subsequently purified, labeled and pooled with the rest of the probe-amplicons. Dot blots of target-amplicons were hybridized with the probe-amplicons tagged with a chemiluminescent label. Resulting hybridization patterns correlated precisely with the PATS profiles for the respective isolates (FIG. 7, Table 7).

This study describes a novel E. coli O157:H7 typing system that utilizes a technique termed PATS, which is based on the presence or absence of specific DNA segments in genomic DNA. The technique is simple, highly reproducible and allows accurate objective interpretation of results.

Typing of pathogenic bacterial strains is important since distinct clones within a species/serotype may be associated with disease outbreaks and the severity and frequency of infection (Musser, Emerg. Infect. Dis. 2, 1-17 (1996)). Contemporary molecular typing techniques in use are based on restriction fragment length polymorphisms or distribution of random short sequence repeats (Olive and Bean, J. Clin. Microbiol. 37, 1661-1669 (1999); van Belkum et al., Curr. Opin. Microbiol. 2, 306-311(1999)). Of these, PFGE is considered to be the “gold standard” for typing, as it generates distinctive profiles that distinguish strains in several serotypes and species, including E. coli O157:H7 (Barrett et al., J. Clin. Microbiol. 32, 3013-3017 (1994); Bohm and Karch, J. Clin. Microbiol. 30, 2169-2172 (1992); Olive and Bean, J. Clin. Microbiol. 37, 1661-1669 (1999)). Since the XbaI restriction enzyme site occurs infrequently in the O157:H7 genome, it is frequently used with PFGE for this organism (Barrett et al., J. Clin. Microbiol. 32, 3013-3017 (1994); Bohm and Karch, J. Clin. Microbiol. 30, 2169-2172 (1992); Harsono et al., Appl. Environ. Microbiol. 59,3141-3144 (1993)). Although PFGE has been successfully used to support outbreak investigations, the technique has its limitations. For example, it may be impossible to fully resolve all bands on a gel under a single set of conditions, making interpretation and comparisons difficult (Harsono et al., Appl. Environ. Microbiol. 59,3141-3144 (1993); Johnson et al., Appl. Environ. Microbiol. 61,2806-2808 (1995); Meng et al., J. Med. Microbiol. 42,258-263 (1995)).

To overcome problems associated with present typing systems, a different typing methodology was developed, which has been termed PATS, based on detecting the presence or absence of the DNA segments containing the polymorphic XbaI sites. PFGE usually resolves about 20-25 XbaI-digested fragments for most E. coli O157:H7 isolates (smaller XbaI fragments are not visualized by PFGE) (Barrett et al., J. Clin. Microbiol. 32, 3013-3017 (1994); Harsono et al., Appl. Environ. Microbiol. 59,3141-3144 (1993); Meng et al., J. Med. Microbiol. 42,258-263 (1995)). A total of 40 XbaI sites between the genomes of two E. coli O157:H7 reference strains were identified, and eight of these 40 DNA segments were shown to be present or absent across a large collection of O157 strains. Reproducibility of PATS was demonstrated by consistency of typing patterns over three repeat PCRs. Compared to PFGE, PATS typed every E. coli O157:H7 isolate tested, matching 21 out of 22 outbreak pairs as identical or related and one pair as different. Four virulence gene primer pairs into the PATS typing system. Pathogenicity of E. coli O157:H7 is linked to these latter genes and their identification would help detect strains with potential for virulence in humans (Kaper and O'Brien, ASM Press (1998); Paton and Paton J. Clin. Microbiol. 36, 598-602 (1998)). Since the regions amplified by the virulence gene primer pairs lacked XbaI sites, polymorphisms in these virulence genes were distinguished by the presence or absence of these amplicons.

In comparison to PATS, PFGE matched fewer E. coli O157:H7 pairs within outbreaks (pairs from 14 of 22 outbreaks were classified as identical or highly related) and was unable to type six isolates, thereby increasing the complexity of interpretation. Since the outbreak strains tested here were collected between 1982 and 1993, it is possible that non-matching PFGE patterns of strains from the same outbreak are due to mutations that occurred during subculturing of the isolates. It is also possible that some of the isolates were misclassified as being outbreak-related, since subtyping was not available at the time of most of the outbreaks.

Unlike PFGE, methylation of XbaI sites does not interfere with PATS typing as it is a PCR based procedure (Dieffenbach and Dveksler, Cold Spring Harbor Press, (1995)), thereby avoiding this potentially confounding variable. One drawback of PATS was that it was less discriminatory than PFGE. While PATS detects the presence or absence of sequences containing XbaI sites, PFGE is also sensitive to insertions/deletions that may occur between XbaI sites, changing the size of the intervening fragment without altering the XbaI sites themselves. Also, two of the XbaI sites used in the PATS procedure are in DNA segments duplicated elsewhere in the genome (data not shown). While PATS is not dicriminate this duplication (it cannot distinguish between one or two copies of identical DNA segments in a genome), such duplications can affect the PFGE pattern. Although PATS was less discriminatory in our study than PFGE, the precision of the PATS procedure would be enhanced by identifying additional insertions/deletions in O157:H7 isolates and designing corresponding PATS primers.

PATS is a particularly powerful epidemiological tool for typing E. coli O157:H7 and other bacteria, even when compared to recently introduced typing techniques, such as MLST and octamer-based genome scanning (OBGS) (Kim et al., Proc. Natl. Acad. Sci. U.S.A. 96, 13288-13293 (1999)). While MLST can provide unambiguous results that are widely accessible over websites, the need for sequencing each isolate may not be cost-effective or provide rapid results (Feil et al., Mol. Biol. Evol. 16, 1496-1502 (1999)). The OBGS technique is similar to enterobacterial repetitive intergenic consensus sequence-PCR (Olive and Bean, J. Clin. Microbiol. 37, 1661-1669 (1999)) and has the inherent disadvantage of relying on repeat sequences; short sequence repeats are apt to undergo variation in composition and position through slipped strand mispairing during DNA replication and hence, techniques based on these repeats should be used with caution (van Belkum et al., Microbiol. Mol. Biol. Rev. 62, 275-293 (1998); van Belkum Curr. Opin. Microbiol. 2, 306-311 (1999)). Most importantly, as with PFGE, multiple DNA fragments generated by OBGS require electrophoretic separation and interpretation using special software (Kim et al., Proc. Natl. Acad. Sci. U.S.A. 96, 13288-13293 (1999)).

Automation according to standard methods would further enhance the applicability of PATS for routine typing of bacterial isolates. The concordance of the results of the DNA dot blot with the results by agarose gel electrophoresis suggests that a variety of techniques including the use of DNA microarrays are useful for such automation of the PATS typing system.

Microarrays

The present invention provides for nucleic acid compositions that can be employed in an array-format for distinguishing between bacterial strains. These methods are particularly useful for typing bacterial strains. Microarrays are useful in the diagnosis of a bacterial infection, in typing the bacterial strain producing the infection, and in determining treatment methods where differing methods of treatment are indicated by infection with particular bacterial strains.

The primers of the invention are useful to produce polymorphic nucleic acid fragments that are hybridizable array elements in a microarray. The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference.

Nucleic Acid Microarrays

To produce a nucleic acid microarray, primers of the invention are used to produce amplicons according to the methods described herein. Such amplicons may be synthesized or bound to the surface of a substrate using a chemical coupling procedure and an ink jet application apparatus, as described in PCT application WO95/251116. Alternatively, a gridded array may be used to arrange and link amplicon fragments or oligonucleotides to the surface of a substrate using a vacuum system, thermal, UV, mechanical or chemical bonding procedure.

A nucleic acid molecule (e.g., RNA or DNA) derived from a biological sample (e.g., a bacterial strain infecting a patient) may be used to produce a hybridization probe using standard methods. The biological samples are generally derived from a patient, from a bodily fluid (such as blood, cerebrospinal fluid, phlegm, saliva, urine, or stool) or tissue sample (e.g., a tissue sample obtained by biopsy). Bacterial nucleic acid molecules (RNA or DNA) are isolated according to standard methods, and a cDNA is produced and used as a template to make complementary RNA suitable for hybridization. The RNA is amplified, for example, in the presence of detectable nucleotides (e.g., fluorescent nucleotides), and the labeled probes are then incubated with the microarray to allow the probe sequence to hybridize to complementary oligonucleotides bound to the microarray.

Incubation conditions are adjusted according to methods known in the art such that hybridization occurs with precise complementary matches or with various degrees of less complementarity depending on the degree of stringency employed. For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least 35% formamide, and most preferably at least 50% formamide. Stringent temperature conditions will ordinarily include temperatures of at least 30° C., more preferably of at least 37° C., and most preferably of at least 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least 25° C., more preferably of at least 42° C., and most preferably of at least 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art.

A detection system may be used to measure the absence, presence, and amount of hybridization for all of the distinct sequences simultaneously (e.g., Heller et al., Proc. Natl. Acad. Sci. 94:2150-2155, 1997). Preferably, a scanner is used to determine the levels and patterns of fluorescence. The hybridization of bacterial nucleic acid molecules to a particular set of amplicons identifies a bacterial strain typing profile.

Diagnostics

The hybridization of nucleic acids molecules derived from a bacteria is useful in determining the bacterial strain profile. Primers (e.g., those listed in Tables 1A and Table 6), or identified according to methods described herein, may be used as targets in a microarray. The microarray is used to assay the bacterial strain typing profile.

In one embodiment, bacteria are isolated from a patient having a bacterial infection. Nucleic acid probes derived from the genome of these bacteria are hybridized with amplicons, or amplicon fragments, derived from known bacterial strains affixed to a microarray. The hybridization pattern of the nucleic acid probes defines a particular bacterial strain profile.

Detailed Materials and Methods

Described below are detailed materials and methods relating to the above-described experimental showing that polymorphic amplified typing sequences provide an approach to E. coli O157:H7 strain typing.

Bacteria, Plasmids and Media Used in this Study.

(i) E. coli O157:H7: Two strains of E. coli O157:H7 were used in the standardization of PATS. Strain 86-24, streptomycin resistant and originally isolated from a human in a Washington State outbreak, was obtained from Dr. A. D. O'Brien (Table 3). Strain 933, a human isolate from a Michigan State outbreak, was obtained from the American Type Culture Collection (ATCC, Manassas, Va.) which has it banked as ATCC 43895 (Table 3) (Wells et al., J. Clin. Microbiol. 18, 512-520 (1983)). Strain 933 is the E. coli O157:H7 isolate sequenced at the University of Wisconsin-Madison, Madison, Wis. (Perna et al., 2001). In addition, 44 isolates of E. coli O157:H7, two each from 22 different outbreaks, were obtained from the Centers for Disease Control and Prevention (CDC), Atlanta, Ga. The CDC numbers assigned to these isolates and the outbreaks they were associated with are indicated in Table 3. These isolates were primarily human isolates with the exception of two; G5308 was isolated from garden manure and G5310 from meat.

(ii) Other E. coli and plasmids: E. coli DH5α (F⁻ endA1 hsdR17 supE44 thi-1 recA1 gyrA96 relA1 Δ(argF-lacZYA)U169 (Φ80d lacZΔM15)) was used as the host strain to propagate recombinant plasmids. The plasmid pBluescribe (Stratagene USA, LaJolla, Calif.) was used as the cloning vector.

(iii) Media: All E. coli O157:H7 were grown in Luria-Bertani (LB) media. A single colony from each isolate was used to prepare −80° C. stocks in LB broth with 15% glycerol.

DNA Extraction, Sequencing, and Probe Labeling.

Genomic DNA was prepared using the Invitrogen Easy-DNA Isolation kit (Invitrogen Corporation, Carlsbad, Calif.) as per the manufacturer's instructions. Plasmid DNA was extracted using Qiagen plasmid purification kits (Qiagen Inc., Valencia, Calif.). Standard spectrophotometric analysis and agarose gel electrophoresis techniques were used to quantitate and evaluate purity of all DNA prepared (Ausubel et al., Current Protocols In Molecular Biology. New York: John Wiley and Sons, Inc.(1993); Maniatis, Fritsch, and Sambrook, Molecular cloning: A laboratory manual. New York: Cold Spring Harbor Laboratory (1989)).

DNA sequencing was done at the DNA Sequencing Core Facility, Department of Molecular Biology, Massachusetts General Hospital. This facility uses ABI Prism DiTerminator cycle sequencing with AmpliTaq DNA polymerase FS and an ABI 377 DNA sequencer (Perkin-Elmer Applied Biosystems Division, Foster City, Calif.) for this purpose.

All DNA probes were labeled using the ECL direct nucleic acid labeling and detection system (Amersham Pharmacia Biotech, Inc., Piscataway, N.J.). This approach is based on the direct labeling of DNA probes with horseradish peroxidase and detection by light generation resulting from the enzymatic cleavage of a chemiluminescent substrate, luminol.

Identification of Genomic DNA Fragments in E. coli O157:H7 Containing an XbaI Restriction Site.

(i) From Sau3AI-digested genomic DNA of E. coli O157:H7 strain 86-24 (FIG. 3): Genomic DNA from strain 86-24 was digested to completion using the 10 restriction enzyme Sau3AI (New England Biolabs, Inc., Beverly, Mass.). The digested fragments were allowed to self-ligate overnight and the circularized DNA then digested with XbaI (New England Biolabs); this ensured that only fragments containing an internal XbaI restriction site would linearize. The linearized fragments were cloned into the XbaI site in the multiple cloning site of pBluescribe. The resulting recombinant plasmids are prefixed as pIK. Plasmids were electroporated into competent E. coli DH5α cells using standard protocols (Maniatis et al., Cold Spring Harbor Laboratory (1989)). Transformants were screened on LB plates supplemented with ampicillin (100 μg/ml; Sigma Chemical Co., St. Louis, Mo.), 5-bromo-4-chloro-3-indolyl-β-D-galactopyranoside (X-Gal; 40 μg/ml; Sigma) and isopropyl-β-D-thiogalactopyranoside (IPTG; 1 mM; Sigma). A total of 100 white E. coli DH5α colonies containing recombinant plasmids were selected for further testing. Each strain containing a recombinant plasmid is prefixed IK in this paper.

(ii) From NlaIII-digested genomic DNA of E. coli O157:H7 strain 933 (FIG. 3): A different strain was used to recover NlaIII fragments of genomic DNA containing XbaI sites, in order to increase the diversity of XbaI sites identified, including those not recovered in Sau3AI fragments above. Genomic DNA from strain 933 was digested to completion using the restriction enzyme NlaIII (New England Biolabs). Subsequent steps leading to the selection of XbaI-containing fragments and the final screening of recombinant clones were as above. A total of 50 white E. coli DH5α colonies containing recombinant plasmids were selected for further testing. Plasmids and colonies were named as indicated above.

(iii) From E. coli O157:H7 strain 933 genomic DNA sequence: A total of 40 XhaI sites were localized in the 933 genomic sequence assembled at the University of Wisconsin-Madison, Madison, Wis., of which two were in duplicated regions and were not included in this study. Of the remaining 38 XhaI sites, 20 were already identified in plasmids described above, and 18 were newly identified from the genome sequence. The sequences surrounding these 18 XbaI sites are referred to with the prefix IKB in this paper. Two additional XbaI-containing genomic segments are unique to strain 86-24 and were recovered in step (i) above.

Evaluation of Recombinant Plasmids.

Plasmid DNA was extracted from isolated colonies of IK1-150 and plasmids pIK1-150 were screened for the presence of an appropriate insert. As a result of the self-ligation at the Sau3AI or NlaIII sites, digestion with XbaI and cloning, an appropriate insert would have XbaI sites at either end, and a single, internal Sau3AI or NlaIII site (FIG. 3). Plasmids were digested with XbaI to check for the release of a single insert. In addition, pBluescribe-specific primers (see below) were used to amplify the insert by PCR. The resulting amplicons were purified using the Qiaquick PCR purification kit (Qiagen, Inc.) and then digested with either Sau3AI or NlaIII, to confirm the presence of these sites within the fragments. DNA fragments were resolved by agarose gel electrophoresis and visualized by staining with ethidium bromide. The pBluescribe-specific primers were in the multiple cloning site on either side of the insert, and were: Reverse (5′-GAAACAGCTATGACC ATG-3′; SEQ ID NO.: 18) and M13-20 (5′-GTAAAACGACGGCCAGT-3′; SEQ ID NO:19). PCR was done on a PTC-100 thermal cycler (MJ Research, Inc., Watertown, Mass.), using 10 ng plasmid DNA, 100 pmoles of each vector primer, 800 μM dNTPs, 1× diluted Ex Taq™ enzyme buffer and 2.5 units of TaKaRa Ex Taq™ DNA polymerase (Takara Shuzo Co., LTD., Panvera Corporation, Madison, Wis.). Denaturation at 95° C. for 5 min was followed by 30 cycles of amplification (1 min at 95° C., 1 min at 45° C., 1 min at 72° C. per cycle) and a final extension at 72° C. for 1 min. Each reaction was done in triplicate.

As more recombinant plasmids were studied, duplicates containing inserts already analyzed were eliminated using Southern blot hybridization. Briefly, XbaI-digested plasmid DNA was electrophoresed on an agarose gel, transferred to Hybond-N+ membranes (Amersham Pharmacia), U.V. crosslinked to the membrane using a Stratalinker (Stratagene), and hybridized with a pool of the previously characterized insert DNAs labeled as described above. Hybridization at 42° C. and post-hybridization washing of blots was done as per the ECL kit manual (Amersham Pharmacia). Autoradiographs were prepared by exposure of processed blots to the Kodak Scientific Imaging X-OMAT AR film (Eastman Kodak Company, Rochester, N.Y.), and plasmids containing inserts hybridizing to the pool of previous inserts were not further evaluated.

Design of PATS and Virulence Gene Primer Pairs.

Plasmids with appropriate inserts were sequenced using pBluescribe specific primers (reverse and M13-20). Insert sequences were used to design PATS primer pairs flanking each XbaI site on the genome and designed to amplify fragments of approximately 200-400 bp (FIG. 3). In the context of the plasmid, these primers appear divergent to each other, since the genomic XbaI site is linearized at either end of the insert (FIG. 3). However, in the undigested genome, each primer pair flanks a single, internal XbaI site. PATS primer pairs were also designed to amplify the 18 XbaI sites specifically identified from the E. coli O157:H7 strain 933 genome sequence.

Primer pairs were also generated to amplify specific virulence genes found in strains of E. coli O157:H7, similar to those designed by Paton et al (Paton and Paton, J. Clin. Microbiol. 36, 598-602 (1998)). The four primer pairs included:

stx₁F
(5′-ATAAATCGCCATTCGTTGACTAC-3′; SEQ ID NO:20)/
stx₁R
(5′-GAACGCCCACTGAGATCAT C-3′; SEQ ID NO:21),
stx₂F
(5′-GGCACTGTCTGAAACTGCTCC-3′; SEQ ID NO:22)/
stx₂R
(5′-TCGCCAGTTATCTGACAT TCTG-3′; SEQ ID NO:23),
eaeF
(5′-GACCCGGCACAAGCATA AGC-3′; SEQ ID NO:24)/
eaeR
(5′-CCACCTGCAGCAA CAAGAGG-3′; SEQ ID NO:25)
and
hlyAF
(5′-GCATCATCAAGCGT ACGTTCC-3′; SEQ ID NO:26)/
hlyAR
(5′-AATGAGCCAAGCTGGTTAAGCT-3′; SEQ ID NO:27).

PATS Typing.

PATS primers were used to assay for the presence or absence of individual XbaI sites in different isolates of E. coli O157:H7. PCR was done using E. coli O157:H7 colony lysate and/or genomic DNA as templates. Colony lysates were prepared by boiling a suspension of colonies in sterile distilled water, followed by centrifugation at 4° C. Each E. coli O157:H7 isolate template was tested with each individual PATS primer pair, in separate reactions.

PCR was done on the GeneAmp PCR system 2400 thermal cycler (PE Biosystems, Foster City, Calif.), using 200 ng genomic DNA or 10 μl of colony lysate, 200 pmoles of each PATS primer, 800 μM dNTPs, 1× diluted Ex Taq™ enzyme buffer and 2.5 units of TaKaRa Ex Taq™ DNA polymerase. Hot start PCR technique was employed in which the polymerase was added only after preheating the rest of the PCR mix (Dieffenbach and Dveksler, Cold Spring Harbor Press (1995)). This technique was used in combination with a Touchdown PCR profile (Lawrence and Hartl, Genetica 84, 23-29 (1991)). To create this profile, the regular PCR program was modified as follows: an amplification segment of 20 cycles was set where the annealing temperature started at 73° C., to touchdown at 53° C. at the end of those cycles. Then, another amplification segment of 10 cycles was set, using the last annealing temperature of 53° C. Each reaction was done in triplicate.

Amplicons obtained by PCR were purified using the Qiaquick PCR purification kit and digested with XbaI to confirm the presence of an XbaI site within the amplicon. Undigested and digested DNA fragments were resolved on a 4% agarose gel prepared with a combination of 3% Nusieve GTG agarose (FMC BioProducts, Rockland, Me.) and 1% agarose (Shelton Scientific Inc., Shelton, Conn.), stained with ethidium bromide. These same amplicons were also used to probe genomic DNA of isolates used in PATS typing, following digestion with Sau3AI.

Pulsed-Field Gel Electrophoresis (PFGE).

PFGE analysis of all E. coli O157:H7 isolates was done at the CDC, Atlanta, Ga. Standard procedures previously described (Ausubel et al., Current Proocols in Moelcular Biology, John Wiley and Sons, Inc. (1993); Barrett et al., J. Clin. Microbiol. 32, 3013-3017 (1994)) were used, with the following modifications. Briefly, genomic DNA of each isolate was embedded in separate agarose plugs and digested at 37° C. for 2 hr with 30U of XbaI per plug (Gibco BRL, Grand Island, N.Y.). The plugs were loaded onto a 1% agarose-Tris buffer gel (SeaKem Gold Agarose, BioWhittaker Molecular Applications, Rockland, Mass.) and PFGE was performed using a CHEF Mapper XA (Bio-Rad Laboratories, Hercules, Calif.). DNA was electrophoresed for 18 h at a constant voltage of 200 V (6 V/cm), with a pulse time of 2.2 to 54.2 s, an electric field angle of 120°, and temperature of 14° C., before being stained with ethidium bromide.

DNA Dot-Blots.

Primer pairs IK8A/B, IK25A/B, IK114A/B, IK118A/B, IK123A/B, IK127A/B, IKB3A/B, and IKB5A/B were used in this assay. Amplicons were first obtained from E. coli O157:H7 strain 86-24 or 933, using each primer pair in a separate reaction. 2.5 μl of each purified amplicon was spotted on Hybond N+ membrane (Amersham Pharmacia) strips and U.V. crosslinked; these constituted the “target-amplicons”. Ten E. coli O157:H7 isolates (G5301, G5302, G5295, G5296, G5323, G5326, G5313, G5314, G5303, and G5304), from five different outbreaks, were selected for analysis by dot-blot using multiplex PCR. For each of these isolates, amplicons were derived using seven of the eight primer pairs in a multiplex PCR reaction, as well as a separate PCR reaction for primer pair IKB5A/B. To ensure equal quantities of all amplicons in the multiplex reaction, primer concentrations were varied. Primer pairs IK25A/B, IK114A/B, IK123A/B, and IK127A/B were used at a concentration of 200 pmoles per primer; primer pairs IK8A/B, IK118A/B, and IKB3A/B were used at 100 pmoles per primer. In the separate PCR reaction, primer pair IKB5A/B was used at a concentration of 200 pmoles per primer. These amplicons were purified, labeled with the ECL kit and pooled; these constituted the “probe-amplicons”. Each membrane strip containing the target-amplicons was hybridized at 42° C. with the pool of purified probe-amplicons generated from a single isolate and autoradiographs prepared by exposure of processed blots to the Kodak Scientific Imaging X-OMAT AR film (Eastman Kodak Company), to detect the presence or absence of hybridizing amplicons in the isolates being analyzed.

Software.

PFGE gels were analyzed using Molecular Analyst Fingerprinting Plus software (Bio-Rad). Dendrograms were constructed using the unweighted pair-group method with arithmetic mean (UPGMA).

### Example 3

Insertions Deletions, and SNPs at AvrII Sites Enhanced the PATS Strain Typing System for E. coli O157:H7

We designed primer pairs to amplify DNA flanking 33 sites in the O157 genome for the rare cutting restriction enzyme, AvrII. Of these 33 sites, 7 sites were identified that were polymorphic between O157 isolates. In the case of the AvrII sites, polymorphisms were due to either insertions, deletions, or single nucleotide polymorphisms (SNPs). The SNPs occurred either within the AvrII site itself, resulting in loss of the site, or in sequences near the site, resulting in the creation of an additional AvrII site.

Of the 7 polymorphic AvrII sites, 5 were in O-islands and 2 were in the backbone (sequences shared with E. coli K12). Adding primer pairs specific for DNA flanking these 7 polymorphic AvrII sites to the primer pairs specific for the 8 polymorphic XbaI sites and four virulence genes (stx₁, stx₂, eae, hlyA), made the PATS typing system highly discriminatory for distinguishing strains of O157.

The primer pairs depicted in Table 6 produced polymorphic results across the isolate set, amplifying products with an AvrII site in some isolates but failing to amplify any product in others.

TABLE 6

| | | | | | Distance | Amplicon | |
|---|---|---|---|---|---|---|---|
| No. | Seq ID | Primer | Length | Sequence (5′→3′) | From AvrII | Size | Tm (° C.) |
| 1 | 29 | IKNR3 A | 24 | GCACCATTCATGATATTCGTTAAC | 254 bp | 380 bp | 66 |
| | 30 | IKNR3 B | 24 | TTGCAATGTTCATTAATATACGTC | 126 bp | | 62 |
| 2 | 31 | IKNR7 A | 24 | TATACTCATTGATAAAATACTAAC | 268 bp | 406 bp | 58 |
| | 32 | IKNR7 B | 24 | AGCACAGAAGAGTAATTATATGTC | 138 bp | | 64 |
| 3 | 33 | IKNR10 A | 24 | ATCAGGATGCCGTFATACTCATTG | 282 bp | 419 bp | 68 |
| | 34 | IKNR10 B | 24 | GCACAGAAGAGTAATTATATGTCC | 137 bp | | 66 |
| 4 | 35 | IKNR12 A | 24 | AAGTTTTGATATTGTACTGGATGC | 304 bp | 443 bp | 64 |
| | 36 | IKNR12 B | 24 | CATTAAAGATAGATGATAAATCAC | 139 bp | | 60 |
| 5 | 37 | IKNR16 A | 24 | TGCTCAACATAGAAACCCACATAG | 144 bp | 444 bp | 68 |
| | 38 | IKNR16 B | 24 | TCGAATCAGTGTTATTTACCAGTG | 300 bp | | 66 |
| 6 | 39 | IKNR27 A | 24 | GTTATTCTGGTACATGAACATCAT | 336 bp | 524 bp | 64 |
| | 40 | IKNR27 B | 24 | TAGATAATTCCACACAGCCCACTA | 188 bp | | 68 |
| 7 | 41 | IKNR33 A | 24 | GTAGTCGAAATCATGGTGCAGAAT | 217 bp | 383 bp | 68 |
| | 42 | IKNR33 B | 24 | CTTCTCTGCTGTTTGGTGTCTTAT | 166 bp | | 68 |

The DNA sequences amplified by the AvrII primer pairs were analyzed using the Genbank database (BLAST search program, NCBI) and the E. coli O157:H7 strain 933 genome sequence database (University of Wisconsin). Of the 32 AvrII-containing genome sequences analyzed, 22 were homologous to E. coli strain K-12 genome sequences (referred to as backbone sequences (Perna et al., Nature 409, 463-466 (2001)), while 10 were in regions of the O157:H7 chromosome not shared with K-12, referred to as O-islands (SEQ ID NO.: 1) (Perna et al., Nature 409, 463-466 (2001)). The majority of the polymorphic regions were localized to the O-islands (5/7), compared to a few in the conserved regions (5/25) indicating again that genetic differences between E. coli O157:H7 strains occur in O-islands. The location of the regions amplified by each primer pair is shown in Table 7.

TABLE 7
E. coli O157 genomic regions amplified by the 32 PATS primer pairs
designed flanking AvrII restriction enzyme sites.

| Regions conserved | | Regions polymorphic | |
|---|---|---|---|
| across all strains | | between strains | |
| | Location in the | | Location in the O157 |
| Primer pair | O157 genome | Primer pair | genome |
| IKNR1A/B | Backbone^{a} | IKNR3A/B | O-island |
| IKNR2A/B | Backbone | IKNR7A/B | O-island |
| IKNR4A/B | Backbone | IKNR10A/B | O-island |
| IKNR5A/B | Backbone | IKNR12A/B | O-island |
| IKNR6A/B | O-island^{b} | IKNR16A/B | Backbone |
| IKNR8A/B | O-island | IKNR27A/B | Backbone |
| IKNR9A/B | O-island | IKNR33A/B | O-island |
| IKNR11A/B | Backbone | | |
| IKNR13A/B | O-island | | |
| IKNR14A/B | Backbone | | |
| IKNR15A/B | Backbone | | |
| IKNR17A/B | Backbone | | |
| IKNR18A/B | Backbone | | |
| IKNR19A/B | Backbone | | |
| IKNR20A/B | Backbone | | |
| IKNR21A/B | Backbone | | |
| IKNR22A/B | O-island | | |
| IKNR23A/B | Backbone | | |
| IKNR24A-1/B-1 | Backbone | | |
| IKNR25A/B | Backbone | | |
| IKNR26A-1/B-1 | Backbone | | |
| IKNR28A/B | Backbone | | |
| IKNR30A/B | Backbone | | |
| IKNR31A/B | Backbone | | |
| IKNR32A/B | Backbone | | |

^{a}Backbone, DNA sequences homologous to E. coli K-12 genome.
^{b}O-island, DNA sequences unique to E. coli O157 genome.

PATS profiles of O157 strain isolates were also identified using primers that flanked AvrII restriction sites and virulence gene primer pairs. Table 8 shows the result of this analysis.

TABLE 8
PATS profiles of O157 isolates based on AvrII restriction sites and virulence genes.

| | PCR amplification and AvrII restriction digestion patterns of amplicons obtained using | | | | | | | | | | | | |
|---|---|---|---|---|---|---|---|---|---|---|---|---|---|
| | 31 PATS - 4 virulence gene primer pairs^{b} | | | | | | | | | | | | |
| PATS type^{a} | IKNR1 | IKNR2 | IKNR3 | IKNR4 | IKNR5 | IKNR6 | IKNR7 | IKNR8 | IKNR9 | IKNR10 | IKNR11 | IKNR12 | IKNR13 |
| Control | 2 | 2 | 2 | 2 | 2 | 2 | 2 | 2 | 2 | 2 | 2 | 2 | 2 |
| Control | 2 | 2 | 2 | 2 | 2 | 2 | 2 | 2 | 2 | 2 | 2 | 2 | 2 |
| A(3) | 2 | 2 | 2 | 2 | 2 | 2 | 2 | 2 | 2 | 2 | 2 | 2 | 2 |
| B(1) | 2 | 2 | 2 | 2 | 2 | 2 | 2 | 2 | 2 | 2 | 2 | 0 | 2 |
| C(2) | 2 | 2 | 2 | 2 | 2 | 2 | 2 | 2 | 2 | 2 | 2 | 0 | 2 |
| D(2) | 2 | 2 | 2 | 2 | 2 | 2 | 1 | 2 | 2 | 1 | 2 | 2 | 2 |
| E(4) | 2 | 2 | 2 | 2 | 2 | 2 | 1 | 2 | 2 | 1 | 2 | 2 | 2 |
| F(4) | 2 | 2 | 2 | 2 | 2 | 2 | 2 | 2 | 2 | 2 | 2 | 2 | 2 |
| G(2) | 2 | 2 | 1 | 2 | 2 | 2 | 2 | 2 | 2 | 2 | 2 | 2 | 2 |
| H(2) | 2 | 2 | 2 | 2 | 2 | 2 | 2 | 2 | 2 | 2 | 2 | 2 | 2 |
| I(2) | 2 | 2 | 1 | 2 | 2 | 2 | 2 | 2 | 2 | 2 | 2 | 2 | 2 |
| J(1) | 2 | 2 | 2 | 2 | 2 | 2 | 2 | 2 | 2 | 2 | 2 | 2 | 2 |
| K(21) | 2 | 2 | 2 | 2 | 2 | 2 | 2 | 2 | 2 | 2 | 2 | 2 | 2 |

| | PCR amplification and AvrII restriction digestion patterns of amplicons obtained using | | | | | | | | | | |
|---|---|---|---|---|---|---|---|---|---|---|---|
| | 31 PATS - 4 virulence gene primer pairs^{b} | | | | | | | | | | |
| PATS type^{a} | IKNR14 | IKNR15 | IKNR16 | IKNR17 | IKNR19 | IKNR20 | IKNR21 | IKNR22 | IKNR23 | IKNR24 | IKNR25 |
| Control | 2 | 2 | 2 | 2 | 2 | 2 | 2 | 2 | 2 | 2 | 2 |
| Control | 2 | 2 | 2 | 2 | 2 | 2 | 2 | 2 | 2 | 2 | 2 |
| A(3) | 2 | 2 | 2 | 2 | 2 | 2 | 2 | 2 | 2 | 2 | 2 |
| B(1) | 2 | 2 | 2 | 2 | 2 | 2 | 2 | 2 | 2 | 2 | 2 |
| C(2) | 2 | 2 | 2 | 2 | 2 | 2 | 2 | 2 | 2 | 2 | 2 |
| D(2) | 2 | 2 | 1 | 2 | 2 | 2 | 2 | 2 | 2 | 2 | 2 |
| E(4) | 2 | 2 | 1 | 2 | 2 | 2 | 2 | 2 | 2 | 2 | 2 |
| F(4) | 2 | 2 | 2 | 2 | 2 | 2 | 2 | 2 | 2 | 2 | 2 |
| G(2) | 2 | 2 | 1 | 2 | 2 | 2 | 2 | 2 | 2 | 2 | 2 |
| H(2) | 2 | 2 | 2 | 2 | 2 | 2 | 2 | 2 | 2 | 2 | 2 |
| I(2) | 2 | 2 | 1 | 2 | 2 | 2 | 2 | 2 | 2 | 2 | 2 |
| J(1) | 2 | 2 | 2 | 2 | 2 | 2 | 2 | 2 | 2 | 2 | 2 |
| K(21) | 2 | 2 | 2 | 2 | 2 | 2 | 2 | 2 | 2 | 2 | 2 |

| | PCR amplification and AvrII restriction digestion patterns of amplicons obtained using | | | | | | | | | | | |
|---|---|---|---|---|---|---|---|---|---|---|---|---|
| | 31 PATS - 4 virulence gene primer pairs^{b} | | | | | | | | | | | |
| PATS type^{a} | IKNR26 | IKNR27 | IKNR28 | IKNR30 | IKNR31 | IKNR32 | IKNR33 | stx₁ | stx₂ | eae | hlyA | Isolates^{c} |
| Control | 2 | 2 | 2 | 2 | 2 | 2 | 2 | 1 | 1 | 1 | 1 | E. coli O157:H7 strain |
| | | | | | | | | | | | | EDL933 |
| Control | 2 | 3 | 2 | 2 | 2 | 2 | 0 | 0 | 1 | 1 | 1 | E. coli O157:H7 strain |
| | | | | | | | | | | | | 86-24 |
| A(3) | 2 | 3 | 2 | 2 | 2 | 2 | 0 | 0 | 1 | 1 | 1 | G5290, G5311, G5312 |
| B(1) | 2 | 3 | 2 | 2 | 2 | 2 | 0 | 0 | 1 | 1 | 1 | G5289 |
| C(2) | 2 | 2 | 2 | 2 | 2 | 2 | 2 | 1 | 1 | 1 | 1 | G5316, G5320 |
| D(2) | 2 | 2 | 2 | 2 | 2 | 2 | 2 | 1 | 1 | 1 | 1 | G5324, G5325 |
| E(4) | 2 | 2 | 2 | 2 | 2 | 2 | 2 | 0 | 1 | 1 | 1 | G5283, G5284, G5307, |
| | | | | | | | | | | | | G5308 |
| F(4) | 2 | 3 | 2 | 2 | 2 | 2 | 2 | 1 | 1 | 1 | 1 | G5291, G5292, G5303, |
| | | | | | | | | | | | | G5304 |
| G(2) | 2 | 2 | 2 | 2 | 2 | 2 | 2 | 0 | 1 | 1 | 1 | G5295, G5296 |
| H(2) | 2 | 3 | 2 | 2 | 2 | 2 | 0 | 1 | 1 | 1 | 1 | G5305, G5306 |
| I(2) | 2 | 2 | 2 | 2 | 2 | 2 | 2 | 1 | 1 | 1 | 1 | G5317, G5318 |
| J(1) | 2 | 2 | 2 | 2 | 2 | 2 | 2 | 1 | 1 | 1 | 0 | G5323 |
| K(21) | 2 | 2 | 2 | 2 | 2 | 2 | 2 | 1 | 1 | 1 | 1 | G5285, G5286, G5287, |
| | | | | | | | | | | | | G5288, G5293, G5294, |
| | | | | | | | | | | | | G5297, G5298, G5299, |
| | | | | | | | | | | | | G5300, G5301, G5302, |
| | | | | | | | | | | | | G5309, G5310, G5313, |
| | | | | | | | | | | | | G5314, G5315, G5321, |
| | | | | | | | | | | | | G5322, G5326, G5327 |

^{a}PATS types are designated arbitrarily with different numbers
^{b}Prefixs of each PATS primer pair A/B and virulence gene primer pair F/R are indicated.
0, no amplicon;
1, amplicon without AvrII site;
2, amplicon with AvrII site;
3, amplicon with an additional AvrII site due to a SNP PATS primer pairs producing polymorphic results between strains are shown in bold.
^{c}Isolates of E. coli O157:H7 from various outbreaks that fell within a given PATS type.

PATS amplicon analysis using AvrII and the virulence gene primer pairs identified eleven different PATS profiles (Table 8) for O157 isolates, compared to the fourteen PATS profiles (Table 5) obtained for the same set of isolates using XbaI and the virulence gene primer pairs. However, PATS amplicon analysis using XbaI, AvrII, and the virulence gene primer pairs was able to discriminate 20 different PATS profiles for the same O157 isolates. The results of this analysis are shown in Table 9.

TABLE 9
PATS profiles of O157 isolates based on polymorphic XbaI and AvrII restriction sites, and virulence genes

| | PCR amplification and restriction digestion | | | | | | | | | | | |
|---|---|---|---|---|---|---|---|---|---|---|---|---|
| | patterns of amplicons obtained using 15 PATS - | | | | | | | | | | | |
| | 4 virulence gene primer pairs^{b} | | | | | | | | | | | |
| | Polymorphic | | | | | | | Polymorphic | | | | |
| | XbaI sites | | | | | | | AvrII sites | | | | |
| PATS type^{a} | IK8 | IK19 | IK25 | IK114 | IK118 | IK123 | IKB3 | IKB5 | IKNR3 | IKNR7 | IKNR10 | IKNR12 |
| Control | 0 | 2 | 0 | 2 | 2 | 2 | 2 | 2 | 2 | 2 | 2 | 2 |
| Control | 2 | 2 | 2 | 0 | 2 | 2 | 0 | 2 | 2 | 2 | 2 | 2 |
| A(3) | 2 | 2 | 2 | 0 | 2 | 2 | 0 | 2 | 2 | 2 | 2 | 2 |
| B(1) | 0 | 2 | 0 | 2 | 2 | 2 | 2 | 2 | 2 | 2 | 2 | 2 |
| C(1) | 0 | 2 | 0 | 2 | 2 | 2 | 2 | 2 | 2 | 2 | 2 | 0 |
| D(1) | 2 | 2 | 2 | 0 | 2 | 2 | 0 | 2 | 2 | 2 | 2 | 0 |
| E(1) | 0 | 2 | 0 | 2 | 2 | 0 | 2 | 2 | 2 | 2 | 2 | 2 |
| F(1) | 2 | 2 | 0 | 2 | 2 | 2 | 0 | 0 | 1 | 2 | 2 | 2 |
| G(2) | 2 | 2 | 0 | 2 | 2 | 2 | 0 | 0 | 2 | 1 | 1 | 2 |
| H(4) | 2 | 2 | 0 | 2 | 2 | 2 | 0 | 0 | 2 | 1 | 1 | 2 |
| I(2) | 2 | 2 | 0 | 2 | 0 | 2 | 0 | 0 | 1 | 2 | 2 | 2 |
| J(2) | 2 | 0 | 0 | 2 | 2 | 2 | 2 | 2 | 2 | 2 | 2 | 2 |
| K(1) | 0 | 2 | 0 | 2 | 2 | 0 | 0 | 2 | 2 | 2 | 2 | 2 |
| L(1) | 2 | 2 | 0 | 2 | 2 | 0 | 0 | 2 | 2 | 2 | 2 | 2 |
| M(2) | 2 | 2 | 2 | 0 | 2 | 0 | 0 | 2 | 2 | 2 | 2 | 2 |
| N(2) | 2 | 0 | 0 | 2 | 2 | 2 | 0 | 2 | 2 | 2 | 2 | 2 |
| O(1) | 2 | 2 | 0 | 2 | 2 | 2 | 2 | 0 | 1 | 2 | 2 | 2 |
| P(1) | 2 | 2 | 0 | 2 | 2 | 2 | 0 | 2 | 2 | 2 | 2 | 0 |
| Q(2) | 2 | 2 | 0 | 2 | 2 | 2 | 0 | 2 | 2 | 2 | 2 | 2 |
| R(6) | 2 | 2 | 0 | 2 | 2 | 2 | 0 | 2 | 2 | 2 | 2 | 2 |
| S(10) | 2 | 2 | 0 | 2 | 2 | 2 | 2 | 2 | 2 | 2 | 2 | 2 |

| | PCR amplification and restriction digestion | | | | | | | |
|---|---|---|---|---|---|---|---|---|
| | patterns of amplicons obtained using 15 PATS - | | | | | | | |
| | 4 virulence gene primer pairs^{b} | | | | | | | |
| | Polymorphic | | | Virulence | | | | |
| | AvrII sites | | | genes | | | | |
| PATS type^{a} | IKNR16 | IKNR27 | IKNR33 | stx₁ | stx₂ | eae | hlyA | Isolates^{c} |
| Control | 2 | 2 | 2 | 1 | 1 | 1 | 1 | E. coli O157:H7 strain EDL933 |
| Control | 2 | 3 | 0 | 0 | 1 | 1 | 1 | E. coli O157:H7 strain 86-24 |
| A(3) | 2 | 3 | 0 | 0 | 1 | 1 | 1 | G5290, G5311, G5312 |
| B(1) | 2 | 2 | 2 | 1 | 1 | 1 | 1 | G5327 |
| C(1) | 2 | 2 | 2 | 1 | 1 | 1 | 1 | G5320 |
| D(1) | 2 | 3 | 0 | 0 | 1 | 1 | 1 | G5289 |
| E(1) | 2 | 2 | 2 | 1 | 1 | 1 | 0 | G5323 |
| F(1) | 1 | 2 | 2 | 1 | 1 | 1 | 1 | G5317 |
| G(2) | 1 | 2 | 2 | 1 | 1 | 1 | 1 | G5324, G5325 |
| H(4) | 1 | 2 | 2 | 0 | 1 | 1 | 1 | G5283, G5284, G5307, G5308 |
| I(2) | 1 | 2 | 2 | 0 | 1 | 1 | 1 | G5295, G5296 |
| J(2) | 2 | 2 | 2 | 1 | 1 | 1 | 1 | G5288, G5299 |
| K(1) | 2 | 3 | 2 | 1 | 1 | 1 | 1 | G5303 |
| L(1) | 2 | 3 | 2 | 1 | 1 | 1 | 1 | G5304 |
| M(2) | 2 | 3 | 0 | 1 | 1 | 1 | 1 | G5305, G5306 |
| N(2) | 2 | 2 | 2 | 1 | 1 | 1 | 1 | G5313, G5314 |
| O(1) | 1 | 2 | 2 | 1 | 1 | 1 | 1 | G5318 |
| P(1) | 2 | 2 | 2 | 1 | 1 | 1 | 1 | G5316 |
| Q(2) | 2 | 3 | 2 | 1 | 1 | 1 | 1 | G5291, G5292 |
| R(6) | 2 | 2 | 2 | 1 | 1 | 1 | 1 | G5297, G5298, G5301, G5302, |
| | | | | | | | | G5309, G5310 |
| S(10) | 2 | 2 | 2 | 1 | 1 | 1 | 1 | G5285, G5286, G5287, G5293, |
| | | | | | | | | G5294, G5300, G5315, G5321, |
| | | | | | | | | G5322, G5326 |

^{a}PATS types are designated arbitrarily with different numbers.
^{b}Prefixes of each PATS primer pair A/B and virulence gene primer pair F/R are indicated.
0, no amplicon;
1, amplicon without XbaI or AvrII site;
2, amplicon with one XbaI or AvrII site;
3, amplicon with an additional AvrII site due to a SNP
^{c}isolates of E. coli O157:H7 from various outbreaks that fell within a given PATS type.

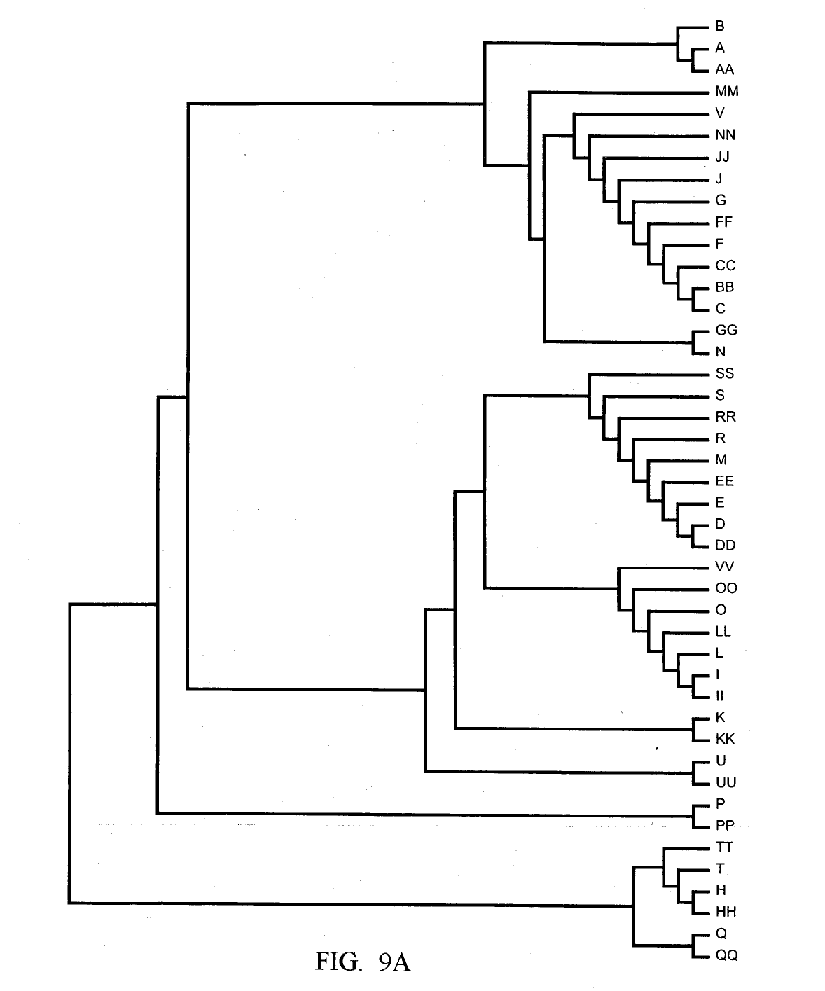

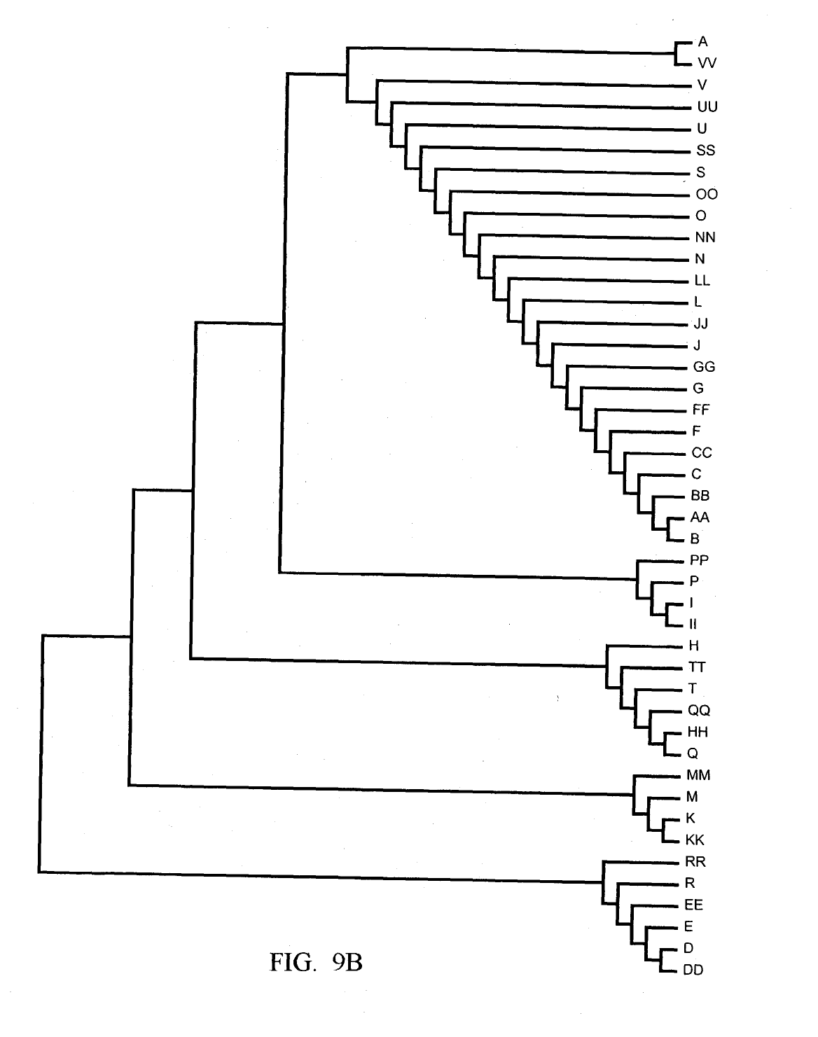

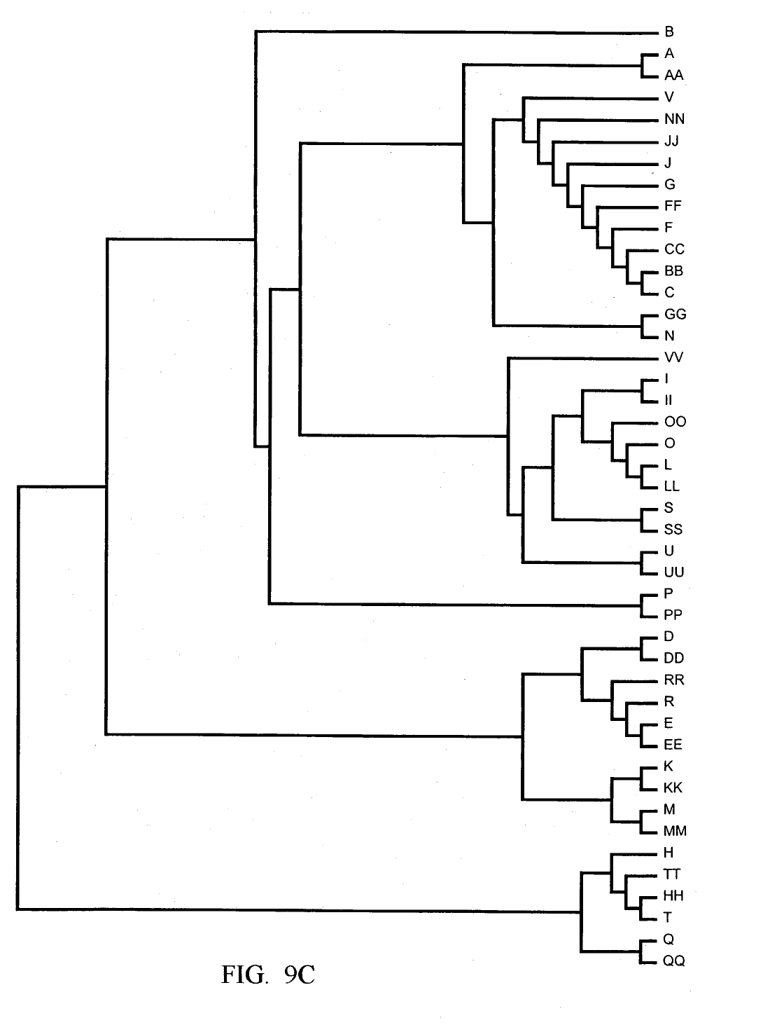

The results of these analyses are also represented as dendrograms. FIGS. 9A, 9B, and 9C show dendrograms based on PATS profiles from XbaI primers, AvrII primers, and a combination of XbaI, AvrII, and virulence gene primers, respectively.

Detailed Materials and Methods

Described below are detailed materials and methods relating to the above-described experiments. In the case of the AvrII sites, polymorphisms were due to insertions, deletions, or single nucleotide polymorphisms (SNPs). The SNPs occurred either within the AvrI site itself, resulting in loss of the site, or in sequences near the site, resulting in the creation of an additional AvrII site.

Design of Primer Pairs Amplifying 0157 AvrII Site

The sequenced EDL 933 genome (GenBank accession number AE005174; Perna et al) was used as the prototype to determine the total number of AvrII restriction sites, and the DNA sequence of the regions flanking these sites in an O157 genome. This sequence was used to design 32 primer pairs that would yield distinct amplicons containing a single AvrII site from O157 strain EDL 933. The primers were assigned a prefix IKNR.

PCR Conditions for AvrII Primer Pairs

PCR was carried out using conditions described previously (Kudva et al., J Clin Microbiol. 40:1152-9, 2002; Kudva et al., J Bacteriol. 184:1873-9, 2002). Briefly, colony lysates were prepared by boiling colonies suspended in sterile distilled water, followed by centrifugation at 4° C. Each O157 strain template was tested with each individual primer pair. PCR was carried out on the GeneAmp PCR system 2400 thermal cycler (PE Biosystems, Foster City, Calif.), using 10 μl of colony lysate, 200 pmoles of each primer, 800 μM dNTPs, 1× diluted Ex Taq™ enzyme buffer and 2.5 units of TaKaRa Ex Taq™ DNA polymerase. The hot start PCR technique (Dieffenbach et al. PCR Methods Appl. December 1993;3(3):S30-71) was employed in combination with a touchdown PCR profile (Don et al Nucleic Acids Res. Jul. 25, 1991;19(14):4008). To create this profile, an amplification segment of 20 cycles was set where the annealing temperature started at 73° C., to touchdown at 53° C. at the end of those cycles. Subsequently, another amplification segment of 10 cycles was set, using the last annealing temperature of 53° C. Each reaction was done in triplicate.

Evaluation of AvrII Amplicons

PCR reactions were initially screened for the presence or absence of amplicons. Amplicons, when present, were purified using the Qiaquick PCR purification kit and digested with AvrII to confirm the presence of an AvrII site within the amplicon. Undigested and digested DNA fragments were resolved on a 4% agarose gel prepared with a combination of 3% NUSIEVE GTG agarose (FMC BioProducts, Rockland, Me.) and 1% agarose (Shelton Scientific Inc., Shelton, Conn.) and stained with ethidium bromide.

DNA Extraction, Sequencing and Probe Labeling

Genomic DNA was prepared using the INVITROGEN EASY-DNA ISOLATION KIT (Invitrogen Corporation, Carlsbad, Calif.) as per the manufacturer's instructions. DNA sequencing was done at the DNA Sequencing Core Facility, Department of Molecular Biology, Massachusetts General Hospital. All DNA probes were detectably labeled using the ECL DIRECT NUCLEIC ACID LABELING AND DETECTION SYSTEM (Amersham Pharmacia Biotech, Inc., Piscataway, N.J.).

Southern Blot

DNA was fractionated by agarose gel electrophoresis, transferred to HYBOND-N+ nitrocellulose membranes (Amersham Pharmacia Biotech, Inc., Piscataway, N.J.), crosslinked to the membrane using ultraviolet light in a STRATALINKER (Stratagene), and hybridized with the appropriate probe, which was detectably labeled using the ECL DIRECT NUCLEIC ACID LABELING AND DETECTION SYSTEM (Amersham Pharmacia). Hybridization at 42° C. and post-hybridization washing of blots was done according to the manufacturer's instructions. Autoradiographs were prepared by exposure of processed blots to Kodak Scientific Imaging X-OMAT AR film (Eastman Kodak Company, Rochester, N.Y.).

Data Analysis for AvrII Amplicons.

Statistical analysis was performed using Epilnfo6 (available from the Center for Disease Control) software. The significance of differences in proportions was calculated with the Fisher's exact test. DNA G+C content was determined using the Wisconsin Package Version 10.0, Genetics Computer Group (GCG), Madison, Wis.

### Other Embodiments

All publications and patent applications mentioned in this specification are herein incorporated by reference to the same extent as if each independent publication or patent application was specifically and individually indicated to be incorporated by reference.

We claim:

1. A method for typing the strain of a bacterial isolate, said method comprising the steps of:
(a) providing genomic DNA from a bacterial isolate;
(b) performing a polymerase chain reaction on the genomic DNA using a first and second primer to amplify genomic DNA comprising a restriction nuclease restriction site, thereby producing an amplicon having the restriction site; and
(c) characterizing the amplicon of step (b), thereby typing the strain of the bacterial isolate.

2. The method of claim 1, further comprising performing a polymerase chain reaction on genomic DNA of a reference strain of a bacterial isolate using the first and second primers of step (b) to amplify genomic DNA of the reference strain of the bacterial isolate, and wherein step (c) is carried out by characterizing the amplicon of the reference strain of the bacterial isolate with the amplicon of step (b).

3. The method of claim 1, wherein said amplicon of step (b) comprises at least 200-400 bp.

4. The method of claim 1, wherein said strain of the bacterial isolate is a pathogenic strain.

5. The method of claim 1, wherein said strain is a strain of E. coli O157:H7.

6. The method of claim 2, wherein an amplicon is present in the bacterial isolate that is not present in said reference strain.

7. The method of claim 2, wherein an amplicon is absent in said bacterial isolate that is present in said reference strain.

8. The method of claim 2, wherein there is an alteration in the size of said amplicon between said bacterial isolate and said reference strain.

9. The method of claim 2, wherein said reference strain of the bacterial isolate is E. coli O157:H7 strain 86-24.

10. The method of claim 1, further comprising digesting the amplicon of step (b) with a restriction nuclease that digests the amplicon at the restriction site and where step (c) is carried out by charactering the digestion products.

11. The method of claim 10, wherein said digestion identifies a single nucleotide polymorphism.

12. The method of claim 10, wherein said single nucleotide polymorphism identifies an additional site of restriction nuclease cleavage in said amplicon.

13. The method of claim 10, wherein said reference strain of the bacterial isolate is E. coli O157:H7 strain 86-24.

14. The method of claim 10, wherein said restriction site occurs infrequently in the genome of the bacterial isolate.

15. The method of claim 10, wherein said restriction nuclease cleaves rarely within the genome of the bacterial isolate.

16. The method of claim 10, wherein said restriction nuclease is XbaI.

17. The method of claim 10, wherein said restriction nuclease is AvrII.

18. The method of claim 10, wherein said amplicon is digested with at least two restriction nucleases.

19. The method of claim 11, wherein said restriction nucleases are XbaI and AvrII.

20. The method of claim 2, further comprising performing a polymerase chain reaction on genomic DNA of a reference strain of a bacterial isolate using at least one pair of primers identified according to step (b) to amplify genomic DNA of the reference strain of the bacterial isolate and digesting said amplicon of the reference strain with at least one restriction nuclease, and where step (c) is carried out by characterizing the digestion products of the cleaved amplicon.

21. A method for identifying a pair of primers for typing a bacterial strain, said method comprising the steps of:
(a) providing genomic DNA of a bacterial strain;
(b) fragmenting the genomic DNA of the bacterial strain into at least two fragments, wherein said fragments include a restriction enzyme site flanked by 5′ and 3′ regions of DNA;
(c) identifying a first primer that hybridizes to the 5′ region flanking the restriction site and a second primer that hybridizes to the 3′ region flanking the restriction site, wherein said first and second primers amplify genomic DNA of the bacterial strain having the restriction site;
(d) performing a polymerase chain reaction (PCR) on the genomic DNA of the bacterial strain using the first and second primers of step (c) to amplify genomic DNA of the bacterial strain, thereby producing an amplicon;
(e) providing a second genomic DNA, the second genomic DNA being from a reference bacterial strain,
(f) performing a polymerase chain reaction (PCR) on the reference genomic DNA using the first and second primers of step (c) to amplify genomic DNA of the reference bacterial strain, thereby producing an amplicon;
(i) comparing the amplicons of step (d) and step (f), wherein a difference between the amplicons of steps (d) and (f) identifies the pair of primers as a pair of primers for typing the bacterial strain.

22. The method of claim 21, wherein said difference is the presence of an amplicon not present in said reference strain.

23. The method of claim 21, wherein said difference is the absence of an amplicon present in said reference strain.

24. The method of claim 21, wherein said difference is a difference in the size of said amplicons.

25. The method of claim 21, further comprising digesting the amplicon of step (d) and the reference amplicon with a restriction nuclease that cleaves the amplicons at the restriction site, and detecting an alteration in the digested amplicon of step (d) relative to the digested reference amplicon, wherein a difference between the products of the digested amplicons further identifies the pair of primers for typing the bacterial strain.

26. The method of claim 25, wherein said digestion identifies a single nucleotide polymorphism.

27. The method of 25, wherein said single nucleotide polymorphism identifies an additional site of restriction endonuclease cleavage in said amplicon.

28. The method of claim 25, wherein said restriction site occurs infrequently in the genome of the bacterial strain.

29. The method of claim 25, wherein said restriction nuclease cleaves rarely within the genome of the bacterial strain.

30. The method of claim 25, wherein said restriction nuclease is XbaI.

31. The method of claim 25, wherein said restriction nuclease is AvrII.

32. The method of claim 25, wherein said amplicon is digested with at least two restriction nucleases.

33. The method of claim 32, wherein said restriction nucleases are XbaI and AvrII.

34. The method of claim 25, wherein said amplicon of step (d) comprises at least 200-400 bp.

35. The method of claim 25, wherein said bacterial strain is a pathogenic strain.

36. The method of claim 25, wherein said bacterial strain is E. coli O157:H7.

37. The method of claim 21, wherein the reference strain of step (e) includes a bacterial strain of E. coli O157:H7.

38. The method of claim 37, wherein said reference strain is E. coli O157:H7 strain 86-24.

39. A kit for distinguishing between bacterial strains comprising a set of primer pairs which, when used in a PCR reaction of genomic DNA from a sample of a bacterial isolate amplify DNA across a site for a restriction endonuclease, the amplified DNA being polymorphic between strains of the bacteria.

40. The kit of claim 39, wherein said kit includes a set of primers identified according to the method of claim 13.

41. The kit of claim 39, said kit comprising primers for identifying a pathogenic bacterial strain.

42. The kit of claim 39, wherein said strain is a strain of E. coli O157:H7.

43. A bacterial strain typing profile, said typing profile produced according to the method of claim 1.

44. The typing profile of claim 43, wherein said profile is depicted on an agarose gel.

45. The typing profile of claim 43, wherein said profile is depicted on a dot blot.

46. The typing profile of claim 43, wherein said profile is depicted on a microarray.

47. A microarray comprising at least two amplicons of a pathogenic bacterial strain.

48. The microarray of claim 47, comprising a collection of amplicons.

49. The microarray of claim 48, wherein said collection comprises at least five amplicons.

50. The microarray of claim 48, wherein said collection comprises at least 10 amplicons.

51. The microarray of claim 48, wherein said collection comprises at least 20 amplicons.

52. The microarray of claim 47, wherein said amplicons, or fragments thereof, are produced according to the method of claim 1.

53. The microarray of claim 47, wherein said pathogenic bacterial strains are strains of E. coli O157:H7.

54. A method for typing a strain of a bacterial isolate, said method comprising the steps of:
(a) providing genomic DNA fragments from a bacterial isolate;
(b) detectably labeling said fragments;
(c) contacting the microarray of claim 44 with said detectably labeled fragments; and
(c) determining the binding pattern of said fragments to said microarray; thereby typing the strain of the bacterial isolate.

55. The method of claim 54, wherein said bacterial strain is a strain of E. coli O157:H7.

56. The bacterial isolate of claim 54, wherein said isolate is from a patient.

57. The bacterial isolate of claim 54, wherein said isolate is from a food source.

58. The bacterial isolate of claim 54, wherein said isolate is from soil.

59. The bacterial isolate of claim 54, wherein said isolate is from a water source.

60. A method of making a microarray, said method comprising the steps of:
(a) providing genomic DNA from at least one bacterial strain;
(b) performing a polymerase chain reaction (PCR) on the genomic DNA of the bacterial strain using first and second primers to amplify genomic DNA of the bacterial strain, thereby producing an amplicon; and
(c) affixing said amplicon to a solid support.

61. The method of claim 60, wherein said polymorphic nucleic acid molecule is an amplicon, or a fragment thereof.

62. The method of claim 60, wherein said bacterial strain is E. coli O157:H7.

63. A method for typing a strain of a bacterial isolate, said method comprising the steps of:
(a) providing genomic DNA from a bacterial isolate;
(b) performing a polymerase chain reaction on the genomic DNA using a first and second primer to amplify genomic DNA comprising a restriction nuclease restriction site; and
(c) assaying for the presence or absence of an amplicon of step (b), thereby typing the strain of the bacterial isolate.


SEQUENCE LISTING (42 sequences)

SEQ ID NO: 1 - DNA, 61662 bp, Escherichia coli
catgtctaaggatatggtttatcattttgaaaaatataattttatttcatcctcctggtcactttggggcacgtctggggcacgggcattaaggacattattcaacatggcaacttgagtcacgctgcactcaggcatccatgcaccataaacattgtagaccatgctggcgctggagtgccccatctgtgatgcaataaatgtcgggtttgctccggaagataaagcccagcacgcataggtatggcgtgactgatacgctttacgggatcggatacccgctctttttattgctgaatcccatgtcgctccgatggagcttaccgcgtagttaatacccgccttgtgattcttgcgaacgatttgcggacagaaaacaaaagtgcactcgtgcaaaattgttcttccgtactcgcgtaattgaacagtgatctgatgctgcctgctaagacgagtaagcatcgcctggtttttaagtgcttcaattgctggtgccagaagatgtataacccggttagtgcctgcgtcggtctttggtagcgtaaaatcacctatttttgtaaaatttcgtcgcactgttatcgtgccagctttcaggtcgatatcctcccatgcaagtgcggcaatttcaccgtgtcgcatccctgtaaaaacagccactgtccagaggtttttggtctgttgatgatggcaggcatcaatgagacggctaaactcatctctggtcagtggatccggcactggttttgatttcctcagcggtgttattgaattaaacgggtttttctccagatacccgttttcggcggcaaagctgaacattccggctgttgttgtcatgtaatagttcactgtgggtacggttcttccttttcgggacgtgctggttttcctgcttcccttttccccggtcagtaaatctttcctgataaacagcagatcttcttttgtaatcgatgacgccagcctgccaggaccaagcctcggtagcatatttttcatcactgattgataacgatttaaggcgttactaccgatttccattgctttcagagtaagccatttctgtgccagttcaccgacggtgatatctttttttaccaggccaaatagcttcaggttaggtgagtcagggaatcgatcggcataatcaaacgttcctgttctgattgcaaagcagaccgaagcccttaactcaccagcgatctttctgtttttcggtgtatcgggcacgcgaagattttcgcgcactcgtttacccctgtacttaaacgttattcggagttttcctccgtgattttcaacgccggctggataggctgaattcgccattgttcctcctgcgtccaagagcacgctcaggttatactcttatggacaagaattaaacatcctgagatggtaagggttggtttttgatccaagtgttgatggtaggaagatgatagagacattcgctgtttttctttggcgttccgtcgggggcgatttgtttatattctctgccgttcatccacgcactgtccctggcgcgtaatattgttccttttctcaggcctgttacagccattaaaagctccagtgttacccattcattcgcatttatctgtacaacaggtgtgatgactggctgaatgccatggttatgttggtttactaactcgcgcataagttgcctctgcatgtgtaagaaaaaaccgccatcaggcggcttggtgttctttcagttcttcaattcgaatattggttacgtcttattcgatgcgcactcctggtatttcgccttttgatattgctaagtcataaatttgcgcggcactatacccatctcgcatccatgaatctaaggcgcgaacagcctcgctacgctttttatcttctctctcatttttgatatcaacgaggacatcaacgcaattaaggcaaatgtggattttgtctttacattcgatcatggcggctttgccatgatttccgccacacagtgagcataaatcttcagggtctggctggtatttctgtaacgttagagggttgaatgttgaacaggccataatcatctccataaaacaaaactcgccgtagcgagttcagataaaagaaatccccgcgagtgcgaggattgttattcattgccgatattcacctttatcgcgaacacctttaccggtttatcaccgaagtgcggatgtgtgattgtcttgatttcatatccgtcatacggaacatcaattcttcggctggaatcgtcgcgcttcggatatccctttgtgataatcaggcggtcatactcgcggaacataattcgcttattccagtagtcattaaacaggcgatactcttccgttttctctccgcgaatcatggcatcgaagtattcacctttaacggcaagttgcaggttagccacggttaacctcctgcggcggttctggtagcggcatccagtgagttgcttgctcaataccattacccggcttaatcgttgcatctccgcgccgaaaggtgcttccggtatagcgtgcggagcatattagcggttcaaccagagagctatcgaaattcaccgaaataagcacgttctggcccttttcaggcattcgatcactacagcttatccaactatccggagttaccggagagttaccagcctcataagcggatttcatccagtgcgtaagcgtttcgatgcttacacatccgcaatcaacgtctattttttctttttgttctgacaaccattcctggaatgacagcttggcagtctggcttgctggatcaattcgtggcaggccgatatatagtggtacatttcccggctccatcgaattgtcgggacaaataaacgtgttacaaccatatttaacgagctcaattcccactgtgtcgatagtggcgaatggttcagtggtcaatgcagtcaacgcaatttcataagcacggcgctcaatattatctcgcacgtccaggctgccgattcgctctttgatttctttaatcatttctttgtcggtgaacgttgtcatgtgttagtccttatccacttcaacgccatctttcagcgtgatgccgtgccaatcatcagcccaactggttaacccaggcgcatcaatgctaggcatatagacgcttgcagtgtggtagcccttatcgttatcaatgctggcaacgtgctcgccgttgtatgcgctcagcgtgtctaggacactataaaactttcctccggctgccctgaaatcctttacagccttcacaagacgattccacgctttttcctgttctggcgtcaggtcgattaattcctgcaaagttgccatttcagttttccttatatgggttaattttattgtgcagtgtgttgaacgacgcccataccacgtcgttatacaactcaataactagctcaattattttcccgattgcccagacaaaaattagcggggatatcggtgtcatcaacacgataaacagaatgagaaacaaaaattctgtcgctctactttttcgcggatattcttttctgaataatgtaggcacatcactctcctttgttgctcctcaaaattttatgccctggcgcaaaagcacgcgttttgtctttgcttattcgccagccatccttgcgcgcctcttttgcacagccagcccatgacgtaccgatatactcaccgaagtctggcgacttatatttgccatctgtacactggaggcaatcacaatagagatgcatggtgtaacttgcagcaatagccatatcactctcctttagtgcgcaagtggtttttccagcggttttgctccgcgctgggctttttgcaaaaaccacaatccatcatcccgtaatatttcatcaaccccatccgtcggttgctgagtctcacccactgccagacgccaggagcgtttctacgaactaacagaatctttgctttacggtttttcatcgttttgctctcctgcatctctttgctgctcgtcgtgccgctgcaataccggtatggcggcgctttggtgtcgggatgatgttctttgcaattagcgcagaagcccaaaaacgagtcggatacggtaacaagccgatacatgccacacgcattactcacctcttttgatgcgaatgccagcggcgcgctcggcttcactttgttcccaaaaccacttgtgaagcgccataagcttttcgtcaatcggtgcatatttgcgattaaagtaggcctgagcatctttctcagattcgtccggtaattcgccagggccaaacagtgtgttataaatccatgctagtccgctcttagcgtcgccagttgcctgccattcgataatggcagcctgcatgaccagaatgtttttcccgattaataggtccagttctttgtaccggttgcggatgtatgcattctcgctttgtaattttgcgttgcgcttttctgaggcttcaagtaacgcctgcttatcgcgtagagcttcttccagttcagcaacatggcattcactatcaataaggttgttctctgctgcttcaagctcaacacgcagcttcccaaccgtaagcgcaatctcctcgttctcctggtcgcggcgtttgatgtattgctggtttctttcctgttcatccagcagtgccagcacggtagccgggttagcctctgctatgaattcagcgtttgcataagcctgatcatctgattcaatcaggcagttaacatgacattccgcaatcacgccaccgggttctcctttccatttttggcaaacaaaaactcctgttaaattgccgtgctggttaacagatgtatgccctacgatgtagcttcctttagttgctttctctgccttttcacgcagtgcctgataattaatttcgctcacttcgaacctctctgtttactgataagctccagatcctcctggcaacttgcacaagtccgacaaccctgaacggccaggcgtcttcgctcatctatgggatcgccacactcacaacaatgagtggcagatatagcctggtggttcaggcggcgcatttttattgctgtgttgcgctgtaattcttcaatttctgatgctgaatcaatgaggtctgccatctttcattaatccctgaattgttggttaatacgcttgagggtgaatgcgaataataaaaaaggagcctgtagctccctgatgattttgcttttcatgttcaccgttccttaaagacgccgtttaacatgccgatcgccagacttaaatgagtcggtgtgaatcccattagcgttaccgtttcgcggtgcttcttcagtacgctacggcaaatgtcatcgacgtttttatccggaaactgctgtctggcttttttgatttcagaattagcctgacgggcaatgctgcgaagggcgttttcctgctgaggtgtcattgaacaagtcccatgtcggcaagcataagcacacagaatatgaagcctgctgccagaaaaatgcattctgttgttgtcatgccgggtctctctcgtttgcttctgctttcgccgccatcatttccagcttttgtgaaagggatgtggctaacgtatgaaattcttcgtctgtttctactggtattggcacaaacctgactccaatttgagcgaggctatgtgccatcccgatactcgttcttaattcaacaggagatgctttgtgcatacagcccctcgtttattatttatctcttcagccagccgctgtgctttcagtggatttcggataacagaaaggccgggaaatacccagcctcgctttgtaacggagtagacgaaagtgatcgtgcctacccggatattatcgtgaggatgcttcattaccattgctccccatatacaaaaccaatttcagccagtgcctcgtccattttttcgatgaactccggcaccatctcgtcaaaactcgccatgtacttttcattccgctcaatcacgacataatgcaggccttcacgcttcatgcgcgggtcatagttggcaaagtaccaggcatcttttcgtgtcacccacatgctgtactgcacctgggccatgtaagccgattttatggcctcgaaaccaccgagccggaacttcatgaaatcccgggaggtaaacgggcatttcagctcaaggccgttgccgtcactgcataaaccatcgggagagcaggcggtgcgcatactttcgtcgcgatagatgatcggggattcagtaacattcacgccggaagtgaactcaaagagggttctggcgtcgttctcgtactgttttccccaggccagagccttagcgttaacttccggagccacaccggtgcaaacctcagccagcagggtgtggaagtaggacattttcatgtcaggccacttctttcctgatcggggttttgctattacgttgtgaatttctgaagctgtgatgacgccgagccgtaatttgtgccacgcatcatctccctgttcgacagctctcacgtcgatcccggtacgctgcaggataatgtccggtgtcatgctgccaccttctgttcagtggctttttgtttcaggaatccaagagcttttactgcttcggcctgtgtcagttctgacgatgcacgaatgtcgcggcgaaatatctgggaacagagcggcaataagtcgtcatcccatgttttatccagggcgatcagcagagtgttaatttcctgcatggtttcatcgttaaccggagtgatgtcgcgttccggctgacgttctgcagtgtatgcggtattttcgacaatgcgctcggcttcatccttgtcatagataccagcaaatccgaaggcgagacgggcacactgaatcatggctttatgacgtaacatccgtttgggatgcgactgccacggccccgtgatttctctgccttcgcgggttttgaatggttcgcggcggcattcatccatccactcggtaacgcagatcggatgattacggtccttgcggtaaatccggcatgtgcaggattcattgtcctgctcaaagtccatgccatcaaactgctggttttcgttgatgatgcgggaccagccatcaacgcccaccaccggaacgatgccgttctgcttatcagggaaggcgtaaatttctttcgtccacggattaaggccgtactggttggcgacgatcagcaatgcgatgaactgcgcatcgctggcatcacctttaaatgccgtctggcgaagagtggtgatcagttcctgtgggtcgacagaatccatgccgacacgttcagccagcttcccagccagcgttgcgagtgctgtactcatccgttttatacctctgaatcaatatcaacctggtggtgagcaatggtttcaaccatgtaccggatgtgttctgccatgcgttcctgaaactcaacatcgtcatcaaacgcacgggtaatggcttttttgctggccccgtggcgttgcaaatgatcgatgcatagcgattcaaacaggtgctggggcaggcctttttccatgtcgtctgccagttctgcctctttctcttcacgggcgatctgctggtagtgacgcgcccagctctgagcctcaagacgatcctgaatgtaataagcgttcatggctgaactcctgaaaatggctgtgaaaatatcgcccgcgaaatgccaggctgattaggaaaacaggaaatggggttagtgaatgcttttgcttgatctcagtttcagcattaatatccattttttataagcgtcgacagcttcacgaaacatcttttcatcgccaataaaagtggcgatagtgaatttagtctggatagccataagtgtttgatccattctttgggactcctggctgattaggtatgtcgataaggcgtttccatccgtcacgtaatttacgggtgattcgttcaagtaaagattcggaagggcagccagcaacaggccaccctgcaatggcatattgcatggtgtgctccttatttatacataacgaaaaacgcctcgagtgaagcgttattggtatgcggtaaagccgcactcaggcggccttgatagtcatatcatctgaatcaaatattcctgatgtatcgatatcggtaattcttattccttcgctaccatccattgaaggccatccttcctgaccatttccatcattccagtcgaactcacacacaacaccatatgcatttaagtcgcttgaaattgctataagcagagcatgttgcgccagcatgattaatacagcatttaatacagagccgtgtttattgagtcggtattcagagtctgaccagaaattattaatctggtgaagtttttcctctgtcattacgtcatggtcgatttcaatttctattgatgctttccagtcgtaatcaatgatgtattttttgatgtttgacatctgttcatatcctcacagataaaaaatcgccctcacattggagggcaaagaagatttccaataatcagaacaagtcggctcctgtttagttacgagcgacattgctccgtgtattcactcgttggaatgaatacacagtgcttattcgtactaataaaatacccaattttctgtttcttggttgtgtccaaagttatattcaatatctggtgttgatgtatcaatattcttcataccatcaacaagagttgatacaacagccaaatcttgtttgattctcattaaatggtatttcttccggcgcaataaactttcaatggcaagtttcttcgttgggaatgcaaaagatctttctgcattttttgctactttcttaattgcatatctatttctcctttgtttccattcctgtaaccactgatttggtgctggtttaaaattaacaatccaatgcgcaggaaccaaccatgcataatgctctgtctgatgaaaagctatatattgaagtgcgaatatttttatcccatcttcttcaactgtcgcctggaatctccagaaaacaggcattccatcatgttcagtttctgattcaggaaaaggtacgctccatgattttgtcatatctcacctcaaataagtggtttgctgcctaatttcattttctggcgaccaacacaagtcacctcgccgtcagttgttttgatttccggtagcctgccgcgtaaatggctacgtttggaagacatacaccagtttctggttgcttatgtccaaactcattcgcgtacacaatggccgctcgctccagattgcgtctgtattctttctgttgccagatcacgtcctgtgccatgaacttaattggcttagcgtcttctatgcgctcaggcgtttcgtgagtacctttagcctgaatctgcgctctgcttagagtagggcggtgtaatacttctgaacttattgcttcttcgcgggccagtacgccgttagctaatgcctttgcctttaaacgctcacgacgacgagaacgtgaattgcctttgaactgagttctgcgtgtcatatagacctcctgatgaactttggtggtgtggtaggtgggagacccatttcgacctgtttcggcctacttcaattcggcaatagtcccgcaggcctcgccgctttacgtgcgacatattcccgtccatgaacccttcaccacaccccaaagttcactttggttattgcgctttgtcagcgccgtagattcatattcgaatcgttgtatattcaccgccctggtgagtaatgcgtcctgctgacgacgataataatgaaccaatagttcgacattatcaagaactattggtacgaattttggtgatttattaactctacgaagtatgattctgatatataaggaaatttatttttgaaaatgtggctgatgaaggttatgcggcagggatcataactgcatggtttagcgagttacatcaataaatacaattggttatgttttttaggtgggcgaacgtgaggcaaagaaaacccggcgctgaggccgggctagattttaaagtatttatcttttagagatgtagatgtaaaacttttcgcctttgaaaattttttgtcatcagaagggcttatgaactcatcttttttgtagggaaccgctaatgctgcatcacgtctgcgaggcagcttgcttacttcctcgcgctttttcatgatcagttatcctttaataacctatacagttttgtaggggtacatcctgaggatattgttaagttcgtagcacgccttttccgcccatcatcgtataaacgaaaaccagtagtagacgaattttctgcgtcaaaaactatagacagtatagcgtccccagactttttttgccattcgcatgtgccgttagttggtttcgtcatctgtagacgccagtcaagaacgccatcacttatagctgagagatcgtttagtacatctagtacggattgatatctttcatttggatctacatgaatgcatttgttcactattgttattaattttttatgtatatggggaggatactcttttaatggatagcagccattaattatcgactctctgagttgttcaatcgtgctaaatgcagatctttctctttcaaaattatcatgtccaacacacattctatatatggttaatcctgcctgatatatgtcatatgtgaaattataatcatttgttgataaagaaaaatattccggtggcacatgaaaatgatatccaaactcaggcgcagctctcgattcctcattgactaactgagataatccaaagtcagatagcatggcctcatttctgtttgatatcataatgttattaggttttatatcaaaatgcataagaccttttgagtgtatatgataaagtccacttaaaaattgaatggaataccgtattatctccctgcttgtaagattattttttttcattaattggtttagcgaaccattatgataaaatggcatggctatatagatattgctctcacattgagcagcatactgaacttgcacaatatttggatgtgcatgtttatagagaagccttgcttcattaaagtagtcgtcgtggttagtgttttcttttttttctatttctttaatcaccaagtcatgagctaggtgtctgtcatgagccagatatacttttgaaaaacaaccctgttcttctagatcactaatccattcgaattctacatcagctcttttgtatggagttagcatccccttacctccgcagatagtgcagccaaaacagcttcatttgtttcagttgtaaaaccagaattatcgattccatttatattgcggtgtgacttcaatatttctttatactcgatctctgttaggttcaatgatgatttcatgccagattttctaatagtgtaatattttcttacatcactgcttgaaaatgcttcttgaataacagcttctatataaaggcggtcaatgctaagattatcagagtttgattcagtaacgcgtatggcagctaattcaacattatataaattaagaatgtcgaggatgttatttctcacatactttaatttttctggtgtgtctaaggtcgaaggtattttaataacatcaacacatttgagtgcagactcattagtgcaatatacaacaaaagatgtaactttgggcgccgctctaacacctagtattctcattttttatatcctattttagaatcaggccgcatctctgcgaccatccatcatccaaacgtctcttcactcatccgaagaagcagcaatccgggttagcacgcacaagctcaagcgcatcagtcagcgaaagttcagtactgtactgatgccatttcatatccttccgcatccaatagattttccatctatccagagaacgtatgtacttgattcttgctgatggcaggatgtttgtttcacctggattgccctgccacacggggcgctgttcgccgatatctatcgtttggtcattgatgctataaacaatatccagttcattgcggatatgttcaggcggccttatgctttcaatgaattggtgaacttctttttttactgcttgatattcaaggtcattgaacgccatctatcctccttacccaaacgtctcttcaggccactggttaccagctatgtgacgatgaagtcacgaacttttcagccactcccttgcctcgatgtcatccagatggcgagattgcttcagaataccagctacatactccacctttgctacttgatgataaggcaacgttataggcctgtgatcctggttaatgcttgtaaattggtattctccatctctgtcatagccaagaaccttaatcatgttgtgtccttcaacggttctgacaaacacctcatcacccgggaatactttggtgttaggctcaatgagtacatattctcctgattttattctgggccacatgctgtctcctttcacacgaagaccaaaggcatctggatcatcgctataaattttgagccacccatcgcgctcttcggtcatctcgatggcaccatcaacaccaagaattgcctcaccaaccacgcgcactaaccctttttttaatttgccaacaaatgaaagagtatcttcatcattcgctccatttaacgaagtgccgtgctgaagccaaacaacatcaacgtttagaaatttcgcaagcgcattcattttttcctgacgcggtaaagactcagcattaaaccatttgctaacgcctttggacgaaagagaaagggcacgggctatagccattcccctaccatgttcatcaagaccagcttctttacaggcttgcgctagccgctgggcgaattctttgcgcactttttcattctgaaccatgagtacgatactaaagcacttgcaaaaactttcagttcaaccataatacgtactgaaagtacgaaaaaggatattcctatgcaaaatcttgatgagccgattaaaggtgtcggcatccctgaagttgcgaaggcttgtggagttagcgaaagggctgtctataagtggctcaaaaacggcttcctccctaagactgagttttttgggaaaactaaatacgcatcaaaaatcgaagagatttctggtggcaaatatcaagcaagcgaaatgcttgaaataagcaaaaagaaccttctggctgcataagtaacaccgctattttcacaatggacattcgtcctacgtcgctgacaaagcgagtcccaatatatctgaccaactaaggccatatgcgtttccacgcatacctttcaactagctattcactattggaaatcttaagaaatggaacaaacaagttacagcaaactatcacagcgagaaattgatcgcgctgaaactgatttactcatcaacctgtcaacgcttacccagcgcggtctggcaaagatgattggctgtcatgaatcgaagataagcagaacagactggaggtttatagcttcggtcttgtgtgcttttggcatggcatcagacatcagtccgattagcagagcttttaagtatgcgcttgatgaaatcacaaagaaaaaatccccggtggccgccggggactctaagcaaattgatatgcaattctgagggaattactggatcaatccacaggagtcattatgacaaatacagcaaaaatactcaacttcggcagaggtaactttgccgaacaggagcgtaatgtggcagatctcgatgatggttacgccagactatcaaatatgctgattgaggcttattcaggcgcagatctgaccaagcgacagtttaaagtgctgcttgccattctgcgtaaaacctatgggtggaataaaccaatggacagaatcaccgattctcaacttagcgagattacaaagttacctgtcaaacggtgcaatgaagccaagttagaactcgtcagaatgaatattatcaagcagcaaggcggcatgtttggaccaaataaaaacatctcagaatggtgcatccctcaaaacgagggaggttcccctaaaatgagggacatccctcaaaacgagggaaaatcccctaaaacgagggataaaacatccctcaaattaggggattgctatccctcaaaacagggggacacaaaagacactattacaaaagaaaaaagaaaagattattcgtccgagaattctggcgaatcctctgaccagccagaaaacgatctttctgtggttaaaccggatgctgcaattcagagcggcagcaagtggggaacagcagaagacctgaccgccgcagagtggatgtttgacatggtgaagaccatcgcaccatcagccagaaaaccgaattttgcagggtgggctaacgatatccgcctgatgcgtgaacgtgacggacgtaaccaccgcgacatgtgcgtgctgttccgctgggcatgccaggacaacttctggtccggtaacgtgctaagtccggccaaactccgcgacaagtggacccaactcgaaatcaaccgtaacaagcaacaggctggcgtgacagctggaaaaccaaaactcgacctgacaaacactgactggatttacggggtggatttatgaaaaacatcgccgcacagatggttaactttgaccgtgagcagatgcgtcggatcaccaacaacatgccggaacagtacgacgaaaagccgcaggtacaacaggtagcgcagatcatcaacggtgtgttcagccagttactggcaactttcccggcgagtctggctaaccgggaccagaacgaactgaatgaaatccgccgccagtgggttctggctttccgggaaaacgggatcacctcgatggaacaggttaacgcaggaatgcgcgtagcccgtcggcagaatcgaccatttcttccatcacccgggcagtttgttgcatggtgccgggaagaagcatccgttatcgccggactgccaaacgtcagcgagctggttgatatggtttacgagtattgccggaagcgaggcctgtatccggatgcagagtcttatccgtggaaatcgaacgcgcactactggctggttaccaacctgtaccagaacatgcgggccaatgcgctgactgacgcggaattacgacgcaaggctgccgatgaactgacctgtatgacagcgcgaattaaccgtggtgagacgatacctgaaccagtaaaacaacttcctgtcatgggcggcagacctctaaatcgtgttcaggcgctggcgaagatcgcagaaattaaagctaagttcggactgaaaggagcaagtgtatgacgggcaaagaggcaattattcattacctggggacgcataatagcttctgtgcgccggacgttgccgcgctaacaggcgcaacagtaaccagcataaatcaggccgcagctaaaatggcacgggcaggtcttctggttatcgaaggtaaggtctggcgaacggtgtattaccggtttgctaccagggaagaacgggaaggaaagatgagcacgaacctgatttttaaggagtgtcgccagagtgcagcgatgaaacgggtattggcggtatatggagttaaaagatgaccatctacatcactgagctaataacaggcctgctggtaatcgcaggcctttttatttgggggagagggaagtgaacgatagctaccgacagtttgaaaactggtggtcaaaagacaaaagccagttcacgggagacgatgaattaaaagagtttgcctgggtgatatggcaggcatcgcgctctgctattgaactggatatcgactggcccgaatcgaatgacgacttttggaaagatggtgaagaaggtgcttatgcgatgggttatgaggatgggcgtgacaaaacggtaattgcagtaatgaaagccatcagggccgcaggaatcaaagaaaagaatttcgattaagcaaatatcacttcaataaatcgcttttaaggcatcacaatcgctctgtagtgaggtaaacgcgtgcaaggcatgccaatagcagcgagaatgaaaaatgcgtcagaatgcgtttgaggaggttttaagaaatgagtacgatagctgagcttgtcagggctaattttcgtgaagagttggtgcgttggtatcggtatcgttcatcgtccagtttgccgcttgatgagttgtatgagcattcacctgccgcacgatgctatccgcgtgaccgtgttcttcgacggttgttcaaactcaacaatgagtttcagcgcaacagaattatccggagtctggattttaagtgaaggagtgagcatgagcgacctatcattaacccagccaaagctaaaagaatgtccgttttgcggcggtaatgctcgtctgtgggttgaggccggaataaatattgatgtgtggggctatgcagaatgtgacctctgtgaagccagggggggcatgggcaccatcagttgctgcggcggctgaaaaatggaaccggagagcaggagatgaagcaaacctttctgcttcgcaacgaagcaatcagaaataacgccatagacgccattctctcactacccatcgacgacaagtcaccccacgaagtccacgttaaagaacccaagcgcagcaaagcgcagaatgaccgtatgtggccgatgctgaacgatgtttcgcgtcaggtgctatggcatggtcaacggctggcgccggaagactggaaagacctgttcactgccctgtggcttaagaccaaaaaactggagcaacgaagtgtgcctggtatcgacggtggcgttgtcatgcttggcgtgcgtaccagcaaaatgcggaaggccagctgactgagcttatcgaaatcatgttctggttcggctcagagcgcaacgtgcggtggagtgatgactcccggcgagagtatgaatggttacaacgaaaaggtagggctgcatgactatcaaatcaaatacaccagcacacgacaaggactgctggcaaacgccgctttggctttttgatgcactggatattgagtttggattctggctggattcagctgcgagcgacaaaaatgctctgtgcgctcactggctaactgaggtcgacgacgcgctcaattctgagtgggtaagccacggtgcaatctggaataacccaccgtacagcaatatcaggccgtgggtggaaaaagccgctgagcagtgcatacaacagcgacagacggtagttatgcttgtaccagaggatatgtcagtcggatggttcagcaaggctctggagagtgttgacgaagttcgtattatcactgatggacggattaattttatcgaaccatcgacagggctggagaagaagggaaacagcaaaggttccatgctgctgatttggcgaccgttcatcagtcctcgacggatgtttactaccgtatccaaagcggcattgatggcgatcgggcagggcgtcagaagggcggcatgaggcgacagcaaagaagcatcaccgacatcatctgcgaaaactgcaaataccttccaacgaaacgctccagaaatttagttttgagcagaataccatgatgtcagtgcaagggggagaaagtctcctcattatctgattcgcaatttacgtgcatatttaaatattgcacgttacaacgtgcatgtgtatgattgacttatcaatcacaacacgagatatgctcatgaaaaatgatgatgttagtgggaaggccaaaggcggtaaggcacgcgcggcaaaaatgacagcagagcaaagaaaagaatcctcaagaaaggctgttgccgcaaaaaaagaaaaagctttattgcccgtatctgcgaatgagggaaagttaaagatcggtgatgcggaattagatgtcgcggttctcgaaaatggacggcgtatcatatcacaagcttctgtttttaaagcatttggccgaccacaaagagggggtagagcacctcaagaagagggggtgatcaatatgcccgcttttatggatgctgcaaaccttaaaaaatatataaatcaagatgttatgggtgtgatcaataaggtcaaatacaagacgattactggctccgtccaggaaggttatgacgcatccataatacctcttgtctgcgatgtttatttaaaggcaagagaggcaggcgctatcaccaggccaaaccagttagagacagccaagaaagctgaaattctggtgcgctcattagctaaagtcggaataatagcgcttgttgatgaagcgacggggtaccagcgagatagagaaaaagatgcgctcgccaaaatactcgaggcctttgtcgcaaaggaaattcaaccttatattacaacatttcctgctgattattatgaagagcttttcaggttaaggggcttagaatacccgccggaaaatccccgcttccggcctcagtattttggcgttttgacaaatgatatcgtctacaagagattggcaccaaacatccttgaggagcttaaaaagcagaacgtaaaggcctcaaaaggtacaaagttgtttcaggggctgacgccaaatattggatatcaaaaattaagagagcatctgtcatcaaccgttacgattatgaagctatctaacgactattcagattttattgcaaaaatgaatcgcctgcatccaaggtttgaggatgtgaaaacagacgaactggatgattcagacaagtaacagtaacccaccttcaggtggtttttttgtacaaatccttcagagtaagtttacctccttcactgcattactactgaccattgacaacttaacaaacccagcttcggctgggttttttattgctgaattttcaatgtgagaggacatgacaatgaatgagctgataaatagcaatgccatcaaaatgacaagcattgaaatcgctgagttggtgggaagccaacacggtaatgtcagaatatcaatagaacgtctggcaaagcgtggggtgattcaacttccttcaatgcaaaaagttgaaaataaacaaacaattagccctaacaaattcacaagcgtgtatatattcgaaggcgaacaaggtaagcgaggcagcattattgtcgtcgctcagttgtcgccggaattcaccgctcgccttgttgaccgctggcgagaactcgaaggggcaaccgcgaaaataccacaaaccttttctgaggcattgcgccttgcggccgaccttgaagaccagaaggctgaactggagaaacagcttgctctcgcagcacctaaagttgagtttgccgatcgagttggcgaggccagcggaattttgattggaaactttgcaaaggttgttggtattggtccaaacaaactgtttgcgtggatgcgcgatcacaaaatccttattgcttcaggtgcccggcgcaatgtgccaatgcaggaatatatggatcgcggctatttcacagtgaaagaaacagcggtcaatacaaatcacggaatacagatatcgttcaccacaaaaatcaccgggcgtggtcaacagtggctgacaagaaagctgctagataacggaatgcttaaagtaacaggggaggctgcttaatggctaaaccagcgcgaaggaaatgcaaaatctgtaaggaatggtttcacccggcattctcaaatcagtggtggtgctgcccggaacacggaactcaattagcactcgaacgacgaagtaaagaacgcgaaaaagcggaaaaagcagcagagaagaaacgacgacgagaggagcagaaacagaaagataaactgaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttgatgaacgcaatattcacaagcaatgcgtggtgtgcaaccagcacaaaagcggaaatctcgttccgtatcgcgtcgaactgattaaccgcatcgggcaggaagcagtagacgaaatcgaatcgaaccataaccgccatcgctggactgtcgaagaatgcagggcgatcaaggcgaagtatcagcagaaacttaaagacctgcgaaacagcagaagtgaggccgcatgacgttcacagtaaaaaccattccagacatgctcgttgaggcatatgaaaatcagaccgaggtagccagaatactgaactgtagtcgcaacacggtcagaaaatacactggcgataaagaagggaaaagacacgctatcgtcaacggtgttcttatggttcaccgcggatggggtaaagatactgatgcgtgatatccggcaggttcttgagcgctggggggcatgggcggcaaataactatgaggatgttacatggtcgcccattgctgccggatttaagggactgatccccgaaaaagtaaaatcacgtccacagtgctgtgacgatgatgcgatggtgatatgcgggtgcatagcccgcctttaccggaacaatcgcgatctgcatgacttgctggttgattattacgtgttgggggagacgttcatggcgttggcacggaaacatgggtgctctgacacctgtataggtaaacgccttcacaaagcggaggggattgttgaaggcatgctgatgatgctgggagtgaggcttgagatggatcggtatgttgagcgtgaattgccgggagggagaacctctgtattttatcagcgaaaaaatagtttacgatcgtaaaaatctgcatatcatgataagagtggttacattgccacgcagtcgaacccgccgatgcgcgggtttttttgtaccccgaatcctgtgagctatacggaaagtacacagaaaggaaggtgcgaccgtaattaataacaaaatcttaaaaatcgcatatagcactattagttttctaaatattgtatattttaagtattgcaggataaccctgtaacgaagtttgcgtaacagcattttgctctacgagtttgccagcctcccccagtggctggcttttttatgtccgtagcgtcaaagcagcaatggcgctagggcgtcgtgcaattggcgttgagctggagagcgggcgttttgagcagacggtcagggaagttcagaatgtagtcagtcagaacggatgatattgcaggattagttacgtaccgttattatcctgcgcccggccctttagctcagtggtgagagcgagcgactcataatcgccaggtcgctggttcaaatccagcaagggccaccatatcacataccgccattagctcatcgggacagagcgccagccttcgaagctggctgcgcggggttcgagtcctcgatggcggtccattatctgcattatgcgttgttagctcagccggacagagcaattgccttctgagcaatcggtcactggttcgaatccagtacaacgcgccatatttatttaccaggctcgcttttgcgggccttttttatatctgcgccgggtctggtgctgattacttcagccaaaaggaacacctgtatatgaagtgtatattatttaaatgggtactgtgcctgttactgggtttttcttcggtatcctattcccgggagtttacgatagacttttcgacccaacaaagttatgtctcttcgttaaatagtatacggacagagatatcgacccctcttgaacatatatctcaggggaccacatcggtgtctgttattaaccacaccccaccgggcagttattttgctgtggatatacgagggcttgatgtctatcaggcgcgttttgaccatcttcgtctgattattgagcaaaataatttatatgtggccgggttcgttaatacggcaacaaatactttctaccgtttttcagattttacacatatatcagtgcccggtgtgacaacggtttccatgacaacggacagcagttataccactctgcaacgtgtcgcagcgctggaacgttccggaatgcaaatcagtcgtcactcactggtttcatcatatctggcgttaatggagttcagtggtaatacaatgaccagagatgcatccagagcagttctgcgttttgtcactgtcacagcagaagccttacgcttcaggcagatacagagagaatttcgtcaggcactgtctgaaactgctcctgtgtatacgatgacgccgggagacgtggacctcactctgaactgggggcgaatcagcaatgtgcttccggagtatcggggagaggatggtgtcagagtggggagaatatcctttaataatatatcagcgatactggggactgtggccgttatactgaattgccatcatcagggggcgcgttctgttcgcgccgtgaatgaagagagtcaaccagaatgtcagataactggcgacaggcctgttataaaaataaacaatacattatgggaaagtaatacagctgcagcgtttctgaacagaaagtcacagtttttatatacaacgggtaaataaaggagttaagcatgaagaagatgtttatggcggttttatttgcattagcttctgttaatgcaatggcggcggattgtgctaaaggtaaaattgagttttccaagtataatgaggatgacacatttacagtgaaggttgacgggaaagaatactggaccagtcgctggaatctgcaaccgttactgcaaagtgctcagttgacaggaatgactgtcacaatcaaatccagtacctgtgaatcaggctccggatttgctgaagtgcagtttaataatgactgaggcataacctgattcgtggtatgtgggtaacaagtgtaatctgtgtcacaattcagtcagttgacagttgcctgtcagactgagcatttgttaaaaaaatttcgcatggtgaatccccctgtgtggaggggcgactggtgaaaaatccttgcttgtgattcattatcgacacgggttcggtggtaccaggccgaactcaccgggaggcacccggcaccatgcagtatacagagattaggcatataccaaggcctctcatagcaggggcctttttacatgtaaaaaagcccgagtgggttcgggcaattgcatgagatactcgttttaataatcgaaatcattttaaccaggattcataaggctgcgcaactgcgcggcctttttcgtatttcgggctgtagtcttccttctgccattgtcctgtaacttccggacttcagcccgctccttattttactcacaatattatcccggccgggaggattcatggcatttaaacactatgatgttgtcagggcggcgtcgccgtcagaccttgcgaaacgaataactcaaaaactgaaggaagggtggcagccttatggtagtgcgctgatttcgacagctggttatggtgcggagttcatccagccagttgtgagtgaggggagcatctcatcaccagaggagccaggcaaccgtccgacgacctcagcgccttctgttgcgccagaatattactatgtgatcgcgcttgctggtcagtccaatggtatgtcatacggtgagggactgccattgccggatacattcgacagccctgatccacgtattaaacagttagcgcgtcgcagtacggtgacaccgggcggtgcagtatgcaaatataacgacatcattccggcggaccattgtctgcatgatgtgcaggacatgagccgtcttaaccatccgaaagcggacctgtcaaaggggcagtacggaaccgtggggcaggggctgcatatcgccaaaaaactgctgccgtttataccggcgaatgcgggcattctgctggttccgtgctgtcgtggtggttcagcgttcaccaccggagccgatggcacatacagtgacgcgagtggtgcctcggagaattcaacccgctggggtgtggacaagccgctgtataaggaccttatcggtcgaacaaaagcagcactgaagaagaatccgaaaaatgtgctgtttgccgtggtgtggatgcagggggaatttgattttggcggtacgccggtaaatcacgccgcacagtttggtgcgctggttgataaattccgtgcagacctggcggatatggcaggccagtgcgtcggtggctctgctggcggtgttccctggatatgcggggacacgacgtatttctggaagcagaagaacgaatccacgtaccagacggtgtatggcagctataaaaacaaaacggaaaagaatatccatttcgtaccgttcatgacggatgagaacggggtgaatgtgccgacgaacaaaccggaagaagacccggacattccgggtatcggatattacggttcgaaatggcgtgacagctcagccacctggacgtcacaggacagggcgagccatttcagttcatgggctcgccgcgggattatttccgaccgtctggcaacggcgattttgcgccatgcgggaagagtggcgctaaacgcgggggcatcatcgacagtatcagaggtgcgcccgtcatcgccttccggtgcagaagccacaggcgtcacaacactgctctcttaccttgccagcgagtcagagggaagcctgaaagtacagggatggtcagccagtggcggcagggcagaagtggtcagcgatgcggagggaaccggaggtaaggcagtgaagctgaccaaggaagccggtaaaagcagctgggtgctggagtacgccgcgggcaacggtgcggctctgttacagaaaggggggcagattcgctgccgctttaaggtttcgggagcgctggctgcgaaccagtatgttatggcgttttactggccggtatcttcactgccacagggcgttgccctgaccggagacggggggaataacctgctggcagcgttctacatccagacagatgcaaaagacctgaatgtgatgtaccacaatgcgaaagtggcgacaaacaacctgaaactgggaacctttggcgcatttgataacgaatggcatacgctggctttccgctttgccgggaataacagcctgcaggtgacgccggttattgatggtcaggatggcacaccgttcacgctgacgcagtcaccggtcagtgcctttgcggcggataaactgcatgtgacagacattaccagaggtgcgacttacccggtactgatagacagcattgcggtggaagtgaacagcacagacactgcggcatgataaaaaaaccgccagcgacaggaatggacgctggcggtggtgatacctatggagaaaaaataaaggaacgatactttcgtactctggtttttaatgaaaacagttcttattgtcaacaataacggaaagaaattatgacatttctgaaccagttaatgctgtacttctgtacggtggtctgtgtgctgtatctcctttcgggtgggtacagggccatgcgtgacttctggcgcagacagattgacaaaagggccgctgagaaaatcagcgccagtcagtcagccggaagcaaacccgaagagccgctcatttagcggcaactttcttaatcacacctttcgacgagaaaatcccatgtcagaaattacatccctggtcactgcagaggcagtgaaggacgtcctgcgctctgaagaagtccggagcgcactgaaacagaaacttcgccataacctggaagcgcgtcttgatgcagaagtggatgccattctggatgaactgcttggtgtacaggcagagccaccgactgaagcgggagataccaccgcagagagcggtgaagttcagcctgaatcaccggtcgccgatgcgactgaacctcaacccgaatcggtcatgatgctgtaacggggagtcagggccatcagtaaacagctgctggcctttttcatgttgtgagcttccggattgcgggagacggggtatgtaccagatggaaaaaatcacaacaggtgtgtcatacaccacgtcagcggtgggaacgggctactggttcctgcagttgctggacagggtttccccgtctcagtgggcggcaataggcgtgctggggagtctgctgtttgggctgctgacatatctgacgaacctgtatttcaagattaaagaagaccggcgtaaggcggcacggggagagtaagctgatgagcaggaaactccgctatggtttatcggctgccgttctggcgctgattgccgcaggtgcttctgcgcctgaaatcctcgaccagtttctggatgaaaaggaaggtaaccacaccacagcataccgtgatggtgcgggtatctggaccatctgccgtggagccacccgggtggatggtaagcctgttattcctggcatgaagctgtcgaaggaaaaatgcgaccgggttaacgccattgagcgtgataaggcgctggcatgggtggagaaaaacatcaaagtgccgctgaccgaaccccagaaagcggggatcgcgtcattctgtccgtacaacattggtcccggtaagtgtttcccgtcgacgttttacagacgaattaatgcaggtgatcgaaaaggtgcctgcgaagctattcgctggtggattaaggacggtggcagagactgccgtattcgttcaaacaactgttacggtcaggtatcccgtcgtgaccaggagagcgcgctggcgtgctggggaatcgacagataagaagaatattttgctgaaaaatgaggtttgcttacctggacggataacacgaaatcctgcaaattggcaaaatgtaagtgaataaagtcaaaacagttgtttaacactcaggcaccgtaatgatgcctttgtcatttctgcgcatctcacgcgcatctcacaacacagaacctttcaggatgacccttgaggataccggtttggctgtcggtgcctttctgtgggctggattcctgtgagacaaggttcatcactaaaaggaaataaccgatgaatatgatggccgtgccgtttcacggcaactctctttatgtagttaaccataatggcgaaccatacgttcccatgaaacctgtcgttgcggggatggggctggcctggcaatcacagttggctaagttaagacagcgttttgcgtcaactataacggaaatcgttatggttgctgaggatgggaaacaacgcaatatggtgtccatgccacttcgaaaacttgccggctggctacaaaccattaatcccaacaaagtaaaacccgaaatccgcgataaggtcatccggtatcaggaagagtgcgacgatgttctttacgagtactggacgaagggttttgtcgttaatccccgtaaaatgagcgtgatggaagaactcaaccaggcttgtgctgacatgaaacgggataaaaacattgccagtgtgtttgctaccgggctgaatgagtggaaacaggttaaagccgcgcatgtatcaaaaatccgtacgctggtaaatgaagcgaatatgctgattgattttgtcctggctgatacaggcaaagggaaaataacaaaggcggattgatggggtggctaatgatatcagataaactcataacgctggtgaagagcctctgtgtacttgtcggcatttcatttttagtcatgctggttgccattttcttttccaccgcctggcgagtcctgacgttatcgggactggtggggtgaaagagagatgaaccgtgttctgtgtgtggtgattattgtcctggcggttggctatggtgcgctgtggctggcaacaaaccattaccgtgacaacgcgctcacctacaaagcgcagcgcgataaaaaagccagagagctggaacaggcgaatgccaccattactgacatgcaggtgcgccagcgtgatgttgctgcgctcgatgcaaaatactcgagggagttagccgatgcgagagctgaaaatgaaactctgcgtgctgatgttgccgctggtcgtaagcgcctgcggatcaacgccacctgctccggtaccgtgcgtgaagccaccggcacctccggcgtggataatgcaaccggcccccgactggcagacaccgctgaacgggattatttcatcctcagagagaggctgatcactatgcaaaaacaactggaaggaacccagaagtatattaatgagcagtgcagatagagttgcccatatcgatgggcaactcatgcaattattgtgagcaatacacccgcgcttccagcggagtataaatgcctaaagtaataaaaccgagcaatccatttacgaatgtttgctgggtttctgttttaacaacattttctgtgccgccacaaattttggctgcatcgacagttttcttctgcccaattccagaaacgaagaaatgatgggtgatggtttcctttggtgctactgctgtctgtttgttttgaacagtaaacgtctgttgagcacatcctgtaataagcagggccagcgcagtagcgagtagcatttttttcatggtgttattcccgatgctttttgaagttcgcagaatcgtatgtgtagaaaattaaacaaaccctaaacaatgagttgaaatttcatattgttaatatttattaatgtatgtcaggtgcgatgaatcgtcattgtattcccggattaactatgtccacagccctgacggggaacttctctgcgggagtgtgcggggataatcaaaaacgatgcacaccgggttttctcatttttcacgagatgggagcgatttcccgcgaagccgcctgtccggtgcggtggtggaagaaaccggataaaacaaccgcattgtgcaaatatcgatcaaatatggtgctgctgtgtgaaatctgaaaaatcacagcggtcattatgcatcagtttttaacacaggacgtcagaacgtgacatggcaaagctggactggaaaaagctggagcaggcattccgacgcgaacatgccgaaacgggaataacattactggactggtgccggaagaaaaagattaattacaacaccgccagaacccgtataaaaatgggcaaaatcgatcatgaaattgatcataaaaccgatcatgaaatcgatcatgacatctcagatgaagaaccctgcaatgacgcgggttccggcgatgaaaaatgtgcaaaaaactctgaaaaaaactgcgcaaattcggcagaaacgaaacggattcgtggttcccgacttttaccaccttcaaacgctttttctcagcgaaacacccacgccgtaagacaccgtggatatgcgaagtatcttgaggcagataacctcatggatgatgcgtccgacatggtgctgttcgatgaactggtgttcacccgggcccgcgcactttcagtaactaaggcacttaaagggatgttcgccgacctggaagaggcaactgacgtggaaacccgtgttgctctttacgacaaaatactcaaagctgaacaggcccttgaccggaatattgcccgtatcgagtcaattgaacgctcattgctgacgctggacgtcctggctgagacagcaccaaaacttcgtgctgaccgggaaagaatcaacgccgccagagataaactcagagctgaaaccgatattctgaccaaccagcgtcggggcgttgttacgcctgtcagtgacatcgtgtcatcgctgcatgaaatgagtaattcggggagactggatgacattccggaagaatgaaccgcgatgtgatgagccgtcagaaatgaccgaggctgaacaacgtctgttcatcatgactaaactgagcaatccctggtggcggctcaatcatctctacaaaatacagaacgaaaaaggtgaactggtcaccttcagaatgcgaccggcgcagcgccagttgttccggagcatgcacaataaaaatattatcctgaaagcgcgccagctgggattttccacagccattgatatttatcttctcgaccaggcattattcattccgcatctcaaatgcgggatcgtcgctcaggataaacaggctgccagtgaaattttccgcacaaaaattgctgtaccgtttgatcatctccctgactggctgagagcctcattcaccatcgttgaacgtcgtagcggtgccagcggtggctatatcctgtttggtcacggctcgagtatccaggtggcaacctcattccgttcaggtacggtgcagcgcctgcatatctcagagcacggcaaaatttgcgcgaaatatccggctaaggcgaaagaactgcgaaccggtacgcttaatgccgtctctgatgaatgcattatttttgatgagtccactgctgaaggcgtgggtggtgatttttacgagatgagtaaccgagcacaggagatcactgcatcaggcttattgctgacggcacaggattataaattccatttttacgcctggtggcaggatcctaaatacagcgccagagtgccggaaagcgggctgaagctgtcacgggaaaaaatgacgtatttttctgcggttgagaaggcaatgaacatcacgcttactgatgaacagaagcagtggtacatcaataaggaaactgaacagcgtgaggaaatgaagcaggagtttccctcaacgccacaggaggcgtttctgacgtccggacgacgtgtgttcagtgccgaaagtacgttgcaggcagaatcattctgttcgccaccgatgattgtttatgacattgaacctgttacaggagcgaagactaaagctcagtctctgcgtgaaggaaataaaaacgagttgcagcggacgctgatgaattatctgctggtatgggaactgccggatccggatgaagagtatgtttgtggggcagatactgccgaagggctggagcacggagaccgctcatcgctggatgttgtcaaacgcagtaatggcgagcaggtggctcactggttcgggcatctcgatgctgaactttttgctcatctcatttcgcaggtctgtcgtatgtataacaacgcgtttgtggggccggagcgtaataatcacggacatgcagttatcctgaaactccgggaactctatccgacacgttatatctacaacgaacagcatcttgaccaggcatatgacgacgatacgccccgccttggctggctgacaacccgtcagagcaaacctgttctgaccgaaggaatgaaaacgcttctgaataatggaatatcagggatccgctggtcaggcacattatcggaaatgaacacctacgtttatgacgcgaaaggctccatgaatgcacaggaaggctgctttgatgatcagctcatgagctacatgattgcccaggagatgcgcgccagaatgccggtgagggtaaaacagaaaacggataaacgcagaaccacacactggatggctcactgatgaaaaatgaaactaacaccatggcgacgaaaaacgacaatggagccacgccgcgtttttctcagcgccagttacaggcgctttgttctgatattgacagccagcctaaatggcgtgatgccgcaaacaaggcctgtgcgtattacgatggcgatcagttgccaccggaagttcttcaggtactgaaagatcgcggtcagccgatgactatccataacctcatcgcgcctaccgtcgatggcgttctgggaatggaggccaaaacacggactgatctggtggtgatgtcagacgagccagatgatgaaactgaaaaactggctgaagctattaatgctgaatttgccgatgcatgccgccttggcaatatgaataaagcccgctctgatgcctatgcggaacaaatcaaggcgggcctcagttgggtggaggtcagacgaaacagcgatccgttcgggcctgaatttaaggtgtctactgtcagccggaatgaggttttctgggactggctgagccgggaggctgatttaagtgactgccgatggctgatgcgtcgccgctggatggataccgatgaggcaaaagctacattcccgggaatggctcaggttatcgattatgccattgatgactggcgtggttttgtcgataccacggttactgaaggccagcccagtccgttgatgagtgcatgggaagagtatcagtcatgggatcgacagcagaacgaatggcttcagcgtgaacgccgtcgtgtgctgcttcaggtggtttattaccgtacattcgagcgtcttccggtgattgaactcagtaatggacgggtggtggcctttgataaaaataatctgatgcaggcggtagctgtggcatccgggcgggttcaggtgaaagtcgggcgggtaagccgtattcgtgaagcctggtttgtcgggccacactttattgtggatcgcccctgtagtgctccgcaggggatgtttccgctggttcctttctggggataccgaaaggataaaaccggggagccatacgggctaatttcccgcgccattccggcacaggatgaggtgaattttcgtcgtatcaagctgacctggttgcttcaggccaaacgcgtgattatggacgaggatgccacccagttgtcagacaacgacctgatggagcagatcgaacgtccggatggcattattaaactgaatccggtccgaaaaaatcagaaaagtgtcgcagatgtttttcgggttgagcaggattttcaggttgccagccagcagtttcaggtcatgcaggaatcggaaaaacttatccaggataccatgggagtgtattccgcatttctcgggcaggattcaggtgcgacgtcaggcgtggctatcagtaacctggtggagcagggggccacaacccttgcggaaatcaacgataactaccagtttgcctgccagcaggtgggaagactgttgctggcttatcttctcgatgacctgaaaaaacgccgtaatcatgcagtggtgattaatcgcgatgatcgccagcgtcgccagaccattgtcctcaatgctgaaggtgataatggtgaactgaccaatgatatttcaaggttaaatacacatattgcgctggcgcctgttcagcagacaccggcgtttaaggcacagcttgcacagagaatgtcagaggttattcaggggctgccgcctcaggtgcaggctgttgtgctcgacctgtgggttaatcttctggatgtgccgcagaaacaggagtttgttgagcgtattcgtgctgcgctggggacgccaaaatcaccggatgaaatgacgccggaagaacaggaagtagcggcacaacaacaggcacttcagcaacaacaggcagaactccagatgcgcgagatggctggcagagtggcaaaactggaagctgacgccgccagggcacatgcagctgcacaacgggataatgccagtgcacagcgggaagtcgccctgacacaggggcagcgttatgtggatgcgcttaaccaggcacatacggcagaaatcattaccggcgtacagaatatggaacaggagcaggacgttcttcagcaacagatgctgtatacgttacaacagcggatgaatgaaatgtcgctctgaaaactctggcttcaactgaaccccgtcatcgtacggggttttttgtttccggaggtaagcgttccgggagcggtgcgcttattcgcgggggcagcgataagccttatttactcaaccattcggatctgtccgataaacagaccatgcggagttatttatggattttgaatttacgggtgaagaaaccccggaacaactggaaaaaatgctggaaggacttggggatgtggatattgacagtcacgcacaggacgtcgtgacggaagataccacggaaaaacatgcggatgaggaagcacagactcagacgggcgataacaatgtggcaccgacgccggatgccagtgtggagcagacgcaggacgtgaaggagccggaagcgaagggggtgctcacccgcgacggtaaacacgtcattccctatgaagtccttgaggctgaacgttccggtaagcaacgggccgaacaggaagccgcacttcttcgtgggcagatagctgaagaaaaacgcagggtggaactgctgacgtctcagatccaccaggccggtatgaagcccacaccgttaccggaaaacgaaaaaatttctgatgagcagattgcccgtatcagggagatgtatccggaaattggtgacgcggtggcttcgctcatccgtaaaaataactatctccagtcccgtgttcagcaatcagcacagcaggcagaaggtaatggtggtgaggatttatcaccggttcttgatgcgatgaatgccgtgccggtgctgaaaacgtggcaggagtccgatccagatcgcttctcggttgctgtatccatcgacgggaagctccagaatgaccccgcatggaaagacaaaacgctcactgaacgtttcgctgaagtggcccgtcgtacgcaggttgctttcggtgaagtcagtgagtcgtctgctgacaacaaggcagacaaaacggatatccggaaaacggcggaagagaaagtgaagaccgctgaacaggagcaggcagtacctgcttccccgtcagatttaggcaccacggcttccgtcggaaccggtgataattttgaacggttacttggcgcttctcattcagaggcagaggcgattatgcgcggtatgacgaatgctgaaatagacgcgcttctggagaagctcgggtaacttactgaaggagaactgaagtaatgacgactgtaacatcagcccaggcgaataagctgtatcaggtggcgctttttaccgctgccaaccgcaaccgctcgatggtaaatatcctcactgaacagcaggaagcgccaaaagcggtttcgccggacaagaaaagcacgaagcagaccagcgcgggtgcgccggttgtccgtatcacagaccttaacaaacaggccggtgatgaagtgaccttcagcatcatgcacaaactctcaaaacgtccgacgatgggagatgagcgtgttgaaggtcgtggtgaggatctcagccatgctgacttctccctgaaaatcaatcagggacgtcacctggtggatgcaggcggacgtatgagtcagcagcgcacgaagtttaacctggcatcctcagccagaacgcttctggggacgtactttaatgacctgcaggaccagtgtgcgatagtgcatcttgctggagctcgtggtgattttgttgctgacgacactattctgccgacagcggagcaccctgaattcaaaaaaatcatgatcaacgatgtactgcctccgacacatgaccgtcacttttttggcggtgatgcgacaagctttgagcagattgaagcggcagatattttttctattggcctggtggacaatctctccctgttcattgacgaaatggcgcatccgttacagccggttcgtctgtccggtgatgaacttcacggagaagatccatattacgtcctgtacgtcacgccgcgtcagtggaatgactggtacacctcgacgtccggtaaggactggaaccagatgatggttcgtgccgtgaaccgtgcaaaaggttttaatcatccgctgttcaaaggtgaatgtgcgatgtggcgcaatatcctggttcgtaagtatgcgggtatgccgatccgtttctatcaggggtcaaaggttctggtatcagagaataacctgacggcaaccacgaaagaggtcgctgctgcaaccaatattgaccgcgccatgttactgggggctcaggcgctggcaaatgcttacggtcagaaggcgggcggtcacttcaacatggttgagaagaaaacggatatggataaccgtactgagatagcaatcagctggatcaacggtctgaaaaaaatccgtttccccgagaagagcggcaagatgcaggatcacggcgtgattgccgttgatacagcagtgaagctctgattttttcctttccctatgccgggttttcgcccggctttttcaggagtcattaattatggcaaagactatccttgccccgtcactgagtgaacgggtctatacgggtacgcacggtaatgagtcggtggcagaaggcgtatttacggtgaatgctgcggaagcggacagtgttattcatcttctctcactgccagtgggcatccgtatcaactcactccaactggtttcaacgggtggtctgggtactgcaaccgtcagcattaagtccggtgagcatgctctcatcgataacagcgaagctgtttctgcaaaatttgccagatatgtgccagtggagccgtacaccacacagcgtgacggggagctggttactgtcaccattaagactgccgctgcaaccggcaccctgaatgttctgctgcgttataccgtggtgggatactgattaaaaccttccggcccgcgtcatgcgggctttttatccggggaattatatgagtgagaaaattgccgttgtctatatcggcccaaaacccgtgaaaaaggacaccattaccggaagtcgcacgctgttcccacgtcttgagccggtgcatgttgacagcgcgatggcctggcaactgctggggtttccggatgtctgggttcgtcatgaagagcttgatgatgttctgaaaaagcaacaacagaatgagcagttgcggcaggcacagcaggcgcaggaaagagtgcttgctgcgctggcagaagcggagaacagttttgttgtttctgttaacgggcaggaggtggatttaagtaagctcacctcagcacggctggcgacgctgtgtgaggcagaagagctggatattcacaaagacccgaaagaaacggctgaggcattccgtatccgggtgcgtgaggcatttcgccgtcgtgttgcggagactgaacagcatggcggaactgagtgattttttaccgtatgtccgtcgtcatatcagcggtccactgaacattatgatgacggatgctctgtcaatggctgccgtggcattcagccgccagtcgttggtgtgccgtcgggaggttactgttgtaccagtagcaggaaaagaaatcgtgcttccgtatgacaaagatgatgaggagtgcgttcatatcatccgtatctctgacgataatcatgagctttttgtcggtcgggatgtggatatcagctccggacgctccctgcgatttgcctgttctcccggtgaggtgagcgtgctttatgccgtcgctccgaaagccggacgcagccagataccggatgaactcctcacatggcctgaagaagtggctgcgggggcacttgagcggttgttcatgcagactggtgtttcatggtcagatccgttacgcgcacagtatttttctgtgcagttttctgaggggatccgtcgggcatatcgtcatacactggcgacaagcccgtactcttcataccgcaaccctgtacgcaggcagaggtttttctgatgacgacgattacagaaatcatcggacgtgtgaatacacaactggttgacccgatgatggttcgctggcccctgcaggaattgtgcgattattacaatgatgctgtgagggcagtgattctggcgagaccggatgctggcgcaagcctggaaacaataagttgtgttcctggcgcccgtcaggttttgcccgatggtgtaatacaacttcttgacgtgatatgcctcagtgacggtagtgcagtcagaccattatcccgggaggtgctggatgcgcagtatcctgagtggcccacaatgaagggtattcctgaatgttttatcagcaacgacctgtccccgcgcgtattctggctgtttcctgctcctgacaaagagataagtattgatgcagtggtaagccggataccggaggcagtgtatgttctgacgcaggacgatgatacgccagttccactggaagaggcttatgttaacccactggtggagtggatgttgtttcgcgctttcagtaaggatgctgccggtggcgcagaatcggggctggctgcgcagcattatcagagttttgttgagcaacttgggatcaaacagggggcagacagtgcattgtatgcccgtaaaaaagtgtttaacggaggtggagtgtgagtgttgttgtttcggggacgctgaaatctcctgatggtgaggcgatatcaggagcaaatattaccctgacggcgctgacagtttcaccggatgcgctcagcggcaccagtgcgtcggcagtgacccgtgaaggtggatattacggaatgacgatggatccgggggagtatgcggtttcggtgacggtgaaagggaagactgctgtctacggacgtgtgcgtattgaggggaccgaaagtacggtgacgctcaatatgctgttacgccgcagtcttgttgaggttagcatacccggagaactgctgacagatttccggcagatacagaataatgtggctgatgaccttgccactattcgtcgcctgaatgaagacacggcgacaaaaaacactcaggccacacagtcaaaagaaagtgcagcagccagtgcgaagagtgcatctgacagtgcaaagacggcaaccagcagggcggctgaagccggacaaaaagcgactgatgccactgaggctgcgacccgtgcagtcacagcagcggggaatgcagaggaaagctcgacccgtgccggagagtctgaaaaagccgccggagctgatgcagaaaaagccagacagcatgctgaaaaggccaggctggcgcaggagagcgccggagagatccttaagcgggcagaggctgccactgtcagtgctgaagaggccagacgtatggctgagaatgcacgggggccccgggggcctcagggagaaactggtccgaagggggatgtcggtcctaaaggcgaaacaggtccagtgggccctcaagggcccgcagggccgaaaggtgagcgtggtgacgttggtgctcagggggctgtagggcctgctggtccgcgtggtgagaagggcgaacagggggagcgaggaccgcagggaataccaggcctgaagggggataccggagagcgggggcctaaaggggaccagggggatatggggccaaaaggcgagaaaggtgatccgggaggtcctgcaggcccgcaaggtcctaaaggcgaacgaggagaagccggaccacagggaccgatgggagcacgaggtgagcgtggggagactggcccccgaggtgaacctggtcctgcaggtccgagaggcgaacgaggagagaccggacctcagggacctcgtggagagccaggtccggcaggcagcgctgcaaatgtggctgatgcaacgacggcacagaagggaattgtgcagttaagcagcgcaacggacagtgatgatgaaacgaaggctgccaccccgaaagcggtgaaagcggcaatggatgtggcaaatgaagcgaaaacaaaggcagaagaggctgcagcaggaggtggtgttcccggtccgaaaggagataaaggggacacggggccagcaggtccggctgggccgaagggtgataagggagagcgcggtgacaccggccctgtcggggcaaccggcgaacggggaccggcaggtgatgctggtccggcaggcccgcaggggccgaaaggtgacaggggagagcggggagagaccggtctgacgggaaatgcaggtccacagggtccaaagggagataccggtgcggcaggcccggcaggcccacagggaccgaaaggagaaacaggtgcggcaggcccggtgggggcgaccggacctcaggggccgaagggcgacccgggggagacgcaaatacggttccgtctggggccgggaaacattattgagacaaacagccatggctggttcccggatacagatggcgcactcatcaccggactgacctttcttgaccccaaagatgccacacgggttcagggtttttttcagcatttgcaggtcaggtttggtgacgggccgtggcaggatgtcaaggggctggatgaagtgggcagtgatacaggcagaacaggagaatgacatgaatattttgagaaagcttatgcagagtctgtgcggttgcggaaagcatgatgactgtgaaaacgggcagtcgcttacagcacaactgcgactgggaccggcagacattctggagtcagatgagaatggcattattccggagcaggacagggtaatcacacaggtggtgatactggatgcagataaaaagcagatacagtgtgtggtaagaccgctgcaaatcctgcgtgctgacgggacgtgggaaaatattggcgggatgaaatagccgacagcttcacaaaaaccggagcccggctccggtttttgttgtcatgtataggggggggttattagagagtgaagtaataaacatgttaatacgatggagtgaaggatgccgtgtaattctggttcaagagttttttatgccggaaaatcgcaggattattctggatagtaaagaatcctggttaataatctgtgatagtcagttgggccatttaatgcgcagtatgtatcagggacgccgttttattcagctgaatctggaaaaattgaaaggggtacatgatgtcgccttgccagtgaaatgggaattcacacgaagacagtgaatagctttctgtatacgggaatggcgaaaaatggactgtatggtgtgagtgtgaaacatcttgcgtgtgcggagtgatgcttctcgttgctacagcggcaatgataatgcagtgaaaaaaggggagcaatatgctcccccaaaccgaaagaaaattgcaataatcaatgaagttatttagtcatcatcagaatgtcatgcaaggcattttgtttcagtgatgccgatcgcgattttagcgaattccatcataaatcccctgatttttaagcctgaagcagtcaaaggaatttctatgccctatatcgatatcaccacgatgcgtgggatgatgccgcgcgttgtgacatccatgctgcccgagcattccgctgtactggcggaggactgccatttccggtttggtgttattacaccagaacgtcagatatccggggttgagaaaacattcacaattaagccaaaaacaatttttcattaccgtgacgatttctggtttgcatggccggatgtggtggatgtgatccgcagtccgatcgctcaggacccccacgggcgtatttactacactgacgggcgttttcctaaagtgacggatgcgactattgccacaaaaggggacgggaatcacccgacatcatcgtatcgtctggggatccccgcgccgacgacagctcctgtctgtactgttcagcagggcggtgatgtttctgacgataacccgaatgatgatgaaacccggttttatacggaaacctttgtctcagattatggtgaagaaggtccgccaggtccggcgtctctggaggtaacactccgtactccgggaactgcggtacaactgacgctggctccggtgccattgcagaatgccagtattaaacgtcgccggatttatcgctctgcatcaggtggaggggaggcggattttttacttgtggctgaactggatgcatccgtgctcagttacacggacaaaataccggcgaaaaaccttgggccttccctggcgacatgggattacctgccgccgccagagaatatgacaggcctttgcctgatggctaacggtattgccgccgggtttgccggtaatgaagtgatgttttcggaagcgtatctgccgtatgcatggccggaagtgaatcgtcacacgacggcagaagatattgtagctatctgtccgctgggaacgtcactggtggtggcgacaaagggggagccttatttgttcagtggggtatcaccgtccacaatttctggttccaaaatcccttcaatgcaggcgtgtctgagcaggcggagtatggttgcgatggagggttttgtgctgtatgcaggaacaaatggcctggtgtctgttgatgcaaacggtaatgtcgcgctggcgacggaacagattgtttcaccggaacagtggcagagtcagtttaatccggcctccattgtggcttatccctggcgtggtgaatacattgcctgttacacgaaaccggatggtaagcaggatgtgtttgtattcagtccggtgaacatggatatccgttatctcagtacaccgtttgactgcgcatgggttgatctcgcgaaagatatgatgcgcgtggtgacaggagacaaaatgtcagtgcttgccgggggggctctgccctccacgataaggtggcattcaaaaattttttcattacctgaaagaacctctttttcctgtatcagggtgaaatctccggcgcctgagcgggtggggatcaccattatggctgatgatgttcctgtgattcattttgcgccgggtacgtttaagggaagtgtggtgagacttccggcagcaaccgggcaaaactggcaggtgatggtatccggattcgggcaggtggaacgaataaccctgagtacatcaatgtcggagatgccggtatgaccagaaaaccgtggcgtgcggggaaggatttatccactgttgtggagaatatggaaattggcaccgggcagcgtggtgacggacgtcacgcatttgtgacccgtgaggaactggttggtcttaaactcgcccggcgtcgaacatcgggtggtgcctcatatgcactgaatccgggtattgagattgacagtactttaatgactgttgattttcccacaaaaccgctgaattttaaggcgaccggtggatttggctcggttcttcttgaatgggatatgcctaattatcgcggacattcactgactgaaatctggcggggtacggaggatgaccttgctgatgcagtgctggttgccacgacgccggggcaggtttacggcgatccggttgaccctggctggtcgggattttactggatacgttttgttaacgcggcaggagtgaaaggtccatggaatgctgaaaaaggcactcaggcacaaacacagatcggcgtgaaggccatcattgaccagatccgcgatgaggctgcaaagtcgccggttgtgtccgagctgcgtaaagaaataaaaaacgcgcaggggcaggctgtaaaggatgctgcaattaagacaaccgaagttgtagggactctcagggaagaaacgacaagaacgattggtggtattgaaacccgcattagcacactggattcgtcaaccagtgaatcgcttaatgaggtcgacaagcgcatcactaaattggataaagaaggcggtgaagcttttcttgcaatgtggtcaaaaaaagcgggagttgatggtatcactgcggggatcgggattgtcgccggaaaagacagtgaaggcaggcctgtaagtcaggttgcaatttctgcgtcgcagttgtttgtctttgacccgaataatccggataacacagcctatccgtttgcggtatcaggtggcaaggtagtgatcccgaaagcgatgatttatgacgcggtgattgaaacactggtgtcgcggaaggttgtggcggatgaggtaaaagccggggtaagtatcacttcgccagttatccggagtgccgttattcagaacggaaactttcaggttgattctcagggtaacctgaatattggaggccttttcagtgttacgtcacaagggcaactgacaattcgttactctaatcagaatgtaggactggtgatccgcaatgataaaattgaggtttatgaccagaatggacgactggctgttcgcataggcagattacgctgatcaggaggtgagtattggaatacggttttgccatttataacagaaataacgttaatgttacgggcgtgctgactccagtatttttcctggacagatttacagcggagtctggctcaaagacgtacactaataaacccgacgggaaatcattgcaggctgtatgttgtttatttccctggaataatgtatttgcggatcggaaagtaccgaagataaccattaatgacaatacggtgacgtggtcgaatcttgagcagggtatgggatcttatatttatacattctggggataagtgttatgtacggtttgagcattatgaagccggatggcagcgtatggataagtccaggttttacgccgcagtgtctgatcaacaaaggcaccataccggcgactgaaaagtctttttttaaaacatcaatcccgtcaggcaaaagttgttttttctttatcagaacagagaagaaggccgatgtcatgtacacgcatgaacagattgatggatatcatgcactaaggcttcatgtaattgtcaggggaacgaaccctggtgttacgacggtttatgctttcgcgaatatggttactccaccttctgagtatggtatcgccatgtataacccggacggtgagatgatttatcatggcgaaatgatgctgcttgacgcgaagttaatacctgttgatatcaaatttgaaaaggaccttggatatccatgcgcaatcatgcctgcactggtcgggtattataactggaaaagaactccttatgatcgaccgatttataccacatccactggtgctacaggaaataaaatatattcctgtgagcattattccggtggtgcaacatgggatattcgaaagccgtatatagataaggtcctggttattaatacatcagtatatgattagttgaagcgagtctttaatattcatttaaaatgtctaaaaagatgtattattaaaaagtttagcgtgttatctgaatacaggatatcttaaatgaagagtatagcaacactggttgtgtgtgcaatctccgggattgcctgtgtaaatttatctgcacatgcagcagaaggagagcatacaatttctctggggtatgcgcactttcagtttccgggactgaaggattttgtaaaggatgcgactgctcataacagggagactttcagtcatttcgtcaacagaaactacttttcttcattgggcgaatatacagatggtcgggtcagtggatatgaaggcaaggataaaaatccacagggcattaatatcaggtatcgctacgagataacggatgattttggcgttatcacctcttttacatggacgcgttctctcactaactcacagacatttattgatgtgcagtcagccgatcataccaggaagattaagaatccggcagcttctgccagaacggatatcagggcgaattactggagtctgttagcggggccttcatggcgggttaatcagtacatgagtttatatgcgatggcagggatgggcgttgctaaagttagcgctgacctgaaaattaaggacaatattaacagtagtggcggattttctgaaagcaacagcacgaaaaaaacctcccttgcgtgggctgcaggtgcacagtttaacctgaatgagagtgttacactggatgtggcttacgaaggttccggctctggcgactggcgcacgagtggcgttactgctggcattggcctgaaattctgacctgtatccggtaaccgtttactacccgctgtgatggcgggttttttattgcccgtacagggcaaaaaccgtaaattatgcgtgggtgcctttcggctgatggctggagggtgaacctgaaggcctgatgtggaaaggccccgagtcaacttaacgttaacccgaggccctaacacttcgtaccttaagcaagtagaaggttagcgcctctctgtaaaaggagtcaagcgctatgtcgcaaaaatcgcttatcaccgtcacaatttgcatgacggttatcttcaccatctggatgttgcacggttcactgtgtgagttccggctgaatttgtggggagcggagtttgcggcgttcttacagtgtaagcagtaggaaaaccgcgacggggacgagagtccccgtcaactggttgctgaggttcagccgatatggcacccgtttcaggtgagagaatgaacgataaaattctctggtatatgcagcgtgttgtgagaaattcccgcaaccctgaatttatgaatgaagttaaagacgcctgccttaaaaagcaggcgttttgttttgaggcacctgatggctttttggtgctgcgttctgtgctcagtgctgatggtatcccttatgttctggtgttgctgggcgtgtgtacggggagtaacagcgttgagcgttacctgccggaggtgaagacattaacccatctggctggcggacgctgggctgagttccatacggcaaggcggggatttatccggctgggaaaacgactgggctttgagcgaatgccggatgatgaggatggcttcatggtgttcaggatagcggtctgactgctacagttttcatcattgtgtttaaaccaacattgtaattcacattctgaccctgctccggcagggttttttgttatccagggggccattatgggtggaagtaaaggcggtggtgataccaaagtaaaaccaacagcagcgcaaatagcacaggaagaagtggcctggaaagggtggcaggattacaaaaatatcctccgcccggctgaagataacttcatggaaaaggtcgatgacctgaacagtgagcagcagtacgacaatattgctggcaccacaaatctggggtatcagaaacagtttggcgaagcacggaaggagcttgcgggtaatcttgctcagtccggcgttgacccatccagtggtcgttttaatgcggtaatgaatgcgaaccagagtgaccaggtaaccgggcagattgacacaaccacacgggggcaggtatcgcaggcagataagtatgttgccgggctacaggatgttgctgctctcggttctggtcagaaggcggatgcgttacagagttttaactctctggcagacagcagtctggcaaaagctaaatcggatgcacaggcggcgtttacgaaacagcaggggcgagcctctcttgttggcgctggtctgggtgcggcaggtgcatatgcgatgcataaggctggcggtagcggaggaagtggcggtgctaaaacacctggcaccggcgctaatgccattcagcatcaggctcagaactggagactgtaattatggagtatggcaaatacgaaactctcgcaagggctggttattcaggagcagaccgcccacagggtgactggcagacgtcagcagcgctgacacgccaacaatacgacgactggcgaaccagatatttgccccgcgtggcaaggctggctgaccttggggagaacaacagtctgatgaatgcacagcttgcacgggtgggaggccttgccacttccagtctccgtacagcgcagatggcgcaggataaccagatggcgagatacggggtaaaccgcccggataatcccgacagtaatacgctggggttacgtaatgccctggcaattgctggcgcgaaaaatggtatccgtgaagccgaacaggatcgccagatgaatattctgacgggggcttctgcaccggcaagacagaaactgagtgttggcggccaactggtggcagcgtaagggggcaatatgggatacggtttactggatattgcaaatcagtcgcggcgtgaggcattacagggaataagtgacgcagacagacgacgtgaagaaattgaggctgcgaacaaacagatggcggcgcaacagaaagcgcagaacaagcagaatatcggtacgggcattggtacgggggcggctattggcgcatccgttggtggtcctgttggtgctgttgctggagcagtaattggcggcattgctggttctttgttttaaggagtggtgaatgagcggatttgcacaggggttacttgccggattcagcaccgttgaccaggcaatgacccgtcgtaaggagcttggtctgcgtgaagcacagcttgcccggcaacagaaaaataacgagcgcgattttgagtttgcgcagtctcagtttgaacataataaaaacgttgatcagcggaactttgattacagagccaaagttgacgaccgtaattatgcactgaaggagagggagtttaacgctaaccagaattaccggaatgcgtcactgggtatggagcagcagcgactccagttgcagaaatacaaccagcgacggcttgagtataacgatatgattgcccatagccagccactaatggaagcgcttggaaaagcaattgaggctggcgatcaggaggctgcaacgcgtctgttcgggcagctgccaaagggacatccattaattcttatgtcaaacgaaggctatgcagcgaaagcgggtcaggccgtgatcaacctgcaaaaaatctttggtgataagccggacatggcgatcgattcgctgaataccccggaaaatctcgatgtgctttccggcgtgtttgccccggaactacaacagcgtattggcatgcctgattcaaccggggacaaaacgataaaagaggccaggattggcagtatcgtaccagcgcagcaggaagggtacgtacttattggccttgatctcacatacagcgatggctccaccgcgcataaacctgtaacagaatacggcagtgcgcaccctgatgatcaaaccgtgctggcgatacccgttgataaggctatcgctcaggtcagggatcgcagcaaatttgcagagatatcgaaaaattatggttattttatgccgaagcagcagggactttctctgaaagagcttcagaagggggccagcaacgtagcggcggacgcgatcaagaatggcggtaatgctcaggctgcggtggatgaatattatgctgcgactggttcacaaccgcatcaacagaaaattcagcaacaaaaacttcagcaacaggttatcaactgggcgggagatgatcctgacaagctgtcatttgccagaaatgtagcggcccgtcagcctgaaatgctggaacctcagaatcagaaattgctggagaacgggtatgcgaattttctccgtattcaaaaggccaggggggaacaggccagagatgaaagtgcttcatctgcatctcagtttatccgtggactgaaacagaattacgcccagtaattcacgatattccattaataccatttcctgatgcccggccattgtgccgggtttttttatggagtctgtatggcctattcagaggaacagcgtcctgaggcgcaactcggtaaccagaatcgtaacagcctgaacattcagcaacccggcgaaactgacagctatgaagcatttttctctgatccgaatcgctggaaggataacagtacgtcgttcagcctgggcgatgtattgccaacaatgggtaaaggtttcgcccagtccgtccggggaacaggggaaatggcccgtggactcggtgatgcgatgattcagagcccggtaaaaacaggggcgcgtattttaaatgagttcagccgtatggggctgccgggtgtcgcaactgtgcaggatatttttgccggtggcagcaggggggctgatgaggtcatcgataccctgcctgatggcaaaaacgcggttactgatattgtcggtaaaggtctgaaggcaaccggtaaggctgtcagtgatggtgccaaagccactgatgaatggctgaccggtaagatgtcgccgggtgcagttcgtgcgctgaatacgccgatgaccgaaggctataatgattctgcggtctgggtggcgaagggtgtaaacctgattggtgcgcttgtacctgatatggttgctggcggtgtggctagaaaggtgggtgatgtcacactgcgaaaaatgctgaccgccgggctggagaaaaaatacatcgcggcagggatgcagccggaaagagccacggcactggcagcagaagctgtcgataaaaaaatgccggatttattccaggcgggcctgatcacccattccactgtaagtgcacaggggcagagtgcaatggcggcagcagatgctgttcttaatgctgattactctgagctggcgcagtcaccgaaatttcagcagacgtttttgtccattgacgccgacccgcagcacgcacagcttactgatcgccagaaaatggatctggcaaaagagcgtgttgccgatgaggtgcgcgcgcagctggcaaccgatcctgaattgctggctgtgaatgccatggcggcaaaactgggtgacgcacaactgtttaatctggtgacacgaggcacagcgaagaccgttaaaagcggcattgtcagaaatgccacggaacagggggcgattaatgcggcgcagggcggctattcacgctatcaggaaaacacggcattgcgtgagaccgccggaatgggtgtgtcaccgtgggagggcgtggctgacgcaacgatcgaaggtgcagcctttggtgctgcgatgggggctccattcggtgcggttgccggatatcgtggcagacgtcaggccgcagaagaaaccgccatgcgtgatgctgaaaccgtgcagcaggacgacgcagccccgcaaccagaatctgttgatccggtggcgcagcagcgtgaatccatgcagggcatgaatcgcgagcagcttctggagcagtatgctgatgcggatatggcaacagagggtgacgcatccgcagctcatcgccgggaagctgccagccagttgttgaatgaactggacgaacagacgaagcgacaggctgtgatgaatgagctgaaggcgaagccgcgttctgaactgcttgaggaataccgcagactcagccagaaagaggggcgcaccgagactgaagaacaacagtttcaggcaatacgagaagtcattcgcccacaacaggaagtgacgccggaagcacagtcacagcctgaaaatgcggaggatggtaacgggagcatttacccgacggtgcggttccgggacccgaatgaagtccgcattgaaattaacgggaatggtgcgtccagaccagcggaacgcattgagaaggtgcgcccggacaaccgttatttcacggatgagaaaagcgccatggggagtgatgttttccgtaatgccgccgccaccggcctgaaaccgtccgtagtgaagaaaggcgagaatcagtatgccgttgaaatggataatcctgcgttctctgaagatgtggcaacggaaaccattaacaccctggctgacggagagcgtattgctgatgctgacccgatggagcagcccgcgttcatgcgtgacccgcgattccgtggtttcacgggggatgatacggaggtacaggcccgccttgcccgtggcaacgcgccgacggcagaggagcttgtacgttcacagatggctgaaggtgatgccggtccgacagcacaggagttaactgagcgtccacgcctgcccgctcccggcgatattcatcccggacagggatatccgttaccgggagaagtggcgcgtacgccggatgaaaatcaggccggacgtggtggtcgttttaccacaaccggtgaggttaagggccagagtttccagaaaggacaagctccggcaccggaaaacgccgctggtcgccagggggaaacactcgagggtgacatggttcgtcgtggtctgccgtcaccggatgcgcagaacgcgacagcaccggtacgtgaagggctaccggctcctgacattgcgcgtaatgttcgtatgcctcagcctgaatcacttccccgcactgtacgggactcactgcctgagcttgcacagcaggcagaagtacgccgacaggccggaggaaatcgtgacatcccgcagcctgagacaatcgcacctgaatctgaaacaactgtctctactgacagggaagctaccgtgcgcggaggtgaagtcaggggcaaaaaaattgaagactttggtgaagaaattaagggcgcggcaaaacaccgttatgcacagcttgctgaaacactgggtaaaacgctggaagacagggattatgccacgcagccgctgagcaaactgttcccgaaaccggactacgcaaaactggcgaacgaaggtgctgatgctgataccctggcaatgatagcgctgtatcgtagcgatattccggcgaagacgaaacacaatacggcaggctggggggagagcataaaaaaagtacgacacagtgtatcggaaatgctgaacggaacggtcagcgcgaaacgcctcgcagaatggatggaaggcagaatgccctcccgttacgcggatacctggcaactgttacgcactctgccaccctcacagatggacagggcttctgcttatcgggtggtatcgggtgtgtatcaggcggcaggagggaagcgttacgatccgccacagaaactttattcactgcgcaataaggacaataaggggagtaacctctttttctcggaaagcagggatgaattactggcaaaggcgaaagtctggtttgcagagcaggaggaaaaatcacaggcgaaaggtgatgaaaaaacagcaccgtcaccggatgacaaaatccgctttgacgtttaccggaatacccgcagtggcgatatttttatcgcttacggtaaaaacaaaatgcgggtgagaggtggctttaagtcagccagtgatgcgcgtaagtacattgattcacatcgtgatgagcttgttcgtcatgtgaaggagatgcgggagatttcgcgtgaggagcagcgcaacgccaccaaccgcgaccgtaccggaccagaacgccgcaaggggaatgtttcaccggagcagttcagtgatgcgtttggtttccgtggtgtgcagtttggtaactacgtggaaggtccgcgtcgtcaggctgatttgaaccgggcttatgactcgctgcatgaccttgcggaagtactgaatgtaccgacaaaagcgctttccctgaacggtcgtcttggcctggcatttggtgcccgtggtaagggtaaggcggcggcacactatgagtcaggtgaggtggcaatcaacctgacaaaaggtaacggaccgggtgcgctggcgcacgaatggttccattctctggataattattttggtcgttatgacgtttccaatgacgggaaaattacgtcaggtggcgactttatgacggaagcacagcgtgtcaggcgcatatttaaagacggcaggtatgttgatgctgaatatccggtacgtcaggaggtttacgacgcttttaaaggtgtgattcaggccattaaaaacagtgacatgccgcgtcgttcagcgcttctcgataaggtgcgctcaaaaccgtactggtcaacggatgttgaaatggcggcacgtgcctttgagcgttatgttcaggataaggcgcgtatggctggcgtggagaatgattatctggtcaatatccgtaaggcacctgagcacaacacagataacacctacgcttatccgacgaatgcggaactggatggcggtattcgtgaggcattcgatcacctgttccgcaccctgaaaacccgtgagacggacaagggcgttgcgttttattcccgtaagggcgttacccgcacacctgaaggtaatctcatttcggatgttaaccgtagtgcggaagccaaaggcagcccggtcccgcaggttgaagcggttgcccgtggcgtgatgagcggcattaaggacagtgacctgaaggtccgtgtggtgaagtcacagaaagaggctgaagcgctggcgggtgaattgttcgatggttacggcagggtgcacgcattctatcgtccggataaacgagaaattgtcctggtggcggataacatccctgacgggcggaccgttcgcgagaagctgcgtcacgagatcattcaccatgccatggagcatgttgtcacaccagcggaatatcagacgattatcaaaaccgtgctgaaaacccgcgacagtgataacgtcaccatccgtgaagcctggcgtaaggttgatgcttcctatggtaaggaatcaccggaagtacaggcgggtgaatttctggcacatatggcggagaaacagccgaataaattcgtggcggcatgggagcgtgttgttgccctggtcaaaggggtactgcgtcgtacggggttactgaagccgacggaactgaacgatatcagacttgttcgcgagaccatccgtacgttaggccagcgtgtgcgggaaggttacacgccgcgtgaggatggcgcgggcgcatcgtttcagtactcccgtagtggtaaacgtgatccgttcaaagtgccggaaggtgagggcgagcgttatcgtgatgaccttgccagaatgatgaaatctctgcgcaccacagatttaacggtaaacatcgggcgtacgccgccggtattgcgtcaccttggtgcaccggatttgccgctggttatttcccgcgatactgtgcggaaggccaccaatggtgtgaaacatgtggtgccgatggatgttatcgagagactaccggaactgatgcacgatccggatgcaatttaccgctcagcgacagaaagaaatgcggttgtgatgctgcttgatgccgtggataaaaatggtgatccggtggtgtcagcggtacacatgaaggctgtccggtcgcgtctggaaatcaacaaggtagcttctgtttacggtacagaaaatggaaaaaaactgaagagtatggaaatgaccgggttaacgttgtaccggagagaaaaattaagccgcgataaccttctgcacagagggctccaattgcccaaaggggaacattcttatcgcggctctgcggataaaatactctatcctgaagatattcgcaaggggccgtattactcccgtaccagcagtctgacaccggaagagacaattgcatcgcgttttgtgcgccagatgcaggataaattccaggtgctgaaagctgttcaggagaatatccgtaaaactggcggcaaagtggacgacagtaacaacgcttatatggcggaagaactcttccacgggaaggcggaaaacgacctgaacgtgatgaaggagcgctacgttcagccactggctaaattactggcggactacaaaattgcgcaggccgatctggatgagtacctctacgcccgtcacgcgccggaacgtaacgcgcatatcgcgaaaatcaacccgaaaatgccggacggcggttcggggatgaccaacgcggaagcggcggaaatcatgcagcgtgtacgtaacagtggcaaacaggcacagtatgaccgtctggcagggattattgacgatatgctggcccgtcgccgtgagcttatccgtgaggccggacttgaagagaacggtgtggtggatgcctggcagaacgcctaccgttactacgttcccctgaaaggtcaggatgttgacggtgtggtgtcactgccccgtacaggtaagggcttcaccatcggcggacgtgaaagcaggcaggccatggggcgtgcatcccgggcacagtctccgtccactcaggcgatacaggacctgagcgaatcgctgatccgccatcgcaaaaacgaagtgggtaacgccttcctgaaactggtgcaggataatcccgacaaggattactggcaggtattcaccgatgacagaccggataccatgcggacgattgcagagcgcaaggaccaggaaactggtgaaaccattcgcgaagttgtcgaacgccctgtaccgatggcaatgatggcagaccggtacttcaccaccaaaaagaacggcaaaacgtactacatcaaactccatgatccgcgcctgatgcgtgcgatgaagagtatgggaccggaaaccagcaatgcctttgttcgtacgctggggaaagttaaccgcttcctggcaacggtgaacacgtcgtataacccggaattcctggtcagtaacttcatccgtgacgtgcagacggcggtgatgaacctgaaggcggagcagggaaggagcgacggtaaactgaaagggctggataacttatccgccctggctgtggtgaaagacagccgttctgccatgtcagccgtatacgccagtctgcgtggtaaaaccctcacgggaaaaggtgcacagtggcagaaggtgtggaaagagtttgttgaggacggagggaaaaccggctggtttaacatgggtgaccttgaaggccagcagaaggaaatggatcgccttgtctcactggcgaaggggggatggaaaggccagagtatcggtgcatggaattcgttccttaaccttgtcgaggatgccaacggtgcggttgaaaacgctctgcgtctttctgcctataagcacgcccgtgatgccggtttgtcacgccagcaggcggcgtctcttgccaaaaacatgacggtgaactttaaccgtcgtggtgagcagggggcgctgatgaattcgctgtacatgttcgccaacgccagcattcagggaacggcaaatctggtgagaacgctcggacatcttaatggtgagggacctttactggagcgccttcgctggaagaatctgaatgttccgcagaaaatcgcgcttgccgctgtgggagcaggttatctgcttggctcgcttaaccgcagcgtggcgggtgaggatgatgacggggttaactggtatgacaaggtgccgtctcatgtgaaagagcgtaacctcgtcattatgaaatcggtgttcgggggcaaggccggagagtactggagtattcctctgccttacgggtacaacgttttcttcctgctcgggcatactgctgaaggtgtggcggcgggtgacctgacggcgtcccgtgctgccggtaatgttgttggtggtgtgcttggtgcattcagcccgattggcagtgagacgtcggaaacactgtccggggcattgctgaaaaatgcagcgccgaccattctgcgtccgtttgcgaaccttgccatgaatgaaaacttcatgggggcgcagatttaccaggagaacatgccgtttggtacaccaaaacctgacagccagctgggaagacgttcaacgccagaagcgtacaaggcgtttgcatcctggctgaatgcgttctcaggtggcagccagtaccgtcccggcgcggtggatatcacaccggaatcgctgaaattctggattgactatatctccggagggacagggcgcttcatttccaaaaccacggatgcggcggtgaaatcgctgaatggtattgatataccggaacagcaggtgcccttcctggggaaaatttcgggggaggtgatgccgtatgcagaccagcagaagatgtacgaccggatgacagagattgcgcagtatcacgcagagctgaagagtctgaccggtgcagaaagaacggcgttcattgacgagaacaacggaaaattgtcgatgaacgggcttatgcaggatacccggaagagactgaaggatttgcgtaaacagcgtgatgccatttacgccgacagtactctcagtctggcgcaacagtcggcgatggtgaaatcggtagagcgggatatgaaaattgccgtggatcggtttaaccgcgagtacaacaaaaaagtgggagtggattaacagaaatggccccgtacggaagtgcggggctgattaagaaataaacactcattgacctgtaataaccggagctattaacatatagtcagaagagcatttcatgtgatacagagagccgatttatgtttaatgaagaaaaagttgcgcaaatggcagcgtatttgctgaaaaagcatggcggatctatgcgtttcattaagctgaagcagcaacggtaatggcgaatagcatttatgagcaaaataatcttgatatgttattaggcgatctgatgtaaccgggatgatgttcaccccttatcgccggggaacaataccggcaatcagaattgcggatggaactattcaggcccacgatgatatcgatgaggagttttttcagccagtattggatggctttcttatatccaaatatacgccatttgacatcctccacgccctgaaggacggggttttacagcgcaccggataagattcggtgttttgttgaaaaatactgtgataacaaacagaaaacccgtcagtaagacgggcttagcaagctgggacggttactttaataatttcagtgcctttacatccacttcaacactgctcaggtctttatcaatttcaccctcaattcttactttgtctttcggagaaacattctgaccggcccatatgctgtcatcgatatccgtgacaattgtcccgctattgtcacgaaactcataacgttcatcacccacttttttaacgatgctcccttcaaggataacccatgcatcatccttcagttcttttgcctgcgctactgttgaacgctctgcttcaggcccttggaaaccgccctgctgtgcaaaagcgccaaaagacacaccagaaataagtgctgcaatcaatacctttttcattcatagtcctctttcagagatgaacattcaaacagcattttcagtatggtaaagcgcgggtgcgttgaggatgcctgacacatcagaggtggcgggagattactcccccgcttggtctcttacttctcagattcgtagtctacgaagacagcgacctccgtctgaccggttcggattcgcacctcgcagaggtctttcctcgttaccagtgccgtcactatgacggttaaacagatgacgatcagggcgattaacatcgccttttgctgcttcatagcctgcttctccttgcctttcggcacgtaagaggctaacctacatgtgttcagcatggattgagcctcagattaatgttaagcgtcttgccggacgcgtaatgttaactggggcttttctctatctgccgttggtgttcatgcccgaggcagatagcctcaagcacccgcagcaattctacttaactctccttttcccgcaaaccgtttttatctccagcgacaaatcgaatacacaaccagcaccaccgccattactgttcctacatttgcgaatgcttcaggccaggtcattgattcaccttctgctcaatatttttaaggtcattttccgcatacagtattgcagttcttgctgcccgcaaccgtgctttggtgttcttttcttcacgctcgaggctggcaacagattctcggagctgatcgtgtctgttatgaagctgtctaatctctctcaccacagcctcaccatcgttcgcacaccgcagaacatactgaaaagggtccacaacacatccgcactgcaagcacaggataacgtgatcctgttcatgaacctcaatggcccgatgcttacagctctgttgtgtgtagttttttctgccagttactgtaatgttcagcagcttctcttcatcgcgctttggctgcaccagtgtgatgatattatcgccttcattttccatcagttcacctcctgcggcggttctggtagcggcatccagtgtgacggtttccacgacgcaccaggaattatccacccatcattagcgtcaggatgccccgggatgtaagtcgcccacttcattcgccagtcacctttcctgtcaaactccacggcaacaagaacggctgttttggtatccggcattcgctcactacaacttatccaaccatccggagttaccggagagttgccagacagcgcattctgcaaccgttccagcttaacgtattcctgaaccctgtttccgtcgcacgcctgaagccattgctcagccttttgcgcatcagtgtgaaaggcacaagtgcgaccgtcatcaaattgcatttcgtagaggttagcaatctgttcaaactgcgtgtgtggcaacttgtaagtttggcttacgagttcggcttccagttctgctatgcgcttctctgatgcttcaagtaacgcctgcttatcgcgtagcgcttcttccagttcagcaacatggcattcactatcaataaggttgttctgtgctgctcccagctccactctcagcttcccaaccgtaagcgcaatctcctcgttctcctggtcgcggcgtttgatgtattgctggtttctttcctgttcatccagcagtgccagcacggtagccgggttagcctctgctatgaattcagcgtttgcataagcctgatcatctgattcaatcaggcagttaacatgacattccgcaatcacgccaccgggttctcctttccatttttggcaaacaaaaactcctgttaaattgccgtgctggttaacagatgtatgccctacgatgtagcttcctttagttgctttctctgccttttcacgcagtgcctgataattaatttcgctcacttcgaacctctctgtttactgataagctccagatcctcctggcaacttgcacaagtccgacaaccctgaacggccaggcgtcttcgctcatctatgggatcgccacactcgcaacaatgagtagcagatggggcattactatcggatttgtatttttgcagggagagattgcgctgcaattcttcgatttcagcggcgttgtcgatgatatctgccattttcctttccttcaggcatgaaaaaaggagccgaagctcctttggttttagaattcgaattgccttgcccgcaggcttttcagcattggtctggcccgctgaactacgaaactcgactggtcaagccgtgccgcctctctcagtagcacatctctgttcttcgtcaccatgtagatggtctcaaacgcaatgtcatacagcttgttcgtgtatgaggagttcagctctttcatgatcggatacaggtgtttgctgaggtcctgggctttttccatccagagttgcatgtagcagaggaggatgatttcctcgtctgtgaattgttgctgtggttctgcttgtgccgactgcatgttgcgaagtttcttttcgcactcgatgaagtatctgcgtatctgtcggcctttttcgttacgctctaccatcgccgtttctttggctgtatcgagggtaaggtggtagtcttttttccctcgcccataacctatttcccgtttctgggaaatagctatatagtcctgattttcaacgaatccgtactcttcaatacgttctgtaatccacgatgcaaagcgtttacctactccaagaaaagtatgtaaatcacgggcattaacgagaagagtggtttcgttggcgatagtgccgttgaatacggggatgcgttgactggtcatgatgacctccttatttgtttagtttataaccgccagttagtagctggcggtcgggtgtcaactgagccaaataagaagctctgggcatattccccttgcgggtgttgtattacgcctctccacccgacctttgtacggatgtaactatgccaaattgcaggcataaaaaagccgcaaagctatcgggtgcggagaccgcttatttgttcagtgcggtcagtatgcgatagctctggaggatttgtcaatcagcggaatattgatgaacgggtaacggtaaatgcctcatccagaaaattaacgcaaattcgcatcgcaaacttgcgttaattatctgaaatatcaagttaaatccccttaaatgtttggggcactattggggcaaaatgtggttgtttggggcactattggggcaaaatgtggttgtttggggcatatttggggcaaaaaagaacgtataaagaacggtgtaaaattgatttcttcatggtcaaaatgaggtgtaacagattgaaaaatatatgctcttggacgatcttcggtaattctgtgtttttaatgcttcatggtttcaa

SEQ ID NO: 2 - DNA, 72480 bp, Escherichia coli
  misc_feature 22519, 22524, 22687, 40171, 48548, 49106, 49254, 49258,49274, 49283, 49308, 49318, 49823, 50165: n = A,T,C or G
cagtgtggtacatggatatcgataccacggctaagtacaaatctggtgttactaccgtaaaagactcggtacgactggatccgtgggtgtttatgttctccgcaggatatcgtttttaattttctctgcaaaacctctgcaaaacccctctgcaaaactggtcatcaaatgaccagttttttccattccttaccgcgtgcgtcgttgtaaatatcggtcattttttgattcgaatggcctagcaaaattttggtatcaaccccctgctctctgaacaatcgctctgataaagatctctgctcatggaaagagggaggggtgccattagcacgccagttgtaatccacagaatcccgggcttttttaaatgcaacggttaatgttgctggcttaaccatcccgccgcgcttagctgtccctttcgcgtgatggtggtgcaatagccacggactaagaacgcaatcgcggcaggatgacaccacatcatccagggtgagatttaatttatcgcaacgcagagccagagggatggcaatccgggttcctgttttttgctgttcgacatgaagataaccatcccggatatccgaaaattgcattttgcaaatatctgaaaggcgctggcctgtcatcagtgccagcagcataccgcgctgtaaaaagtaaccatccttttccgctgcgttataaatcatcatccactcatcaaaagtcagtcgctgtcttgatatccgcacctgcggtttttttgccgattctgcagggttaaagcctggcgggacatcgcccgtttgctgagcttcccggaaaacatcgatcagtactttcctgaaaatttgtcccattctgttatgtcctctggccttgtactcttccagtactgataccacatcttttacggttatggcatctaacggtctggtgccaaaacgttcatcaaataccctgagaggggccgctttctgtttcagcgtgttgagtttgatctcgccgttttcatatctttcctgttgaatttttctgtaattattcagaaaaatggtaacggttgatgaaccgccggtatcactaataattttctcctgcagactgagcatttgttccatttgctgccgggcaagacggctgttcgcttctgctgcaatagtttctgccagtttctggtcaatactgccgagaccgtgatttttgcctgttatgggatgcctgtaacgccagtaaactttgttatttcttttgtcaaaatacggagataatcccggaacatcggttttatattttcgcgggcgcgccatcttccagtatcctcttcaaagcagggtgatctgtggcgatcacctccggcttgtttaccattccgacaaagcgagcttgcggatccactcgccagcgtcttccaacttttttggggagaggaaatatcattccggctttagcgtatttacttaacgtactcggagttgggaccggttcactgaattcctcttttgcccactcagtgagcagaataagtcttgccatgagcgtcgttcgctaatcatggtcgccgccactatagctggtgggcaacgaccggggttgaacattaaaaatcagcctgattcgggatcagtttttgccagataactgaaacgtattttgcctggtaacgggcgtcatcaagtgcattatggcgctcaccttcgaatggaatagccgttctggcatcgaagtctatggctttccccagctcaacgattgtgcgtacatcgcgatcgttgtagtaacgccacgggcaggggatcccctgccgttcgtatgaacggcgcaaaatcgtgttgtcgaagttggctccatttccccagacctgaacaaaaaattcaccggagttttcgtcgataaattcccgcaattgtaatagtgcatcatctaacgggatttcatcggtcataatggcagattgcgcttcgcgtgattgcttaagccaccatttaatggtgtcccgatcaatgaccccgccagcagtttccagatcgatagtcttactaaattccggtcccatatctccggtttgcggatcgaaaaatattgcacctattgagatgatcggggcatcaggattttttcccatggtttcaaggtcgatcattagatggtcacacgtcctgctggtggatgtgatttcttgatgaccgttcaccttaattgagtgatctgccgtctcgccagtttcattatcgctatcgtgatgctgattgccgtcagtgttctccttgtgtggatgttcagcgccttccatttcctccggatcatcttcctgaacttcaacctgatactcttcatcgaatgtttcctggtatgttgcgtcgcccatcaccgcgccacaatcagggcagttgccgccgccggtctgaccgcaggcggtgcagactttttccggttcctgttgcgctactggctcaggttgtttcgtttctggctcgttttgttgcgtatttgggctgttttgttccgctttctggtcgttctgttccgtttcttgctggttctggttcacagaatcgcgggtttcaatccccttcacccatttcggatcattcgggtcgctaatccctgcaacaaattcaccacgtgatgcggcgagcaactgattggcgtcaggctggctgatattggctgcctgcataattttctttacttcgtcagcggtaacttttaccggttctggttgttcagaattttgtgcggtatttgcattttgcggtaagcctgtgtatgtgccattttttcgggcaaaatattcttcttttgtgatttcagtagccccggcagccagcgccttatccagaccagaaagtttgtttgcgcgaccgtatttttcgccatccttgtcggtgaagaggaagtagaacggcccctcacgctctacagatggttcgacttccactttgcattcggttttttcgttgtccggaattgccgtttccactgcatcagtttctggtactggcgacgagagagtgtcagttgcgctctgatttgttccttcatcttcaaacacgccctttgtagtcaggtattcagtaatgtatttgttcagtgccacagggtctttgtgaatgtcgatcggacgttcacggacaaggccaaaaatagtctggcggtcgtagcgaagggcatcaggctgtttgcgcattgatgccgagatacgcttccagtcttcgcggtcgttgtcgataacttcattttttgcccagcgatggatgctgccgtcaatgtttccggcatccacatcaccaggccagagagcgtaggccagttcgtcatccagtgttttccatgtctgcttatattcgcgacgaatgactgcagtgacaggggggattttttctgctgagttttcagtgtgctgtcggtggactctggcgcgggcgagatcaacaacagacgtgtattttccggtttccttgcgttcaccttcgcgacgttttttccagatgcgcatttctgcctgaatttcgggccatttggcaccaggcttacatttatgcttaacccacccgatggcatgcagcttaagctccggatacatggcgttaacttctggcattttcatcaacgcttcaacgatatgtccgtcgaatgttgccatgtcttcctgcaacaattcctgcgcgctaatcaccatatcaacggtgatgttttcacatgtgtcgaacttaaccatgacagcgttctgtacttcaggggccagcttgtcaaaagcgacgttcatcggatcggattcagtctcaaccgggacaaaggaagcagccccctcatcccagcggttttcctgcatatattcagcatcccaggaatcgagggcagggcggggtataccgggtttatcctcgcaaacaagaaatttataagcgcagtcctgagcagccggataatgctccaggaattgccagtgaaattttgcgcgggcgcgacgttcatcaccggcttcaatggcagtggctacagcgactgcaccttcttcctttattgcctgttcgtccggaatggcggcgcaaataaagactttactcattttgttttacctcattacagatttaagggtgaacaaatccctgccattgctggcatataagaatgaaatcggatgtttattacggaactgttttaaagacctgccgggatttcgttattatcctggtgaataactttatcgacagggtaacagttaccgggaattttctgttcggttgctgcagtcacacactcctgcattgtcctgtgaacactgactgcaatatcaactggctctccggaaacaagaaaaactgtcagaacaagtgcaaatgctgtattcattgccagcatcctttttgtatcggacgtaaacgggccagcattgaaagaatgcatattttatttaataactcccgttcgtgttttctcttgttaatggcatcttcagtaaatacagggttactgatagtgacaccaatttcaaaacaaccttcagacgtattaacgtttggtaataacgtttccattatcgcgtcctcaacaatgaattttgtgatgcggtgcctggtgcctccaggtgacgttaaccagttaacaattaacgccggatacagagaatccccccataacactgtttttggttttaactgttccgcgtgcgcttagccgcattcactgcatcacaaaattcactttaaaaagggcggacatcagttcatgggcaaacagatgccgccaaacgtcaccagaaaattgataacagagggcgttgcagcggggttgtcacttaagcgtatggtcaacctgacaacccggtgtcctcaacggggaaggaataaccccgccatacttaccgctgcgccatttcgcgttatgctctgacttttcagagaaatatcctttcagtaaactgtcagtgccggatgttcacccgtgtccggcgcacgcactccacttcacccgtggagaactccttaattaccaaccctcaggagggtgaatgttaaaatcaactcttattgctaaatgcctttatcaaaatcgcatggtaagcagcatttcaataggcgagtctgcagttaaaagtattttcgaagagtactttcccgggcatgattttaataaatggaataccaaattaccgccagcagtttcaacgcgtattctgaaagcaaccgaaagagcaagtacaattcgcgttaactatttcattaaagatttgtgggatctttgatatccacagagcctaaagtatgtgcatatggatgtgctattatgcgccctcgcagatttgcatcattttctaaattcactgaacgaaacagggcatcaacaaggctctgtacaatgcaaaggcaatcgaagactgtcgccgtttctgttttgattgatgaaagaacatggccattcacgcaaacagaaattacccgtttattaacatcgctttcctgcttttgattatcagaaccatatagcccagaaaaagcattgcgcacattacgaaccatattatcgatggtttctttttctgcggtactaaggtcaagagtagccagttgtgaacgaactatattcgatgccatttcctgtaatggcgttggtaaatctttaaattccattattagcctcgttggttagctattaacgtgggtatgtaatcattctggcaatgcttaatgccgctgctttttccagattggtgatatcctgctccagagcggacagattttcagcctgcttagccctggcttcattggcccatttcaggtcctgcaccgccttaattttctggtgcatccactcataaagttcatcatcggtatagtctggcgcgatgatgacggggtctcgtttctgcatgtcggctccttgtggttagcgttgcctgctttttaaccacgtcaggcgaggtggtatcctctgaggggtctgttactcgagaggaaattggttatgaatacaatcaagttttcttgcccagaatgtggtggctaagtctttgacacatcctttaagccgcagggctctgacagtttcgcgggagccatctgcaaaaattgtggtcaccttgtaactgaagatgagtcctcgcagttcgatgacgaaatcgttgacaatatcttcggtgcactcaccagagactttctgaagtaaaggcgcataccgcttagttaccgctctgataactcttacctgtccggcaatggcgctgatatcaatataaagcgccatcgctgtttctttgctgatccctggacgccttccattctgatgtttgactcgcccactgagaaatcctctgcttccccttaacgccgggtagcggaactgtttgctgagaacaccgtgcggtgtcttgatgagtagaatttagaatagcctaagagttatggtcaagctttttgtgtagaaaaacctaagcttcttgatgtaaaaaacacaagtatttgaaagtttgtgctttttattacagagagttgcgaaaaaaaggggggttatttatttgcgcttcttttgcgagctttgagtagttcttcaaaaagtttgttgaaattctcaactcgagcacgcatctctgacaacagagccttttgctctgactcaggcagtgcgtcgaacagttgaagcaactctttttgatcttctgtcagattgactggctgattatctgggatcggttcgcctggttgcttatcttcatctccaaaaagaagccaagtcggcgagcactgaagcgcctggctcagtgcgaataatctcttccccgctggctgtgtttcatctctttcccattgagaaattgttacgtgagcgactttgaccagcttacctaatgcggcctgagacagttttaatttttttcgcctgtataagaggcgagcaccgaaggtttcgtttttcatattaggtaattctaatttttcttgacttaggtttctctacgatctagtttccttaggaaaatctaagggtttcgatatgttgaaaattgatgctatagcgttttttggcagcaaaacaaagcttgccaatgtcgcaggagttaggctggcaagcgttgctgcatggggggaactggttcctgaaggtcgcgcgatgcgcctgcaagaggcatccggcggggaacttcagtacgaccccaaagtttatgacgaatatcgtaaggcaaagcgggcggggcggttgaacaatgaaaatcaccactgaacaggtttgtgaggctctggatacctgggtatgccgaccaggaatgacacaggagcaggcgacgatattaatcacggaagcattctgggctctgaaagaacgcccgaacatcgatgttcaacgcgtcacgtttaatgatggcgaggttgatcaacgggcgctgggcgttaaccgggtgaagatattcgaacgctggaaagctatcgacaccagagataagcgtgacaaattcacggcgctgattccggcaattatggaggctatccggatcagcgatttcagattgtattgtgaaattactgacggaaaaagcattacgtacatgatcgccgggttaaacaaagaatatggcgatgtggtggagtccgggctgctttttgcggatccagttgttgtggaacgtgagactgacgagcttatagaaaaagctattgctttcaagcacgcgtatcgtcagcaataccaatattactttgcagataaacaaatgtctgccaggggggcgtatgagtatcgatgcactacgatgggctaaaaaggtgaaaaccggcagttcatccagtaagtcagtattgacctggcttgctgatatgtgcggtgccgatttgtgtgcatacccgtctgtatctgcactggcagaagtaacggaactgaacaaaaagactgtgcaggacagcttacgacacctgatggagattgggttaattgttgataccggtgagagaaaaggcagaacaaagcaaattgtggtgtaccgacttatcggtgtagaagaaagtgttgccgagcctgaatacacccaaaaacgggagtctttaaaggtgggtaaaattggtgctgttaataaaaacagtaccgaaaatggttatgtttcagcacaaaacagacccaaaaacagaactcttagctgcatggaaaataaccaaagacacccaaattttccatcaaagacacccaaaaacggatcacggaacccaaaggaacccaaagatctaaaccccacacataacgcacgcgagagtgctccgaccagtgagcaggaagttttgtcgttacaggcagccccccctgtattcctgtatggcctgagcgaacccatcggaaaattcccgatgagcgatagctggtatccgtcacgggattttcgacgacgggctgcgttgtgggggatggctttgccggagacagaatttacacctgctgaacttgccgccttccgggactactgggcagcggaggggaaagtgtttacgcagattcagtgggagcagaaattcgcccgtcacgtaaatcacgtcagggcgcaggttaaaccagtcagcaaaggggtaaaccatgcagcagcaccaggtggcaccgcatcacgggcagttcaggaaattcgggcagcacgtgagcagtgggaacgtgaaaacggatttatcagcgacggaaacggcctggaagctgtgggaactcatgggggtggtttattcgaaccgctggatccagaagaacggggccgcaccttcgaagctctggattgcacagattggcgcgatgactgagcagcaaatccggcaggtctgccgccagtgcatggaccgctgccgggcgggtgaaacatggcctccggacctggctgagtttgtggcactgatttcgaaaagcggagccaatccattcggtctgacggtggatgctgtgatggaggagtaccgccgctggcgcaatgagtcctggcgatacgacggaagtgataagtacccgtggtctcagcctgtgctgtatcacatttgcctcgagatgcgttcaaaggggattgagcgccagatgaccgaaggggaattaaaacggcttgcagaacggcaactgacgaaatgggcaaagcatgttagtaacggcctgagcgttccgccagtccggcgacaactggcggcccccaaacgcccgtcggggccaacgccaattgagttgctgaaacaggaatatgaacgccggaaagcggctggttttgtttgagttgagaagtgattttttaccgggaggaaatttatggagactgtttttgacgcactgaaagcgatgggaaaagccacgtcggtatagctggctgcgcgacttgatatcagtcgtgaagaagtactgaacgagctgtgggaactgaaaaaggctggcttcgttgataaaagcgtatacacctggcgtgtggctgataacaacgttcagcaggaacagccagcgccagaagaaaccaccacggcaacagaagcgaaaatctcagagtgcgatttaaccgcgacgattgaacaacgcggaccacaaacggcggatgaactggctacgctgttcggtacaacatcccgcaaagttgcttcaacgctggcaatggcaatcagcaaaggtcgtctgattcgcgtaaaccagaacggtaaaattcgttactgcataccaggtgataatttaccagcagagccgaaagttgaatcggtagcggaaaccgatggtaaagcctttcctcagccagccggtgttgcgttaccggtacagaaagatgcaacacaggaagatattaaaacagaaactgtggcggacattgtgcagtcgctgccatcgtttactgcaacgcgagaagatgatttgattttgccatcgctgcatatggcaaatcgcgaactgcgtagggcgaagaatcatgtccagaagtgggagcgagtctgcgccgcgctgcgggagctgaacaagcaccgggatatggttgccgggatttgtcggaagtccgggcaatgagcggatggtgcaggcctgaaatcatgatactaacaatgaaggtaaaatgcatcggcagtctgattggtcgtagtgaggcggcggtcaggatgaaagcccaggttaagggaataagcctgattctgcggggtgattttcaccagtcaacaaaatatccgtagcgcgataacggtcaaaaattatggcgctgacacttttgtgccactggagatgactgtacctaagttcaggggagaagaacacgtccggtgggatggtcgggccakatttaaagggcaggtcatggctccagcctgtacgctggcaatggargctgcctggcgggaaattgatatgggaaccacgccactcagggatttactgccggtccagagaataaattcctgttacggttacaccactgtgatcttgcaagtgcaggaaagtaggtctacacggtaacgcgagtgcgtgtaacttttgatgtcattcccgtagaaacaccggacaaattttcgctgacaggtcatgcagaaggtataaatctgcagattatggacaattacggatatccggcaagagccggaaaaagcatgccgcctctaattctcagtggaagatggacttgattatactcattgcattgtcagaaatagttatccattaaaggctggctattccaaacaggatgttgattacaaaaatgtaatcaacatgtaaggtttatactcttcaatatgcgtataattttccttattttgttgactttaaataacaagctatgcacgaggtaaagtcsgataagtttatctggatgtaatatatattatttgtagtgtttataactttatttcatgataaccaataaaaggagttttttatgaggaacataatggcaggttttttaatattcctgtcttctgctgcttatgctgatatcaatctgtatggtcctggtggcccgcatacagccttgcttgatgcagccaaactttatgccgaaaaaacaggtattatagtgaacgttcattacggcccacagaacaaatggaatgaagatgccaaaaaaaatgcagatatcttgtttggcgcatcagaacaatctgctctggctatcattcgggaccataaagacagcttcagtgaaaaagatattcagcctctttatctgcgaaaaagtattttactggtaaagaaaggtaatcctaaaaatatccggagtattgacgacctgaccagacctgggattggcgtaattgttaatgatggtggtggtaccagtaatacatcaggcactggcgtctgggaagatattgccggacgtaaagggaatatagaaactgtcgccgcaatccgaaaaaatattattttatatgcgcccaatagcggaactgcacgtaaggctcttgagaatcagcctggagcagatgtctggataacctgggctgactgggcagccagtaatccagaaattggtgatgtcgtggaaatagcgccagactacgtgatatggcgtgatatgaacattacagtacgtcaggatgcaaatgatgaaacccgtcgatttgcagaatggctacaaaccgatgaagcggcgcctgcattcaaaaaatatggctggaccaggaaaggcacttgacatcctcgtccttcaggacgtggattctttttccggatgccgcgccagcggcatgtaggggcagctcacaaaacggaaaaaattgtacgctaagcctcgccaggtgaactgaattcattccgatatgggaattcccatatcgggcgaaaacggtttgctgtaacggcagagttaagtaggattgctgcgggtgcttgaggctatctgcctcgggcatgaacaccaacggcagatagataaaagccccacccgactataaatcgaagtgaggcccctatatgctcgtcacatatagattgcctcttacggaccgaaaggtcaaggagaagcaggctatgaagcagcaaaaggcgatgttaatcgccctgatcgtcatctgtttaatcgtcatagtgacggcactggtaacgaggaaagacctctgcgaggtacgaatccgaaccggccagacggaggtcgctgtcttcacagcttacgaacctgaggagtaagagaccaggcgggggagaaatccctcgccacctctgatgtgtcaggcatcytcaacgcacccgcacttaacccgcttcggcgggtttttgtttttattttcaacgcgtttgaagttccggacggcgccggaatagaatcaaaaatacttaagtagcgcgcagggagaagagggatggaccccgaacaggggagtgctatttatctggaaggattctgttgatgagaatcgaaraattacgtgaaatttttagtgaagatggcctctatactgtgcgcgttgagaagggcgctattgtcagccactgccgtattaaatgtttacagtctcaacaaaggaagagtggagctgcgttaattcattttgtggatgggcttgtgacggatggttttattttgcgtgcaaatgaatttgtcacatcgttgccgtctctgaaagaagctgggattaaggctggtttttctgcttttgaagattagtgaattcatctacaattcagcgcagggctgaacccctgttgagtaacactgtgccaccggagaaagccgatggcgcaaaattccagactacacaattctgataattcagccgtctttgccagcaggcacgggcggcgttctcatgcattcaaatctgactggttccggcacgccccatgcactgaagaacaggccgaatggctgattcagaactaccgcagacgtgggtatgagtttaggaaagccctcagcctcgattatcgtcactggataatctacgtcaggctgccgtactccgagcgcccaccgcgtccgtcccgcacattccagcaacgcatctggaggtaacgtgcgggtattacttcgacctgttccggtaccggaacttggggtggtggtccttaaaccaggccgtgaatccattgcaggtattccataaccctcgagtgcttgtggagccggagccgaaaagcatgcgcggtctgccgtccggcatcgtccctgccgttcgccagccgctggcggaggataaatcattactgccgtttttcagcaatgagcgtgtgattcgtgctgttggtggcgctggtgcactgtctgactggctgttgcgtcatgtcaaatcctgccagtggcctcatggtgactatcatcacagtgaaaccgttattcaccgttatggtaccggcgcaatggtgttgtgctggcactgcgacaaccagctgcgtgaccagacatccgaatcactcgagcaacttgctcatcaaaacctgtcagcatggatgattgacgtcatcggtcacgcaataagcggtacgcaggagcgtgaattatctctggctgaattatcctggtgggcggtccgcaatcaggtggcggacgcgctaccggaagcggtattacgtcgttcgctggggttgcgtgcggaaaaaatccgctcaatgtaccgtgaaagcgacatcgtaccgggagagcagaccgccaccatcatactgaagcagcgcacaaaaaatcttgcgccgctgcctcacgcccaccagcaaaacccaccacaggaaaagacggtggtcagcattgccgttgatccggagtctccggaatccttcatgaaacgacctaaacgtcgccgctgggtaaatgagaaatacacacgctgggtaaagacacagccgtgtgcgtgttgtggtaagccagcggacgatcctcatcatctgattggtcatggtcagggcggaatgggaacaaaatcccacgatattttcacgctaccgctgtgtcgggagcatcacaacgagcttcatgcggatccgctggcgttcgaagaaaagcatggttcccaggttgatttaatttttcgttttcttgatcacgcctttgcgactggcgtgctcgggtaaaagaggtgactgatgctcatagatttggttttaccttacccgccgacggtgaacacttactggcgacgccgtggcagcacatattttgtatcaaaagccggggagcgttatcgccgggcagtggtgcttattgttcgccagcagcgactgaaattaagcctgtccggacggctggcaataaaaattattgcagagccaccggataagcgtcgtcgtgacctggacaatattctgaaagcaccgctggatgcgctgacgcatgcgggagtgttaatggacgatgagcagtttgatgaaatcaatattgtacgtggtcagccagtatctggtggacggctgggtgtgaagatttacaaaattgagagtgagtgagcgtaaatatgatatatccggaaattacaggcaaaagcggcgaacatttacgcctgaacacgctggaagcagtctggatccaggggaaattacggatgtgggggcggtggtcgtatatcggtgggggtaaatccggaaatatgtttaaccggttactggtttcgaaaaagctgacgaaaacagcagttaatgaggttttacgcagcatgaagaaatccgggctggaaaaaccggaacttgaggcattttttcgggatatgaccagagggaagcagaagagctggttgtcacattgtacagacacagaggcgttgattattgatcgcgttatcagtgaggtgcttggggaatatcccgggctaatcaatattctccggcaaaggtacgaaggacggggaatgagtaagagaaaaatggcagaatgtttaaatcgtactcacccggaatggtgtttcagcacatgtgagaaacgtattgcaggttggttagccgtggctgaacacatgctttatgtacctatgcacgattcatttcgataaaaaaagcttgcttttttacgcagaaacagcttgaattcctgtaagcttcgcaaagctgtatcgcgaggcgaaatgcaagttttttcgcacaaggaagccaccggaaggtggtttttttgtgtccgtaatatacagcagcgcaataaattcgctggtggttattaataccgttctttcaggttgctggctttttcgacaagagttattggtgtgtcacgttaaccggaaaagggaaaaagacatgctgaaacagcaggatatgacagaaaccgccagagtggtgtttaatgaattaagcgttaccgacccggcgacagtcggggagattgcgcagaatacttacctttcacgcgaacgctgccagttaatactgacccagctggttatggcgggtctggcagactatcagtttggttgttacagacgccttcagtcctgaaggcttttttatttgtggtaaatgggcggctggtgggtgtaaggggcacccaccagccatctgctcatgcgttgggttcacaagcaaacctcaggcccactgctttgcgcaaaagcagaatgagcctatcagagacaggcttaatgatccatgcttaatactgtaaaaatatccagttgtgagttaatcaacgccgactgcctggaatttatgcggtcgttacccgaaaattctgttgacctgatagtcacggacccgccgtacttcaaagtgaaacccgagggctgggataaccagtgggcgggtgatgaagattacctgaagtggctggaccagtgtcttgcgcagttctggcgggtgctgaaacctgccggaagtctttacctgttctgtggccatcgtctggcatctgacaccgaaatcatgatgcgtgagcggtttaacgtgctgaaccatatcatctgggcaaagccgtccggacgctggaacgggtgcaacaaggaaagcctgcgggcgtatttccccgccacagagcgcattctgttcgcagagcattatcaggggccgtatcgtccgaaagatgccgggtatgaggcgaagggtaggacactgaaacagcatgtgatggccccgctgattgcttactttcgtgatgcgcgcgctgtcctggggataacggcaaaacagattgcagatgccacaggaaagaaaaacatggtgtcgcactggttcagtgccggtcagtggcagctgccgaacgaaagcgattatctgaaattacaggcactgtttgcccgggtggcagaagagaagcatcagcggggtgaactggaaaagccccaccaccagctggtggatacgtatgcctctctgaaccgacagtatgcggagctgcagagtgaatataagcatctgcggcggtatttcggtgtgacggtgcaggtgccgtacaccgatgtgtggacgtataaaccggtgcagtactatccagggaaacatccgtgcgaaaaaccggcagaaatgttgcagcagataatcagcgcaagcagtcgtccgggagacctggttgcagatttcttcatggggtcggggtcgacagtgaaagcagcgatggcgctgggacgtcgtgcaactggcgttgaactggagactgaacgttttgagcagacggtgcgggaagtacaggatttaatcattcgtaacggatgagattgcggagttaatcatgcgtcgttattattcagcaatcggccctttagctcagcggtgagagcgagcgactcataatcgccaggtcgctggttcaaatccagcaagggccaccaaccgccactagctcatcaggaaagaacgtcaccctgtgcgagattcggagtccccggtggcggtccattatcggtattctgcgttgttagctcagccggacagagcaattgccttctaagcaatcggtcactggttcgaatccagtacaacgcaccacaccacacttatctgccctgactctcttttgcgggctttttattacaggaaagacaccggacagtgaaatgttaaatgcctcacaattcaggcagttgactgttgcctgacatgctgagcgtttgttaaaaaaatcctgcatgatgaatccccctgagcggcggggcataatgacagatgtttggttgcgtattgtataggcaagttgcggattctgtctggtcattgcagaattcaccgggaggcacccggcatcatgctgtatacagagattaggcatatatccaggcttctcatcgcaggagcctttttacatgcaaaaaaaagcccgagtgggttcgggcaacagcatgagatacttgcattgtcatttttatcgtgtggattttaaccagggtttataaggctgcgcaactgcgcggcctttttcgttttgcgggctgcggttctcctcttttgattctccttgtggccggaccgtggcccgcaactgttgaggaaaatcccggaaaggggaggaataatgacatttaaacattatgatgttgtcagggcggcgtcgccgtcagaccttgcggaaaagctgacacacaaactgaaagagggctggcagccgtttggtagtcckgtggccataaccccttataccctgatgcaggtgattacagcagaaggtgatgtggtggtcagtggtgcaactgagccggattggtactacgtcatcgtactggccgggcagtccaatgccatggcttacggtgaagggcttccgctgccggattcatacgatgctccggatccgcgcattaaacagctggcgcgccgcagtacagtkacgccgggtggggctgcctgcagatataacgatattattccggccgaccactgcctgcatgatgtgcaggatatgagtacgctgaatcatccgaaggcagacctgagcaaagggcagtacggctgtgtcggccagggsttacatattgccaaaaaactgctyccgtatatcccgaataacgcggggatcctgctggtaccatgctgtcgtggtggttcggcattcacccagggcgcggaggggacattcagtgcggacgcgggggccagccaggattcggcgcgctggggtgtgggtaaaccgttatatcaggacctgattgcgcgcactaaagctgcattacagaagaacccgaaaaatgtgttgctggcggtgtgctggatgcagggagagtttgacatgagcgccgccacccactgcacagcaacctgcgcttgtttacagccatgctggcacagtttcgtgctgacctctccgtgtttaacgcgcagtgccatggtggcagtgctgcagatgtgccgtggatttgtggtgacacgacgtattactggaaaaatacmtacgsyacccagtacracaccrtktacggsgsgtayaaaaacagggagagtgagggcgtttattttgtgcccttcatgacagacggtaacggcgtcaataccgccactaacgcgccggcagaagatccggatattccggcatcaggatattacggtgcggcatcgagaacgaatggaaaccaggtatcatcaaaccgcccgacacatttcagttcatgggcgcgcaggagcattattccggatcgtatggcaaccgctattctgaacgcagccgggcgcacctcagccttcatcagtggtaaggcaccggaaatcaaaccctcgcccggcggcaacacgccatcgggtccgtctgcagatacgtccgttcgcacaatctccctgctgccggcagccggagaggctgctgcgcagggctggagcattaaggatggcggaattcagttgtcagatggtgtatttaagatcaccargcagarcaataaaacctggtccctgacgcatccggtggatgacgcaattaccctgctgacacagggcggcagactgacctgtaagttccgcctgtcaggcgcactgaccaacaatcagttcgggctggggatttatctgtatacggatgctcccgttcctgatggtgtggcgatgacgggtaccggtaatccgttcctgatgtcgtactttactcagaccactgacggcagagtgaatctgatgcatcacaggaaagccggaaacacgaagctgggggagttcggcgattacggtaaccgactggcagacgctggagctggtgttcaccgccggcagtgccacggttactccgaaactgaatggagtggctggcccggcattccaggttataaaagacagtctgacactgggactgaatgcgctgacgctgacggatgttacaaaaaatgcagcgtatggcgttgagatagaaagtctggtgctggagataaatgcaccggcagcataataaaaaaagagccagcgactgacctgaaagaagacgctggctaaaaggccttatatgtttgtagagacttatttttcacagacagcaatgatgcctgtcaatatattatcaatatgcggattgtttcagttacagatgctttattaaggaaaaaaacagccagcactgactttcggtggagaggtgctggctcagaaggatagttggatttcacatgatacttatgcctggcggtatattttctgacagacagtgacgggtgttgtcaagatattgtgtcatttataacctgaatcagggggtggccggaatgttatctggcatttttagcagagcctgaatgccataatcacggctcccggcgttggccgtcagtgggtgacactggcggcttttttgttttyctttactttcattttctgtcggcggtgacggagacatacatcagatggaaaaaatcacaacaggtgtgtcatacaccacgtcagcggtggggacgggatactggttactgcagctgctggacaaagtctctccgtcccagtgggtggcgataggcgtgctggggagtctgctgtttgggctgctgacatatctgacgaacctgtatttcaagattaaagaagaccggcgtaaggcggcgcggggagagtaaagtgatgaagaaaaaatacgaactggttgttaaagggataaataattacccggataagattactgttactgtggcacyggaaattggtgggtatccgtcactgttgttgccagatgtggcgattagtcttgaccgtactgaaggtgccacgctggagttttacgaagctgaggcgaaaaagcaggcgaagcagtttttcatggatgttgctgccgggttatgtgaaggggatggtccgttaccggaaaagcgtcccgtaattttagaggcgcaggatgtgttgataacctacagaggaaaactaccgggaataattacgggttctctgaagactccaccgctggcctgaagacttaacatatccagggatttgaaatcgataaaccctgataaatatccatgaacgcaaaaatcagatacggcctgtcggctgccgttctggcgctgattggtgcaggggcgtctgcgcctgaaatcctcgaccagtttctggatgaaaaggaaggtaaccacaccacagcataccgtgatggtgcgggtatctggaccatctgccgtggagccacccgggtggatggtaagcctgtgattcctggcatgaagctgtcgaagggtaagcgtacagcctgaaccgtctggtcagaatctgacgaattagacaaagtggtgtccaccaaataagtagtgggaaccaaagtatcagatatgcagaaaaatgtgactcccggcaggcgaaaaggctgccctaattatcctcccgaatttaaacagcagctcgttgctgcctcctgtgaacccgggatatccatctcaaaacttgctcttgaaaatggcattaacgccaatctgttgttcaaatggcgacaacaatggcgcgagggaaagctgctattaccttcttcagagagcccccagctacttcctgtgactctcgatgcagctgccgaacagccagaatcgctcgcagaggacccggaaaccctcagtatcagctgtgaggtaacgttccggcacgggacgctccgcttcaatggcaatgtcagcgaaaagctcctgactctgctgatacaggaactgaagcgatgatcccgttaccttccgggaccaaaatttggctggttgccggtatcaccgatatgagaaatggcttcaacggcctggctgcgaaagtacaaacggcgctgaaagacgatcccatgtccggccatgttttcattttccggggccgcagcggcagtcaggttaaactgctgtggtccaccggtgacggactgtgcctcctgaccaaacggctggagcgtgggcgcttcgcctggccgtcagcccgtgatggcaaagtgttccttacgcaggcgcagctggcgatgctgctggaaggtatcgactggcgacagcctaagcggctgctgacctccctgaccatgctgtaaatctctttatcctggttgtcacagaataagcccggtaaaatacgggcttatgaacgacatctcttctgacgacatcttcctgctgaaacagcgcctggccgaacaggaagcgctgatccacgccctgcaggaaaagctgagcaaccgggagcgcgaaatagaccatctgcaggcgcagctggataaactccgccggatgaacttcggcagtcgttccgaaaaagtctcccgccgtatcgcacaaatggaagccgatctgaaccggcttcagaaagagagcgatacgctgactggtagggtgtatgacccggcagtacagcgtccgttgcgtcagacccgcacccgtaagccgttccctgaatcactaccccgtgacgaaaagcgactgttgcctgcggcgccgtgctgcccgaactgcggcggttcactgagctatctgggcgaggataccgccgaactgaaccgccccggttttcctggagagtgttttatctgtgaactcaggctgccagatcatcgtttccgatggaagcataataagctttttctgcttctgccggaggagtatggcccagccttcccagcaatcgtcgattgttataccagtccacccacgttagtgtggccagttccacttctgcacggtttttccagctcttacggtgtattacctccgctttgtaaagaccattgatgctctcagccatcgcgttgtcatacgagtcgcctgtactccctgttgatgccagtaatccggcttcttttagtcgctccgtataggccagtgacacatactgagagcctttatcgctgtgatggatggtgccagacggacgacgggcccacaacgcctgctccagcgcatccagcacgaatgtcgtttccatagacgatgagacccgccaccccacgatgtatccggcaaacacatcaatgataaacgccacatagacgaagccctgccatgtgctgacgtaagtaaaatcagccacccacagctggtcaggtcgttctgccacgaactgacggtttacgcggtcgcctgcggcaacggctttccggctgatggtcgtacggacctttttaccccggagaacaccggcaagtcccataaccgccatgagacgtgccactgtacatctggccaccctgattccttcccgtaacaactgacgccagactttacgcacaccgtacacctgatgattttcatcgtatacgcgctgtatttttctcttcagccagtcgtcgtgctgcgcacgggcnctgcntttatccggatgatgtcgctgttgctgacaatggtaatacgttgacggggcaatatgcagttcgctgcataccggtccgaccccgtactgctcacgcagcttatccagcagtggcatcattttttccagaggcggtcgaactccgccttcgcaaaataagcgnaagcctggcgaaggatatcgttactgcggcgcagttcacgattttcacgttccagctctttcagacgctgacgttcagcgctggtgagcccaccatcaccgcccccggtatcccgctcatgctggcgaacccagacacgcagagtctccggcgtacagccaatctttggggcaatggaacaaattgccgcccactgtgagtcatattcatcctgactttccagaaccatacgaatcgcccgctgacggacttcgggggaaaaacgagtatttttagtcatcctgtttacctctttctcagggagtttagtctccaggatttccggggcggttcacagggtaatcacgcaggtggtgatactggatgcggataaaaagcagatacagtgcgtggtaagaccgctgcaaatcctgcgtgctgacgggacgtgggaaaatattggcgggatgaartarcccgacagcttcacaaaaaccggagtccggctccggtttttgttgtcatgtatgggggctgtttgttatgactccctgtgtttggaatgaatatttaaatagggagttttgtcatgccagttaacatcagctattgcatccaattcattttccacctgaatgcaggttcttcgtgctcaaatggccgccagtggcggtggagaaattacagtaggcgggcagacggtccgtatcacatatagtgaaacggatggtcgctttctggcgagtgggggcaataacagtttgctttctggattattacttacagggcttaatggtggtcctgaagcgctcagggatataatgttaagaatggtttcaggttcaggtaacacacaatcacatggtgatattgaggggaaaatatcacaatgtaagttttctgttaatacggagagccttcagtgtccatccgaggcggttcgatgcccaattatactggataaaccagaagaaggtgtgtttgttaaaaattcagaaggttctttggtttgtaccttatttgattcggtttctttttctcatttggttcgcgacggtgggaagcacccgctaacacgagaaccaataacygtcatcaatgattgtaagtcaagaacaatgtatttatgaccaaaccaaaggaaactttgtcataaaggataagtgaaataaatattacccaagctatatgttaactgccagttgcttatatgaaatgctacagatgttcagggtataaggatgtggtaattggtgtactggatgcagctaaaaagcatatacagtgagtggtgaggccgctgcaaatctggcgtgctgacggggaaatattggcgggatgaaatagccgacggattcacaaaaaccggagtccggctccggtttttgttgtcatgtcagggagatgtttgttatgaagcccagaggaaatatttatctgtatgaaggaatatggtaatgcctggattagtatcatatatatcatcgacttcattcgcgaatgagatggcggagatgcgtcagcaggtaatggaagggcagattggtggatttctcctgggaggggagagagttagagtttcttatttatttcaattgcattaatatcttgaatggattacatagagttaatgtatccagtactccctattctcttgcatataatacatgttgcaacttacatctcagcgctatgaaaaaacaccacctctctctttatgaaattctggatttaccaagcgctaatttatcatttcaatctacctttaagtattgcatttatctccctacgaggtcatactttaggaagttaaatatgaatgataatatacctacagcgcgaaatcacaaacaatcgacttgtattacagaaaaaacatgcctatatttttaaacttcacagcaggaagtatcttacctgagaatgagctagcatctttacgttatattgtgcagcaaaatcaaaatgatactgtaatcataaaagaacgttataaaatggatatccgttatatcgaatcagtcaatggttttacagtaaatcctgtatgcagtaatcatttctccatatttatggcgagacaaaacactattgctcgcaacctggaacagcagatcaacaacggacgaagttttgcacaaatatctcaggattttatgcttcaattatcttcaaatataggatggaaaaaaggggccgaaaacgcccttaaaaataaaatccattctcattcatttgttgtaaatcctgatgaattctcttgcgacacacaatttcttaagtgcccaataacattatgcgttccagaaaaaggggtttttgtcaagaatgcactgaactccaacatatgcactctttatgataagtctgcgttcatgaatctcacaagagaacatctaccccaccctctcagcagggaaaagatagtaaaagaaatgattattgaaaggaatatgtgttattttgacaccataagtcagcatttcataattatggatgcagaccaacaagaaacagcattgtaaataaaatgtaataattacatactattagtgattctcatgcatcgtaagcggctcgccagaaccgtattgatatttactgaggagcctgtacatagatttgtgtwattgcctgattttgatatgttcaatccaacatcaaaagcaggttaatttatggacgaaaaacagttgcaggctctggctaacgaactggccaaaaatytcaaaacccctgaagatctcagccagttcgatcgcctgctgaagaaaatcagcgttgaggcagctctcaacgctgaaatgtcccaccatctgggctacgataaaaaccagcctaaaccgggtgccaactcccgcaatggctattccacaaagaccgttatcaccggtgatggccacctggaactacgcactccgcgcgatcgtgatggctcttttgaaccgsaacttgtgaagaaaaaccagacccggatcaccgggatggataaccagatcctgtcgttgtacgccaaaggcctgacaacccgcgaaatagcggctgcgtttaaagagctgtatgacgctgatgtcgtaagcgtacagcgagggccgtattgacggggatgtgttattcagctggcagtgctatgcgccacggaagcagttcgctgacccggttgaccggccagtcttgctattgacgccaagcacatggcgaaggtagctttctggatccacgtcatttcagtttgcacgtcccgatcaggctgtacagtagcgctccccgctcaccaccatgatcagagccgaagaacaggaagtttttacgacccagactgaccgcccgcagggcattttcagcgatgttgttgtcgatttccacccagccatcgttcgcatagtacgtcagtgccggccactggttaagtgcgtacgcgaacgccttcgccaactctgagtgtcgcgacagggtcttcatcttttcacgcaaccagctttccagggatttcaacaacggtttcgtttttcgctgacgttcagcaagccgctgctctgccggcattccccttatatccgcctctatggcgtacaactgaccgatctgctccagggcttcttccgtcagtgctgacgggatgcggacgtgcacatcgtggatctttcggcgggcatgagcccagcaggcagcttccwgttatcccaccattgcgatacagctcgttgaacccggcgtacgcatccgcttgcagcacaccgctgaagcaggcaagatgagtctgcggatggatgccttttctgtccgggctgtaagcgaaccacactgcaggtgccaacgctgaccctgcattgcggtcatcacgaacatacgcccacaaccgcccggtcttcgtcttcttattacccggcagcagtacctggaccggggtatcatcggcatggagtttgccgtcagtcatgacatagccatgaagcgcctcttccagcggagacagcagccggcagcatgcatccacccagcccgacagcagtgaacgcctcagctccacaccttgccggccgtatatttctgactggcgatacagcggggtgtgctctgcatacttcgaggtcagcacgcgggccagcagccccggtccggcgataccccgctcgatgggccgcgaaggtgcaggtgcctgcacgatggcatcgcactgagtacaggcatgtttttcccgtaccgtccggataacccggaaggctgctacgcatcaactccagctgttcggcggtatcctcgcccagatagctcagtgaaccgccgcagttcgggcagcacggcgccgcaggcaacagtcgcttttcgtcacggggtagtgattcagggaacggcttacgggtgcgggtctgacgcaacggacgctgtactgccgggtcatacaccctaccagtcagcgtatcgctctctttctgaagccggttcagatcggcttccatttgtgcgatacggcgggagactttttcggaacgactgccgaagttcatccggcggagtttatccagctgcgcctgcagatggtctatttcgcgctcccggttgctcagcttttcctgcagggcgtggatcagcgcttcctgttcggccaggcgctgtttcagcaaggaagatgtcgtcagaagagatgtcgttcataagcccgtattttaccgggcttattctgtgacaaccaggataaagagatttacagcatggtcagggaggtcagcagccgcttaggctgtcgccagtcgataccttccagcagcatcgccagctgcgcctgcgtaaggaacactttgccatcacgggctgacggccaggcgaagcgcccacgctccagccgtttggtcaggaggcacagtccgtcaccggtggaccacagcagtttaacctgactgccgctgcggccccggaaaatgaaaacatggccggacatgggatcgtctttcagcgccgtttgtactttcgcagccaggccgttgaagccatttctcatatcggtgataccggcaaccagccaaattttggtcccggaaggtaacgggatcatcgcttcagttcctgtatcagcagagtcaggagcttttcgctgacattgccattgaagcggagcgtcccgtgccggaacgttacctcacagctgatactgagggtttccgggtcctctgcgarcgattctggctgttcggcagctgcatcgaragtcacaggaagtagctgggggctctctgaagaaggtaatagcagctttccctcgcgccattgttgtcgccatttgaacaacagatttgcgttaatgccattttcaagagcaagttttgagatggatatcccgggttcacaggaggcagcaacgagctgctgtttaaattcgggaggataattagggcagccttttcgcctgccgggagtcacatttttctgcatatctgatactttggttcccactacttatttggtggacaccactttgtctaattcgtcagattctgaccagacggttcaggctgtacgcttacctgatgtctcaccggcgctggtatcgaaggtcaccgatgccgttatggagcaggtagtcgaatggcaaaaccggccactggatgcggtctatcccattgtttatcttgactgtattgtcctgaaggttcggcaggacagtcgcgtaatcaataaatccgtattcctcgcactgggtatcaatatcgaaggccagaaagagttactgggtatgtggctggccgaaaacgagggagcgaagttctggctcaatgtgctgactgaactaaaaaaccgcggcctgaacgatattctcatagcctgtgtcgacgggctgaaaggcttcccggacgctatcaacacggtgtatccggaagcccgcatccagctgtgcatcgtgcatatggtgcgcaacagcctgcggttcgtttcctggaaggactacaaagccgtcacccgcgacctgaaagccatctaccaggcccccacagaagaagcaggccagcaggcgctggaagcgttcgccagcgcctgggacagtcgctacccgcagataagccggagctggcaggcaaactggaccaacctggcgatgttcttcgcttacccggcagatatccgcaaagtcatctacacaaccaacgccatcgagtcgctgaacagcgtaatccggcatgccatcaaaaaacgcaaggtgttcccgacggatgactcagtgaaaaaggtcgtgtggctggcaatccaggcggcttcacagaaatggacgatgccactgcgggactggcgtatggcaatgagccgctttattatcgagttcggtgaccgcctggacggtcacttctgagaaaaggcatttacacagaatcgtgtacagggtcgattttactcgttaattagcgaacaaaccactcgtcagcagactcccaggtatctttcagcgtctcctgaacaaaagtttttgcagaatctttatcggcggtgcgagtaactgaaaggccatcgttgctggtggatttgatgatcacttctacatcgtcataacgtttactgacacgtcgcagcatttcttgttgcagggcaggaacagaaccttttggcattttgttgattttggctgcggggtatctgtggggcatatatgggacataaaaaggcctcaaataaacagcaaaatcgagattagggatttttagaaaaaatacaaccatctgaaaaaacttagaaaacacccaaaaacccacagaatagtaaaaaacatctatgaattatggatttcaagagtcattcaatctaactaattgataaataaaaaattaacttttcatcgcttcgttatggggcatggttggggcaaactcgcttaactgtgtatttaacagcgttacctgcgcattattattttctgacatccatttcccgtacacctgaaacaccatttgcgcatctgcatggcccatctggtttgcaatgaatgccgggttagctcctgctgttaacgaccagcaggcataagtgtgtctcgactgatacgatttgcgatggcggattccggcacgctttaccgctgcgtcccacatctgccttattgagtcaacggtaaaatggtcgccataattttttactttcgctgacacttcaggttgaaaaacaaaggtacatttttgtttctctgttctgccgaattctctgaggtgaacatcgatgatatgctctttgctcagtctcgttagtgccatctgactccggagagcgtcgattgcgggcttaataaggtgaatcacacgattggttcccgcctgtgtttttggtactgtaaaacggtcttttgccagatttcttctgattatcattctgccattgcgcaagatggttgcggtttttcttcatcattttcttcttctgtttcctgcttagcagacagttcgcggttaacttcttccaggatatctttttccggtgtatgtcgtgcagcagtgagagtttccttgctggggttctcgtgatcagtctccgttaagtaggcgttgatatacccctgaaggcgtcccgggtagtgataaaactcagggtgagcgctccggataagtgcaaaaatagcggcgcgggaatagtccagaatacccggggttgcgcgaagtgctgcggaccattctttgaacggactttctcttttcaggacgatttcttttgcgcgacgataaacgctgcccggaatttcataaatattaaaatccatcggaagtgtggctgctgcaatctccacatccagcgtatcgaaggtgtgtactaaattcggattgcgatcggttttgttcccgccaccggcatttgcaccggaagccgtacgggtgatacgcgaaacacgatttcctttcatccactcttttgtcagcagaccccgatcagtgtagtcagcgtccaggtatgcctcgaaaaaagcagttatcagtcccaggtctgaattaccgggattagggaaaactttgtcagtgtcgcgtaccagtttgtgaaggtcgcgaatctccagcgggtcgagcagctttgttttgtgagaaatggccaaggcagtaacagccggcagttcttccgcccgtgctatatgtaatgcctgaagttcttcccgtgcaacgtgcgttactggtttttcgctgccgtgttgcgcaagccagcgaatgggcagctcctgaccagaaaccggcaggagcatattctcctcaatctccgtcatgtcttcgccgttgacgttggtattgtcagtactggctggtttctcctgcacggagggagagggcgcgataaataccattgtaatgccatcttccccgcctttttcgtatcggttgcagaattcggtatcaaacacgccttcaggtggaaggtcattcacaacgggtaaatggacgcgaacgggttttttaaagtcgtcttcatcataatcgttgtcatccattgcggtaatgcagcgggagatggcaacagataatttttttgctgtagtccagtaaaaaccacctttaattcccagacgttttctgactttgtcattttttgcttcgcaatatagcgcaaattcttctttatcagtgctcattgataaacctcattacagatttaagggtgaacaaatccctgccattgctggcatttttaatccgttggtatggtgttaatatggctggagggttatccagccggtgtttcgttattcaggtacagcgatactttttttaccgggaggcattcaccagaaattttttgctcgtctcttgcctggaggcaggattctttactggcataaattccggtaatcacattctgtgattcacccgttataagaaaaaccgtcatcatcagtgcaaatgctgaagtcattgacgttctccgaaaataccaagttcaagaagagcaattcgggaaagtatggaattatcattgagcagataaggctcatatttcctcatgttaatggcatcttcagtaaactcccggttactgagcagaacaccaatatcaaaacaaccttcagacgtattaacgtttggtaataacgtttccattatcgcgtcctcaacaatgaattttgtgatgcagtgcctggtgcctccaggtgacgttaaccagttaacaattaacgccggatacagagaatccacccataacactgtttttggttttaactgttccgcgtgcgcttagccgcattcaccgcatcacaaaattcactttaaaaagggcggcagagcagccacggagtaaaactgataccgccaaacgtcaccagaaaattgataacagagggcgttgcagcggggttgtcacttaagcgtatggtcaacctgacaacccggtgtcctcaatggggaaggaataaccccgccatacttaccgccgcgccatttcgcggagtgccacaaccggaagcgcacgttcgaagaaatctaacgacaagccttctaagggaaagarcttcgccgtacgctttcgcgttatgcactgacttttcagggaaatatcctttcagtaaactgtcagtaccggattcttatccgtgtccggcgcacgaccacacgtgacagcgtgttggtctccatttttaacccagaacctcaatggaggataaaatgccaaacaaaaaaagaaatccgcttattgaaaaacagattgaatgcctggtaaatcaactcaggcaatcagggttattaaaaactcattcagagttgaggctcacagaatcagcattcgacgataaattaaataatgtcctttataatggcattattgattttaatcgttctgttggtcgccgcggccctgctggtgtttccttataattaccagtcaatccagagtggaccgtgttcagcgtaaatataactgtacacatccagattatatttgtggtctgttaagaacaggccgcaaatacatgccgaagcttccagtgcagcggctctgttactgaataaccatgtagcaacattccagcgtttttctgcatcccagtctttctcaaggcctgataccatgaagaaaccgttagtgttgccatcaaataattctgtttccaaatttttaagcaatgcctgatggactcttgccaggtattccgccggaatttcgccacgaattctgatgagattgtcataaacaaacatgttccccgcatatggcgatttttctttcttgttttttaaaccagcatcatgagcaaactgatcaatttcttcttccgttggtttcgtattgatgttttgcgctgtcgtttctgcaattttatttgccacactctctgagtcgtgtttatttatagacgcacagaaatacaatccggtaaacgcatcgcgcacattacgagccatattatcagtgtcttttttcgttaccgattccaattcaagttcgttcagacgatgacgaagtgtgtgtgctgcaatctcctggattgaaggaggtaaatctttaaattccatcgtcaacctcatcagtcagtgtttctggctaaccagcgacgcgcgccagcttcagttttaaacgttttgcttttggtatacgtcatcgcggtgaacgtaccgtcctggttggggaacacgccacataccagagattcgttgttgccaagattgagcgtatccatgttgacctcatttccccttaacgccggggtagcggaacaaaaacctgctgcatagttattaaagttgaaccctgccgtcatgttcttacgcctcgggctggctacttaacccctgaccactgcctggtaactcgaagtattgccctgcattctgtggggcggggtgagggaatgaatgaagtttagaaaaacgaacatttaaggtcaatgtttttttatcaaaacattttaagcaggcagctgttacgccatcactacgatggcatacagttaatcaaatagatgaggttggttaaatatcttgttgaattttaaagcatactcccaatatgcaagatagatcatccagcataattgaagggtagcgaggattcgtggggactaaaagaatatccggcccttctatctccagtttgcgaatgacaggcgttgtggtccctttgggtaaggcaaggacaatatttcctggttgtacgattcgatcgggatcaacaaaaactgttgaaccatttgggatggaaactccaccaccagatgtcgacatactgtcactctctagaacaacagcaaaggtattggccgggatttctccgacaagctgcacacaagaggttattgaggaatttttcatataatcactccagcttgctgcctgctgaagtgatagtagcggaaccgtttttatcggcggtaaagatagatcaagcgaatcacctgtatttaactctcctccattaagaagccaattttcgtttactttcaatatctttgccagtgaacttatgtaacgcgaggacggcgctcctccaccgttcatccattgacttacggagccttttgatgcgccagtggcattgacaaggtctttgcctttcaagtttagcgcatgcatacgttgggttatgcgttcagatattgtttgcttgttcatgttttgattttaaaacacagatggttttgtttcttgactttctttggttttgattattaaacttttggcgttcagttttatggagcgactcatgaaaaaatcagaagtattaggctattttggcggagttgttaaaacagccgccgctctaggaacgtcaaaaaccacagtcagcatgtggggggaagacgttccgtggaaatgggcgttgctaattcaggcagtcactgccggggcgctcaaatatgagttacacataccgacggttgtcattcccgattctgatcataatccgccttctaaccaaggggggattcatgaaaatcaagcatgaacacatccgcatggcgatgaatgcctgggcgcgtcctgatggcgaaaaagttccggcagctggaataacccaggcttattttgagttgggtatgacgtttcctgaactgtacgacgacagccatccggaagccctggctcgcaatacccagaaaattttccgctggatagagaaagacacccctgatgcagttgaaaaaatgcaggctctgttaccggcgatcgaaaaggcaatgccgcctctgctggtggcccgtatgcgcagtcacagctctgaatattaccgtgagatcgtcgaacggagggatcggctggtgaaagatgtggatgattttgtcgcagcggcgatcgcctggggcaccctgactaacagtgggggtcagcctggtaatgctgttgtcgtgcattgaccaacaatattcatgccggatttcttccggatgttcgagggtaaagttcggtatcagaagaggtgagtatggctaatgcctggctcagattgtggcatgacatgccaaatgaccccaagtggcgaacgattgccagggtatcaggacagccaatcgcaacagtgatggcagtgtatatccatcttctggtgagcgcgtcacgaaatgtcacgacatgtcacggcgtgtcactacgtggtcacattgatgtcacgacggaagatttagcaagtgcgcttgatgtgacggaagacgtaattgattcaattttgcatgcaatgcaggggcgggttctggatggtgaccttatttccggatgggaaaaacgtcaggtgctgaaagaggacaatggtaacgtttcgcaaacggcaaaatccccggcagagcgcaagagagcgcagcgggagcgggaaaagctgcggaaacataatgctgattgtcacgatgagtcacgacgtgtcacgcatctgtcacgacaagtcacgacagataaagatacagataaagatacagatacagaattaaaccccacacataacgcgcgcgagagtattccgaccagtgagtcgaatggtgcgccgttgcagacagccgaacctgaatacctggacggcytgagcgaaccgatcgggaaattttcgatgactactgtctggcagccgtcgccggattttcgacaacgggcagcagtgtggggtatggctctgcctgagccggaatttacacctgctgagcttgccgcattccgggattactggatggcggaggggaaggttttcacgcaggttcagtgggagcagaaatttgcccgccacgtgcagcacgtcagggcacaggtaaaaccagtcagcaaggggggaagccatgcagcatcaggtggcacggcatcacgggcagttcaggaaatccgggctgcacgcgaacagtgggaacgtgacaacggatttatcagcaacggaaacggcctggaagctgtgggagctcatgggggaggtgtattcgaaccgctggactcagaagaacggggccgcaccttcgaagctctggattgcccagattggtgcgatgactgaacagcaaatccgtctggtgtgccgtcagtgcatggaccgctgccgggcgggtgaaacgtggcccccggacctggctgagtttgttgcgctgatttcggagagtggggcaaatccatttggtcttacggtggatgccgtgatggaagagtaccggcgctggcgcaatgaatcctggcgatacgacgggagtgataaatacccgtggccacagcctgtgctgtaccacatctgcctcgaaatgcgtaccagagggattgagcgccagatgacgcaaggtgagttaaaacgacttgcggaacggcaactgacgaaatgggcaaagcatgttggtaacgggatgagtgttccgccagtgcgacgacaactggaaggggcgaaacacccgcaagggccaacgccaattgaacggctgaaacaggaatacgaacgccggaaggcagctggttttatttgaatctgagaaacgattttgtcggaggaaatattaatggaaaccgtatttgacgcactgaaagcactgaaaaaagcctcttcacaggtagtggcatcgcgccttggaatcagccgcgaagatgctgtcaacgaactgtggaaactgaagcgccgtggtgaagcggataacaaggggtcgatgtggtggctgactcagactggtgaaagtgaaccagtgtcaccggtaccgaaagtgacagcgcaaatgctgactgaggcgattgaacatcatggcccacaaaacggcggatgagctggcactgatgttcgggattacctcccgccgggcgaattcatcactggccatggcaatcagcaaagggcgtctgattcgcgtgaatcagggcggtaaatttcggtactgcataccgggcgctgatttaccggcagagccgaaagccgcatccataacggaaacggatggtaaagcctttcctcagccagcaggtgttgcgttaccagtcggggaagcggaaacacaggaagaaataaaaacggaaagtgtggcggtcacagtgcagtcacagccgtcgttcaccagaaagcatccggatggtctgattttaccatcgctgcatgtggctaaccgcgagctgcgccgggcaaaaggtcaggttcagaagtgggagcgagtctgcgccgcgctgcgggagctgaacaagtgccgggatattctccgggatattaccgccaccagagaacagcagcggtgagtgggtggaagacgtggtgccgggcggaaatcatgatactccggcagtgtgcgggaacgatgaaggtaaaaagcgttggcgcacttatcggacgaactgaagcggcagtgagaacgaaggcacgggagctgggcatcagcatgatgttacgtggtgattttcacccgtcggcaaaatattctcagcgtgatattgagctggcgcggcaactgcatcagagaggcatgcaaagaagggaaattgccagaaaattaggcatgccgctgcgcatagtgaataactacgtttatttcgacaggagggtgtctgcgtgaaaatcctgtatcaggattacggcccggtggggcaggtggttatcagcagtactgtaatggagtttcggaagcataaccgtgtggtggatgctgtgctgttaacctgtccggggatatcggcgagtcgtgcaggtgtgtttattatgaagacgaaattatatggcagtaaggcgtggataaagaaggcgtatcgtgtagcgttgcaggaggttaacagtgagtgaaattaaagaaatgccggtagttcgtgacggatatggctactggacacatcctgaatatgaaaaattctgtgatggtcgggaatatatttcaacggaagagtttaacgcctggatggaggaaaataatcttcaatacgtcctctgcttcagagatgaaggatgtgctgaccttgatgcgtgtgatgctgatatttctgcatgggaaccggaacgaccagagggcgatggctggtttattggttccattcatgatacggaagatggcccggtttgtgtctggttgcgaaataaggctgaagcataaaggcgataaaccaactaacaactaaatactgaagatttaaatcagaaacgatttttattaaatccttaaccggagggattcctgcaccctcagaacatcaggaggccgcccgaaagggcggtaagaaatgaaacattatttagaaaaaaattacccacgaaagagcagaacaacagagtttctgtttttcattctgtttatagtgttgatgataccgatatccccgctattactggtctggataattggaaggacatttgaaccagttattgagctatataccgatgtgacatgggaatcattcagcgcactgcacaataaaattaatccgtataaggaaaactgatatgagcactattaccagagaacgcgcggagattaaatcatacatcacaggcttcctgagcgactcggcgcacgataacaagtcttcagacagcctgctggctaatgtgtttcgtatcgcgctggcatcactggaagcagagccgatagcaatggtagtgcctgatgaaatggatttgcttacctgccatctcgacggtgtaactaaaacatatgctgatggctggaacgcctgccgcgtcgccatgcttcaggccggaaactttcgggaaaataagaattcgtcaaccaacaattttcgggaaatctcggaaacgtcaaccagatctccgataactctggatggctggataagctgtactgagcgaatgcctgaaaagagccagaacgtgcttatttcgatgaatatcgatagcgaggctgggccattaatatattccgcacgctatctcggaggcacgttccggcgcggaggtatagcagttagtccgggtaatgatcttaggcaagcaacccactggatgtcgctaccagaaccgccgcaggaggtgaatcaatgacctggcctgaagcattcacaacggtaggaattgcgatggcggtggcgctggtggtgtattcgatttgccgctggggataaaaacggtttgcgggaaaaggatagttaagtagaattgctgcgggtgcttgaggctatctgcytcgggcatgaacaccaacggcagatagagaaaagccccagttaacattacgcgtcckgcaagacgcttaacattaatctgaggctcaatccatgctgaacacatgtaggttagcctcttacgtgccgaaaggcaaggagaagcaggctatgaagcagcaaaaggcgatgttaatcgccctgatcgtcatctgtttaaccgtcatagtgacggcactggtaacgaggaaagacctctgcgaggtgcgaatccgaaccggtcagacggaggtcgctgtcttcgtagactacgaatctgagaagtaagagaccargcgggggagtaatctcccgccacctyctgatgtgtcaggcatcctcaacgcacccgcgctttaccatactgaaaatgctgtttgaatgttcatctctgaaagaggactatgaatgaaaaaggtattgattgcagcacttatttccggtgtgtcttttggcgcttttgcacagcagggtggtttccaggggccagaagcagagcgttcaacagtagcgcaggcaaaagaactgaaggatgatgcatgggttatccttgaagggagcatcgttaaaaaagtgggtgatgaacgttatgagtttcgtgacaatagcgggacaattgtcacggatattgatgacagcgtatgggccgggcagaatgtttctccgaaagacaaagtaagaattgagggtgaaattgataaagacctgagcagtgttgaagttgatgtaaaggcactgaaattattaaagtaaccgcccctgcttgttaagcccgtcttactgacgggttttctgtttgtacattccggcgtattgccttacaattcgcgcagtcagcctgaacaactgacacctgctgtcaccggagaatccgatgacacaacacataaaatcccacaattctgaagccgacccggaaattaagcaggggaggcgttttcgtgcgcctcagtatggctggtttcactatctgttnctgtacgatcgatgargcagatatgcttcaagaggcgtatctgcgtcgcggtgtccgtgtggagcggagtctgaacgctgatcgtctgacctggaccgtttctgtatatcttcctgttcgtgcacatctgccacggacacatgcctgctaccgtcagcgcgtctggaggtaatgtgcgggtattacttcgacctgttctggttccggaactcgggctggtggtccttaagcccggtcgtgaatcattgccagtttttcatcgcggcagggtgctggtggagccggaaccgaaaaacatgcgggcgctgccatctggagcggttcctgctgttcgccagccgctggcggaagataaatcactgctgccatttttcagcgatgagcgggtgattcgtgcagctggcggcgctggtgcactgtctgactggttattacgtcacgtgaaatcctgccagtggccacacggcgattatcatcacagcgaaaccgttattcacagttacggtgctggcgcaatggtgttgtgctggcactgcgacaaccagctgcgcgaccagacctccgaatcacttgagcaacttactcaacaaaatctgacagcctggatgattgacgtcatacgccatgtaatgaatggcacgcaggagcgggaattatcgctggctgaattatcctggtgggcagtctgcaatcaggtggtggacgcattacctgargcagtatcgcgtcgctctctgggattaccggcggaaaaaatccgctccgtataccgtgaaagcgacatcataccgggagaacagaccgccaccagcatactgaagcagcgcacaaaaaatattgcgctaccgcctcacacccaccagcaacagaacccaccacaggaaaagacggtggtcagcattgccgttgatccggagtctccggaatccttcatgaaacgacctaaacgtcgccgctgggtaaatgagaaatacacacgctgggtaaagacacagccgtgtgcgtgttgtggtaagccagcggacgatcctcatcatctgattggtcatggtcagggcggaatgggaacaaaatcccacgatattttcacgctaccgctgtgtcgggagcatcacaacgagcttcatgcggatccgctggcgttcgaagaaaagcatggttcccaggttgatttaatttttcgttttcttgatcacgcctttgcaaccggcgtgctcgggtaaaagaggttactgatgcgtatagagtttgttttgccttacccgccgacggtgaacacctactggcgacgtcgtggcagcacatattttgtatcaaaagccggtgagcgttatcgccgggatgtggcacttattgttcgccagcagcggctgaaattaaacctgtccggaaggctggcgataaagattattgcagagccaccggataagcgccgtcgtgacctggacaatatcctgaaagcaccactggatgcgctgacgcatgccggacttctcatagacgacgagcagtttgatgaaatcaatattgtgcgcggctagctcgtttctggtgggcggctgggcgtgaagatttacaaaattgaaagtgagtgagcataaatatgatatacccggaaattacaggcaaaagcggtgagcatttacgcctgaaaacgctggaaagtgtctggatccaggggaaactgcgtatgtgggggcgttggtcgtatattggcgacggtaagacgggaaatatgttcaaccaattactgacctctaaaaagctgacaaaaacggcaattaacgaggcgctccggaggatgaaaaaagcgggtctggacaaacctgaacttgaggcttttttgcgggatatgatcaacggcaatcaaaaaagctggctggcacattgtaccgattcagaggcgttaataatcgatagggttattggtgaagtactggcaggttatcccgggctgctcaatgttctgagtcagcgttatgtggggcgggggatgactaagcgcaaaatggctgaattgctgaatgatgcacatccggaatggagtttaagaacctgtgaaagacgcattgagcattggctaaaggtggcagaatttattttgtacaaaccaatggttatggcttttggtatagagaaaaaagttattgctttttgacgtaaaaactgcttcaattccggtaagcttcgcaaagctgtaccgcgaggcgaatagcagacatggacatttgaaagagcccgctttttgcgggtttttttatgactgaaaaacggcacggggcgttaaacgcgctggtggttgctaataccggtctttcaacttgctggctttttcgacaagagttattggtatgtcacgttaaccggaaaagggaaaaagacatgctaaaacagcaggatatgacagaaaccgccagagtggtgtttaatgaattaagcgttaccgaaccggcgacagtcggggagattgcgcagaatacttacctttcacgcgaacgctgccagttaatactgacccagctggttatggcgggtctggcagactatcagtgcggttgttacagacgcattcagtcctgaaggctttttatttgtggtaaatgggcggctggtggggggggcggcacctgtcagtcctttgcttatgtgttgatgataatttaccttttggggctataattgagctaaccaattgctaatgaaagtaaaattataatggctgttgtctgttcagttatcatggtttgctccccaattaatatttttcttgaaaaggatacgttgtcacttaagccaggctcagtcgttctggccaccaaatgcatcagggagcttttccttatgcattatggcaaagttaaaattgtcgatataagcsaatccgtcgtaagtcaatatctggaaagtcagcataagctgacgaggactcgtctgactgacattccgctttacctgttrctggaacccaacaaycctgcgttggctgcggctttaattaccagccagggattttccggagaggtcacggatatgtttcttatgatggcctgcctgtctctgtttgaaacagatgaacggatgtcattgtttttaagtggatgtttatccagcataagtgccaaagtcagggcgataattcagacagatatatcagcaagctggacgcttggtgcgattgctctacagttgcatatgagtgagagtttgttaaagacaaaactgaaaaatgaagggggcatgttcagtcgcttgttgctggaagagcggatgcgtgttgctgtaaatatgttatgttcccggtatggatatggacaggctgtagcagaaaaatgtggttattcaagccggtcctactttatttctgtatttcaccgctattatggcttcccgccagacagatatgtatccaggcaagggcttgattattgattttcatctgattattattttttggctcggccctttagctcagtggtgagagcgagcgactcataatcgccaggtcgctggttcaaatccagcaagggccaccatcacaaaccgccattagcttatcaggaagagcagacgacaccataacagggttgttggtgcggggccgggtccccgatggcggtccattatcggtattctgcgttgttagctcagccggacagagcaattgccttctaagcaatcggtcagtggttsgactccactacaacgcgccacacttattttccaggctcgcttcggcgggccttttttgtatctgcgccacgcccggcgcataccaaccacagagcctttcgggggtgagcttacggagtggtcagtgtgactttctctgtgggcagatcgctcccgggcgttggctcacccacccaaaggaacgtcacgatgtttggaatcttcaaaaagaaaacccgcagagcggcaacggaaattaaaaagtttgaaaaacgcgatctggcacaggtggtgattaacgccgcatacctggtggcctgtgcagatggtgaatgtgaggcatccgagaaagcgaagatcgaacaggtactgcggtaatcagcctgcgctgtacgcgtttacgtcagaaattaatgcgattagcgcaaccattatcggtcagctggatacgaactttaaaactggtcgtcgtgcggcgttacgtgagatygaggatgtgaaacacgatacgcgtgaagcggaagatgtgctggatgtggcggtggccattgcggaggcagacggcgaaattgagccggaagagcgcaaggtgctggaagagattgccggtgttctgggtcttcgtctggagaatcacctgtgacggtaaaactgcgcctgrctgtggctgcactcctgctgtttctggtggtgatggtggatttcaccagcagaatcatgtcggtgctggcggatggggtgctggtctgcggcattgtggtattgctgtggccggtgataaaaagaaacagcctgcataatgcttgatttttttgtttgctgtttattaaaaacacttctgcatggtgaatccccctgtgcggaggggcgatcagcaasmaggtatatgggataatcgcggattcaggtgctgatactgaattcaccgggaggcacccggcaccatgcaagaaaaagaatgtgcatgcaaacatgcccctctccggaggggcttttttatgggtaaaaaatgcccgaatgggttcgggcaatagcatgagatactgatattgttgtgttgttatcgtgtggattttaaccagggtttatcaggctgcgcaactgcgtggccttttttcatttcttgggctgtagtccccgagtgtcattcaggcttccggactacagccagcccactccatatctgatttaatmcactatcccggccgggaggaataatgacatttaaacattatgatgttgtcagggcggcgtcgccgtcagaccttgcggaaaagctgacacacaaactgaaagagggctggcagccatacggcggaccggttgccattacgccgtacacactgatgcaggcggtggctattgaaggagagccacaggtcggcccttcatctgagccggattggtactacgtcatcgtactggccgggcagtccaatgccatggcttacggtgaagggcttccgctgccggattcatacgatgctccggatccgcgcattaaacagctggcgcgccgcagtacagtgacgccgggcggggctgcctgcagatataacgatattattccggctgaccactgtctgcatgatgtgcaggatatgagtacgctgaatcatccgarggctgacctgagcaaagggcagtacggctgtgtcggccagggkttacatattgccaaaaaactgctcccgtatatcccgaataacgcggggatcctgctggtaccatgctgtcgtggtggttcggcatttacccagggcgcggaggggacattcagcgagtccacgggggccagycaggattcggcacgctggggggtgggcaagccgttatatcaggatctgatttcccgcacaaaagcggcattgcagaaaaatcccaaaaacgttctgctggccgtctgctggatgcagggwgagtttgacatgagcgccgccacccacgcacagcaacctgcgctgtttacagccatgctgrcacagtttcgtgctgacctctccgtgtttaacgcgcagtgccatggtggcagtgctgcagatgtgccgtggatttgtggtgayacgacgtattactggaaaaatachtacgsyacccagtacracaccrtgttacggsgsgtayaaaaacargggagagtgagggcgtttattttgtgcccttcatgacagacggtaacggcgtcaataccgccactaacgcgccggcagaagatccggatattccggcatcaggatattacggtgcggcatcgagaacgaatggaaaccaggtatcatcaaaccgcccgacacatttcagttcatgggcgcgcaggagcattattccggatcgtmtggcaaccgctattctgaacgcagccgggcgcacctcmgccttcatcagtggtaaggcaccggaaatcaaaccctcgcccggcggcaacacgccatcgggtccgtctgcagatacgtccgttcgcacaatctccctgctgccggcagccggaraggctgctgcgcagggctggagcattaaggatggcggaattcagttgtcagatggtgtatttaagatcaccaggcagagcaataaaacctggtccctgacgcatccggtggatgacgcaattaccctgctgacacagggcggcagactgaactgtaagttccgcctgtcaggcgcactgaccaacaatcagttcgggctggggatttatctgtatacggatgctcccgttcctgatggtgtggcgatgacgggtaccggtaatccgttcctgatgtcgtacttcactcagaccactgacggcagagtgaatctgatgcatcacaggaaagccggaaacacgaagctgggggagttcggcgattacggtaacgactggcagacgctggagctggtgttcaccgccggcagtgccacggttactccgaaactgaatggagtggctggcccggcattccaggttataaaagacagtctgacactgggactgaatgcgctgacgctgacggatgttacaaaaaatgcagcgtatggcgttgagatagaaagtctggtgctggagataaatgcaccggcagcataataaaaaaagagccagcgactgacctgaaagaagacgctggctaaaaggccttatatgtttgtagagacttatttttcacagacagcaatgatgcctgtcaatatattatcaatatgcggattgaaccgccccggaaatcctggagactaaactccctgagaaagaggtaaacaggatgactaaaaatactcgtttttcccccgaagtccgtcagcgggcgattcgtatggttctggaaagtcaggatgaatatgactcacagtgggcggcaatttgttccattgccccaaagattggctgtacgccggagactctgcgtgtctgggttcgccagcatgagcgggataccgggggcggtgatggtgggctcaccagcgctgaacgtcagcgtctgaaagagctggaacgtgaaaatcgtgaactgcgccgcagtaacgatatccttcgccaggcttccgcttattttgcgaaggcggagttcgaccgcctctggaaaaaatgatgccactgctggataagctgcgtgagcagtacggggtcggaccggtatgcagcgaactgcatattgccccgtcaacgtattaccattgtcagcaacagcgacatcatccggataaacgcagtgcccgtgcgcagcacgacgactggctgaagagagagatacagcgcgtatacgatgaaaatcatcaggtgtacggtgtgcgtaaagtctggcgtcagttgttacgggaaggaatcagggtggccagatgtacagtggcacgtctcatggcggttatgggacttgccggtgttctccggggtaaaaaggtccgtacgaccrtcagccggaaagccgttgccgcaggcgaccgcgtaaaccgtcagttcgtggcagaacgacctgaccagctgtgggtggctgattttacttacgtcagcacatggcagggcttcgtctatgtggcgttcatcattgatgtgtttgccggatacatcgtggggtggcgggtctcatcgtctatggaaacgacattcgtgctgaatgcgctggagcaggcgttgtgggcccgtcgtccgtctggcaccatccatcacagcgataaaggctctcagtatgtgtcactggcctatacggagcgactaaaagaagccggattactggcatcaacagggagtacaggcgactcgtatgacaacgcgatggctgagagcatcaatggtctttacaaagcggaggtaatacaccgtaagagctggaaaaaccgtgcagaagtggaactggccacactaacgtgggtggactggtataacaatcgacgattgctgggaaggctgggccatactcctccggcagaagcagaaaaagcttattatgcttccatcggaaacgatgatctggcagcctgagttcacagataaaacactctccaggaaacccggggcggttcacccatttcattcagcagacgttcacgggccatttccagcgaacagtcaggatcagccacgcactgtgcctgaagcgtctgatagcgaccgccgaacatggcaaacagatcgttaatgcctgacatgcgggctttctgctcagccataacgcgggcgcgaatggtcgcctcatcagacactgccggtaccggtgatggttctgttacngccggtgcagggattgtcactgtggtatcacgcggggcactgttgcgtggcggagtaatcatgtttcggatggattccggcatctttttaaattcctctgtacgttttgactgaatacatgccattgcctcaacagcgggtgtcacctggtcagcaaatccgtgtgccagacattcggcaccggacatccaggtttcatccgccagcatggcggcaatttcatcggtggtttttccggttttctgcgcataggctggcaacagtaccgattcgactttatccagcaaatcggcataactgcgcatatcctcagcatccccgccactgaatccccatggcttatggatcatcatgaaggcattttccggcataatgaccgtatcaccggccatcgcaatcacagatgccatcgaggcggcaacgccatccacatacacggtaatggtcgccccctgatttttcagggcattaaaaatggcgatgccttcaaagacatcgccacccggtgaattgatattggagattaatgtgggtgatatcacccagtgcattcagttngctgacaaactgcttcgcggtaactccccagaaaccaatctcgtcataaatataaatatccgcgtacccggccccccagcttgcatctgaaccaggatttattcttcatgctggctttcggtgtcgcgctgattgtcgtcagtcggcntgtnctctttgtgtgagggncagataaanacagcccagtttgtgtttatcattnaaccttgggngagttgactcatcggattggcccacggacgcaccagtcagattctgcgttgcccacccggatctgaattctccaggcttcagcttccttaacsgggtcgatccacggcatcaccggaccggaatacgtcgcgttatatagcgttttcatctccacatccgccggaattttcagsrgacctgccgcaaccaccatattcagccatgtccggtacaccgggcgggttaccgcgccaataaaacagtcctgcaggatcaggtaaccatccgtggactcgaccagctcctgccgctgggcgctgtaggtgccgttatagttacgcgccgcactggaaaaactcagacgactgcctgctgccactgcacgcaactggccgttgcggaaagtttcaaggttgggattgggacggtcagatttgaccatgccgatatcctcgcccttgcgcaaatcgtcataaataatacccggggtgatatggacttcccgctcggtctctttgatccccnggatcttcatagtcctgtccgtcacctttacggatatacagtcccagcgccgcagcaatacmgcgccgctgtcagttccgcatcctcatactccttaagggcactgatccgcatcagcacccccgataacatggatgagcctcgcgtctgatgcagacgacgagtgaacttcaggtggatcatttttccggcagcgatttctttcgtatcactctgscggccgctgaccggataatttttataaaccagatattttttcggtcttccccactcatcaaagaaaaacccccctgattcagttccggcggattcaatcagtgcgcatgggaaacaaaatccggnctccatccgcctcaagccagaaatggcactcccgccgtccgttccagaccgtttcccgcaccactgaccatctgcgcaaacacttcaccatcccgcagccaggtccgcagcagtaaacgttcaagcacaggacgggtatactgccctgtcacatccggactcacggaccattcagcccacaaacggcggatatccgcagccagctcagccgccatttccccgttttttygtaatggctgaggctccacaataattcccctggcaccaatcacccgctcttccagcttgtcaaacacaccaatcaccaggtcatgattgatatccagaaaacgggcctgctcccgcagggaaaccgcaccgtatttactgagctgatcagcagagcgattttcccgccgggctttatgtgtccgggtcggtttcaccgcctcataggccatgattaacgcccttgaacgcagtctggctgctttccacccgggggaaaacacgccgatcacatcatcaataattgccattaaaacctcgccagtttaaatcccggttttccccgcctgcggctcaccatcgcggcaagcctgcgttcccactcctgacgtccggcgcggatctgagaaaggctttccagcgtcagttgctgcccgttgaagatgacagactttccctccagtacggccatttccgcttcacggtaccgctgtatcatttctctggcttcttctgtgctcacaaccagcctcctgatgttatccatggattatcttccgcacgctccgtccgcagttttttcttccggcgacggcgtttttctgccccggccgtcagttccggggataccgtttcaccagaacgctcctgcgggaagacgagccacgtttcccgctgtgcccagtccggtgcggagggccagcggatcttttcgtaaccatgcagaacggcaagcgcatccgcataaaccarcaggtcaaacgcttcgttagcgcccctgcccggttttcgccattttccgtcactgccgcgctcttcataggtcagctcatcgtaaaaccaccgccccagccagtcgggaaagtggatatagttcggccctggtgtgtcacgccacagggcattatttacacgatccttaaacgcatccgtctgaaccagccacagcgcgacatcgccactggctctggcacggcgggcacttctgccggtattatccgggaaggttcggttaatcagcctgtcacgggcgaagtccatcccccttgaacagaaacaccctgttgcccagtccgtcactccggcaacgacgccagaaacgataggcgttatctgtcaccccggcttcccctcccgtatccaccgccatggccatcagacgcatgcgcacatccggatcagaagccagcggccatgttttatggaacacatccgtcagcaacaaatcccagtcctccggatrtgccgccggatcaaccggcagactttcaccgttgggactgcagcgcagtgaatgccggatgttgtagcgatcaacaatccagcgttccccctgctctccgtatccggtgatctgcacaacaaaacggcgatttttaccgccctgtacgtcaaccgttgcctcaataaaacgcacaccatccggcacagatcgccggggaaacggctcggcacgctgttcaagcagttcacttttacgctgttccgtggctgaacggggcagatagggtcgtccgatatcggtgttcaccaccgctttcagggtctcttcactgccggttcgctcatactcttcttctgccgccagcagtttaaaaatcagttgttcccaggtctgaaacgccgcagctggcccctccatccaaaatgacgcaatccgggagtttcgtggcgttccggtgatactgccgtccgccgccgcccgttcaccttcacgaagccagatcccctggttattcagttcgcgtttctgctcaggggcaatcagcccgcgacaatgcggacacatcagacgggcagcctgaccggcagccacaaaatccgggttattccggtatccggtcatgttatccatcaccggctgaaaatattccccgcagtgcggacacggccagtaccaccggcggcggtctccccggttatacagtgacaggatccccgttgttggcggtgcctcatgtgcgccaccacaacgccatttggtatcggtgatatcccgccccggtgaactctcgaccagggtcatccccgaggacataaaggtggtggtacgctttgaggccagcgtgaaggcatccccttccccgtccacattttcagggaaacggtcataatccgtcagcgccacacgacggtaatccgaagaggaaaagacggtgatcgacggccagccaatcttcaggaaggagccgtcaagaaacattttatcgtggacgttgttgtcattacgggaaggactgaggcgcttgctgacctccggactgtggcgaaacgtcctggaaagacgcgttctggaatgctcacgcgccttcgtctcagtcatctgcaccaccagcatatccgccggatcacagatgatgccgtacacaatccagccatcaatcagcccttcggttttcccggttcgcgcaggtcccacaaacaccaccgcgtcatattcacgggctgataatgtattaatggggtcaatcatatagggcgtcagcgatgactcccacggaccggaagtattggctccccgtggaacccgcatataacgcctgatggcttccgctactggtaaccggctgggtgggcgaaacagcgaggccacttcgcgccagatatcggatgcgcggctatggctctcgttcacctgattcacatatcggcctcatcacaacagtcaatgactgccttttccagtgtgtcgcggatctcatcaaccacaatctgtacttcattcagttgtgatgcagtccaccccctgtccctctccagccggtcaggccaggtttccagtacctgaactatcgctttcaccacgacagaaaaggaccgcctgacatcactgactggcacaagctgaacagtttcatgctgaaatttaagacgctcgcgctcggactgataccatgccttacgagcgtgaggatccatatcctcatcttcggaagatggtggtttttccagcaacgaagttatcaaatccgtcaggagatacagttttttcttttcattactgcctggtgcaagaggaacatccgccattctggcggcaacagtctgccggtgcagacctgaaagggctgccagttgattaatatttaacttcatatttttcagctcgccgtccatttacatccctccacataaaccgcagaacagaagtgactctgtttttttgtaaagaaatgccgccatataaagatgtcgaacaaaaaacaaccacaatcatcatctttttaatactaacagcattaaaaacaacaagttaccatcatgatgatgatgacgataaaatcacaaaaatgcgcctttttccgcgcccgcccgccccgtgttcaggcccaccccaccaggaggacccgcaaaatgataatggttatcatttgcaacaaaatccagtttcttccaccatcgcaccggactggcgactatgaggggacaacaccgcgctccgttaacgcggtaaaccccggtgtgtatcgtttttgattatccccgcacactctcgcagaggagtctccctgtctggctgcggtctctgttaatgcaggaatacggtgacgatacggcgcatcagcaaaacttagttcaggcactgagtgcggatatagtcctgtgccccttccagctgcttctgcattgtcatcaaccgttctctgaggatgaaataatcccgttcagcggtgtctgccagtcgggggccggttgcattatccacgccggaggtgccggtggcttcacgcacggtaccggagcaggtggcgttgatccgcaggcgcttacgaccagcggcaacatcagcacgcagagtttcattttcagctctcgcatcggctaattccctcgagtatctggcatcaagtgcagcgacatcacgctggcgtatctgcatatcagtaattgtcgcgttcgccagctccagctcactggcttttttatcgcgctgctctttgtagatgatggcgtgatcacggtaatgattcagccccagactaagcgcaccacaggccaccagcaggacaatgataaccacacacagaacacggttcatatcaccaccaacggattgcccagaccagaacagcaatggccacaatacgaatggcaaaagctgccgctcttgttaaatccagactggctggcgtctccacttcaatgcctttcataatggacaacctcagaaagaatcttttatacttcctcacagggaaagtacctccctacccataatttctcccttgccttactcaaggtcagaaaacacaaaaccccgcttgctgccaacaaacggggtttttacttttattcacttaggttttaccagttttcaggatttcgtgttatccacccgcgttggccaacgtcatttttcaggaaaatattctgctatctgtcgatgtcccagcacgccagcgcgctctcctggtcacgccgtgagacctgaccgtagcaattatttgaacggatacggcagtctctgccaccgtccttaatccaccagcgaatcgcctcacaggcaccttttcgatcgcctgcattaattcgtttataaaacgtcgacgggaagcacttaccggggccaatgttgtacggacagaatgacgcgatccccgctttctggggttcggtcagcggcacccggatgtttttctccacccatgccagcgccttgtcacgttcgatggcattaacccggtcgcatttttcctttgacagcttcatgccaggaataacaggcttaccatccaccagaatggcaccacggcagatggtccagatccccgcaccatcacggtatgccgtggtgtggttaccttccttttcatccagaaactggtcgaggatttcaggcgcagaagcacctgcaccaatcagcgccagaacggcagccgacaggccgtattttattttttcgttcatggggatttatcgatttctaatcccttgatatgttaggtatataatccaacactcatggtcgctctcataaacatatcccttgagacgcagcagattacaacaaatgaagccatataaatgaacagtaaagaaagtttgcgcagaagatttttacaactaatgacagaaaacgttaaatcagagttacttcttctgatggcagataataacgaagcaacaagcagcattcttgcagacccttacggtaagatctcacataaaacgctggatattattaccacaacattaacaccgctgatgcttcaacggctgaaacataatatcaacgcatgggttaatgaagaattaagtcctccctgcttatgggattctcgttacgcatgtcagcaaaaaatgcgaattttcaacttactatcaccaaagctcaggtagccataaaatcctgcccttcatggcatacaggatttcaatggaatcacaatgaccaactcttgcacagctgtatccctgactccccgacaactcagattttcagtatctgctgctatctaaagagaaagcgcacaaatgcaagggtctttcatcacgtcctgttattgattgcctgtgaccttttcttacctcatggaacgttttttcagttagaaatattcattttacaaccagttcgtattgtttattcatcgactactctccccgcgccaccttacgacggtcctctctgattttgaaatacaggttagtcagatacgtcagcaggccaaacagcagacttcccagcactcctattgccacccactgggacggagagactttgtccagcagctgcagtaaccagtattccgtccccaccgctgacgtggtgtatgacacacctgttgtgattttttccatctgatgtatgtctccgtcaccgccgacagaaaatgaaagtaaagaaaaacaaaaaagccgccagtgtcacccactgacggccaactccgggagccgtgattatggcattcaggctctgctaaaaatgccagataacattccggcctcccctgattcaggttataaatgacacaatatcttgacaacacccgtcactgtctgtcagaaaatataccgccaggcataagtatcatgtgaaatccaactatccttctgagccagcacctctccaccgaaagtcagtgctggctgtttttttccttaataaagcatctgtaactggttgttgttctgcgggttctgttcttcgttgacatgaggttgccccgtattcagtgtcgctgatttgtattgtctgaagttgtttttacgttaagttgatgcagatcaattaatacgatacctgcgtcataattaattatttgacgtggtttgatggcgtagatgcacgttgtgacatgtagatgataattattatcattttgcgggtcctttccggcgatccgacaggttacggggcggcgacctcgcgggttttcgctatttatgaaaattttccgggatccatgtccggtttctcttcaagttaactatatgaaaaatataaaaacaggtcttctgtgaaccggacatgaacaaaaaacagacatgtaaaccggacatgaccggttttgttgtgattgtgaggtgagagtttttgcgaggtgaggagtggctacgcagactgaagttgccaggcatttaagtctgaccgatcgccagcttcgcagattgcagaaattgccgggtgccccgatatcgaataagcgagggcaactggatctggatgcctggcgcgatttttacatatcgtatctgaggagaagtaaaaacgatgtgcctgatggcgatagcgaagacgactatgaggagaaattgcttattgccagatgggaactgacagcagaacaggctgttacacagcagttaaaaaagcgtacagcctgaaccgtctggtcagaatctgacgaattagacaaagtggtgtccaccaaataagtagtgggaaccaaagtatcagatatgcagaaaaatgtgactcccggcaggcgaaaaggctgccctaattatcctcccgaatttaaacagcagctcgttgctgcctcctgtgaacccgggatatccatctcaaaacttgctcttgaaaatggcattaacgccaatctgttgttcaaatggcgacaacaatggcgcgagggaaagctgctattaccttcttcagagagcccccagctacttcctgtgactctcgatgcagctgccgaacagccagaatcgctcgcagaggacccggaaaccctcagtatcagctgttaggtaacgttccggcacgggccgctccgcttcaatggcaatgtcagcgaaaagctcctgactctgctgatacaggaactgaagcgatgatcccgttaccttccgggaccaaaatttggctggttgccggtatcaccgatatgagaaatggcttcaacggcctggctgcgaaagtacaaacggcgctgaaagacgatcccatgtccggccatgttttcattttccggggccgcagcggcagtcaggttaaactgctgtggtccaccggtgacggactgtgcctcctgaccaaacggctggagcgtgggcgcttcgcctggccgtcagcccgtgatggcaaagtgttccttacgcaggcgcagctggcgatgctgctggaaggtatcgactggcgacagcctaagcggctgctgacctccctgaccatgctgtaaatctctttatcctggttgtcacagaataagcccggtaaaatacgggcttatgaacgacatctcttctgacgacatcttcctgctgaaacagcgcctggccgaacaggaagcgctgatccacgccctgcaggaaaagctgagcaaccgggagcgcgaaatagaccatctgcaggcgcagctggataaactccgccggatgaacttcggcagtcgttccgaaaaagtctcccgccgtatcgcacaaatggaagccgatctgaaccggcttcagaaagagagcgatacgctgactggtagggtgtatgacccggcagtacagcgtccgttgcgtcagacccgcacccgtaagccgttccctgaatcactaccccgtgacgaaaagcgactgttgcctgcggcgccgtgctgcccgaactgcggcggttcactgagctatctgggcgaggataccgccgaacagctggagttgatgcgtagcgccttccgggttatccggacggtacgggaaaaacatgcctgtactcagtgcgatgccatcgtgcaggcacctgcaccttcgcggcccatcgagcggggtatcgccggaccggggctgctggcccgcgtgctgacctcgaagtatgcagagcacaccccgctgtatcgccagtcagaaatatacggccggcaaggtgtggagctgaggcgttcactgctgtcgggctgggtggatgcatgctgccggctgctgtctccgctggaagaggcgcttcatggctatgtcatgactgacggcaaactccatgccgatgataccccggtccaggtactgctgccgggtaataagaagacgaagaccgggcggttgtgggcgtatgttcgtgatgaccgcaatgcagggtcagcgttggcacctgcagtgtggttcgcttacagcccggacagaaaaggcatccatccgcagactcatcttgcctgcttcagcggtgtgctgcaagcggatgcgtacgccgggttcaacgagctgtatcgcaatggtgggataacggaagctgcctgctgggctcatgcccgccgaaagatccacgatgtgcacgtccgcatcccgtcagcactgacggaagaagccctggagcagatcggtcagttgtacgccatagaggcggatataaggggaatgccggcagagcagcggcttgctgaacgtcagcgaaaaacgaaaccgttgttgaaatccctggaaagctggttgcgtgaaaagatgaagaccctgtcgcgacactcagagttggcgaaggcgttcgcgtacgcacttaaccagtggccggcactgacgtactatgcgaacgatggctgggtggaaatcgacaacaacatcgctgaaaatgccctgcgggcggtcagtctgggtcgtaaaaacttcctgttcttcggctctgatcatggtggtgagcggggagcgctactgtacagcctgatcgggacgtgcaaactgaatgacgtggatccagaaagctaccttcgccatgtgcttggcgtcatagcagactggccggtcaaccgggtcagcgaactgcttccgtggcgcatagcactgccagctgaataacacatccccgtcaatacggccctcgctgtacgcttacttaaaaaatgaggtgtcaaaaggaaaactgattgacaccgggttctgtatttttgccctcagtaagctggcaatggcgttatccagtacgcttgattccatccctttatccatgcagcgacagtttcctgatttaacaccgcgccatcttgaccatctgaaaacccttattgctaagggggcaaatcagtgtgcgcgggcaggggataaattaccggatttacttgatgaatatatcagagcaacaactgaataatatgatgagcgctgtcacaactgcattacagcccctgataagggcattgccggtgacgccagttgaatgggctgatcaaaattattatctgcctaaagaatcttcatatggtgagggcgaatggaaaacgctgccattccagatcgccatcatgaacagtatggggaatgatcagatccgcactgttaatctgattaaatctgcccgtgttggctatacaaagatgttgctgggggggggcgggtattttattgagcataaatcccgaaacagtctgctttttcagcccacggattctgccgctgaagattttatgaagtctcacgtggaggcgacgattcggaacgtgccatgcctgaaagacctttccccatggctgggtcgtaaacatcgtgacaatactctcacgctgaaacgcttttcatcgggcgtcggtttctggtgcctgggcggcgctgccgccaaaaactaccgtgaaaaatccgttgacgtggtctgctatgacgaactttcctcgttcgagccggatgtcgaaaaagagggctcgccaaccctgctgggggataagcgtattgaggggtcggtgtggccaaaatccattcgcggctcgacgcctaaaatcaaaggcacctgccagatcgaaaaagccgctaacgagtcggcgcattttatgcgtttttatgtgccctgcccgcactgtggggaggcgcagtatctgaaatttggcgatgagtccacgccttttgggcttaaatgggagaaggacagccctgaaagtgttttctacctctgtgaacatcatggctgcgtgatccatcagtctgaactggaccagagcaacgggcggtggatctgtgaaaacacgggcatgtggacccgtgacggtctgacgtttttcagcgcccggggtgatgaaattccgccgccgcgctccatcatgttccatatctggacggcgtacagtccgttcaccacctgggtacagattgtctatgactggctggatgcactgaaagatcccaacggcctgaaaacctttgtgaacaccacgctgggcgagacctgggaagaggccgtgggcgaaaaactcgatcaccaggtactgatggataaggtggtgcgttacacggcggcggtgcctgcccgggtggtttatctgacggcgggcattgactcgcagcgaaaccgttttgagatgtatgtctggggatgggctccgggagaggaagcctttctggtggataaaatcatcattatggggcgtcctgatgaggaagagacgctgttacgtgtggatgcggcgatcaacaaaaaataccgccatgcggatggcaccgaaatgactatttcccgtgtctgctgggacaccggggggatcgatggtgaaattgtttatcagagatcaaaaaaacacggtgttttccgggtgctgccggtaaaaggcgcatctgtctatggcaagccggtgatcaccatgccaaaaacccgcaatcagcggggcgtgtatctgtgtgaagtgggaacggacaccgcaaaagaaattctctatgcccgtatgaaagccgatccctcgcctgcggatgaagccacgtcgtatgccatccgttttcctgatgatccggagattttttcgcagacagaggcgcagcaactggtggcggaagagctggtggagaagtgggaaaaaggaaagatgcgtctgctgtgggataacaaaaagcggcgtaacgaagcgctggactgcctggtgtatgcctacgcggcattacgtgtgtccgtgcaacgctggcagcttgatctggctgtactggcaaaatcccgggaagaagagacgacccggccaaccctgaaagaactggcagcgaagctgtccggaggagtgaatggttacagtcgctgaactgcaggcgctgcgtcaggcgcgccttgatttattaaccggtaaacgggtggtgtctgtccagaaagatggtcgcagaattgaatatacggcggcttctctggatgagcttaaccgggcgatcaatgatgcggagtcggtactggggacaacccggcgtcgccgtcgtccgctgggagtgaggttatgaaacgaacgcctgtcctgattgatgtgaacggcgttccgcttcgtgagagtctcagctacaacgggggcggtgcaggatttggcgggcaaatggcggagtggttgccaccggcgcagagtgccgatgcggccctgctgcccgcgttgcgtctggggaatgcccgggcagatgatctggtgcgcaataacggaatagcggccaatgcggtggcactgcataaggatcacattgtcgggcatatgtttcttatcagctaccgtccgaactggcgctggctggggatgcgggagaccgcagcaaaaagctttgtcgatgaggtggaggcggcctggtcggaatacgccgaagggatgtctggcgagatcgacgtggaaggaaaacgcacgttcacggaatttatccgtgaaggtgtgggcgttcatgcgtttaacggcgaaatctttgtgcagccggtctgggatacggaaaccacgcagttattccgtacgcgttttaaagccgtgagtccgaaacgggtggacacgccaggacacggtatggggaaccgttttctgcgggvcggggtgraggtcgatcgatatggccgtgccgttgcgtaccatatctgtgaggatgattttcctcgctccgggagtggacgatgggaacggatcccgcgtgaacttcccaccgggcgtccggccatgctgcatattttcgagccggtggaggacgggcagacccgtggggccaaccagttttacagcgtcatggaacggctgaagatgctcgattccctgcaggcaacacagcttcagtcggccattgtgaaagccatgtatgcagcgacgattgaaagtgaccttgataccgaaaaggcctttgaatatatcgccggtgcgccgcaggggcagaaggataatccgcttattaatattctggagaagttctccagctggtatgacacgaataacgtgacgctgggtggtgtcaaaattccgcaccttttccccggggatgatctgaaactacagactgcgcaggattcagacaatggattttcggcgcttgaacaggcgctgctgcggtatatcgccgccggtcttggcgtttcctacgaacagttgtcccgtgattactcgaaggtcagttattcaagtgccagggcctctgccaatgagtcgtggcgctattttatggggcggcgaaaatttattgcggcccggctggccacgcagatgttttcctactggctggaagaggcacttcttcgggggattatccgtccgccacgggcgcgttttgatttttatcaggcgcgatcagcctggtcacgggcagagtggattggtgccggaagaatggccattgacgggctcaaggaggttcaggaatcggtgatgcgcattgaggccggactgagcacgtatgagaaagagccggcgctgatgggcgaggattatcaggacattttccgccagcaggtcagggaatctgcagagcggcaaaaagccggactctcacgtccggtgtggatagcgcaggcgtatcagcagcagatagcggagagtcgcaggccggaagaggagacaacaccccgtgagacgtaatctttcacacattattgccgcagcattcaatgaaccgctgcttcyggagcccgcctatgcgcgggttttcttttgcgcgctcgggcgcgagatgggggcagcaagtctttcggtaccacagcagcaggtacagcttgatgctcccggaatgctggctgraacggacgagtacatggccggaggtaaacgaccggcccgtgtttaccgggtggtgaacggtattgctgtactgccggtgaccggcacgctggtgcaccggctggggggtatgcggccattttccggaatgacaggctatgacggcattgtcgcctgtcttcagcaggcaatggcggatagccaggtgcggggcgtactgctggacattgacagtccgggcgggcaggccgccggcgcgtttgactgcgctgacatgatttaccgcctccgtcagcagaagccggtctgggcactgtgcaatgacacggcctgttctgcagccatgctgctggcgtcggcctgctcccgacggctggttacccagacatcccgtatcggctccattggcgtgatgatgagccatgtcagctatgccggtcatctggcgcaggccggtgtggatatcacgctgatttactcaggggcgcacaaggtggatggcaatcagtttgaagccttaccggcagaggttcgccagaacatgcagcagcgcattgatgcggcgcgccggatgtttgccgaaaaagtggccatgtttaccggtctgtctgttgatgccgtcacgggaacagaggccgccgtttttgaaggtcagtccggcattgatgccgggctggcggatgaattagtcaatgcgtcggatgccatcagtgtgatggccacggcgctgaacagtaatgtcagaggaggcactatgccgcaattaactgcaacggaagccgccgcgcaggagaaccagcgagtgatggggatcctgacatgccaggaagcgaaaggacgtgaacagcttgccacgatgctggcaggacaacagggcatgagcgttgaacaggcccgggcgattctggccgcggcggcaccgcagcagccggtggcatccacgcagagtgaagccgatcgcattatggcgtgtgaagaagcgaacggtcgtgaacaactggcggcaacgctggcggcgatgccggagatgacggtggaaaaagcccgcccgatcctggctgcttcaccgcaggcggatgccggaccctcactccgtgatcagatcatggcactggatgaggcaaaaggggctgaggcgcaggctgaacagctggctgcctgcccgggaatgactgtggagagcgcccgggctgtgctggctgcgggatcaggtaaggcagaaccggtctctgcatccacaaccgccctgtttgaacgcatcatggcgaaccattcaccggctgcggtacagggtggcgtgccacagacgtcagcagacggtgatgcggacgtgaaaatgctcatggccatgccatgaagtcagtgctgaccatcaacaggaggtttttacaatatggtaacgaaaaacatcactgaacagcgtgcggaagtacgtatttttgccggtaatgatccggctcataccgccacaggcagcagcggkatttcctcggcaacaccggcactgacgcccctgatgctggatgaagccagcgggaaactggtggtctgggacggacagaaagccggtagtgcagttggcatactggtactgccgcttgaaggcacagagacggtactgacctattacaagtcggggacctttgcgacggaggcaatccgctggcctgaaagtgtggatgaacacaaaaaggcaaatgcctttgccggcagtgccctgagtcacgcggcgctgccgtaacacgttatcaggccaccgcggtggcctgactgatttctgaatgaaaggaactgatttatgggattgtttacgacccgccagttactcggttataccgaacaaaaagtgaaatttcgtgcgctgtttctggagctgtttttccgccgtacggtgaatttccataccgaagaggtgatgctggacaaaattaccggaaaaacgccggtggcggcctatgtctccccggttgttgaaggaaaagtgctgcgtcatcgcggtggtgaaacccgcgtgttacgtccgggctacgtcaagccgaaacacgaattcccctggagccggtaaaaggagccggtaccaccctgtgggtttataacggtcagggtgacgcctatgcaaacccgttgtcagacgatgactggcagcgactggctaaggtgaaggatctgacgccgggcgagatgacggcagaatcctacgatgataactacctggatgatgaagacgcggactggaccgcgaccgggcaggggcagaaatctgcaggtgataccagttttacgctggcctggaaaccgggagaggaaggccagaaagggcttataggctggtttgaaagcggcgatgtccgggcctataaaatccgttttccgaatggcacggtggatgtgtttcgtggctgggtcagcagtatcggtaaggccgtgacggcgaaagaagtgatcacccgcacggtgaaagtcactaacgtgggtaaaccttctgtagcggaagaacgcagcaaaattacgccggtcagtgcgattaaggtgacgccgacatccggtacggtggcaaaagggaaaacaaccaccctgacggtttcttttgagccggaaagtgcaacmgacaagacgttcagagcggtttccgccgatccgtcgaaagccaccattagtgtgaaagatatgacaattacggtaaacggcgtggcgacaggtaaggtgcagatccctgtggtgagcggaaatggtcagttcgccgcagtggctgaagtcaccgttactgaagcgggcgctgcagggtaaacggaggtcatacatgtttctgaaaacagaacaatttgaatataacggtgtgtctgtcacgctttccgaattgtctgcgctgcagcgtatcgagcatcttgccctcctgaaacggcgtgcagaacaggcagaatccagcggcaacctgcaggtaagcgtggaagatctcgtcagaaccggcgcgtttctggtggcgatgtccctgtggcataaccatccacagaaaacgcagtcaccgtcaatgaatgaggccgtgatgaagatagagcaggaagtgctcaccacctggcctgccgatgccattgcccgggcggaagacgtggtgttgtgcctgtccgggatgagcggggctgttcgtccggatactgatattactgaagtggcgaaaaataacacgctgactgatgatgatttttctgcgggaaagtcttcgacggcgagctgaactttgccctcagactggcgcgtgagatggggagacccgactggcgcgccatgcttgccgggatgacatccaccgaatatgccgactggcaccgtttttaccgcacgcattattttcaggatacccagctggatatgcatttttccgggctgacgtacgctgtactcagcctgtttttttgcgatccggatatgcatccctctgatttcagtctgcttgtcccccggcatgaggaagagcaggtggagaggccggatgaggacaaaatgctgatgcagaaagcggcaggacttgccggaggcgtccggttcggtggggacggagggcgcgatattttatcgtctgcggatgtggcggatgtcatggtggatgatgccgcattaatgatggcttcagcggggattccgggaggtgtgagatatgtcccagccggttggtgatcttgttattgacctgagtctggatgctgtccgtttcgatgagcagatgagccgggtaaggcgtcatttttcaggtctggataccgacgtcagaaaaaccgccagtgctgttgaacagggcctgagccgccaggcgctggctgcacaaaaagccgggatttccgtcgggcagtataaagcggccatgcgaaccctgcccgcacagtttacggatatcgccacgcagcttgccggtggtcagaatccctggctgatcctgctgcaacagggcggtcaggtgaaggactccttcggcgggatgatccccatgttcagggggcttgccggtgcgatcaccctgccgatggtcggggtcacctcgctggcggtggcgacaggtgcgctggtgtacgcctggtaccagggagattccacgctttcagcgtttaataaaaccctggttctttccggtaatcagtccggactgactgccgatcgcatgctgacgctctcaagagccgggcaggcagcagggctgacgtttaaccaggcgagagagtcactggcagccctggtgaatgccggtgtgcgtggtggtgaacagtttgatgccatcaaccagagtgtcgcgcgttttgcttctgcatccggtgtggaggtggacaaggttgcagaggctttcggaaaactgaccaccgaccctacgtcggggctgattgcgatggtgcgccagttccgtaacgtgacggcagagcagattgcgtatgttgcgcagctgcagcgttccggtgatgaggccggggccttacaggcggcgaacgatatcgccacgaaaggctttgatgagcagacccgtcgcctgaaagaaaacatggggacactggagacctgggcggataaaaccgggaaggcattcaaatcgatgtgggatgccattctggatatcggtcgtcctgagtcctcagcggatatgctcgccagtgcacagaaggcatttgatgaggcggataaaaaatggcagtggtaccagagccggagccagcgccggggaaaaaccgcctctttccgggccaaccttcagggcgcatggaatgaccgggaaaatgcccgtctggggctggcagcggccacgctgcagtcggatatggaaaaagccggtgaactggccgccagggaccgggccgaacgggacgcatcacagctgaagtataccggagaggcgcagaaggcgtatgagcgtctgctgacgccgctggagaaatataccgcccgtcaggaagaactgaataaggccctgaaagacgggaaaatcctgcgggcggattacaacacgctgatggcggcggcgaaaaaggattatgaatcgacgctgaaaaagccgaagtcgtcaggagtcaaagtgtcagccggtgagcgtcaggaagaccaggcgcatgctgccctgctggcgcttgaaaccgagctcaggacgctggaaaaacacagcggtgcgaatgagaaaatcagccagcagcgtcgcgatttatggaaagcggaaaatcagtatgcggtcctgaaagaggctgccacgaaacggcagttatctgagcaggaaaaattcctgctggcgcataaagacgagacgctggagtacaaacgccagctggctgagctgggagacaaagttgaacaccagaaacgcctgaatgagctggcacagcaggcggtgcggtttgaagagcagcagagcgcgaagcaggccgccatcagcgcaaaagcccgcggtctcactgaccgtcaggcgcagcgggagtctgaagcgcagcgtcttcgggacgtgtacggtgataatccggctgcgctggcgaaggccacatctgcactgaagaacacctggtctgcggaggagcagcttcgtggaagctggatggccgggctgaagtccggctggggcgagtgggcggaaagtgcgacggacagtttttcgcaggttaaaagtgctgccacgcagacctttgacggtattgcacagaatatggcggcgatgctgaccggtgcagaggcagactggcggggattcacccgttcggtgctgtccatgatgacagaaatcctgcttaaacaggccatggtgggcattgtcgggcgtatcggcagcgccattggcggtgctttcggtggtggtgcatctgcttcctcggggacggccattcaggctgcggcggcgaacttccatttcgcgatcggaggatttacggggacgggcggcaaatatgagcctgcgggaattgttcatcgcggggagtttgttttcacgaaagaggcaaccagccggataggtgtggggaatctttaccgtctgatgcgcggctatgcggaaggtggttatgtgggtggtgccggaagtccggcgcagatgcggcgggcggaaggtattaattttaatcagaacaatcacgtggtgattcagaacgacggcaccaacggacaggcggggccgcagctgatgaaggcggtgtatgacatggcccgcaagggggcgcaggatgagctccggctgcagttgcgtgatggcggtatgttatcggggagcgggcgatgaaaacctttcgctggaaagtgaagccggatatggaggtgaactcgcagccatcggtgcgtgaagtgcgttttggtgacgggtactcacagcgtatggcggcagggctgaatgctgacctgaaaacataccgtgtgacgctttccgtgacccgggaggaggcccggcatctggaagcgttcctggcagagcacggaggctggaaggcatttttgtggaagccaccctatgcataccggcagataaaggtgacctgtgccgggtggtctgcgcgggtcgggatgttgcgcgttgagttcagcgcggagtttaagcaggtggtgaactgatgcaggatattcacgaagaaagtctgaacgagtcggttaaatcagagcagtcaccgcgggtggtactctgggaaatcgacctgacggcgcagggcggtgagcggtattttttctgcaatgagctgaatgaaaaaggggaggcggttacctggcaggggcggcaatatcaggcatacccgattgacggcagtggctttgagatgaacgggaagggcagcagtgccagaccgtcgctgacggtgtcgaatctgtttggtctggtcaccgggatggcggaggacctgcagagcctggtgggggccacggtggtccgccgccgggtgtatgcccgttttctggatgcggtgaattttgtggcaggcaaycctgaggcagaccctgagcaggagctgacggaccggtgggtggtggagcagatgtcatcgctgacggccatgacggcctcgtttgtgctggcgacaccgacggagacggacggagcgctgtttcccggtcgcattatgctggcgaatacctgtatgtgggattaccggggagatgaatgcgggtataacggtcctgcggtggcggatgagttcgacaaccccaccacggatatccgtaaggacagatgcagcaagtgcatgcgcgggtgtgagatgcgcggcatggtggctaattttggcggtttcctttccatcaataaactttcgcagtaaatcctgttttatgacacagactgaatcagcgattytggtgcatgcccggcggtgtgcgcctgcggagtcgtgcggcttcgtgataggcaccccggagggcgaacggtaccagccctgcgtgaatatctccgcagagccggaggcgtattttcgtattgcgccggaagactggctgcaggcagagatgcagggggagattgtggcgctggttcacagccaccctggtggtctgccctggctgagcgaggccgaccggcggctgcagataaagagtgccctgccctggtggctggtctgccggggggaaattcataaattccgctgtgtgccgcacctgaccgggcgtcgttttgagcacggggtgacggactgttacaccctgttccgggatgcataccatctggcggggataacgctgccggattttgagcgtgaggatgactggtggcgcaacggtcaraacctttacctggacaatatggcggcsactggtttttaccgggtgcccctgtcctctgcacaggcgggcgatatcctgctgtgctgctttggcgcatcggtggccaatcatgccgccatatactgcggcaacggtgaactgcttcaccatctgcctgaacaactgagtaaacgggagaggtattctgaaaaatggcaacgacgaacgcattctgtctggcgtcaccgccactggcacgcatctgccttcacggggatttgcaacgatttggccgccgcctcagcctgtacgtgaacacggcagcggaagccatccgtgccctgtcgctgcagatgccgggattccgccgtcagatgaacgaaggctggtaccagatacgtattcgcggtgaggacacggcaccggaggcggtgtacgcccgtcttcacgaacctctgggtgagggggcggtcatccatattgtgccgcgactggccggagccggaaagggcggactgcagattgtgctgggggcagcagccatcgtgggctctttcttcaccgcckgcgcaacgatggcgttgtggggcgcagccctgagtgccggagggctgactgccaccacgatgctgttctcactgggtgccagcatgatactgggcggtgtggcccagatgctggcaccgaaggcaaaaacaccggagtacagggcgacggataacggtaaacagaacacgtatttttcgtcactggataacatgattgcccaggggaacccgatgccggtgccttatggtgaaatgctggttggttcacgacggatatcccaggacatcagcacccgtgatgagggcggagacgggaaagtggtggttatcgggcggggatgaaaataaaaaaatcccgcagagttagcggagctgcgggagagaacgatgaagattaacgttatggagttatttttcaggcatcaaaaaagtaatgcagcgtcattattgcggctacaggcaattgccggaaatgtgaagagtttcagaaattttattccgtcatgacacaggcaccctccggggtgcctgttgttttct

SEQ ID NO: 3 - DNA, 24 bp, Artificial Sequence
  based on Escherichia coli
gatcttcttttttagagcgccttg

SEQ ID NO: 4 - DNA, 24 bp, Artificial Sequence
  based on Escherichia coli
tgcctgagttcacagataaaacac

SEQ ID NO: 5 - DNA, 24 bp, Artificial Sequence
  based on Escherichia coli
gcgtaatgacttaatgattttcgt

SEQ ID NO: 6 - DNA, 24 bp, Artificial Sequence
  based on Escherichia coli
catcacattcctgacgcagtgctt

SEQ ID NO: 7 - DNA, 24 bp, Artificial Sequence
  based on Escherichia coli
gagaatattatcagcgacttgata

SEQ ID NO: 8 - DNA, 24 bp, Artificial Sequence
  based on Escherichia coli
ctagatcaactgagacagattata

SEQ ID NO: 9 - DNA, 20 bp, Artificial Sequence
  based on Escherichia coli
catgattggctggcgtccct

SEQ ID NO: 10 - DNA, 20 bp, Artificial Sequence
  based on Escherichia coli
accaatgaaatgagttcaga

SEQ ID NO: 11 - DNA, 24 bp, Artificial Sequence
  based on Escherichia coli
tgaaagtaaacgaaaattggcttc

SEQ ID NO: 12 - DNA, 24 bp, Artificial Sequence
  based on Escherichia coli
aaagaatatccggcccttctatct

SEQ ID NO: 13 - DNA, 24 bp, Artificial Sequence
  based on Escherichia coli
atgttgagtatattgggcaagaca

SEQ ID NO: 14 - DNA, 24 bp, Artificial Sequence
  based on Escherichia coli
gaaatatcgataacagacgctctc

SEQ ID NO: 15 - DNA, 24 bp, Artificial Sequence
  based on Escherichia coli
gagaagccttgcttcattaaagta

SEQ ID NO: 16 - DNA, 24 bp, Artificial Sequence
  based on Escherichia coli
atgaagctgttttggctgcactat

SEQ ID NO: 17 - DNA, 24 bp, Artificial Sequence
  based on Escherichia coli
atctgaaagatctgcatttgatat

SEQ ID NO: 18 - DNA, 24 bp, Artificial Sequence
  based on Escherichia coli
gattgtaagctaatatcagctcat

SEQ ID NO: 19 - DNA, 18 bp, Artificial Sequence
  Synthetic Oligonucleotides
gaaacagctatgaccatg

SEQ ID NO: 20 - DNA, 17 bp, Artificial Sequence
  Synthetic Nucleotides
gtaaaacgacggccagt

SEQ ID NO: 21 - DNA, 23 bp, Artificial Sequence
  based on Escherichia coli
ataaatcgccattcgttgactac

SEQ ID NO: 22 - DNA, 19 bp, Artificial Sequence
  based on Escherichia coli
gaacgcccactgagatcat

SEQ ID NO: 23 - DNA, 21 bp, Artificial Sequence
  based on Escherichia coli
ggcactgtctgaaactgctcc

SEQ ID NO: 24 - DNA, 22 bp, Artificial Sequence
  based on Escherichia coli
tcgccagttatctgacattctg

SEQ ID NO: 25 - DNA, 20 bp, Artificial Sequence
  based on Escherichia coli
gacccggcacaagcataagc

SEQ ID NO: 26 - DNA, 20 bp, Artificial Sequence
  based on Escherichia coli
ccacctgcagcaacaagagg

SEQ ID NO: 27 - DNA, 21 bp, Artificial Sequence
  based on Escherichia coli
gcatcatcaagcgtacgttcc

SEQ ID NO: 28 - DNA, 22 bp, Artificial Sequence
  based on Escherichia coli
aatgagccaagctggttaagct

SEQ ID NO: 29 - DNA, 24 bp, Artificial Sequence
  based on  Escherichia coli
gcaccattcatgatattcgttaac

SEQ ID NO: 30 - DNA, 24 bp, Artificial Sequence
  based on  Escherichia coli
ttgcaatgttcattaatatacgtc

SEQ ID NO: 31 - DNA, 24 bp, Artificial Sequence
  based on  Escherichia coli
tatactcattgataaaatactaac

SEQ ID NO: 32 - DNA, 24 bp, Artificial Sequence
  based on  Escherichia coli
agcacagaagagtaattatatgtc

SEQ ID NO: 33 - DNA, 24 bp, Artificial Sequence
  based on  Escherichia coli
atcaggatgccgttatactcattg

SEQ ID NO: 34 - DNA, 24 bp, Artificial Sequence
  based on  Escherichia coli
gcacagaagagtaattatatgtcc

SEQ ID NO: 35 - DNA, 24 bp, Artificial Sequence
  based on  Escherichia coli
aagttttgatattgtactggatgc

SEQ ID NO: 36 - DNA, 24 bp, Artificial Sequence
  based on  Escherichia coli
cattaaagatagatgataaatcac

SEQ ID NO: 37 - DNA, 24 bp, Artificial Sequence
  based on  Escherichia coli
tgctcaacatagaaacccacatag

SEQ ID NO: 38 - DNA, 24 bp, Artificial Sequence
  based on  Escherichia coli
tcgaatcagtgttatttaccagtg

SEQ ID NO: 39 - DNA, 24 bp, Artificial Sequence
  based on  Escherichia coli
gttattctggtacatgaacatcat

SEQ ID NO: 40 - DNA, 24 bp, Artificial Sequence
  based on  Escherichia coli
tagataattccacacagcccacta

SEQ ID NO: 41 - DNA, 24 bp, Artificial Sequence
  based on  Escherichia coli
gtagtcgaaatcatggtgcagaat

SEQ ID NO: 42 - DNA, 24 bp, Artificial Sequence
  based on  Escherichia coli
cttctctgctgtttggtgtcttat